(19) United States
(12) Patent Application Publication
(10) Pub. No.: US 2025/0281634 A1
(43) Pub. Date: Sep. 11, 2025

(54) CONJUGATES COMPRISING A PHOSPHORUS (V) AND A DRUG MOIETY

(71) Applicant: Tubulis GmbH, Planegg/Martinsried (DE)

(72) Inventors: Christian HACKENBERGER, Berlin (DE); Marc-André KASPER, Munich (DE); Philipp OCHTROP, Munich (DE); Jahaziel JAHZERAH, Berlin (DE); Paul MACHUI, Munich (DE); Dominik SCHUMACHER, Munich (DE); Jonas HELMA-SMETS, Munich (DE); Isabelle MAI, Munich (DE)

(21) Appl. No.: 19/207,970

(22) Filed: May 14, 2025

Related U.S. Application Data
(62) Division of application No. 18/054,018, filed on Nov. 9, 2022, now abandoned.

(30) Foreign Application Priority Data
Nov. 9, 2021 (EP) 21207195.5

(51) Int. Cl. A61K 47/68; C07F 9/30

(52) U.S. Cl. CPC A61K 47/6889; C07F 9/30

(57) ABSTRACT

The present invention relates to a conjugate having the formula (I):

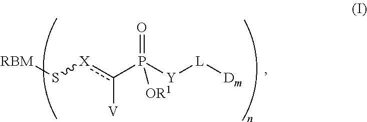

wherein a receptor binding molecule (RBM) is connected with a drug moiety (D). The present invention also relates to intermediates for producing the same, methods of preparing the same, pharmaceutical compositions comprising the same, as well as uses thereof.

### CROSS-REFERENCE TO RELATED APPLICATIONS

The present application is a divisional of U.S. application Ser. No. 18/054,018, filed on Nov. 9, 2022, which claims the benefit of priority of European Patent Application No. 21207195.5 filed Nov. 9, 2021. U.S. application Ser. No. 18/054,018 and European Patent Application No. 21207195.5 are hereby incorporated by reference in its entirety for all purposes.

### TECHNICAL FIELD

The present invention relates to conjugates of a receptor binding molecule with a drug moiety, intermediates for producing the same, methods of preparing the same, pharmaceutical compositions comprising the same, as well as uses thereof.

### BACKGROUND

Brentuximab vedotin (tradename Adcetris®) is an antibody drug conjugate which has been approved for medical use in 2011. Brentuximab Vedotin consists of the tumour-targeting chimeric IgG1 antibody component brentuximab and a linker-payload component, comprising the payload moiety monomethyl auristatin E, which induces apoptosis upon intracellular delivery and release.

However, although Brentuximab vedotin is an approved and marketed ADC, certain drawbacks remain. As an example, it has turned out that in Brentuximab vedotin the number of drug molecules, which can be attached to the antibody, is subject to certain limitations; see, e.g., Hamblett et al., “Effects of Drug Loading on the Antitumor Activity of a Monoclonal Antibody Drug Conjugate”, Clinical Cancer Research vol. 10, pp. 7063 to 7070, Oct. 15, 2004. Developments include, e.g., variation of the linker connecting the antibody to the payload, and the introduction of a polyethylene glycol substituent; see, e.g., Lyon et al., “Reducing hydrophobicity of homogeneous antibody-drug conjugates improves pharmacokinetics and therapeutic index”, Nature Biotechnology volume 33, pages 733-735 (2015), doi: 10.1038/nbt.3212; WO 2015/057699; Burke et al., “Optimization of a PEGylated Glucuronide-Monomethylauristatin E Linker for Antibody-Drug Conjugates”, Molecular Cancer Therapeutics 2017, 16(1), 116-123, doi: 10.1158/1535-7163.MCT-16-0343; and Simmons et al., “Reducing the antigen-independent toxicity of antibody-drug conjugates by minimizing their non-specific clearance through PEGylation”, Toxicology and Applied Pharmacology 2020, 392:114932, doi: 10.1016/j.taap.2020.114932.

Accordingly, there is an ongoing need for further conjugates which have good properties for pharmaceutical applications.

### SUMMARY

This need is addressed by the subject-matter as defined in the claims and in the embodiments described herein.

Accordingly, the present invention relates to a conjugate having the formula (I):

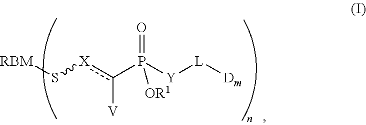

or a pharmaceutically acceptable salt or solvate thereof;
wherein:
RBM is a receptor binding molecule;
[symbol] is a double bond; or
[symbol] is a bond;
V is absent when [symbol] is a double bond; or
V is H or (C₁-C₈)alkyl when [symbol] is a bond;
X is R₃—C when [symbol] is a double bond; or
X is

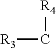

when [symbol] is a bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic residue or an optionally substituted aromatic residue
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
L is a linker;
D is a drug moiety;
m is an integer ranging from 1 to 10; and
n is an integer ranging from 1 to 20.

The present invention also relates to a compound having the formula (II):

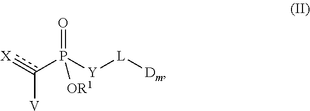

or a pharmaceutically acceptable salt or solvate thereof;
wherein:
[symbol] is a triple bond; or
[symbol] is a double bond;
V is absent when [symbol] is a triple bond; or
V is H or (C₁-C₈)alkyl when [symbol] is a double bond;
X is R₃—C when [symbol] is a triple bond; or
X is

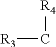

when [symbol] is a double bond;
Y is NR⁵, S, O, or CR⁶R⁷;
R¹ is a first polyalkylene glycol unit comprising at least 3 alkylene glycol subunits;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
L is a linker;
D is a drug moiety; and
m is an integer ranging from 1 to 10.

The present invention also relates to a method of preparing a conjugate of formula (I), said method comprising:
reacting a compound of formula (II)

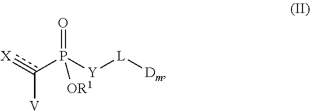

or a pharmaceutically acceptable salt of solvate thereof;
wherein:
[symbol] is a triple bond; or
[symbol] is a double bond;
V is absent when [symbol] is a triple bond; or
V is H or (C₁-C₈)alkyl when [symbol] is a double bond;
X is R₃—C when [symbol] is a triple bond; or
X is

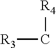

when [symbol] is a double bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
L is a linker;
D is a drug moiety; and
m is an integer ranging from 1 to 10;
with a thiol-containing molecule of formula (III)

RBM [symbol] SH)ₙ (III)
wherein RBM is a receptor binding molecule; and
n is an integer ranging from 1 to 20;
resulting in a compound of formula (I)

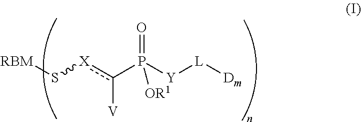

or a pharmaceutically acceptable salt or solvate thereof;
wherein:
[symbol] is a double bond when [symbol] in a compound of formula (II) is a triple bond; or
[symbol] is a bond when [symbol] in a compound of formula (II) is a double bond;
V is absent when [symbol] is a double bond; or
V is H or (C₁-C₈)alkyl when [symbol] is a bond;
X is R₃—C when [symbol] is a double bond; or
X is
when

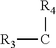

is a bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
L is a linker;
D is a drug moiety;
m is an integer ranging from 1 to 10; and
n is an integer ranging from 1 to 20.

The present invention also relates to a conjugate of formula (I) obtainable or being obtained by a method of the invention.

The present invention also relates to a pharmaceutical composition comprising a conjugate of the invention.

The present invention also relates to a conjugate of the invention for use in a method of treating a disease. The disease may be cancer.

The present invention also relates to a pharmaceutical composition of the invention for use in a method of treating a disease. The disease may be cancer.

### BRIEF DESCRIPTION OF THE DRAWINGS

FIG. 1 shows an analytical HPLC chromatogram of the compound methyl 4-azido-2-(dodecaethyleneglycol)benzoate. The horizontal axis depicts the retention time in minutes.

FIG. 2 shows an analytical HPLC chromatogram of the compound methyl 4-azido-2-(dodecaethyleneglycol)benzoate. The horizontal axis depicts the retention time in minutes.

FIG. 3 shows an analytical HPLC chromatogram of the compound P5(PEG12)-COOH.

FIG. 4 shows an analytical HPLC chromatogram of the compound P5(PEG24)-OSu.

The horizontal axis depicts the retention time in minutes.

FIG. 5 shows an analytical HPLC chromatogram of the compound P5(PEG12,PEG24)-COOH. The horizontal axis depicts the retention time in minutes.

FIG. 6 shows an analytical HPLC chromatogram of the compound P5(PEG24,PEG24)-COOH. The horizontal axis depicts the retention time in minutes.

FIG. 7 shows an analytical HPLC chromatogram of the compound NH₂—VC-PAB-MMAE TFA salt. The horizontal axis depicts the retention time in minutes.

FIG. 8 shows an analytical HPLC chromatogram of the compound P5(PEG12)-VC-PAB-MMAF. The horizontal axis depicts the retention time in minutes.

FIG. 9 shows an analytical HPLC chromatogram of the compound P5(PEG12)-VC-PAB-MMAE. The horizontal axis depicts the retention time in minutes.

FIG. 10 shows an analytical HPLC chromatogram of the compound P5(PEG24)-VC-PAB-MMAE.

FIG. 11 shows an analytical HPLC chromatogram of the compound P5(PEG12,PEG24)-VC-PAB-MMAE. The horizontal axis depicts the retention time in minutes.

FIG. 12 shows an analytical HPLC chromatogram of the compound P5(PEG24,PEG24)-VC-PAB-MMAE. The horizontal axis depicts the retention time in minutes.

FIG. 13 shows an analytical SEC chromatogram of Trastuzumab. SEC means size-exclusion chromatography.

FIG. 14 shows an analytical HIC chromatogram of Trastuzumab. HIC means hydrophobic interaction chromatography.

FIG. 15 shows an analytical SEC chromatogram of Brentuximab.

FIG. 16 shows an analytical HIC chromatogram of Brentuximab.

FIG. 17 shows an analytical SEC chromatogram of Brentuximab-P5(PEG12)-VC-PAB-MMAE (DAR8).

FIG. 18 shows an analytical HIC chromatogram of Brentuximab-P5(PEG12)-VC-PAB-MMAE (DAR8).

FIG. 19 shows an analytical SEC chromatogram of Brentuximab-P5(PEG12)-VC-PAB-MMAE (DAR4).

FIG. 20 shows an analytical SEC chromatogram of Brentuximab-P5(PEG24)-VC-PAB-MMAE(DAR8).

FIG. 21 shows an analytical HIC chromatogram of Brentuximab-P5(PEG24)-VC-PAB-MMAE (DAR8).

FIG. 22 shows an analytical SEC chromatogram of Brentuximab-P5(PEG12, PEG24)-VC-PAB-MMAE (DAR8).

FIG. 23 shows an analytical HIC chromatogram of Brentuximab-P5(PEG12, PEG24)-VC-PAB-MMAE (DAR8).

FIG. 24 shows an analytical SEC chromatogram of Brentuximab-P5(PEG24, PEG24)-VC-PAB-MMAE (DAR8).

FIG. 25 shows an analytical HIC chromatogram of Brentuximab-P5(PEG24, PEG24)-VC-PAB-MMAE (DAR8).

FIG. 26 shows an analytical HIC chromatogram of Brentuximab-P5(PEG12)-VC-PAB-MMAF (DAR8).

FIG. 27 shows an analytical SEC chromatogram of Trastuzumab-P5(PEG12)-VC-PAB-MMAE (DAR8).

FIG. 28 shows an analytical HIC chromatogram of Trastuzumab-P5(PEG12)-VC-PAB-MMAE(DAR8).

FIG. 29 shows an analytical SEC chromatogram of Trastuzumab-P5(PEG12)-VC-PAB-MMAF(DAR8).

FIG. 30 shows an analytical HIC chromatogram of Trastuzumab-P5(PEG12)-VC-PAB-MMAF(DAR8).

FIG. 31 shows a screening experiment to identify optimal conditions for antibody modifications with PEGylated phosphonamidates. Drug to Antibody Ratio (DAR) has been measured by MS. Left graph: 10 eq. of P5(PEG12)-VC-PAB-MMAE have been used under the above stated conditions and TCEP equivalents were varied. A maximum degree of modification has been reached with 8 equivalents of TCEP. Right graph: Those 8 equivalents have been carried over to a second experiment in which the P5(PEG12)-VC-PAB-MMAE equivalents were further increased in order to achieve a maximum DAR of 8. Optimal conditions to achieve DAR8 were Identified as 8 eq. of TCEP and 12 eq. of P5(PEG12)-VC-PAB-MMAE with respect to the antibody (=only 1.5 eq. per Cys).

FIG. 32 shows a Hydrophobic Interaction Chromatography of Brentuximab conjugated to P5(PEG2)-, P5(PEG12)- and P5(PEG24)-VC-PAB-MMAE in direct comparison to commercially available Adcetris (Brentuximab-Maleimidocapryl-VC-PAB-MMAE, DAR4av, black).

FIG. 33 shows a Hydrophobic Interaction Chromatography of Brentuximab conjugated to P5(PEG24,PEG12)-, P5(PEG24,PEG24)- and P5(PEG24)-VC-PAB-MMAE in direct comparison to commercially available Adcetris (Brentuximab-Maleimidocapryl-VC-PAB-MMAE, DAR4av, black). # Additional peaks, not identified.

FIG. 34 shows an Analytical Size-Exclusion-Chromatography (SEC) (left) and HIC (right) after storage of a DAR 8 Brentuximab-P5(PEG12)-VC-PAB-MMAE over several weeks. No Aggregates are visible in the SEC and no drug loss in the HIC.

FIG. 35 shows the in vitro cytoxicity of Brentuximab (anti-CD30) ADCs on an antigen positive cell line (Karpas 299, left) and an antigen negative cell line (HL-60, right). Shown is a comparison of 3 different DAR8 ADCs that only differ in the length of the PEG substituent (PEG2 vs. PEG12 vs. PEG24) against unmodified Brentuximab.

FIG. 36 shows the in vitro cytoxicity of Brentuximab (anti-CD30) ADCs on an antigen positive cell line (Karpas 299, left) and an antigen negative cell line (HL-60, right). Shown is a comparison of Brentuximab-P5(PEG24)-vc-PAB-MMAE (DAR8) against commercial Adcetris (DAR4).

FIG. 37 shows In vitro cytoxicity of Brentuximab (anti-CD30) ADCs on an antigen positive cell line (Karpas 299, left) and an antigen negative cell line (HL-60, right). Shown is a comparison of Brentuximab-P5(PEG24)-vc-PAB-MMAE, modified with either 4 (DAR4) or 8 (DAR8) linker payload molecules per antibody.

FIG. 38 shows the in vitro cytotoxicity of Brentuximab (anti-CD30) ADCs on an antigen positive cell line (Karpas 299, left) and an antigen negative cell line (HL-60, right). Shown is a comparison of Brentuximab-P5(PEG12)-vc-PAB-MMAE (DAR8) versus the same construct, carrying a MMAF payload.

FIG. 39 shows the in vitro cytoxicity of Trastuzumab (anti-Her2) ADCs on an antigen positive cell line (SKBR3, left) and an antigen negative cell line (MDAMB, right). Shown is a comparison of Trastuzumab-P5(PEG12)-vc-PAB-MMAF (DAR8) versus unmodified Trastuzumab.

FIG. 40 shows the evaluation of the bystander effect in dependence of differently PEGylated Brentuximab-P5-VC-PAB-MMAE constructs. Top: In vitro cytoxicity of Brentuximab (anti-CD30) ADCs on two antigen positive cell line (Karpas 299, left and L-540, right) and an antigen negative cell line (HL-60, bottom left). Transfer of the supernatant after incubation of L-540 with the ADCs to HL-60 has been performed in order to evaluate bystander killing (HL60, bottom right).

FIG. 41 shows the in vivo evaluation of Brentuximab-(PEG12)-VC-PAB-MMAE (DAR8 and DAR4), Adcetris (DAR4) and a non-treated control in a Karpas299 based tumor xenograft model in SCID mice with 10 animals per group. Mice were treated with 0.5 mg/kg of the constructs four times, every four days. Left graph shows the mean tumor volume of all 10 mice per group. Last observation point of sacrificed animals has been carried forward (LOCF). Right graph shows the Kaplan-Meier plot of survival in each group.

FIG. 42 shows the quantification of the amount of total antibody in blood circulation after treatment of female Spraque-Dawley rats with either Brentuximab-P5(PEG24)-VC-PAB-MMAE or Adcetris via ELISA.

FIG. 43A shows conjugation of P5(PEG12)-VC-PAB-SB743921 and P5(PEG24)-VA-PAB-SB743921 to Trastuzumab. FIG. 43B shows conjugation efficiency estimated via mass spectrometry (MS). The Drug-to-Antibody-Ratio was calculated from the MS-Intensities of the modified and unmodified heavy and light chain species; it is noted that in this example only little or slow conjugation was observed when using a short PEG2 residue (i.e. a PEG residue comprising 2 PEG units), while a more efficient conjugation reaction could be achieved when using PEG12 (i.e. a PEG residue comprising 12 PEG units), and even more when using PEG24 (i.e. a PEG residue comprising 24 PEG units) as indicated by the higher drug to antibody ratio (DAR). FIG. 43C shows exemplary MS spectrum of the conjugation of P5(PEG24)-VA-PAB-SB743921 to Trastuzumab. The mass spectrum shows unmodified light chain (23438 Da), modified light chain (25439 Da), unmodified heavy chain (49149 Da), triple modified heavy chain (55452 Da).

FIG. 44 shows efficacy of the ADC Trastuzumab-P5(PEG24)-VA-PAB-SB743921 on a target negative cell line (L-540) and several Her2+ cell-lines. Trastuzumab-P5(PEG24)-VA-PAB-SB743921 shows only an effect on the non-targeted L-540 at the highest tested concentration, while a much better efficacy could be shown on all tested target-positive cell lines.

FIG. 45 shows A) conjugation of P5(PEG12)-VC-PAB-Emetin to Trastuzumab; B) Conjugation efficiency estimated via mass spectrometry (MS). The Drug-to-Antibody-Ratio was calculated from the MS-Intensities of the modified and unmodified heavy and light chain species; C) Exemplary HIC spectrum of the conjugation of 16 equivalents of P5(PEG12)-VC-PAB-Emetin to Trastuzumab, yielding a DAR 8.0 ADC. No unconjugated Trastuzumab was visible at the known retention time of the unmodified antibody (ca. 8-9 minutes).

FIG. 46 shows normalized HIC chromatograms of Tras-P5(PEG12)-VC-PAB-Emetin DAR8 and Adcetris (Brentuximab vedotin).

FIG. 47 shows A) conjugation of P5(PEG12)-VC-PAB-AT7519 to Trastuzumab; B) Analysis of the purified DAR8 ADCs by Size-Exclusion Chromatography (SEC) and Hydrophobic interaction chromatography (HIC); C) MS analysis of the conjugation of 16 equivalents of P5(PEG12)-VC-PAB-AT7519 to Trastuzumab, yielding a DAR 8.0 ADC. No unconjugated Trastuzumab was observed.

FIG. 48 shows A) conjugation of P5(PEG24)-VA-PAB-Panobinostat to Trastuzumab; B) Analysis of the purified DAR8 ADCs by Hydrophobic interaction chromatography (HIC).

FIG. 49 shows A) conjugation of P5(PEG12)-GlcA-AT7519 to Trastuzumab; B) Conjugation efficiency estimated via MS in dependency on the equivalents of P5(PEG12)-GlcA-AT7519. Reaction was carried out as described herein with 8 eq. of TCEP. The Drug-to-Antibody-Ratio was calculated from the MS-Intensities of the modified and unmodified heavy and light chain species; C) Exemplary MS spectrum of the conjugation of 6 eq. P5(PEG12)-GlcA-AT7519 to Trastuzumab to yield a DAR 3.9 ADC.

FIG. 50 shows normalized HIC chromatograms of Tras-P5(PEG12)-GlcA-AT7519 and Adcetris (Brentuximab vedotin); and an SEC chromatogram of Tras-P5(PEG12)-GlcA-AT7519.

FIG. 51 shows A) conjugation of P5(PEG12)-GlcA-MMAE to Brentuximab; B) Efficacy on a target negative cell line (HL-60, bottom) and a target positive cell line (Karpas299, top); Brentuximab-P5(PEG12)-GlcA-MMAE shows no effect on the target negative cell line (HL-60), while a much better efficacy could be shown on target-positive cell line Karpas299.

FIG. 52 shows A) conjugation of P5(PEG12)-GlcA-SB743921 to Trastuzumab; B) Conjugation efficiency estimated via mass spectrometry (MS) in dependency on the equivalents of P5(PEG12)-GlcA-SB743921. Reaction was carried out as described herein with 8 eq. of TCEP. The Drug-to-Antibody-Ratio was calculated from the MS-Intensities of the modified and unmodified heavy and light chain species. C) Analysis of the purified DAR8 ADCs by Size-Exclusion Chromatography (SEC) and Hydrophobic interaction chromatography (HIC).

FIG. 53 shows the efficacy test results of Trastuzumab-P5(PEG12)GlcA-SB743921 on a target negative cell line (MDA-MB468) and a target positive cell line (SKBR3). Trastuzumab-P5(PEG12)-GlcA-SB743921 shows no effect on the target negative cell line (MDA-MB468), while a much better efficacy could be shown on SKBR-3.

### DETAILED DESCRIPTION

The present invention is described in detail in the following and will also be further illustrated by the appended examples and figures.

### Definitions

Unless otherwise indicated, the term “alkyl” by itself or as part of another term in general refers to a substituted or unsubstituted straight chain or branched, saturated hydrocarbon having the indicated number of carbon atoms; e.g., “—(C₁-C₈)alkyl” or “—(C₁-C₁₀)alkyl” refer to an alkyl group having from 1 to 8 or 1 to 10 carbon atoms, respectively). When the number of carbon atoms is not indicated, the alkyl group may have from 1 to 8 carbon atoms. Representative straight chain —(C₁-C₈)alkyl groups include, but are not limited to, -methyl, -ethyl, -n-propyl, -n-butyl, -n-pentyl, -n-hexyl, -n-heptyl and -n-octyl; branched —(C₁-C₈)alkyl groups include, but are not limited to, -isopropyl, -sec-butyl, -isobutyl, -tert-butyl, -isopentyl, and -2-methylbutyl. In some aspects, an alkyl group may be unsubstituted. Optionally, an alkyl group may be substituted, such as e.g. with one or more groups.

Unless otherwise indicated, the term “alkylene” by itself or as part of another term, in general refers to a substituted or unsubstituted branched or straight chain, saturated hydrocarbon radical of the stated number of carbon atoms, preferably 1-10 carbon atoms (—(C₁-C₁₀)alkylene-) or preferably 1 to 8 carbon atoms (—(C₁-C₈)alkylene-), and having two monovalent radical centers derived by the removal of two hydrogen atoms from the same or two different carbon atoms of a parent alkane. When the number of carbon atoms is not indicated, the alkylene group may have from 1 to 8 carbon atoms. Typical alkylene radicals include, but are not limited to: methylene (—CH₂—), 1,2-ethylene (—CH₂CH₂—), 1,3-n-propylene (—CH₂CH₂CH₂—), and 1,4-n-butylene (—CH₂CH₂CH₂CH₂—). In some aspects, an alkylene group may be unsubstituted. Optionally, an alkylene group may be substituted, such as e.g. with one or more groups.

Unless otherwise indicated, the term “alkenyl” by itself or as part of another term in general refers to a substituted or unsubstituted straight chain or branched, unsaturated hydrocarbon having a double bond and the indicated number of carbon atoms; e.g., “—(C₂-C₃)alkenyl” or “—(C₂-C₁₀)alkenyl” refer to an alkenyl group having from 2 to 8 or 2 to 10 carbon atoms, respectively). When the number of carbon atoms is not indicated, the alkenyl group may have from 2 to 8 carbon atoms. Representative —(C₂-C₃)alkenyl groups include, but are not limited to, -ethenyl, -1-propenyl, -2-propenyl, -1-butenyl, -2-butenyl, -isobutenyl, -1-pentenyl, -2-pentenyl, -3-methyl-1-butenyl, -2-methyl-2-butenyl, and -2,3-dimethyl-2-butenyl. In some aspects, an alkenyl group may be unsubstituted. Optionally, an alkenyl group may be substituted, such as e.g. with one or more groups.

Unless otherwise indicated, the term “alkenylene” by itself of as part of another term, in general refers to a substituted or unsubstituted unsaturated branched or straight chain hydrocarbon radical of the stated number of carbon atoms, preferably 2-10 carbon atoms (—(C₂-C₁₀)alkenylene-) or preferably 2 to 8 carbon atoms (—(C₂-C₃)alkenylene-), and having a double bond, and having two monovalent radical centers derived by the removal of two hydrogen atoms from the same or two different carbon atoms of a parent alkene. When the number of carbon atoms is not indicated, the alkenylene group may have from 1 to 8 carbon atoms. Typical alkenylene radicals include, but are not limited to: -ethenylene-, -1-propenylene-, 2-propenylene-, -1-butenylene-, -2-butenylene-, -isobutenylene-, -1-pentenylene-, -2-pentenylene-, -3-methyl-1-butenylene-, -2-methyl-2-butenylene-, and -2,3-dimethyl-2-butenylene-. In some aspects, an alkenylene group may be unsubstituted. Optionally, an alkenylene group may be substituted, such as e.g. with one or more groups.

Unless otherwise indicated, the term “alkynyl” by itself or as part of another term in general refers to a substituted or unsubstituted straight chain or branched, unsaturated hydrocarbon having a triple bond and the indicated number of carbon atoms; e.g., “—(C₂-C₃)alkynyl” or “—(C₂-C₁₀)alkynyl” refer to an alkynyl group having from 2 to 8 or 2 to 10 carbon atoms, respectively). When the number of carbon atoms is not indicated, the alkynyl group may have from 2 to 8 carbon atoms. Representative —(C₂-C₃)alkynyl groups include, but are not limited to, -acetylenyl, -1-propynyl, -2-propynyl, -1-butynyl, -2-butynyl, -1-pentynyl, -2-pentynyl and -3-methyl-1-butynyl. In some aspects, an alkynyl group may be unsubstituted. Optionally, an alkynyl group may be substituted, such as e.g. with one or more groups.

Unless otherwise indicated, the term “alkynylene” by itself of as part of another term, in general refers to a substituted or unsubstituted, branched or straight chain, unsaturated hydrocarbon radical of the stated number of carbon atoms, preferably 2-10 carbon atoms (—(C₂-C₁₀)alkynylene-) or preferably 2 to 8 carbon atoms (—(C₂-C₃)alkynylene-), and having a triple bond, and having two monovalent radical centers derived by the removal of two hydrogen atoms from the same or two different carbon atoms of a parent alkyne. When the number of carbon atoms is not indicated, the alkynylene group may have from 2 to 8 carbon atoms. Typical alkynylene radicals include, but are not limited to: -ethynylene-, -1-propynylene-, -2-propynylene-, -1-butynylene-, -2-butynylene-, -1-pentynylene-, -2-pentynylene- and -3-methyl-1-butynylene-. In some aspects, an alkynylene group may be unsubstituted. Optionally, an alkynylene group may be substituted, such as e.g. with one or more groups.

Unless otherwise indicated, the term “aryl,” by itself or as part of another term, in general means a substituted or unsubstituted monovalent carbocyclic aromatic hydrocarbon radical of 6 to 20 carbon atoms (preferably 6 to 14 carbon atoms, more preferably 6 to 10 carbon atoms, in very preferred embodiments 6 carbon atoms) derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, and biphenyl. An exemplary aryl group is a phenyl group. In some aspects, an aryl group may be unsubstituted. Optionally, an aryl group may be substituted, such as e.g. with one or more groups.

Unless otherwise indicated, the term “arylene”, by itself or as part of another term, in general is an aryl group as defined above wherein one of the hydrogen atoms of the aryl group is replaced with a bond (i.e., it is divalent) and can be in the para, meta, or ortho orientations as shown in the following structures, with phenyl as the exemplary group:

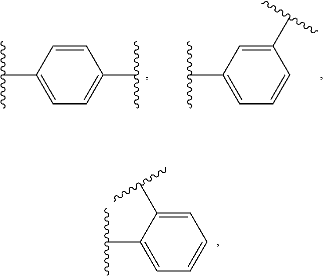 [SMILES: *c1ccc(*)cc1 ; *c1cccc(*)c1 ; *c1ccccc1*]

In selected embodiments, e.g., when a parallel connector unit comprises an arylene, the arylene is an aryl group as defined above wherein two or more of the hydrogen atoms of the aryl group are replaced with a bond (i.e., the arylene can be trivalent). In some aspects, an arylene group may be unsubstituted. Optionally, an alkynylene group may be substituted, such as e.g. with one or more groups.

Unless otherwise indicated, the term “heterocycle” or “heterocyclic ring”, by itself or as part of another term, in general refers to a monovalent substituted or unsubstituted aromatic or non-aromatic monocyclic or bicyclic ring system having the indicated number of carbon atoms (e.g., “(C₃-C₈)heterocycle” or “(C₃-C₁₀)heterocycle” refer to a heterocycle having from 3 to 8 or from 3 to 10 carbon atoms, respectively) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure. Representative examples of a (C₃-C₈)heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl. In some aspects, a heterocycle group may be unsubstituted. Optionally, a heterocycle group may be substituted, such as e.g. with one or more groups.

Unless otherwise indicated, the term “heterocyclo” or “heterocyclic ring”, by itself or as part of another term, in general refers to a heterocycle group as defined above and having the indicated number of carbon atoms (e.g., (C₃-C₈)heterocycle or (C₃-C₁₀)heterocycle) wherein one of the hydrogen atoms of the heterocycle group is replaced with a bond (i.e., it is divalent). In selected embodiments, e.g., when a parallel connector unit comprises a heterocyclo, the heterocyclo is a heterocycle group as defined above wherein two or more of the hydrogen atoms of the heterocycle group are replaced with a bond (i.e., the heterocyclo can be trivalent). In some aspects, a heterocyclo or heterocyclic ring may be unsubstituted. Optionally, a heterocyclo or heterocyclic ring may be substituted, such as e.g. with one or more groups.

Unless otherwise indicated, the term “carbocycle” or “carbocyclic ring” by itself or as part of another term, in general refers to a monovalent, substituted or unsubstituted aromatic or non-aromatic monocyclic or bicyclic carbocyclic ring system having the indicated number of carbon atoms (e.g., “(C₃-C₈)carbocycle” or “(C₃-C₁₀)carbocycle” refer to a carbocycle having from 3 to 8 or from 3 to 10 carbon atoms, respectively) derived by the removal of one hydrogen atom from a ring atom of a parent ring system. As illustrative but non-limiting examples the carbocycle may be a 3-, 4-, 5-, 6-, 7- or 8-membered carbocycle. Representative (C₃-C₃)carbocycles include, but are not limited to, phenyl, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. In some aspects, a carbocycle may be unsubstituted. Optionally, a carbocycle may be substituted, such as e.g. with one or more groups.

Unless otherwise indicated, the term “carbocyclo” or “carbocyclic ring”, by itself or as part of another term, in general refers to a carbocycle group as defined above having the indicated number of carbon atoms (e.g., “(C₃-C₈)carbocyclo” or “(C₃-C₁₀)carbocyclo” refer to a carbocyclo or carbocyclic ring having from 3 to 8 or from 3 to 10 carbon atoms, respectively), wherein another of the hydrogen atoms of the carbocycle groups is replaced with a bond (i.e., it is divalent). In selected embodiments, e.g., when a parallel connector unit comprises a carbocyclo or carbocyclic ring, the carbocyclo or carbocyclic ring is a carbocycle group as defined above, wherein two or more of the hydrogen atoms of the carbocycle group are replaced with a bond (i.e., the carbocyclo or carbocyclic ring can be trivalent). In some aspects, a carbocyclo or carbocyclic ring may be unsubstituted. Optionally, a heterocyclo or heterocyclic ring may be substituted, such as e.g. with one or more groups.

Unless otherwise indicated, the term “heteroalkyl”, by itself or in combination with another term, may mean, unless otherwise stated, a stable straight or branched chain hydrocarbon, or combinations thereof, fully saturated or containing from 1 to 3 degrees of unsaturation, consisting of the stated number of carbon atoms (e.g., (C₁-C₈)heteroalkyl or (C₁-C₁₀)heteroalkyl) and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the alkyl group is attached to the remainder of the molecule. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —NH—CH₂—CH₂—NH—C(O)—CH₂—CH₃, —CH₂—CH₂—S(O)₂—CH₃, —CH═CH—O—CH₃, —Si(CH₃)₃, —CH₂—CH═N—O—CH₃, and —CH═CH—N(CH₃)—CH₃. Up to two heteroatoms may be consecutive, such as, for example, —CH₂—NH—OCH₃ and —CH₂—O—Si(CH₃)₃. In preferred embodiments, a (C₁-C₄)heteroalkyl or heteroalkylene has 1 to 4 carbon atoms and 1 or 2 heteroatoms and a (C₁-C₃)heteroalkyl or heteroalkylene has 1 to 3 carbon atoms and 1 or 2 heteroatoms. In some aspects, a heteroalkyl or heteroalkylene is saturated. In some aspects, a heteroalkyl or heteroalkylene may be unsubstituted. Optionally, a heteroalkyl or heteroalkylene may be substituted, such as e.g. with one or more groups.

Unless otherwise indicated, the term “heteroalkylene” by itself or as part of another substituent means a divalent group derived from heteroalkyl (as described above) having the indicated number of carbon atoms (e.g., (C₁-C₈)heteroalkylene or (C₁-C₁₀)heteroalkylene), as exemplified by —CH₂—CH₂—S—CH₂—CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied. In selected embodiments, e.g., when a parallel connector unit comprises a heteroalkylene, the heteroalkylene is a heteroalkyl group defined above wherein two or more of the hydrogen atoms of the heteroalkyl group are replaced with a bond (i.e., the heteroalkylene can be trivalent). In some aspects, a heteroalkyl or heteroalkylene may be saturated. In some aspects, a heteroalkylene is unsubstituted. Optionally, a heteroalkylene may be substituted, such as e.g. with one or more groups.

The term “halogen”, unless defined otherwise, in general refers to elements of the 7^{th} main group; preferably fluorine, chlorine, bromine and iodine; more preferably fluorine, chlorine and bromine; even more preferably, fluorine and chlorine.

The term “substituted”, “optionally substituted”, “optionally may be substituted” or the like, unless otherwise indicated, in general means that one or more hydrogen atoms can be each independently replaced with a substituent. Typical substituents include, but are not limited to, —X, —R, —O—, —OR, —SR, —S—, —NR₂, —NR₃, ═NR, —CX₃, —CN, —OCN, —SCN, —N═C═O, —NCS, —NO, —NO₂, ═N₂, —N₃, —NRC(═O)R, —C(═O)R, —C(═O)NR₂, —SO₃⁻, —SO₃H, —S(═O)₂R, —OS(═O)₂OR, —S(═O)₂NR, —S(═O)R, —OP(═O)(OR)₂, —P(═O)(OR)₂, —PO₄₃—, —PO₃H₂, —C(═O)R, —C(═O)X, —C(═S)R, —CO₂R, —CO₂H, —C(═S)OR, —C(═O)SR, —C(═S)SR, —C(═O)NR₂, —C(═S)NR₂, or —C(═NR)NR₂, where each X is independently a halogen: —F, —C, —Br, or —I; and each R is independently —H, —(C₁-C₂₀)alkyl (such as e.g. —(C₁-C₁₀)alkyl or —(C₁-C₈)alkyl), —(C₆-C₂₀)aryl, (such as e.g. —(C₆-C₁₀)aryl or, preferably, —C-aryl), —(C₃-C₁₄)heterocycle (such as e.g. —(C₃-C₁₀)heterocycle or —(C₃-C₈)heterocycle), a protecting group, or a prodrug moiety. Typical substituents also include (═O).

The term “aliphatic or aromatic residue”, as used herein, in general refers to an aliphatic substituent, such as e.g. but not limited to an alkyl residue, which, however, can be optionally substituted by further aliphatic and/or aromatic substituents. As non-limiting examples an aliphatic residue can be a nucleic acid, an enzyme, a co-enzyme, a nucleotide, an oligonucleotide, a monosaccharide, a polysaccharide, a polymer, a fluorophore, optionally substituted benzene, etc., as long as the direct link of such a molecule to the core structure (in case of R⁵, e.g., the link to the nitrogen atom of the Y) is aliphatic. An aromatic residue is a substituent, wherein the direct link to the core structure is part of an aromatic system, e.g., an optionally substituted phenyl or triazolyl or pyridyl or nucleotide; as non-limiting example if the direct link of the nucleotide to the core structure is for example via a phenyl-residue. The term “aromatic residue”, as used herein, also includes a heteroaromatic residue.

The term “peptide”, unless otherwise indicated, in general refers to an organic compound comprising two or more amino acids covalently joined by peptide bonds (amide bond). Peptides may be referred to with respect to the number of constituent amino acids, i.e., a dipeptide contains two amino acid residues, a tripeptide contains three, etc. Peptides containing ten or fewer amino acids may be referred to as oligopeptides, while those with more than ten amino acid residues, e.g. with up to about 30 amino acid residues, are polypeptides.

The term “amino acid”, as used herein, in general refers to an organic compound having a —CH(NH₃)—COOH group. In one embodiment, the term “amino acid” refers to a naturally occurring amino acid. As illustrative examples, naturally occurring amino acids include arginine, lysine, aspartic acid, glutamic acid, glutamine, asparagine, histidine, serine, threonine, tyrosine, cysteine, methionine, tryptophan, alanine, isoleucine, leucine, phenylalanine, valine, proline and glycine. However, the term in its broader meaning also encompasses non-naturally occurring amino acids.

Amino acids and peptides according to the disclosure can also be modified at functional groups. Non limiting examples are saccharides, e.g., N-Acetylgalactosamine (GalNAc), or protecting groups, e.g., Fluorenylmethoxycarbonyl (Fmoc)-modifications or esters.

The term “antibody”, as used herein, is intended to refer to immunoglobulin molecules, preferably comprised of four polypeptide chains, two heavy (H) chains and two light (L) chains which are typically inter-connected by disulfide bonds. Each heavy chain is comprised of a heavy chain variable region (abbreviated herein as VH) and a heavy chain constant region. The heavy chain constant region can comprise e.g. three domains CH1, CH2 and CH3. Each light chain is comprised of a light chain variable region (abbreviated herein as VL) and a light chain constant region. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs and up to four FRs arranged from amino-terminus to carboxy-terminus e.g. in the following order: FR1, CDR1, FR2, CDR2, FR3, CDR3, FR4.

As used herein, the term “Complementarity Determining Regions” (CDRs; e.g., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (e.g. about residues 24-34 (L1), 50-56 (L2) and 89-97 (L3) in the light chain variable domain and 31-35 (H1), 50-65 (H2) and 95-102 (H3) in the heavy chain variable domain; and/or those residues from a “hypervariable loop” (e.g. about residues 26-32 (L1), 50-52 (L2) and 91-96 (L3) in the light chain variable domain and 26-32 (H1), 53-55 (H2) and 96-101 (H3) in the heavy chain variable domain). In some instances, a complementarity determining region can include amino acids from both a CDR region defined according to Kabat and a hypervariable loop.

Depending on the amino acid sequence of the constant domain of their heavy chains, intact antibodies can be assigned to different “classes”. There are five major classes of intact antibodies: IgA, IgD, IgE, IgG, and IgM, and several of these maybe further divided into “subclasses” (isotypes), e.g., IgG1, IgG2, IgG3, IgG4, IgA1, and IgA2. A preferred class of immunoglobulins for use in the present invention is IgG.

The heavy-chain constant domains that correspond to the different classes of antibodies are called [alpha], [delta], [epsilon], [gamma], and [mu], respectively. The subunit structures and three-dimensional configurations of different classes of immunoglobulins are well known. As used herein antibodies are conventionally known antibodies and functional fragments thereof.

A “functional fragment”, or “antigen-binding antibody fragment” of an antibody/immunoglobulin, or “antigen-binding fragment of an antibody”, or an “antibody fragment”, or a “fragment of an antibody” in general relates to a fragment of an antibody/immunoglobulin (e.g., a variable region of an IgG) that retains the antigen-binding region. An “antigen-binding region” of an antibody typically is found in one or more hyper variable region(s) of an antibody, e.g., the CDR1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320).

“Functional fragments”, “antigen-binding antibody fragments”, “antigen-binding fragments of an antibody”, or “antibody fragments” or “fragments of an antibody” of the disclosure may include, but are not limited to, those which contain at least one disulfide bond that can be reacted with a reducing agent as described herein. Examples of suitable fragments include Fab, Fab′, Fab′-SH, F(ab′)₂, and Fv fragments; diabodies; single domain antibodies (DAbs), linear antibodies; single-chain antibody molecules (scFv); and multispecific, such as bi- and tri-specific, antibodies formed from antibody fragments. An antibody other than a “multi-specific” or “multi-functional” antibody is understood to have each of its binding sites identical. The F(ab′)₂ or Fab may be engineered to minimize or completely remove the intermolecular disulfide interactions that occur between the CH1 and CL domains.

The term “Fc region” herein is in general used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index.

Variants of the antibodies or antigen-binding antibody fragments contemplated herein are molecules in which the binding activity of the antibody or antigen-binding antibody fragment is maintained.

“Binding proteins” or “proteinaceous binding molecules with antibody-like binding properties”, as used herein, are generally known to a person skilled in the art. Illustrative non-limiting examples include affibodies, adnectins, anticalins, DARPins, and avimers.

A “human” antibody or antigen-binding fragment thereof is in general defined as one that is not chimeric (e.g., not “humanized”) and not from (either in whole or in part) a non-human species. A human antibody or antigen-binding fragment thereof can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising a polypeptide sequence utilizing the data obtained there from. Another example of a human antibody or antigen-binding fragment thereof is one that is encoded by a nucleic acid isolated from a library of antibody sequences of human origin (e.g., such library being based on antibodies taken from a human natural source).

A “humanized antibody” or humanized antigen-binding fragment thereof is in general defined herein as one that is (i) derived from a non-human source (e.g., a transgenic mouse which bears a heterologous immune system), which antibody is based on a human germline sequence; (ii) where amino acids of the framework regions of a non-human antibody are partially exchanged to human amino acid sequences by genetic engineering or (iii) CDR-grafted, wherein the CDRs of the variable domain are from a non-human origin, while one or more frameworks of the variable domain are of human origin and the constant domain (if any) is of human origin.

A “chimeric antibody” or antigen-binding fragment thereof is in general defined herein as one, wherein the variable domains are derived from a non-human origin and some or all constant domains are derived from a human origin.

The term “monoclonal antibody” as used herein in general refers to an antibody obtained from a population of substantially homogeneous antibodies, i.e., the individual antibodies comprising the population are identical except for possible mutations, e.g., naturally occurring mutations, that may be present in minor amounts. Thus, the term “monoclonal” indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. The term “monoclonal” is not to be construed as to require production of the antibody by any particular method. The term monoclonal antibody specifically includes chimeric, humanized and human antibodies.

An “isolated” antibody is in general one that has been identified and separated from a component of the cell that expressed it. Contaminant components of the cell are materials that would interfere with diagnostic or therapeutic uses of the antibody, and may include enzymes, hormones, and other proteinaceous or nonproteinaceous solutes.

As used herein, an antibody “binds specifically to”, is “specific to/for” or “specifically recognizes” an antigen of interest, e.g. a tumor-associated polypeptide antigen target, is in general one that binds the antigen with sufficient affinity such that the antibody is useful as a therapeutic agent in targeting a cell or tissue expressing the antigen, and does not significantly cross-react with other proteins or does not significantly cross-react with proteins other than orthologs and variants (e.g. mutant forms, splice variants, or proteolytically truncated forms) of the aforementioned antigen target. The term “specifically recognizes” or “binds specifically to” or is “specific to/for” a particular polypeptide or an epitope on a particular polypeptide target as used herein can be exhibited, for example, by an antibody, or antigen-binding fragment thereof, having a monovalent K_{D} for the antigen of less than about 10⁻⁴ M, alternatively less than about 10⁻⁵ M, alternatively less than about 10⁻⁶ M, alternatively less than about 10⁻⁷ M, alternatively less than about 10⁻⁸ M, alternatively less than about 10⁻⁹ M, alternatively less than about 10⁻¹⁰ M, alternatively less than about 10⁻¹¹ M, alternatively less than about 10⁻¹² M, or less. An antibody “binds specifically to,” is “specific to/for” or “specifically recognizes” an antigen if such antibody is able to discriminate between such antigen and one or more reference antigen(s). In its most general form, “specific binding”, “binds specifically to”, is “specific to/for” or “specifically recognizes” is referring to the ability of the antibody to discriminate between the antigen of interest and an unrelated antigen, as determined, for example, in accordance with one of the following methods. Such methods comprise, but are not limited to surface plasmon resonance (SPR), Western blots, ELISA-, RIA-, ECL-, IRMA-tests and peptide scans. For example, a standard ELISA assay can be carried out. The scoring may be carried out by standard color development (e.g. secondary antibody with horseradish peroxidase and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative is more than 5-fold, 10-fold, 50-fold, and preferably more than 100-fold. Typically, determination of binding specificity is performed by using not a single reference antigen, but a set of about three to five unrelated antigens, such as milk powder, BSA, transferrin or the like.

“Binding affinity” or “affinity” in general refers to the strength of the total sum of non-covalent interactions between a single binding site of a molecule and its binding partner. Unless indicated otherwise, as used herein, “binding affinity” refers to intrinsic binding affinity which reflects a 1:1 interaction between members of a binding pair (e.g. an antibody and an antigen). The dissociation constant “K_{D}” is commonly used to describe the affinity between a molecule (such as an antibody) and its binding partner (such as an antigen) i.e. how tightly a ligand binds to a particular protein. Ligand-protein affinities are influenced by non-covalent intermolecular interactions between the two molecules. Affinity can be measured by common methods known in the art, including those described herein. In one embodiment, the “K_{D}” or “K_{D} value” according to this invention is measured by using surface plasmon resonance assays using suitable devices including but not limited to Biacore instruments like Biacore T100, Biacore T200, Biacore 2000, Biacore 4000, a Biacore 3000 (GE Healthcare Biacore, Inc.), or a ProteOn XPR36 instrument (Bio-Rad Laboratories, Inc.).

The term “antibody drug conjugate” or abbreviated ADC is well known to a person skilled in the art, and, as used herein, in general refers to the linkage of an antibody or an antigen binding fragment thereof with a drug, such as a chemotherapeutic agent, a toxin, an immunotherapeutic agent, an imaging probe, and the like.

The present disclosure also relates to a “pharmaceutically acceptable salt”. Any pharmaceutically acceptable salt can be used. In particular, the term “pharmaceutically acceptable salt” refers to a salt of a conjugate or compound of the invention that is pharmaceutically acceptable and that possesses the desired pharmacological activity of the parent compound. In particular, such salts have low toxicity and may be inorganic or organic acid addition salts and base addition salts. Specifically, such salts include, but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, purely by way of example, sodium, potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like. A counterion or anionic counterion can be used in a quaternary amine to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, and the like), and carboxylate ions (e.g., acetate, ethanoate, propanoate, benzoate, glycerate, lactate, tartrate, glycolate, and the like).

As used herein, the term “solvate” may refer to an aggregate that comprises one or more molecules of a conjugate or compound described herein with one or more molecules of solvent. The solvent may be water, in which case the solvate may be a hydrate. Alternatively, the solvent may be an organic solvent. Thus, the conjugates or compounds of the present disclosure may exist as a hydrate, including a monohydrate, dihydrate, hemihydrate, sesquihydrate, trihydrate, tetrahydrate and the like, as well as the corresponding solvated forms. The compounds of the invention may be true solvates, while in other cases, the compounds of the invention may merely retain adventitious water or be a mixture of water plus some adventitious solvent.

### Conjugate of Formula (I)

As set out above, the present invention relates to a conjugate having the formula (I):

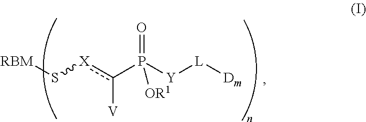

or a pharmaceutically acceptable salt or solvate thereof,
wherein:
RBM is a receptor binding molecule;
[symbol] is a double bond; or
[symbol] is a bond;
V is absent when [symbol] is a double bond; or
V is H or (C₁-C₈)alkyl when [symbol] is a bond;
X is R₃—C when [symbol] is a double bond; or
X is

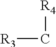

when [symbol] is a bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
L is a linker;
D is a drug moiety;
m is an integer ranging from 1 to 10; and
n is an integer ranging from 1 to 20.

Conjugates of formula (I) comprise a receptor binding molecule which is connected to a drug moiety via a phosphorus (V) moiety (also sometimes denoted as “P5”) and a linker. The residue R¹ is first polyalkylene glycol unit which is bound to the oxygen atom, which is attached to the phosphorus atom of the phosphorus (V) moiety. Moreover, conjugates that carry a second polyalkylene glycol, orthogonal to the orientation of RBM and D within in the linker L, are also described.

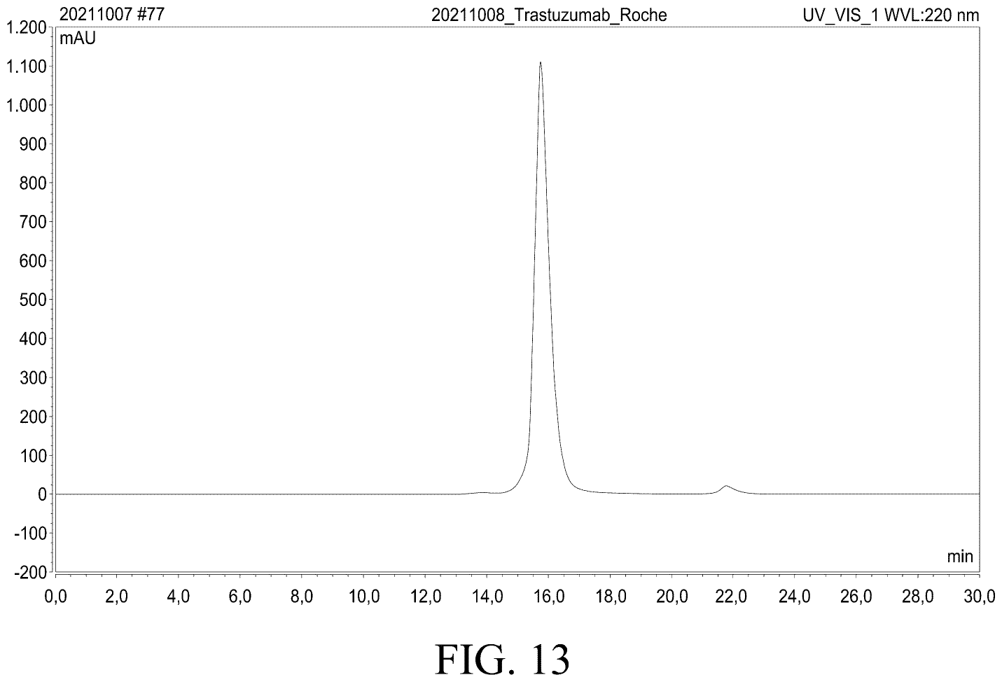

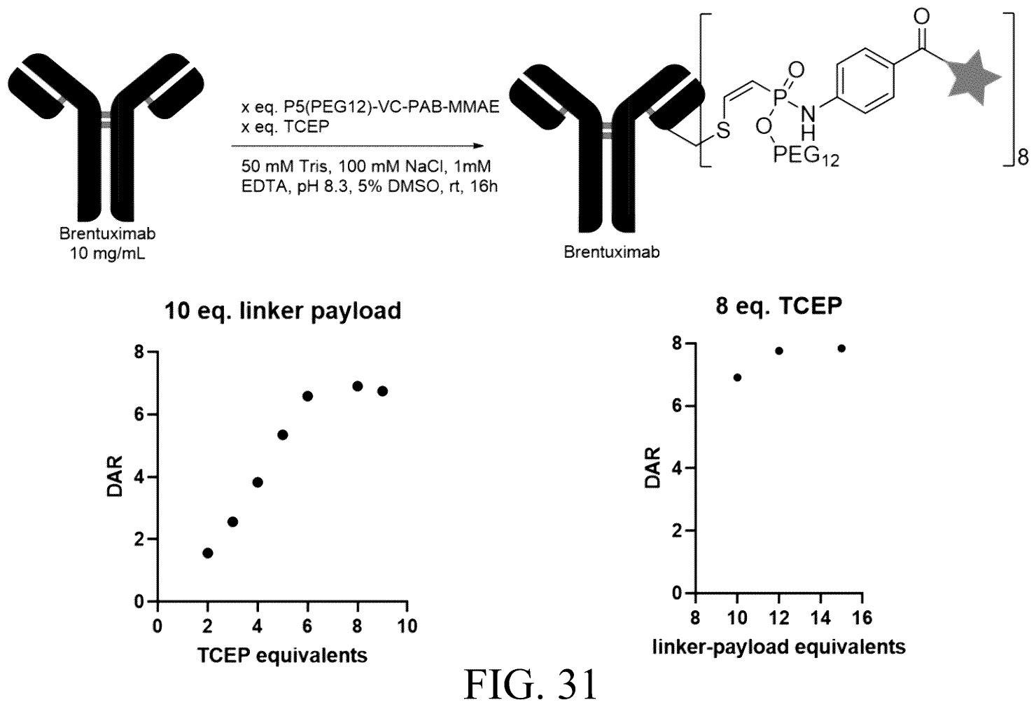

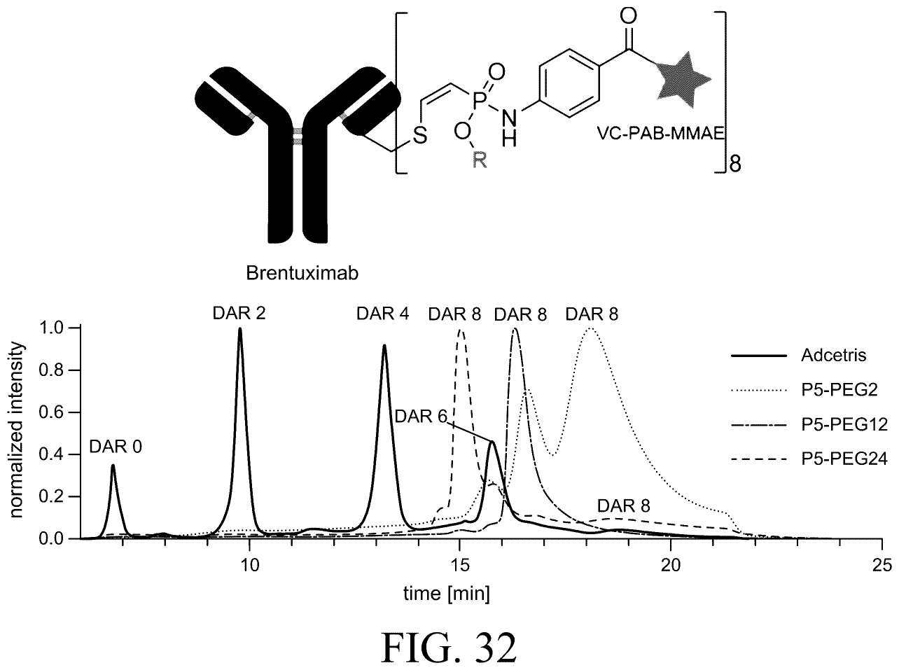

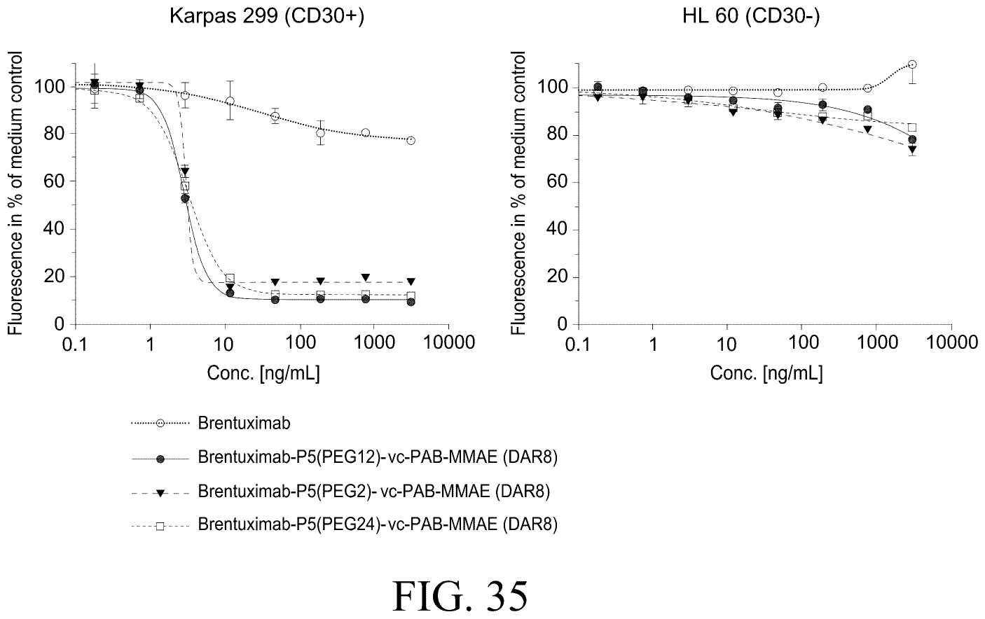

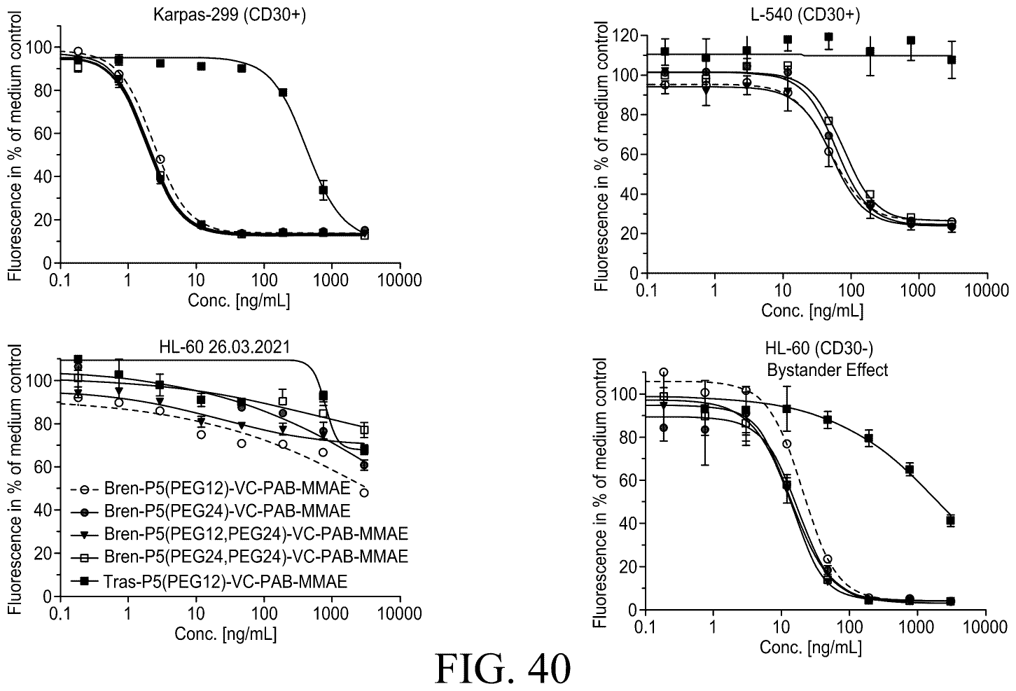

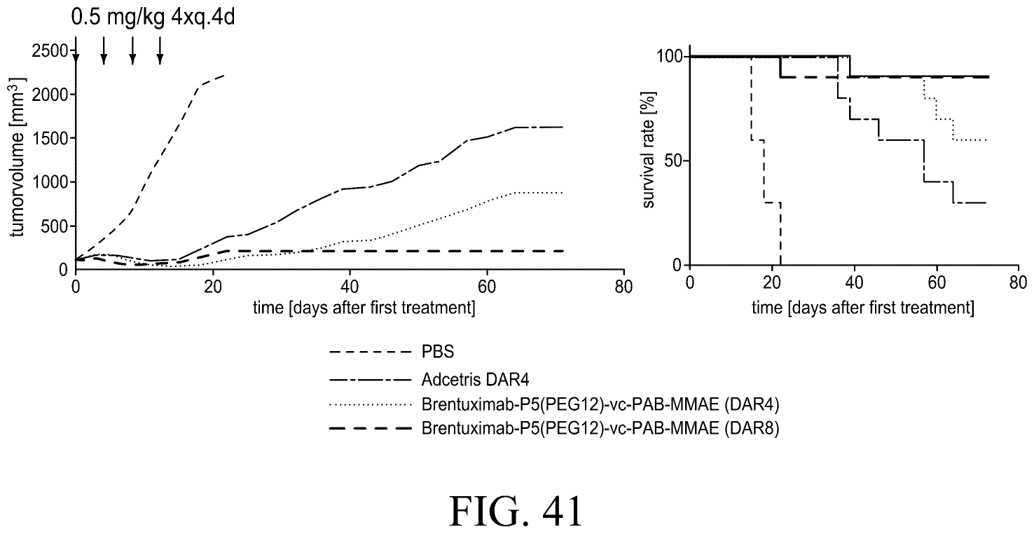

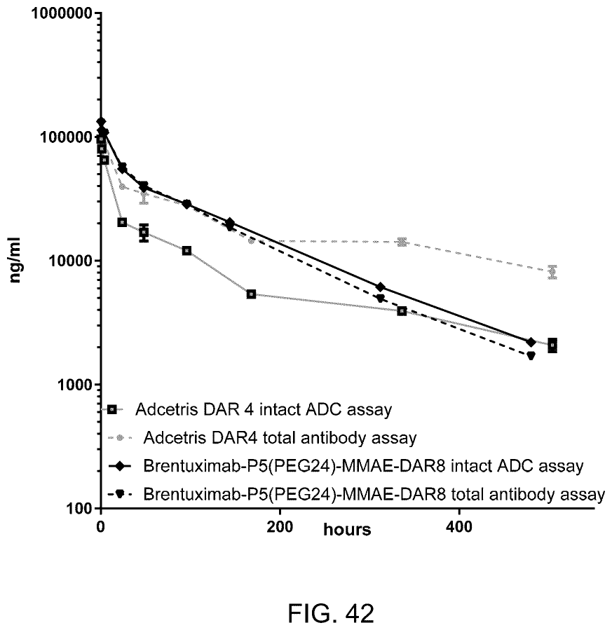

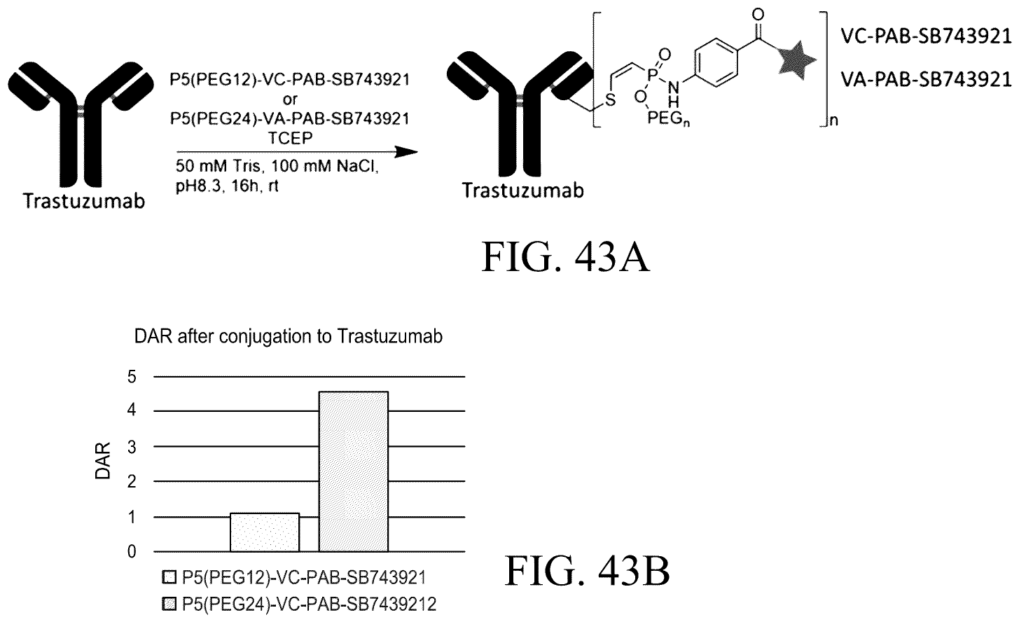

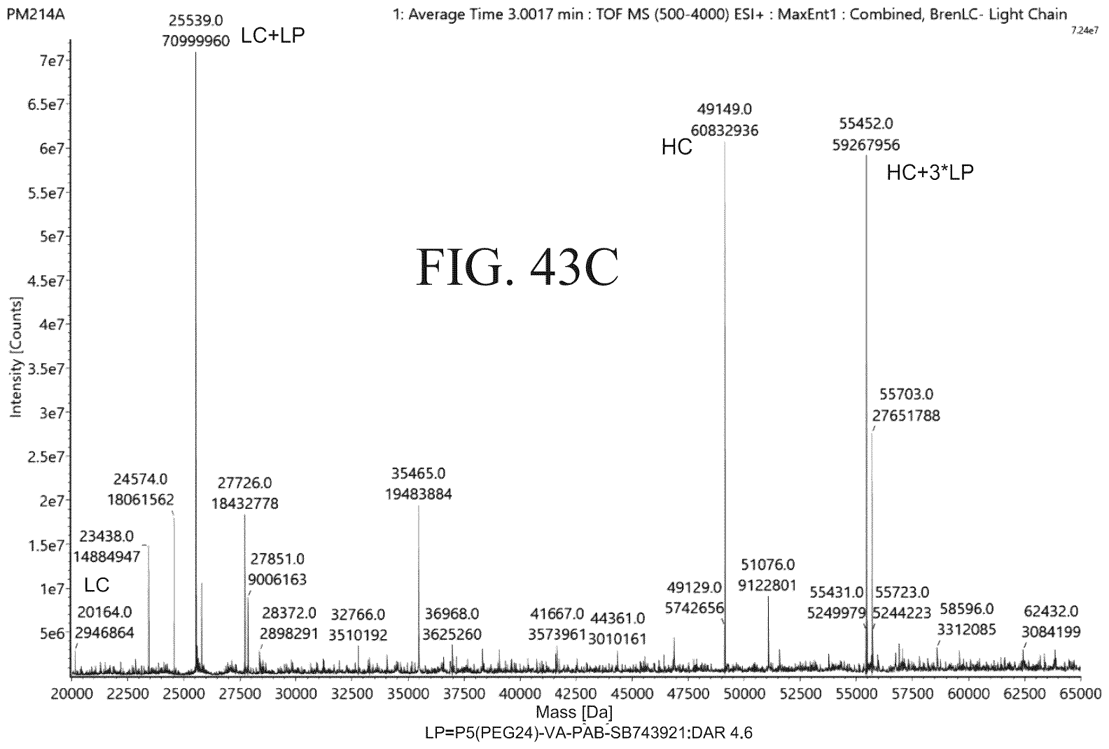

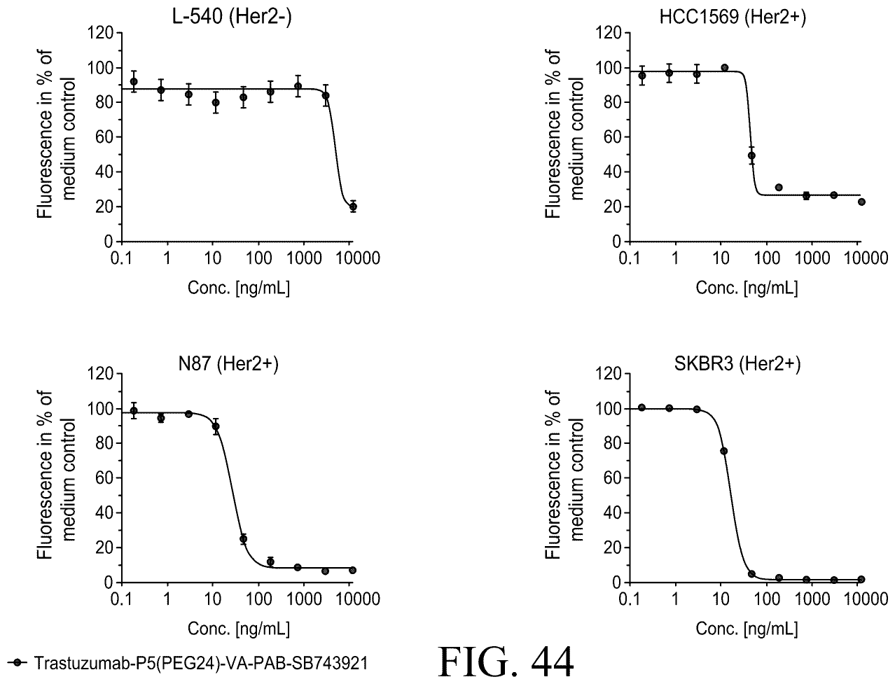

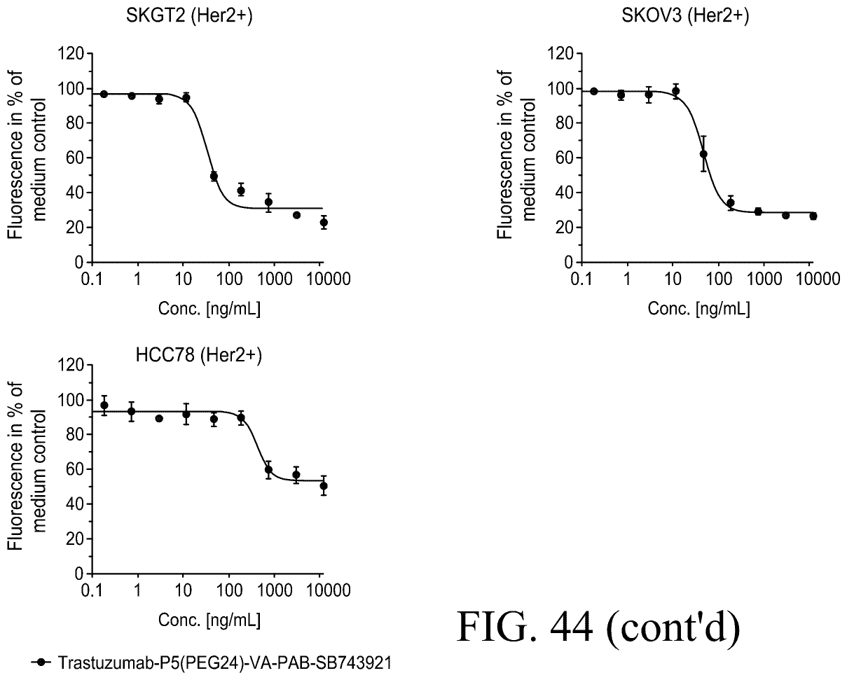

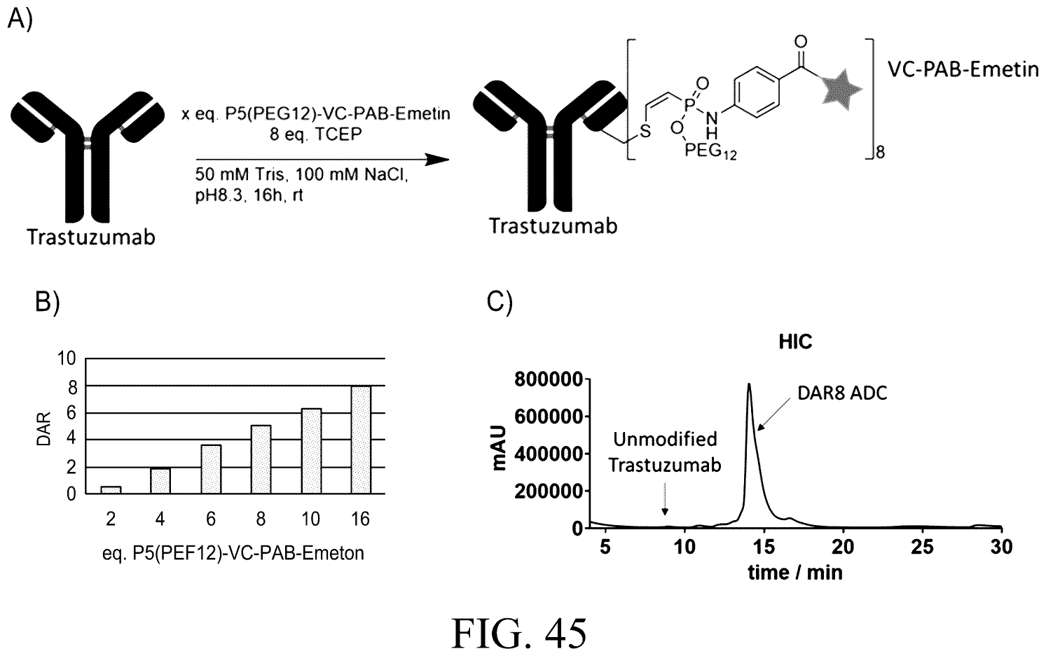

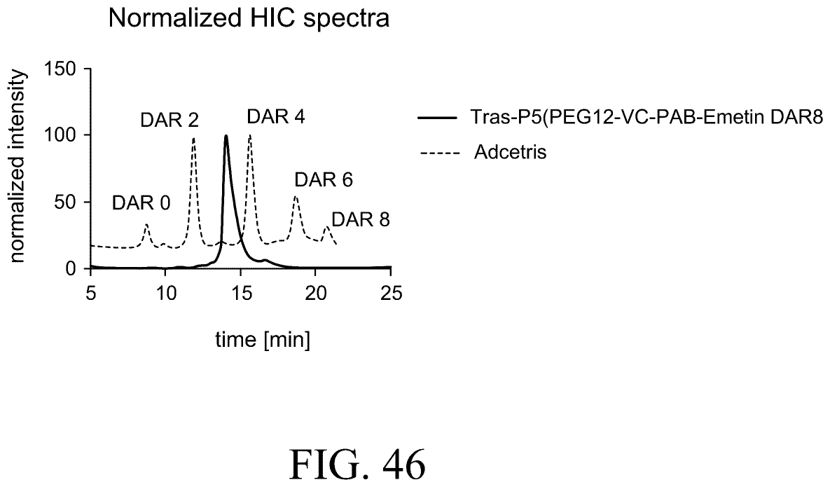

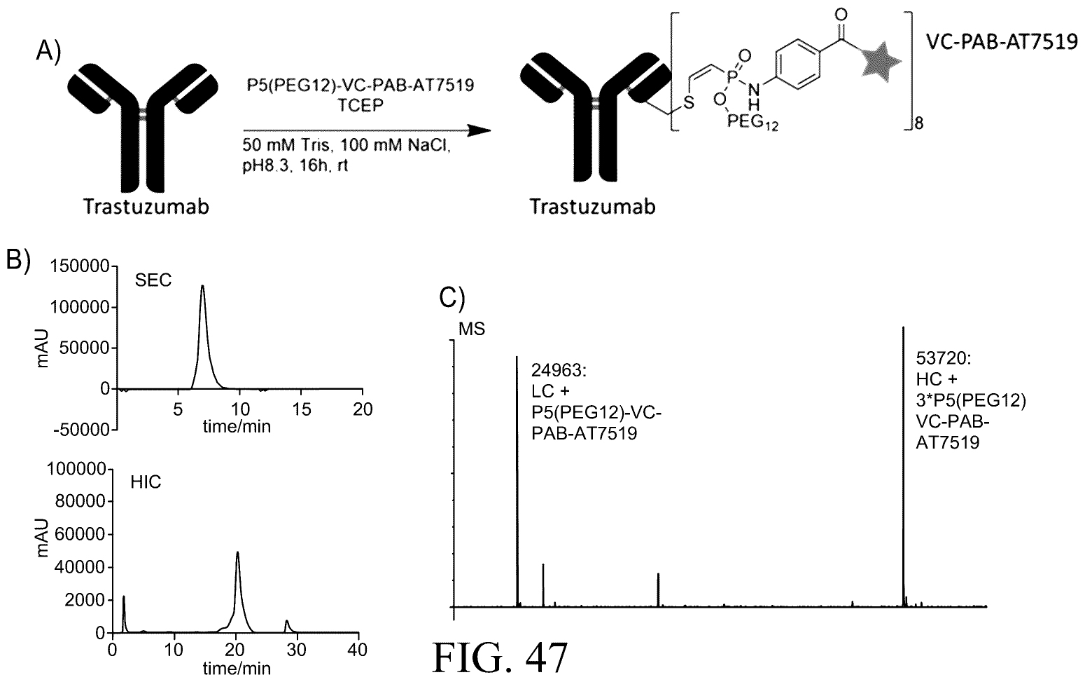

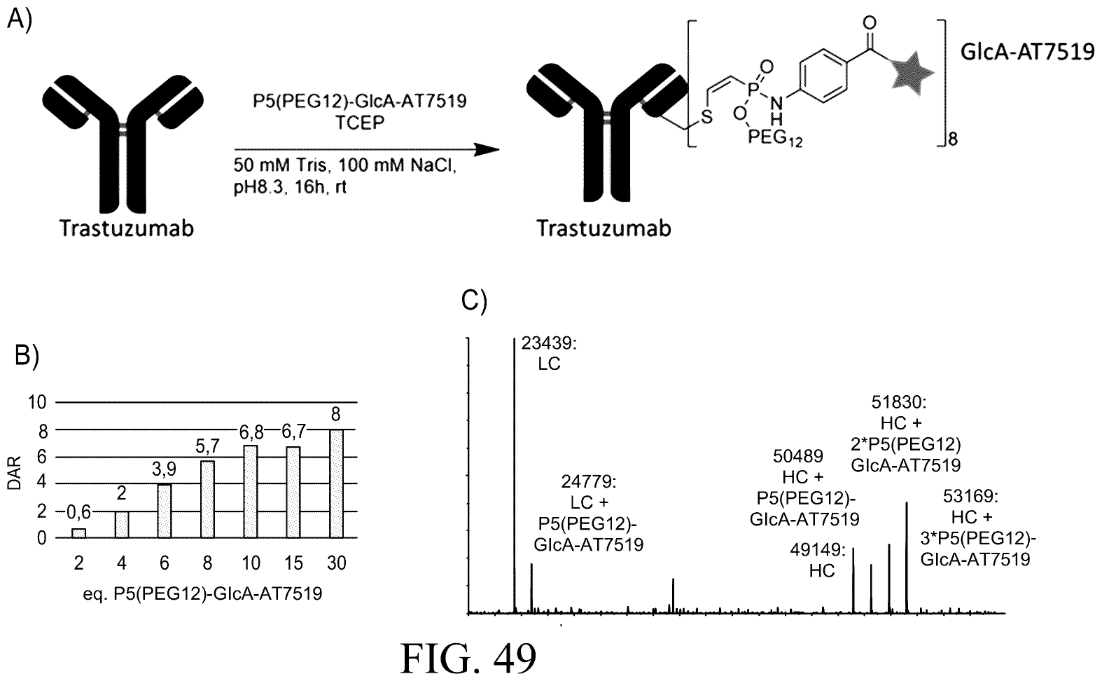

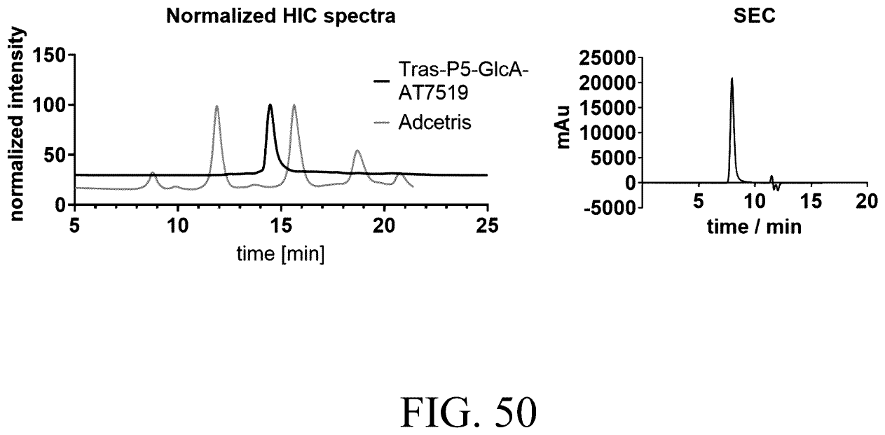

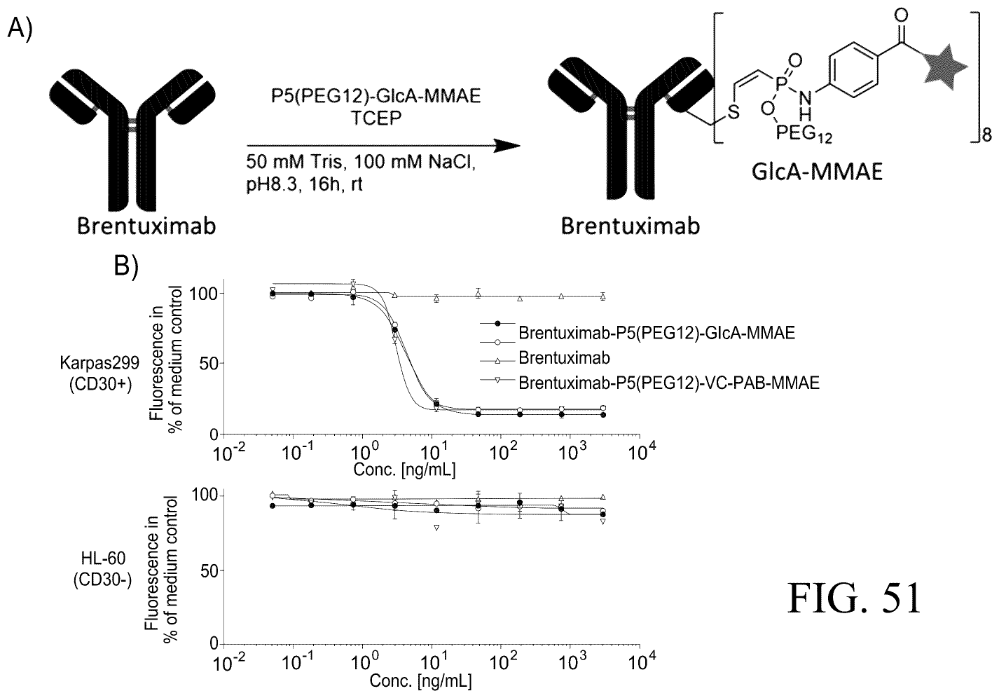

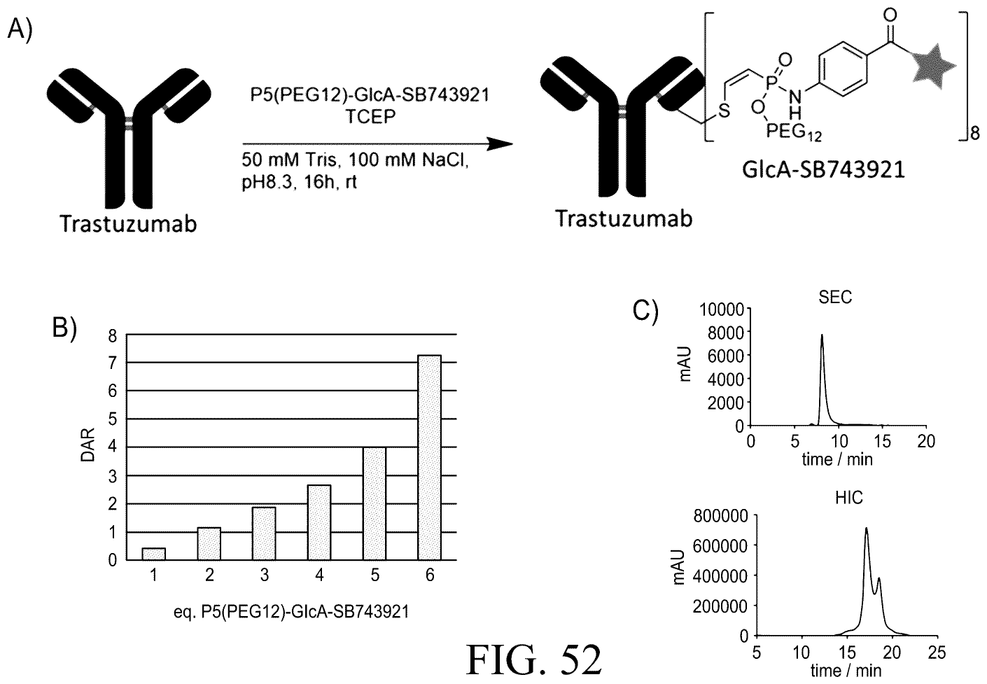

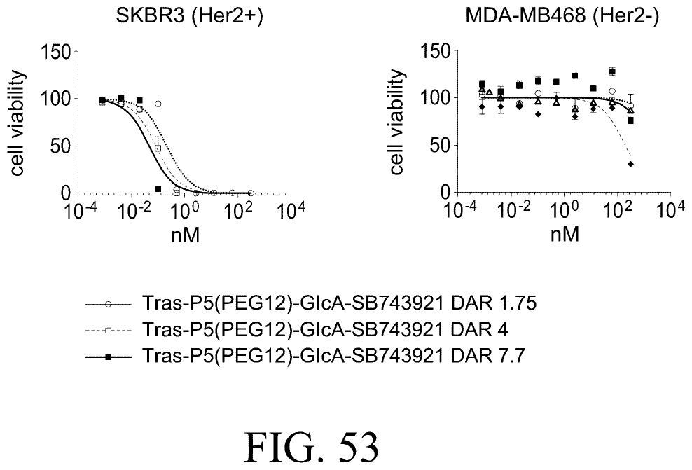

The inventors have found that conjugates of formula (I) have various advantageous properties, as shown in the following. Conjugates of formula (I) have been prepared with different linkers and drugs, and have been tested (the table of Example 2, e.g., shows an overview over the conjugates prepared and tested in Examples 3 to 7, and conjugates with further linkers and drugs are described in Examples 8 to 14). It has been found that conjugates of formula (I) have good hydrophilicity and show low aggregation in solution (Example 2 and FIGS. 13 to 30, Example 9 and FIGS. 45 and 46, Example 10 and FIG. 47, Example 12 and FIG. 50, and Example 14 and FIG. 52). Further, conjugates of formula (I) show a good cytotoxicity towards target-positive cancer cells (Example 4 and FIGS. 35 to 39, Example 8 and FIG. 44, Example 13 and FIG. 51, and Example 14 and FIG. 53). Conjugates of formula (I) also show a favorable bystander effect (Example 5 and FIG. 40). Also, conjugates of formula (I) show favorable in vivo efficacy, in particular when directly compared to the efficacy of the commercial product Adcetris (Example 6 and FIG. 41). Further, conjugates of formula (I) also show good pharmacokinetic behaviour in vivo, exemplified by the very narrow concentration courses of total antibody and intact ADC quantification over time, clearly speaking for a highly stable conjugate in vivo. The stability exceeds the one of the commercial product Adcetris. Moreover, the ADC clearance from blood circulation for a DAR8 VC-PAB-MMAE construct is not enhanced and similar to Adcetris, which is only DAR4. (Example 7 and FIG. 42). Also, conjugates of formula (I) can be efficiently prepared with various ratios of the drug moiety to the receptor binding molecule (Examples 2 and 3 and FIG. 31, Example 9 and FIG. 45, Example 12 and FIG. 49, and Example 14 and FIG. 52). Conjugates of formula (I) can be also prepared with various chain length of polyalkylene glycol unit(s) (Examples 2 and 3, and FIG. 32, and Example 8 and FIGS. 43A to 43C). In this context, the inventors have also found that the efficiency of the conjugation reaction of the receptor binding molecule with the linker-drug molecule resulting in conjugates of formula (I), in particular the yield and drug to antibody ratio (DAR), could be improved when using longer PEG residues (as illustrative examples, PEG12 having 12 PEG units at the phosphorus atom, and even more when using PEG24 having 24 PEG units at the phosphorus atom) (Example 8 and FIGS. 43A to 43C). In sum, the inventors have surprisingly found that conjugates of formula (I) exhibit excellent properties which make them useful as pharmaceuticals, such as, e.g., good efficiency of the conjugation reaction, good cyctoxocicity against target-positive cancer cells, a favorable bystander effect, excellent in vivo efficacy and in vivo pharmacokinetics. It is noted that conjugates, which comprise a phosphorus (V) moiety and a drug moiety, are e.g. described in WO 2018/041985 A1, WO 2019/170710, and Kasper et al., Angew. Chem. Int. Ed. 2019, vol. 58, pp. 11631 to 11636, which are hereby incorporated by reference.

Preferably R³ is H or (C₁-C₈)alkyl; more preferably R³ is H. Preferably R⁴, when present is H or (C₁-C₈)alkyl; more preferably R⁴, when present, is H. Preferably R⁵, when present is H or (C₁-C₈)alkyl; more preferably R⁵, when present, is H. Preferably R⁶, when present is H or (C₁-C₈)alkyl; more preferably R⁶, when present, is H. Preferably R⁷, when present is H or (C₁-C₈)alkyl; more preferably R⁷, when present, is H.

Preferably, [symbol] is a double bond; V is absent; X is R₃—C; and R³ is H or an optionally substituted aliphatic residue or an optionally substituted aromatic residue; preferably R³ is H or (C₁-C₈)alkyl; more preferably R³ is H.

More preferably, [symbol] represents a double bond; V is absent; X represents R₃—C, and R₃ represents H or (C₁-C₈)alkyl. Preferably, R³ represents H or (C₁-C₆)alkyl, more preferably H or (C₁-C₄)alkyl, still more preferably H or (C₁-C₂)alkyl. In preferred embodiments, R₃ is H.

In some embodiments, [symbol] may be a bond; V is H or (C₁-C₈)alkyl, preferably V is H; X is

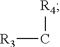

R₃ is H or an optionally substituted aliphatic residue or an optionally substituted aromatic residue; more preferably R³ is H or (C₁-C₈)alkyl, more preferably R³ is H; R⁴ is H or an optionally substituted aliphatic residue or an optionally substituted aromatic residue; preferably, R⁴ is H or (C₁-C₈)alkyl, preferably R⁴ is H.

In some embodiments, [symbol] may represent a bond; V may be H or (C₁-C₈)alkyl; X may represent

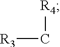

and R₃ and R₄ may independently represent H or (C₁-C₈)alkyl. Preferably, R₃ and R₄ independently represent H or (C₁-C₈)alkyl, more preferably H or (C₁-C₄)alkyl, still more preferably H or (C₁-C₂)alkyl. Preferably, R₃ and R₄ are the same; even more preferably, R₃, R₄ and V are the same. More preferably, R₃ and R₄ are both H. Preferably, V is H or (C₁-C₆)alkyl, more preferably H or (C₁-C₄)alkyl, still more preferably H or (C₁-C₂)alkyl. Even more preferably, V is H. In preferred embodiments, R₃, R₄ and V are each H.

The integer m ranges from 1 to 10. Accordingly, the integer m may be 1, 2, 3, 4, 5, 6, 7, 8, 9 or 10. Preferably, the integer m ranges from 1 to 4. More preferably, the integer m is 1 or 2. Even more preferably, the integer m is 1.

The integer n ranges from 1 to 20. Accordingly, the integer n may be 1, 2, 3, 4, 5, 6, 7, 8, 9, 10, 11, 12, 13, 14, 15, 16, 17, 18, 19 or 20. Preferably, the integer n ranges from 1 to 10. More preferably, the integer n ranges from 2 to 10. Still more preferably, the integer n ranges from 4 to 10. Still more preferably, the integer n ranges from 6 to 10. Still more preferably, the integer n is 6, 7, 8, 9 or 10. Still more preferably, the integer n ranges from 7 to 10. Still more preferably, the integer n is 7, 8 or 9. Still more preferably, the integer n is 7 or 8. Still more preferably, the integer n ranges from 7 to 10. Even more preferably, the integer n is 8.

The integer n ranges from 1 to 20. Preferably, the integer n ranges from 1 to 10. More preferably, the integer n ranges from 2 to 8. Still more preferably, the integer n is 2, 3, 4, 5 or 6. Still more preferably, the integer n ranges from 3 to 6. Still more preferably, the integer n is 3, 4 or 5. Still more preferably, the integer n may be 4 or 5. Still more preferably, the integer n is 4.

Preferably, m is an integer ranging from 1 to 4, more preferably 1 or 2, still more preferably 1; and preferably n is an integer ranging from 1 to 20, more preferably from 1 to 10, still more preferably from 2 to 10, still more preferably from 4 to 10, still more preferably from 6 to 10, still more preferably n is 6, 7, 8, 9 or 10, still more preferably n ranges from 7 to 10, still more preferably from 7 to 10; still more preferably n is 7, 8 or 9, still more preferably n is 7 or 8, even more preferably n is 8.

Preferably, m is an integer ranging from 1 to 4, more preferably m is 1 or 2, still more preferably m is 1; and preferably n is an integer ranging from 1 to 20, more preferably from 1 to 10, still more preferably from 2 to 8; still more preferably n is 2, 3, 4, 5 or 6; still more preferably n ranges from 3 to 6; still more preferably n is 3, 4 or 5; still more preferably n is 4 or 5; even more preferably n is 4.

Preferably, m is 1; and preferably n is an integer ranging from 1 to 20, more preferably from 1 to 10, still more preferably from 2 to 10, still more preferably from 4 to 10, still more preferably from 6 to 10, still more preferably n is 6, 7, 8, 9 or 10, still more preferably n ranges from 7 to 10, still more preferably n is 7, 8 or 9, still more preferably n is 7 or 8, even more preferably n is 8. Accordingly, preferably, m is 1 and n is an integer ranging from 1 to 20. More preferably, m is 1 and n is an integer ranging from 1 to 10. Still more preferably, m is 1 and n is an integer ranging from 2 to 10. Still more preferably, m is 1 and n is an integer ranging from 4 to 10. Still more preferably, m is 1 and n is an integer ranging from 6 to 10. Still more preferably, m is 1 and n is 6, 7, 8, 9 or 10. Still more preferably, m is 1 and n is an integer ranging from 7 to 10. Still more preferably, m is 1 and n is 7, 8 or 9. Still more preferably, m is 1 and n is 7 or 8. Even more preferably, m is 1 and n is 8.

Preferably, m is 1; and in some embodiments n is an integer ranging from 1 to 20, more preferably from 1 to 10, still more preferably from 2 to 8; still more preferably n is 2, 3, 4, 5 or 6, still more preferably n ranges from 3 to 6; still more preferably n is 3, 4 or 5; still more preferably n is 4 or 5; even more preferably n is 4. Accordingly, preferably, m is 1 and n is an integer ranging from 1 to 20. More preferably, m is 1 and n is an integer ranging from 1 to 10. Still more preferably, m is 1 and n is an integer ranging from 2 to 8. Still more preferably, m is 1 and n is 2, 3, 4, 5 or 6. Still more preferably, m is 1 and n ranges from 3 to 6. Still more preferably, m is 1 and n is 3, 4 or 5. Still more preferably, m is 1 and n is 4 or 5. Even more preferably, m is 1 and n is 4.

Preferably, the number of drug moieties D per receptor binding molecule may be from 1 to 20. More preferably, the number of drug moieties D per receptor binding molecule is from 1 to 14. Still more preferably, the number of drug moieties D per receptor binding molecule is from 2 to 14. Still more preferably, the number of drug moieties D per receptor binding molecule is from 4 to 14. Still more preferably, the number of drug moieties D per receptor binding molecule is from 5 to 12. Still more preferably, the number of drug moieties D per receptor binding molecule is from 6 to 12. Still more preferably, the number of drug moieties D per receptor binding molecule is from 7 to 10. Even more preferably, the number of drug moieties D per receptor binding molecule is 8.

Preferably, the number of drug moieties D per receptor binding molecule may be from 1 to 20. More preferably, the number of drug moieties D per receptor binding molecule is from 1 to 14. Still more preferably, the number of drug moieties D per receptor binding molecule is from 1 to 12. Still more preferably, the number of drug moieties D per receptor binding molecule is from 2 to 10. Still more preferably, the number of drug moieties D per receptor binding molecule is from 2 to 8. Still more preferably, the number of drug moieties D per receptor binding molecule is from 2 to 6. Still more preferably, the number of drug moieties D per receptor binding molecule is from 3 to 5. Even more preferably, the number of drug moieties D population per receptor binding molecule is 4.

### Receptor Binding Molecule (RBM)

RBM is a receptor binding molecule. The term “receptor binding molecule” in general refers to any molecule which is capable to bind to a receptor. As illustrative but non-limiting example, the receptor, to which a receptor binding molecule may bind, may be expressed on a cell surface. As illustrative but non-limiting example, the cell which expresses the receptor, may be a cancer cell. A person skilled in the art knows to select a suitable receptor binding molecules.

The receptor may be a tumor associated surface antigen. Accordingly, the receptor binding molecule may be capable to specifically bind to a tumour associated surface antigen. The term “tumour associated surface antigen” as used herein in general refers to an antigen that is or can be presented on a surface that is located on or within tumour cells. These antigens can be presented on the cell surface with an extracellular part, which is often combined with a transmembrane and cytoplasmic part of the molecule. These antigens can in some embodiments be presented only by tumour cells and not by normal, i.e. non-tumour cells. Tumour antigens can be exclusively expressed on tumour cells or may represent a tumour specific mutation compared to non-tumour cells. In such an embodiment a respective antigen may be referred to as a tumour-specific antigen. Some antigens are presented by both tumour cells and non-tumour cells, which may be referred to as tumour-associated antigens. These tumour-associated antigens can be overexpressed on tumour cells when compared to non-tumour cells or are accessible for antibody binding in tumour cells due to the less compact structure of the tumour tissue compared to non-tumour tissue. In some embodiments the tumour associated surface antigen is located on the vasculature of a tumour. Illustrative but non-limiting examples of a tumour associated surface antigen include CD19, CD30, Her2 or PMSA. Tumor associated surface antigens, are known to a person skilled in the art. In particular, those which have been found useful for the development of ADCs are described, e.g., in the review article of Criscitello et al., “Antibody-drug conjugates in solid tumors: a look into novel targets”, Journal of Hematology and Oncology, (2021) 14:20.

The receptor binding molecule may be selected from the group consisting of an antibody, an antibody fragment, and a proteinaceous binding molecule with antibody-like binding properties.

Preferably, the receptor binding molecule is an antibody. More preferably, the antibody is selected from the group consisting of a monoclonal antibody, a chimeric antibody, a humanized antibody, a human antibody, and a single domain antibody, such as a camelid or shark single domain antibody. Still more preferably, the antibody is a monoclonal antibody. Preferably, the antibody is capable to specifically bind to a tumour associated surface antigen. In some embodiments, the antibody may be Brentuximab. In some embodiments, the antibody may be Trastuzumab.

The receptor binding molecule may be an antibody fragment. Preferably, the antibody fragment is a divalent antibody fragment. More preferably, the divalent antibody fragment is selected from the group consisting of a (Fab)₂′-fragment, a divalent single-chain Fv fragment, a dual affinity re-targeting (DART) antibody, and a diabody. Alternatively, preferably the antibody fragment is a monovalent antibody fragment. More preferably the monovalent antibody fragment is selected from the group consisting of a Fab fragment, a Fv fragment, and a single-chain Fv fragment (scFv). It is also possible that the monovalent antibody fragment is a fragment of a single domain camelid or a shark single domain antibody. Preferably, the antibody fragment is capable to specifically bind to a tumour associated surface antigen.

The receptor binding molecule may be a proteinaceous binding molecule with antibody-like binding properties. Examples of proteinaceous binding molecules with antibody-like binding properties that can be used as receptor binding molecule include, but are not limited to, an aptamer, a mutein based on a polypeptide of the lipocalin family, a glubody, a protein based on the ankyrin scaffold, a protein based on the crystalline scaffold, an adnectin, an avimer, a EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or a an immunoglobulin-like domain (for example, domain antibodies or camel heavy chain antibodies), a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, “Kappabodies” (III. et al. “Design and construction of a hybrid immunoglobulin domain with properties of both heavy and light chain variable regions” Protein Eng 10:949-57 (1997)), “Minibodies” (Martin et al. “The affinity-selection of a minibody polypeptide inhibitor of human interleukin-6” EMBO J 13:5303-9 (1994)), “Janusins” (Traunecker et al. “Bispecific single chain molecules (Janusins) target cytotoxic lymphocytes on HIV infected cells” EMBO J 10:3655-3659 (1991) and Traunecker et al. “Janusin: new molecular design for bispecific reagents” Int J Cancer Suppl 7:51-52 (1992), a nanobody, a adnectin, a tetranectin, a microbody, an affilin, an affibody or an ankyrin, a crystallin, a knottin, ubiquitin, a zinc-finger protein, an autofluorescent protein, an ankyrin or ankyrin repeat protein or a leucine-rich repeat protein, an avimer (Silverman, Lu Q, Bakker A, To W, Duguay A, Alba B M, Smith R, Rivas A, Li P, Le H, Whitehorn E, Moore K W, Swimmer C, Perlroth V, Vogt M, Kolkman J, Stemmer W P 2005, Nat Biotech, December; 23(12):1556-61, E-Publication in Nat Biotech. 2005 Nov. 20 edition); as well as multivalent avimer proteins evolved by exon shuffling of a family of human receptor domains as also described in Silverman J, Lu Q, Bakker A, To W, Duguay A, Alba B M, Smith R, Rivas A, Li P, Le H, Whitehorn E, Moore K W, Swimmer C, Perlroth V, Vogt M, Kolkman J, Stemmer W P, Nat Biotech, December; 23(12):1556-61, E-Publication in Nat. Biotechnology. 2005 Nov. 20 edition. Preferably the proteinaceous binding molecule with antibody-like binding properties is selected from the group consisting of a mutein based on a polypeptide of the lipocalin family, a glubody, a protein based on the ankyrin scaffold, a protein based on the crystalline scaffold, an adnectin, an avimer, a DARPin, and an affibody. Preferably, the proteinaceous binding molecule with antibody-like binding properties is capable to specifically bind to a tumour associated surface antigen.

### Group Y

The group Y is selected from the group consisting of NR⁵, S, O, and CR⁶R⁷. R⁵ is H; or an optionally substituted aliphatic residue or an optionally substituted aromatic residue; preferably R⁵ is H or (C₁-C₈)alkyl; more preferably R⁵ is H. R⁶ is H; or an optionally substituted aliphatic residue or an optionally substituted aromatic residue; preferably R⁶ is H or (C₁-C₃)alkyl; more preferably R⁶ is H. R⁷ is H; or an optionally substituted aliphatic residue or an optionally substituted aromatic residue; preferably R⁷ is H or (C₁-C₈)alkyl; more preferably R⁷ is H.

Preferably, Y is selected from the group consisting of NH, S, O and CH₂. More preferably, Y is NH, S or O. In some embodiments, Y is CH₂. In some embodiments, Y is O. In some embodiments, Y is S.

In very preferred embodiments, Y is NH.

### Group R¹:

### First Polyalkylene Glycol Unit R^{F}

R¹ is a first polyalkylene glycol unit R^{F}. The term “first polyalkylene glycol unit”, as used herein, refers to a polyalkylene glycol unit bound to the O atom, which is attached to the phosphorus of the phosphorus (V) moiety. The first polyalkylene glycol unit R^{F} comprises at least 3 alkylene glycol subunits. Preferably, the first polyalkylene glycol unit R^{F} comprises three or more alkylene glycol subunits having the following structure:

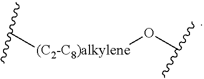

More preferably, the first polyalkylene glycol unit R^{F} comprises three or more alkylene glycol subunits having the following structure:

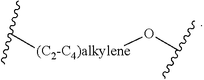

Accordingly, the first polyalkylene glycol unit R^{F} may be a polytetramethyl ene glycol unit, a polypropylene glycol unit, or a polyethylene glycol unit. Still more preferably, the first polyalkylene glycol unit R^{F} comprises three or more alkylene glycol subunits having the following structure:

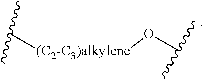

Preferably, the first polyalkylene glycol unit R^{F} comprises of from 3 to 100 alkylene glycol subunits as described herein. More preferably, the first polyalkylene glycol unit R^{F} comprises of from 3 to 50 alkylene glycol subunits as described herein. Still more preferably, the first polyalkylene glycol unit R^{F} comprises of from 3 to 45 alkyleneglycol subunits as described herein. Still more preferably, the first polyalkylene glycol unit R^{F} comprises of from 4 to 40 alkylene glycol subunits as described herein. Still more preferably, the first polyalkylene glycol unit R^{F} comprises of from 6 to 35 alkylene glycol subunits as described herein. Even more preferably, the first polyalkylene glycol unit R^{F} comprises of from 8 to 30 alkylene glycol subunits as described herein.

Preferably, the first polyalkylene glycol unit R^{F} comprises of from 3 to 20 alkylene glycol subunits as described herein. More preferably, the first polyalkylene glycol unit RE comprises of from 3 to 12 alkylene glycol subunits as described herein. Still more preferably, the first polyalkylene glycol unit R^{F} comprises of from 3 to 11 alkylene glycol subunits as described herein.

The first polyalkylene glycol unit R^{F} may be a polyalkylene glycol unit comprising of from 3 to 100, preferably of from 3 to 50, more preferably of from 3 to 45, still more preferably of from 4 to 40, still more preferably of from 6 to 35, even more preferably of from 8 to 30 subunits having the structure:

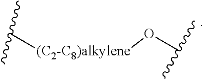

Preferably, the first polyalkylene glycol unit R^{F} may be a polyalkylene glycol unit comprising of from 3 to 100, preferably of from 3 to 50, more preferably of from 3 to 45, still more preferably of from 4 to 40, still more preferably of from 6 to 35, even more preferably of from 8 to 30 subunits having the structure:

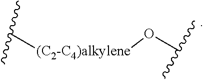

More preferably, the first polyalkylene glycol unit R^{F} may be a polyalkylene glycol unit comprising of from 3 to 100, preferably of from 3 to 50, more preferably of from 3 to 45, still more preferably of from 4 to 40, still more preferably of from 6 to 35, even more preferably of from 8 to 30 subunits having the structure:

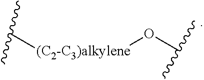

In very preferred embodiments, the first polyalkylene glycol unit R^{F} may be a polyethylene glycol unit comprising of from 3 to 100, preferably of from 3 to 50, more preferably of from 3 to 45, still more preferably of from 4 to 40, still more preferably of from 6 to 35, even more preferably of from 8 to 30 subunits each having the structure:

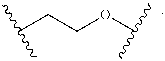 [SMILES: *CCO*]

The first polyalkylene glycol unit R^{F} may be a polyalkylene glycol unit comprising of from 3 to 20, preferably of from 3 to 12, more preferably of from 3 to 11 subunits having the structure:

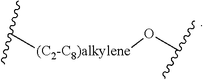

Preferably, the first polyalkylene glycol unit R^{F} may be a polyalkylene glycol unit comprising of from 3 to 20, preferably of from 3 to 12, more preferably of from 3 to 11 subunits having the structure:

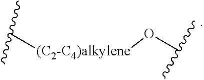

More preferably, the first polyalkylene glycol unit R^{F} may be a polyalkylene glycol unit comprising of from 3 to 20, preferably of from 3 to 12, more preferably of from 3 to 11 subunits having the structure:

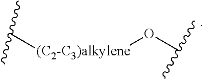

In very preferred embodiments, the first polyalkylene glycol unit R^{F} may be a polyethylene glycol unit comprising of from 3 to 20, preferably of from 3 to 12, more preferably of from 3 to 11 subunits each having the structure:

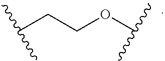 [SMILES: *CCO*]

Preferably, the first polyalkylene glycol unit R^{F} is:

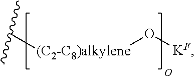

wherein:
[symbol] indicates the position of the 0 attached to the phosphorus;
K^{F} is H or a first capping group; preferably K^{F} is selected from the group consisting of —H (hydrogen), —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₈)alkyl and —(C₂-C₁₀)alkyl-N((C₁-C₈)alkyl)₂; more preferably K^{F} is H; and
is an integer ranging from 3 to 100.

The “first capping group”, when referred to herein, may be any moiety which is capable to function as a terminal group of the first polyalkylene glycol unit. Examples for first capping groups, which can be used in the present disclosure, include —PO₃H, —(C₁-C₁₀)alkyl, -(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₃)alkyl and —(C₂-C₁₀)alkyl-N((C₁-C₃)alkyl)₂. In some embodiments, the first capping group may be —(C₁-C₁₀)alkyl, in particular methyl.

Preferably, K^{F} is H (hydrogen).

The integer o denotes the number of repeating units

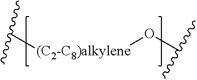

in the first polyalkylene glycol unit. The integer o may range from 3 to 100. Preferably, o ranges from 3 to 50. More preferably, o ranges from 3 to 45. Still more preferably, o ranges from 4 to 40. Still more preferably, o ranges from 6 to 35. Even more preferably, o ranges from 8 to 30. Even more preferably, o ranges from 4 to 16. Even more preferably, o ranges from 8 to 16. Even more preferably, o is 10, 11, 12, 13 or 14. Even more preferably, o is 11, 12 or 13. In preferred embodiments, o is 12 or about 12. Even more preferably, o ranges from 16 to 30. Even more preferably, o ranges from 20 to 28. Even more preferably, o is 22, 23, 24, 25 or 26. Even more preferably, o is 23, 24 or 25. In preferred embodiments, o is 24 or about 24. Preferably, the repeating unit is

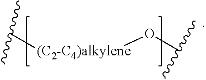

More preferably, the repeating unit is

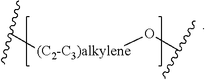

In the first polyalkylene glycol unit, the integer o may range from 3 to 20. Preferably, o ranges from 3 to 12. More preferably, o ranges from 3 to 11. Preferably, the repeating unit is

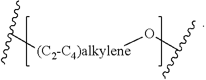

More preferably, the repeating unit is

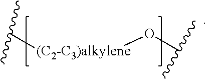

Preferably, the first polyalkylene glycol unit R^{F} comprises ethylene glycol subunits each having the following structure:

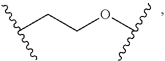 [SMILES: *CCO*]

i.e. this subunit is denoted an ethylene glycol subunit. Accordingly, preferably the first polyalkylene glycol unit is a first polyethylene glycol unit. The first polyethylene glycol unit comprises at least one ethylene glycol subunit.

Preferably, the first polyalkylene glycol unit R^{F} may be a first polyethylene glycol unit comprising of from 3 to 100, preferably of from 3 to 50, more preferably of from 3 to 45, still more preferably of from 4 to 40, still more preferably of from 6 to 35, even more preferably of from 8 to 30 ethylene glycol subunits each having the structure:

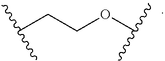 [SMILES: *CCO*]

Preferably, the first polyalkylene glycol unit R^{F} may be a first polyethylene glycol unit comprising of from 3 to 20, preferably of from 3 to 12, more preferably of from 3 to 11 ethylene glycol subunits each having the structure:

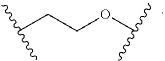 [SMILES: *CCO*]

Preferably, the first polyalkylene glycol unit RE is a first polyethylene glycol unit having the structure:

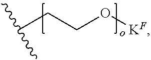

wherein:
[symbol] indicates the position of the O attached to the phosphorus;
K^{F} is H (hydrogen) or a first capping group as described herein; preferably K^{F} is selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₃)alkyl and —(C₂-C₁₀)alkyl-N((C₁-C₃)alkyl)₂; more preferably K^{F} is H; and
is an integer ranging from 3 to 100.
   The integer o denotes the number of repeating units

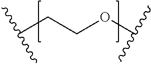

in the first polyethylene glycol unit. The integer o may range from 3 to 100. Preferably, o ranges from 3 to 50. More preferably, o ranges from 3 to 45. Still more preferably, o ranges from 4 to 40. Still more preferably, o ranges from 6 to 35. Even more preferably, o ranges from 8 to 30. Even more preferably, o ranges from 4 to 16. Even more preferably, o ranges from 8 to 16. Even more preferably, o is 10, 11, 12, 13 or 14. Even more preferably, o is 11, 12 or 13. In preferred embodiments, o is 12 or about 12. Even more preferably, o ranges from 16 to 30. Even more preferably, o ranges from 20 to 28. Even more preferably, o is 22, 23, 24, 25 or 26. Even more preferably, o is 23, 24 or 25. In preferred embodiments, o is 24 or about 24.

In the first polyethylene glycol unit, the integer o may range from 3 to 20. Preferably, o ranges from 3 to 12. More preferably, o ranges from 3 to 11.

In general, in the first polyalkylene glycol unit R^{F}, (preferably, first polyethylene glycol unit), polydisperse polyalkylene glycols (preferably, polydisperse polyethylene glycols), monodisperse polyalkylene glycols (preferably, monodisperse polyethylene glycol), and discrete polyalkylene glycols (preferably, discrete polyethylene glycols) can be used. Polydisperse polyalkylene glycols (preferably, polydisperse polyethylene glycols) are a heterogenous mixture of sizes and molecular weights, whereas monodisperse polyalkylene glycols (preferably, monodisperse polyethylene glycols) are typically purified from heterogenous mixtures and therefore provide a single chain length and molecular weight. Preferred first polyalkylene glycols units are discrete polyalkylene glycols (preferably, discrete polyethylene glycols), i.e. compounds that are synthesized in step-wise fashion and not via a polymerization process. Discrete polyalkylene glycols (preferably, discrete polyethylene glycols) provide a single molecule with defined and specified chain length.

The first polyalkylene glycol unit (preferably, first polyethylene glycol unit) provided herein comprises one or multiple polyalkylene glycol chains (preferably, polyethylene glycol chains). The polyalkylene glycol chains (preferably, polyethylene glycol chains) can be linked together, for example, in a linear, branched or star shaped configuration. Optionally, at least one of the polyalkylene glycol chains (preferably, polyethyleneglycol chains) may be derivatized at one end for covalent attachment to the oxygen atom bound to the phosphorus.

The first polyalkylene glycol unit (preferably, first polyethylene glycol unit) will be attached to the conjugate (or intermediate thereof) at the oxygen atom which is bound to the phosphorus. The other terminus (or termini) of the first polyalkylene glycol unit (preferably, first polyethylene glycol unit) will be free and untethered and may take the form of a hydrogen, methoxy, carboxylic acid, alcohol or other suitable functional group, such as e.g. any first capping group as described herein. The methoxy, carboxylic acid, alcohol or other suitable functional group acts as a cap for the terminal polyalkylene glycol subunit (preferably, polyethylene glycol subunit) of the first polyalkylene glycol unit (preferably, first polyethylene glycol unit). By untethered, it is meant that the first polyalkylene glycol unit (preferably, first polyethylene glycol unit) will not be attached at that untethered site to a drug moiety (D), to a receptor binding molecule, or to a component of the linker (L) linking a drug moiety and/or a receptor binding molecule. For those embodiments wherein the first polyalkylene glycol unit (preferably, first polyethylene glycol unit) comprises more than one polyalkylene glycol chain (preferably, polyethylene glycol chain), the multiple polyalkylene glycol chains (preferably, polyethylene glycol chains) may be the same or different chemical moieties (e.g., polyalkylene glycols, in particular polyethylene glycols, of different molecular weight or number of subunits). The multiple first polyalkylene glycol chains (preferably, first polyethylene glycol chains) are attached to the oxygen atom bound to the phosphorus at a single attachment site. The skilled artisan will understand that the first polyalkylene glycol unit (preferably, first polyethylene glycol unit) in addition to comprising repeating polyalkylene glycol subunits (preferably, polyethylene glycol subunits) may also contain non-polyalkylene glycol material (preferably, non-polyethylene glycol material) (e.g., to facilitate coupling of multiple polyalkylene glycol chains (preferably, polyethylene glycol chains) to each other or to facilitate coupling to the oxygen atom bound to the phosphorus. Non-polyalkylene glycol material (preferably, non-polyethelyne glycol material) refers to the atoms in the first polyalkylene glycol unit (preferably, first polyethylene glycol unit) that are not part of the repeating alkylene glycol subunits (preferably, —CH₂CH₂O— subunits). In embodiments provided herein, the first polyalkyleneglycol unit (preferably, first polyethyleneglycol unit) can comprise two monomeric polyalkylene glycol chains (preferably, polyethylene glycol chains) linked to each other via non-polyalkylene glycol (non-polyethylene glycol) elements. In other embodiments provided herein, the first polyalkylene glycol unit (preferably, first polyethylene glycol unit) can comprise two linear polyalkylene glycol chains (preferably, polyethylene glycol chains) attached to a central core that is attached to the oxygen atom bound to the phosphorus (i.e., the polyalkylene glycol unit (preferably, polyethyleneglycol unit) is branched).

There are a number of polyalkylene glycol (preferably, polyethylene glycol) attachment methods available to those skilled in the art, [see, e.g., EP 0 401 384 (coupling PEG to G-CSF); U.S. Pat. No. 5,757,078 (PEGylation of EPO peptides); U.S. Pat. No. 5,672,662 (Polyethylene glycol) and related polymers mono substituted with propionic or butanoic acids and functional derivatives thereof for biotechnical applications); U.S. Pat. No. 6,077,939 (PEGylation of an N-terminal .alpha.-carbon of a peptide); and Veronese (2001) Biomaterials 22:405-417 (Review article on peptide and protein PEGylation)].

In preferred embodiments, the first polyalkylene glycol unit, more preferably the first polyethylene glycol unit, is directly attached to the oxygen atom bound to the phosphorus. In these embodiments, the first polyalkylene glycol unit, preferably first polyethylene glycol unit, does not comprise a functional group for attachment to the oxygen atom bound to the phosphorous, i.e. the oxygen atom is directly bound to a carbon atom of the first polyalkylene glycol unit, preferably to a CH₂ of the first polyethylene glycol unit.

In one group of embodiments, the first polyalkylene glycol unit comprises at least 3 alkylene glycol subunits, still more preferably at least 4 alkylene glycol subunits, still more preferably at least 6 alkylene glycol subunits, even more preferably at least 8 alkylene glycol subunits. In some such embodiments, the first polyalkylene glycol unit comprises no more than about 100 alkylene glycol subunits, preferably no more than about 50 alkylene glycol units, more preferably no more than about 45 alkylene glycol subunits, more preferably no more than about 40 alkylene glycol subunits, more preferably no more than about 35 subunits, even more preferably no more than about 30 alkylene glycol subunits. In any one of these embodiments, the alkylene glycol subunit may be any alkylene glycol subunit as described herein. Preferably, in any one of these embodiments, each alkylene glycol subunit is an ethylene glycol subunit having the following structure:

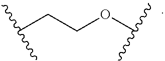 [SMILES: *CCO*]

Preferably, when each alkylene glycol subunit is an ethylene glycol subunit, in any one of these embodiments the first polyalkylene glycol unit is a first polyethylene glycol unit.

In one group of embodiments, the first polyalkylene glycol unit comprises one or more linear polyalkylene glycol chains each having at least 3 alkyleneglycol subunits, still more preferably at least 4 alkyleneglycol subunits, still more preferably at least 6 alkylene glycol subunits, even more preferably at least 8 alkylene glycol subunits. In preferred embodiments, the first polyalkylene glycol unit comprises a combined total of at least 3, still more preferably at least 4, still more preferably at least 6, or even more preferably at least 8 alkylene glycol subunits. In some such embodiments, the first polyalkylene glycol unit comprises no more than a combined total of about 100 alkylene glycol subunits, preferably no more than a combined total of about 50 alkylene glycol subunits, more preferably no more than a combined total of about 45 subunits, still more preferably no more than a combined total of about 40 subunits, still more preferably no more than a combined total of about 35 subunits, even more preferably no more than a combined total of about 30 subunits. In any one of these embodiments, the alkylene glycol subunit may be any alkylene glycol subunit as described herein. Preferably, in any one of these embodiments, each alkylene glycol subunit is an ethylene glycol subunit having the following structure:

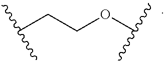 [SMILES: *CCO*]

Preferably, when each alkylene glycol subunit is an ethylene glycol subunit, in any one of these embodiments the first polyalkylene glycol unit is a first polyethylene glycol unit comprising one or more linear polyethylene glycol chains.

In another group of embodiments, the first polyalkylene glycol unit comprises a combined total of from 3 to 100, preferably of from 3 to 50, more preferably of from 3 to 45, still more preferably of from 4 to 40, still more preferably of from 6 to 35, even more preferably of from 8 to 30 alkylene glycol subunits. In any one of these embodiments, the alkylene glycol subunit may be any alkylene glycol subunit as described herein. Preferably, in any one of these embodiments, each alkylene glycol subunit is an ethylene glycol subunit having the following structure:

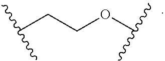 [SMILES: *CCO*]

Preferably, when each alkylene glycol subunit is an ethylene glycol subunit, in any one of these embodiments the first polyalkylene glycol unit is a first polyethylene glycol unit.

In another group of embodiments, the first polyalkylene glycol unit comprises one or more linear polyalkylene glycol chains having a combined total of from 3 to 100, preferably 3 to 50, more preferably 3 to 45, still more preferably 4 to 40, still more preferably 6 to 35, even more preferably 8 to 30 alkylene glycol subunits. In any one of these embodiments, the alkylene glycol subunit may be any alkylene glycol subunit as described herein. Preferably, in any one of these embodiments, each alkylene glycol subunit is an ethylene glycol subunit having the following structure:

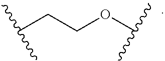 [SMILES: *CCO*]

Preferably, when each alkylene glycol subunit is an ethylene glycol subunit, in any one of these embodiments the first polyalkylene glycol unit is a first polyethylene glycol unit comprising one or more linear polyethylene glycol chains.

In another group of embodiments, the first polyalkylene glycol unit is a linear single polyalkylene glycol chain having at least 3 subunits, still more preferably at least 6 subunits, even more preferably at least 8 subunits. In any one of these embodiments, the alkylene glycol subunit may be any alkylene glycol subunit as described herein. Preferably, in any one of these embodiments, each alkylene glycol subunit is an ethylene glycol subunit having the following structure:

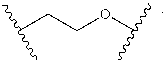 [SMILES: *CCO*]

Preferably, when each alkylene glycol subunit is an ethylene glycol subunit, in any one of these embodiments the first polyalkylene glycol unit is a first polyethylene glycol unit which is a linear single polyethylene glycol chain. Optionally, in any one of these embodiments the linear single polyalkylene glycol chain may be derivatized.

In another group of embodiments, the polyalkylene glycol unit is a linear single polyalkylene glycol chain having from 3 to 100, preferably 3 to 50, more preferably 3 to 45, more preferably 4 to 40, more preferably 6 to 35, more preferably 8 to 30 alkylene glycol subunits. In any one of these embodiments, the alkylene glycol subunit may be any alkylene glycol subunit as described herein. Preferably, in any one of these embodiments, each alkylene glycol subunit is an ethylene glycol subunit having the following structure:

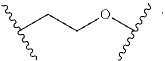 [SMILES: *CCO*]

Preferably, when each alkylene glycol subunit is an ethylene glycol subunit, in any one of these embodiments the first polyalkylene glycol unit is a first polyethylene glycol unit which is a linear single polyethylene glycol chain. Optionally, in any one of these embodiments the linear single polyalkylene glycol chain may be derivatized.

Exemplary linear polyethylene glycol units that can be used as first polyalkylene glycol unit, in particular as a first polyethylene glycol unit, in any one of the embodiments provided herein are as follows:

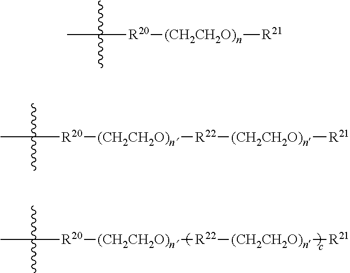

wherein the wavy line indicates the site of attachment to the oxygen atom bound to the phosphorus;
R²⁰ is a PEG attachment unit; preferably, R²⁰ is absent;
R²¹ is a PEG capping unit (herein, R²¹ is also denoted as “K^{F}”);
R²² is a PEG coupling unit (i.e. for coupling multiple PEG subunit chains together;
n is independently selected from 3 to 100, preferably from 3 to 50, more preferably from 3 to 45, more preferably from 4 to 40, still more preferably from 6 to 35, even more preferably from 8 to 30;
e is 2 to 5;
each n′ is independently selected from 1 to 100, preferably from 2 to 50, more preferably from 3 to 45, more preferably from 4 to 40, still more preferably from 6 to 35, even more preferably from 8 to 30. In preferred embodiments, there are at least 3, more preferably at least 4, more preferably at least 6, even more preferably at least 8 ethylene glycol subunits in the polyethylene glycol unit. In some embodiments, there are no more than 100, preferably no more than 50, more preferably no more than 45, more preferably no more than 40, more preferably no more then 35, even more preferably no more than 30 ethylene glycol subunits in the polyethylene glycol unit. When R²⁰ is absent, a (CH₂CH₂O) subunit is directly bound to the oxygen atom, which is attached to the phosphorus.

Preferably, the linear polyethylene glycol unit is

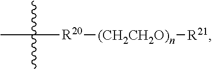

wherein the wavy line indicates the site of attachment to the oxygen atom bound to the phosphorus; R²⁰, R²¹ (also denoted herein as “K^{F}”) and n are as defined herein; more preferably R²⁰ is absent. In preferred embodiments, n is 12 or about 12. In preferred embodiments, n is 24 or about 24. Preferably, R²¹ is H.

The polyethylene glycol attachment unit R²⁰, when present, is part of the first polyethylene glycol unit and acts to link the first polyethylene glycol unit to the oxygen atom bound to the phosphorus. In this regard, the oxygen atom bound to the phosphorus forms a bond with the first polyethylene glycol unit. In exemplary embodiments, the PEG attachment unit R²⁰, when present, is selected from the group consisting of *—(C₁-C₁₀)alkyl-^{#}, *-arylene-^{#}, *—(C₁-C₁₀)alkyl-O—^{#}, *—(C₁-C₁₀)alkyl-C(O)—^{#}, *—(C₁-C₁₀)alkyl-C(O)O—^{#}, *—(C₁-C₁₀)alkyl-NH—^{#}, *—(C₁-C₁₀)alkyl-S-^{#}, *—(C₁-C₁₀)alkyl-C(O)—NH—^{#}, *—(C₁-C₁₀)alkyl-NH—C(O)—^{#}, and *—CH₂—CH₂SO₂—(C₁-C₁₀)alkyl-^{#}; wherein * denotes the attachment point to the oxygen bound to the phosphorus, and # denotes the attachment point to the ethylene glycol unit.

The PEG coupling unit R²², when present, is part of the polyethylene glycol unit and is non-PEG material that acts to connect two or more chains of repeating —CH₂CH₂O-subunits. In exemplary embodiments, the PEG coupling unit R²², when present, is independently selected from the group consisting of *—(C₁-C₁₀)alkyl-C(O)—NH—^{#}, *—(C₁-C₁₀)alkyl-NH—C(O)—^{#}, *—(C₂-C₁₀)alkyl-NH—^{#}, *—(C₂-C₁₀)alkyl-O—^{#}, *—(C₁-C₁₀)alkyl-S-^{#}, or *—(C₂-C₁₀)alkyl-NH-^{#}; wherein * denotes the attachment point to an oxygen atom of an ethylene glycol subunit, and # denotes the attachment point to a carbon atom of another ethylene glycol subunit.

The group R²¹, also denoted herein as “K^{F}”, in exemplary embodiments is H (hydrogen), or may be a first capping group, as described herein; preferably R²¹ is independently selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₃)alkyl and —(C₂-C₁₀)alkyl-N((C₁-C₈)alkyl)₂. In some embodiments R21 may be —C₁-C₁₀)alkyl, in particular methyl. More preferably, R²¹ is H.

Illustrative linear first polyethylene glycol units, which can be used as first polyalkylene glycol units in any one of the embodiments provided herein, are as follows.

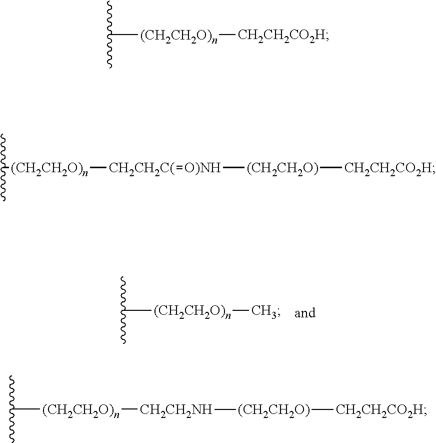

wherein the wavy line indicates the site of attachment to the oxygen atom which is bound to the phosphorus; and each n is from 3 to 100, preferably from 3 to 50, more preferably from 3 to 45, still more preferably from 4 to 40, still more preferably from 6 to 35, even more preferably from 8 to 30. In some embodiments, n is about 12. In some embodiments, n is about 24.

In some embodiments, the first polyalkylene glycol unit is from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the first polyalkylene glycol unit may have at least 6 alkylene glycol subunits or at least 8 alkylene glycol subunits. In some such aspects, the first polyalkylene glycol unit may have at least 6 alkylene glycol subunits or at least 8 alkylene glycol subunits but no more than 100 alkylene glycol subunits, preferably no more than 50 alkylene glycol subunits. In some embodiments, the first polyalkylene glycol unit is a first polyethylene glycol unit being from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the first polyethylene glycol unit may have at least 6 ethylene glycol subunits or at least 8 ethylene glycol subunits. In some such aspects, the first polyethylene glycol unit has at least 6 ethylene glycol subunits or at least 8 ethylene glycol subunits but no more than 100 ethylene glycol subunits, preferably no more than 50 ethylene glycol subunits.

In some embodiments, when R¹ is a first polyalkylene glycol unit R^{F}, there are no other alkylene glycol subunits present in the conjugate of formula (I) (i.e., no alkylene glycol subunits are present in any of the other components of the conjugate, such as e.g. in the linker L as provided herein). In other aspects, when R¹ is a first polyalkylene glycol unit, there are no more than 8, no more than 7, no more than 6, no more than 5, no more than 4, no more than 3, no more than 2 or no more than 1 other alkylene glycol subunits present in the conjugate of formula (I) (i.e., no more than 8, 7, 6, 5, 4, 3, 2, or 1 other alkylene glycol subunits are present in other components of the conjugates, such as e.g. in the linker L as provided herein).

Preferably, in other embodiments, when R¹ is a first polyalkylene glycol unit R^{F}, the conjugate further comprises a second polyalkylene glycol unit R^{S}, as described herein. Preferably, when R¹ is a first polyethylene glycol unit and the conjugate further comprises a second polyalkylene glycol unit R^{S}, the second polyalkylene glycol unit is a second polyethyleneglycol unit, as described herein.

It will be appreciated that when referring to alkylene glycol subunits, in particular ethylene glycol subunits, and depending on context, the number of subunits can represent an average number, e.g., when referring to a population of conjugates or intermediate compounds, and using polydisperse polyalkylene glycols, in particular polydisperse polyethylene glycols.

### “L”: Linker

The present disclosure provides conjugates, where a receptor binding molecule, as described herein, is linked to a drug moiety. In accordance with the present disclosure, the receptor binding molecule may be linked, via the group Y and covalent attachment by a linker L, to the drug moiety. As used herein, a “linker” L is any chemical moiety that is capable of linking a group Y, such as e.g. NH, to another moiety, such as a drug moiety. In this regard, it is again referred to the formula (I) described herein:

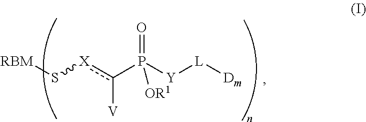

Accordingly, the drug moiety D can be linked to Y through a linker L. In formula (I) RBM, [symbol] V, X, Y, R¹, L, D, m and n are as defined herein. The linker L serves to connect the Y with the drug moiety (D). The linker L is any chemical moiety that is capable of linking Y to the drug moiety D. In particular, the linker L attaches Y to the drug moiety D through covalent bond(s). The linker reagent is a bifunctional or multifunctional moiety which can be used to link a drug moiety D and Y to form conjugates of formula (I). The terms “linker reagent”, “cross-linking reagent”, “linker derived from a cross-linking reagent” and “linker” may be used interchangeably throughout the present disclosure.

Linkers can be susceptible to cleavage (cleavable linker) such as enzymatic cleavage, acid-induced cleavage, photo-induced cleavage and disulfide bond cleavage. Enzymatic cleavage includes, but is not limited to, protease-induced cleavage, peptidase-induced cleavage, esterase-induced cleavage, glycosidase-induced cleavage, phosphatase-induced cleavage, and sulfatase-induced cleavage, preferably at conditions under which the drug moiety and/or the receptor binding molecule remains active. Alternatively, linkers can be substantially resistant to cleavage (e.g., stable linker or non-cleavable linker). In some aspects, the linker may be a procharged linker, a hydrophilic linker, a PEG-based linker, or a dicarboxylic acid based linker. Accordingly, in some embodiments of any one of the antibody drug conjugates disclosed herein the linker (L) is selected from the group consisting of a cleavable linker, a non-cleavable linker, a hydrophilic linker, a PEG-based linker, a procharged linker, a peptidic linker, and a dicarboxylic acid based linker. Preferably, the linker L is a cleavable linker. In some embodiments, the linker L is a non-cleavable linker.

Preferably, as described herein, the linker L is cleavable. In some embodiments, L is a linker susceptible to enzymatic cleavage. In some embodiments, L is an acid-labile linker, a photo-labile linker, a peptidase cleavable linker, a protease cleavable linker, an esterase cleavable linker, a glycosidase cleavable linker, a phosphatase cleavable linker, a sulfatase cleavable linker, a disulfide bond reducible linker, a hydrophilic linker, a procharged linker, a PEG-based linker, or a dicarboxylic acid based linker. Preferably, the linker L is cleavable by a protease, a glucuronidase, a sulfatase, a phosphatase, an esterase, or by disulfide reduction. Preferably, the linker is a peptidase cleavable linker. Other preferred linkers are cleavable by a protease.

A non-cleavable linker is any chemical moiety capable of linking a drug moiety to Y in a stable, covalent manner and does not fall off under the categories listed herein for cleavable linkers. Thus, non-cleavable linkers are substantially resistant to acid-induced cleavage, photo-induced cleavage, peptidase-induced cleavage, protease-induced cleavage, glycosidase-induced cleavage, phosphatase-induced cleavage, esterase-induced cleavage and disulfide bond cleavage. Furthermore, non-cleavable refers to the ability of the chemical bond in the linker or adjoining to the linker to withstand cleavage induced by an acid, photo labile-cleaving agent, a peptidase, a protease, a glycosidase, a phosphatase, an esterase, or a chemical or physiological compound that cleaves a disulfide bond, at conditions under which the drug moiety or the receptor binding molecule does not lose its activity.

Acid-labile linkers are linkers cleavable at acidic pH. For example, certain intracellular compartments, such as endosomes and lysosomes, have an acidic pH (pH 4-5), and provide conditions suitable to cleave acid-labile linkers.

Some linkers can be cleaved by peptidases, i.e. peptidase cleavable linkers. In this regard, certain peptides are readily cleaved inside or outside cells, see e.g. Trout et al., 79 Proc. Natl. Acad. Sci. USA, 626-629 (1982) and Umemoto et al. 43 Int. J. Cancer, 677-684 (1989). Peptides are composed of α-amino acids and peptidic bonds, which chemically are amide bonds between the carboxylate of one amino acid and the amino group of a second amino acid.

Some linkers can be cleaved by esterases, i.e. esterase cleavable linkers. In this regard, certain esters can be cleaved by esterases present inside or outside of cells. Esters are formed by the condensation of a carboxylic acid and an alcohol. Simple esters are esters produced with simple alcohols, such as aliphatic alcohols, and small cyclic and small aromatic alcohols.

Procharged linkers are derived from charged cross-linking reagents that retain their charge after incorporation into an antibody drug conjugate. Examples of procharged linkers can be found in US 2009/0274713.

Preferably, as described herein, the linker L is cleavable. As illustrative examples, the linker may be cleavable by a protease, a glucuronidase, a sulfatase, a phosphatase, an esterase, or by disulfide reduction. Preferably, the linker L is cleavable by a protease. More preferably, the linker is cleavable by a cathepsin, such as, in particular, cathepsin B. The linker may comprise a dipeptide moiety, such as e.g a valine-citrulline moiety or a valine-alanine moiety, which can be cleaved by a cathepsin such as cathepsin B. Accordingly, in some embodiments the linker comprises a valine-citrulline moiety. In some embodiments the linker comprises a valine-alanine moiety. The linker may comprise a cleavage site. The term “cleavage site” may refer to a chemical moiety which is recognized by an enzyme, followed by cleavage, e.g. by way of hydrolysis. As an illustrative example, a cleavage site is a sequence of amino acids, which is recognized by a protease or a peptidase, and hydrolyzed by said protease or peptidase. In some embodiments, the cleavage site is a dipeptide. In some embodiments, the cleavage side is a valine-citrulline moiety. In some embodiments, the cleavage site is a valine-alanine moiety.

### Second Spacer Unit

In preferred embodiments, the linker (L) comprises a second spacer unit -A-which is bound to the —Y—. The second spacer unit serves to connect a —Y— to another part of the linker, when present, or to a drug moiety (-D). As readily appreciated by a person skilled in the art, this depends on whether another part of the linker is present or not. The second spacer unit (-A-) may be any chemical group or moiety which is capable to connect a —Y— to another part of the linker, when present, or to a drug moiety (-D), depending on whether another part of the linker is present or not. In this regard, the —Y—, as described herein, is bonded to the second spacer unit (-A-). The second spacer unit (-A-) may comprise or may be a functional group that is capable to form a bond to another part of the linker, when present, or to the drug moiety (-D). Again, this depends on whether another part of the linker is present or not. Preferably, the functional group, which is capable to form a bond to another part of the linker, or to a drug moiety (-D), is a carbonyl group which is depicted as, e.g.,

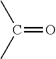

### or —C(O)—.

The second spacer unit may be any spacer known to a person skilled in the art, for example, a straight or branched hydrocarbon-based moiety. The second spacer unit can also comprise cyclic moieties, such as e.g., but not limited to, aromatic moieties. If the second spacer unit is a hydrocarbon-based moiety, the main chain of the second spacer moiety may comprise only carbon atoms but can also contain heteroatoms such as oxygen (O), nitrogen (N) or sulfur (S) atoms, and/or can contain carbonyl groups (C═O). The second spacer unit may comprise or may be, for example, a (C₁-C₂₀) carbon atom chain. In typical embodiments of hydrocarbon-based second spacer units, the spacing moiety comprises between 1 to about 150, 1 to about 100, 1 to about 75, 1 to about 50, or 1 to about 40, or 1 to about 30, or 1 to about 20, including 2, 3, 4, 5, 6, 7, 8, 9, 10, 11, 12, 13, 14, 15, 16, 17, 18 and 19 main chain atoms. A person skilled in the art knows to select suitable second spacer units.

In some embodiments, the second spacer unit (-A-), when present, is selected from the group consisting of *—(C₁-C₁₀)alkylene-C(O) #, *—(C₃-C₈)carbocyclo-C(O)—^{#}, *-arylene-C(O)—^{#}, *—(C₁-C₁₀)alkylene-arylene-C(O)—^{#}, *-arylene-(C₁-C₁₀)alkylene-C(O)—^{#}, *—(C₁-C₁₀)alkylene-(C₃-C₈)carbocyclo-C(O)—^{#}, *—(C₃-C₈)carbocyclo-(C₁-C₁₀)alkylene-C(O)—^{#}, *—(C₃-C₃)heterocyclo-C(O)—^{#}, *—(C₁-C₁₀)alkylene-(C₃-C₈)heterocyclo-C(O)—^{#}, and *—(C₃-C₈)heterocyclo-(C₁-C₁₀)alkylene-C(O)—^{#}; * denotes the attachment point to the —Y—; and # denotes the attachment point to another part of the linker, when present, or to a drug moiety (-D), depending on whether another part of the linker is present or not. Preferably, the second spacer unit (-Aₐ-), when present, is selected from the group consisting of *—(C₃-C₈)carbocyclo-C(O)—^{#}, *-arylene-C(O)—^{#}, and *—(C₃-C₈)heterocyclo-C(O)—^{#}; * denotes the attachment point to the —Y—; and # denotes the attachment point to another part of the linker, when present, or to a drug moiety (-D), depending on whether another part of the linker is present or not.

In other embodiments, the second spacer unit (-A-), when present, may be selected from the group consisting of *—(C₁-C₁₀)alkylene-^{#}, *—(C₃-C₈)carbocyclo-^{#}, *-arylene #, *—(C₁-C₁₀)alkylene-arylene #, *-arylene-(C₁-C₁₀)alkylene-^{#}, *—(C₁-C₁₀)alkylene-(C₃-C₈)carbocyclo-^{#}, *—(C₃-C₈)carbocyclo-(C₁-C₁₀)alkylene-^{#}, *—(C₃-C₈)heterocyclo-^{#}, *—(C₁-C₁₀)alkylene-(C₃-C₈)heterocyclo-^{#}, and *—(C₃-C₈)heterocyclo-(C₁-C₁₀)alkylene-^{#}; * denotes the attachment point to the —Y—; and # denotes the attachment point to another part of the linker, when present, or to a drug moiety (-D), depending on whether another part of the linker is present or not. Preferably, the second spacer unit (-A-), when present, may be selected from the group consisting of *—(C₃-C₈)carbocyclo #, *-arylene-^{#}, and *—(C₃-C₈)heterocyclo-^{#}; * denotes the attachment point to the —Y—; and # denotes the attachment point to another part of the linker, when present, or to a drug moiety (-D), depending on whether another part of the linker is present or not.

Preferably, the second spacer unit -A- is

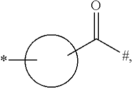

wherein

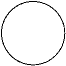

is a five- or six-membered carbocyclic ring; * denotes the attachment point to the —Y—; and # denotes the attachment point to another part of the linker, when present, or to a drug moiety (-D), depending on whether another part of the linker is present or not. The carbocyclic ring may be aromatic or non-aromatic. Preferably, the second spacer unit -A- is

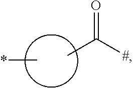

wherein

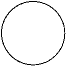

is a five- or six-membered heterocyclic ring comprising 1, 2, or 3 heteroatoms independently selected from the group consisting of N, O and S; * denotes the attachment point to the —Y—; and # denotes the attachment point to another part of the linker, when present, or to a drug moiety (-D), depending on whether another part of the linker is present or not. The heterocyclic ring may be aromatic or non-aromatic.

More preferably,

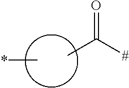

is selected from the group consisting of

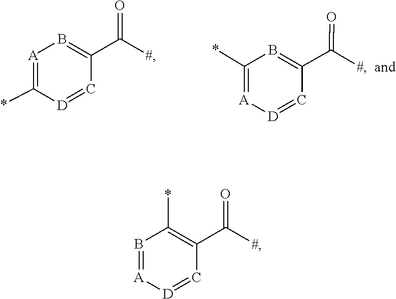

wherein each of A, B, C and D is independently selected from N (nitrogen) and C—H; preferably, at least one of A, B, C and D is C—H; more preferably, at least two of A, B, C and D are C—H; still more preferably, at least three of A, B, C and D are C—H, even more preferably, each of A, B, C and D are C—H; * denotes the attachment point to the —Y—; and # denotes the attachment point to another part of the linker, when present, or to a drug moiety (-D), depending on whether another part of the linker is present or not. Still more preferably,

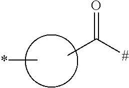

is

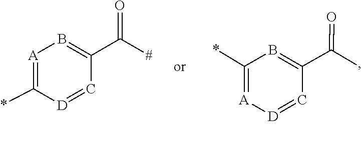

wherein each of A, B, C and D is independently selected from N (nitrogen) and C—H; preferably, at least one of A, B, C and D is C—H; more preferably, at least two of A, B, C and D are C—H; still more preferably, at least three of A, B, C and D are C—H, even more preferably, each of A, B, C and D are C—H; wherein * denotes the attachment point to the —Y—; and # denotes the attachment point to another part of the linker, when present, or to a drug moiety (-D), depending on whether another part of the linker is present or not. Even more preferably,

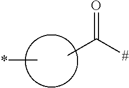

is

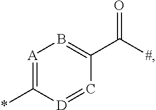

wherein each of A, B, C and D is independently selected from N (nitrogen) and C—H; preferably, at least one of A, B, C and D is C—H; more preferably, at least two of A, B, C and D are C—H; still more preferably, at least three of A, B, C and D are C—H, even more preferably, each of A, B, C and D are C—H; wherein * denotes the attachment point to the —Y—; and # denotes the attachment point to another part of the linker, when present, or to a drug moiety (-D), depending on whether another part of the linker is present or not. In very preferred embodiments, the second spacer unit A is

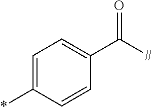

wherein * denotes the attachment point to the —Y—; and # denotes the attachment point to another part of the linker, when present, or to a drug moiety (-D), depending on whether another part of the linker is present or not.

In other embodiments, the second spacer unit (-A-) may be

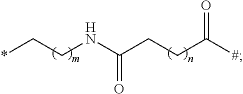

and m and n are each, independently, an integer of e.g. from 0 to 20, 0 to 15, 1 to 10, 1 to 8, 1 to 6, 1 to 4, 1 to 3, 1 to 2, or 1, preferably m is 1 and n is 1; * indicates the position of the —Y—, and # denotes the attachment point to another part of the linker, when present, or to a drug moiety (-D), depending on whether another part of the linker is present or not. Such second spacer unit may be optionally substituted, e.g. with one or two (C₁-C₈)alkyl, in particular at the carbon adjacent to the asterisk (*).

### Group Z

In preferred embodiments, the second spacer unit -A- is a group Z, the group Z having the following structure:

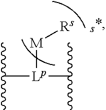

wherein:
L^{P} is a parallel connector unit;
R^{S} is, each independently, a second polyalkylene glycol unit;
M is, each independently, a bond or a moiety that binds R^{S} with L^{P};
s* is an integer ranging from 1 to 4; and
the wavy lines indicate the attachment point to the —Y— and to another part of the linker, when present, or to a drug moiety (-D), depending on whether another part of the linker is present or not.

As indicated in the formula

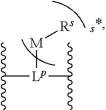

the second polyalkylene glycol unit R^{S} is bonded to the parallel connector unit L^{P} via a suitable moiety M. In some embodiments, M is a bond. In some embodiments, M may be any moiety which is capable to bind a polyalkylene glycol unit with the parallel connector unit L^{P}. As illustrative examples, M may be, each independently, selected from the group consisting of —NH—, —O—, S, —C(O)—O—, —C(O)—NH— and —(C₁-C₁₀)alkylene. Preferably, M is, each independently, selected from the group consisting of —NH—, —O— and —S—. More preferably, each M is —O—.

The integer s* may have a range from 1 to 4. Preferably, the integer s* has a range of from 1 to 3. More preferably, the integer s* is 1 or 2. Even more preferably, the integer s* is 1. The integer s* denotes the number of groups -M-R^{S} which are attached to the parallel connector unit L^{P}.

The parallel connector unit (L^{P}) serves to connect a —Y— to another part of the linker (L) and, via M, to one or more second polyalkylene glycol unit(s), as indicated by the integer s*. Accordingly, when present, L^{P} may be any chemical group or moiety which is capable to connect a —Y— to another part of the linker and, via M, to the second polyalkylene glycol unit. Alternatively, the parallel connector unit (L^{P}) may link the Y to the drug moiety (D), in case no other part of the linker is present, and via M, to the second polyalkylene glycol unit. In this regard, the Y, as described herein, is bonded to the parallel connector unit (L^{P}). The parallel connector unit (L^{P}) may comprise or may be a functional group that is capable to form a bond to another part of the linker (L), or to a drug moiety (D), depending on whether another part of the linker (L) is present or not. Preferably, the functional group, which is capable to form a bond to another art of the linker (L), or to a drug moiety (-D), is a carbonyl group which is depicted as, e.g.,

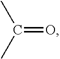

### or —C(O)—, or —(C═O)-.

The parallel connector unit (L^{P}) may be, for example, a straight or branched hydrocarbon-based moiety. The parallel connector unit (L^{P}) can also comprise cyclic moieties. If the parallel connector unit (L^{P}) is a hydrocarbon-based moiety, the main chain of the second spacer moiety may comprise only carbon atoms but can also contain heteroatoms such as oxygen (O), nitrogen (N) or sulfur (S) atoms, and/or can contain carbonyl groups (C═O). The parallel connector unit (L^{P}) may comprise or may be, for example, a (C₁-C₂₀) carbon atom chain. In typical embodiments of hydrocarbon-based parallel connector unit (L^{P}), the linking moiety comprises between 1 to about 150, 1 to about 100, 1 to about 75, 1 to about 50, or 1 to about 40, or 1 to about 30, or 1 to about 20, including 2, 3, 4, 5, 6, 7, 8, 9, 10, 11, 12, 13, 14, 15, 16, 17, 18 and 19 main chain atoms. The parallel connector unit L^{P} is capable to bind to a second polyalkylene glycol unit R^{S} via M. A person skilled in the art knows to select suitable parallel connector units (L^{P}).

In some embodiments, the group Z

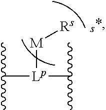

when present, is selected from the group consisting of *—(C₁-C₁₀)alkylene-C(O)—^{#} substituted, each independently, with 1 to 4, preferably 1 to 3, more preferably 1 or 2, still more preferably 1 group(s) -M-R^{S}; *—(C₃-C₈)carbocyclo-C(O)—^{#} substituted, each independently, with 1 to 4, preferably 1 or 2, more preferably 1 group(s) -M-R^{S}; *-arylene-C(O)—^{#} substituted, each independently, with 1 to 4, preferably 1 or 2, more preferably 1 group(s) -M-R^{S}; *—(C₁-C₁₀)alkylene-arylene-C(O)—^{#} substituted, each independently, with 1 to 4, preferably 1 or 2, more preferably 1 group(s) -M-R^{S}; *-arylene-(C₁-C₁₀)alkylene-C(O)—^{#} substituted, each independently, with 1 to 4, preferably 1 or 2, more preferably 1 group(s) -M-R^{S}; *—(C₁-C₁₀)alkylene-(C₃-C₈)carbocyclo-C(O)—# substituted, each independently, with 1 to 4, preferably 1 or 2, more preferably 1 group(s) -M-R^{S}; *—(C₃-C₈)carbocyclo-(C₁-C₁₀)alkylene-C(O)—^{#} substituted, each independently, with 1 to 4, preferably 1 or 2, more preferably 1 group(s) -M-R^{S}; *—(C₃-C₈)heterocyclo-C(O)—^{#} substituted, each independently, with 1 to 4, preferably 1 or 2, more preferably 1 group(s) -M-R^{S}; *—(C₁-C₁₀)alkylene-(C₃-C₈)heterocyclo-C(O)—^{#} substituted, each independently, with 1 to 4, preferably 1 or 2, more preferably 1 group(s) -M-R^{S}; and *—(C₃-C₈)heterocyclo-(C₁-C₁₀)alkylene-C(O)—^{#} substituted, each independently, with 1 to 4, preferably 1 or 2, more preferably 1 group(s) -M-R^{S}; * denotes the attachment point to the —Y—; and # denotes the attachment point to another part of the linker, when present, or to a drug moiety (-D), depending on whether another part of the linker is present or not. Preferably, the group Z

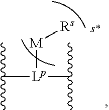

when present, is selected from the group consisting of *—(C₃-C₈)carbocyclo-C(O)—^{#} substituted, each independently, with 1 to 4, preferably 1 or 2, more preferably 1 group(s) -M-R^{S}; *-arylene-C(O)-^{#} substituted, each independently, with 1 to 4, preferably 1 or 2, more preferably 1 group(s) -M-R^{S}; and *—(C₃-C₈)heterocyclo-C(O)—^{#} substituted, each independently, with 1 to 4, preferably 1 or 2, more preferably 1 group(s) -M-R^{S}; * denotes the attachment point to the —Y—; and # denotes the attachment point to another part of the linker, when present, or to a drug moiety (-D), depending on whether another part of the linker is present or not.

In other embodiments, the group Z

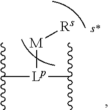

when present, may be selected from the group consisting of *—(C₁-C₁₀)alkylene-^{#} substituted, each independently, with 1 to 4, preferably 1 to 3, more preferably 1 or 2, still more preferably 1 group(s) -M-R^{S}; *—(C₃-C₃)carbocyclo-^{#} substituted, each independently, with 1 to 4, preferably 1 or 2, more preferably 1 group(s) -M-R^{S}; *-arylene-^{#} substituted, each independently, with 1 to 4, preferably 1 or 2, more preferably 1 group(s) -M-R^{S}; *—(C₁-C₁₀)alkylene-arylene-^{#} substituted, each independently, with 1 to 4, preferably 1 or 2, more preferably 1 group(s) -M-R^{S}; *-arylene-(C₁-C₁₀)alkylene-^{#} substituted, each independently, with 1 to 4, preferably 1 or 2, more preferably 1 group(s) -M-R^{S}; *—(C₁-C₁₀)alkylene-(C₃-C₈)carbocyclo-^{#} substituted, each independently, with 1 to 4, preferably 1 or 2, more preferably 1 group(s) -M-R^{S}; *—(C₃-C₈)carbocyclo-(C₁-C₁₀)alkylene-^{#} substituted, each independently, with 1 to 4, preferably 1 or 2, more preferably 1 group(s) -M-R^{S}; *—(C₃-C₈)heterocyclo-^{#} substituted, each independently, with 1 to 4, preferably 1 or 2, more preferably 1 group(s) -M-R^{S}; *—(C₁-C₁₀)alkylene-(C₃-C₈)heterocyclo-^{#} substituted, each independently, with 1 to 4, preferably 1 or 2, more preferably 1 group(s) -M-R^{S}; and *—(C₃-C₈)heterocyclo-(C₁-C₁₀)alkylene-^{#} substituted, each independently, with 1 to 4, preferably 1 or 2, more preferably 1 group(s) -M-R^{S}; * denotes the attachment point to the —Y—; and # denotes the attachment point to another part of the linker, when present, or to a drug moiety (-D), depending on whether another part of the linker is present or not. Preferably, the group Z,

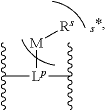

when present, may be selected from the group consisting of *—(C₃-C₃)carbocyclo-^{#} substituted, each independently, with 1 to 4, preferably 1 or 2, more preferably 1 group(s) -M-R^{S}; *-arylene-^{#} substituted, each independently, with 1 to 4, preferably 1 or 2, more preferably 1 group(s) -M-R^{S}; and *—(C₃-C₈)heterocyclo-^{#} substituted, each independently, with 1 to 4, preferably 1 or 2, more preferably 1 group(s) -M-R^{S}; * denotes the attachment point to the —Y—; and # denotes the attachment point to another part of the linker, when present, or to a drug moiety (-D), depending on whether another part of the linker is present or not.

In some embodiments, the L^{P} in the group Z

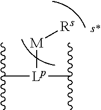

may be one or more amino acid, which comprises a suitable moiety M so that a second polyalkylene glycol unit can be attached; preferably s* is 1. The amino acid may be a natural or non-natural amino acid. For example, the amino acid may be selected from the group consisting of lysine, glutamic acid, aspartic acid, serine, tyrosine, threonine, cysteine, selenocysteine, glycine, and homoalanine. In particular, the amino acid may be selected from the group consisting of tyrosine, serine, threonine, glutamic acid, lysine and glycine. Other suitable moieties L^{P} may be selected from the group consisting of amino alcohols, amino aldehydes, and polyamines. Suitable amino acids and further groups for attaching a polyalkylene glycol unit are described, e.g. in WO 2015/057699.

Preferably, the group Z

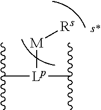

is

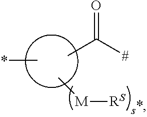

wherein

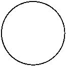

is a five- or six-membered carbocyclic ring; the carbocyclic ring may be aromatic or non-aromatic; M is, each independently, as defined herein; preferably, each M is —O—; R^{S} is, each independently, a second polyalkylene glycol unit as defined herein; preferably, each R^{S} is, independently, a second polyethylene glycol unit as defined herein; s* is an integer ranging from 1 to 3, preferably s* is 1 or 2, more preferably s* is 1; * denotes the attachment point to the —Y—; and # denotes the attachment point to another part of the linker, when present, or to a drug moiety (-D), depending on whether another part of the linker is present or not;

More preferably,

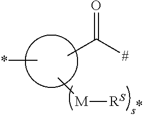

is selected from the group consisting of

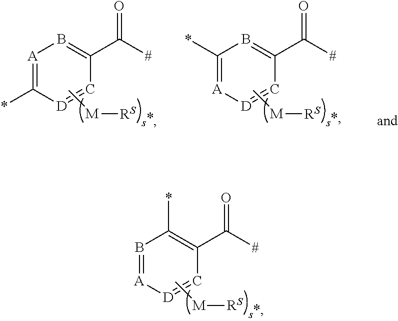

wherein each of A, B, C and D is C—H; R^{S} is, each independently, a second poly(alkylene)glycol unit as defined herein; preferably, each R^{S} is, independently, a second polyethylene glycol unit as defined herein; M is, each independently, as defined herein; preferably each M is —O—; the integer s* is 1 or 2, preferably s* is 1; as indicated by the

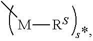

in two C—H, independently, the H is replaced with -M-R^{S} when s* is 2, or in one C—H the H is replaced with -M-R^{S} when s* is 1; * denotes the attachment point to the —Y—; and # denotes the attachment point to another part of the linker (e.g., an amino acid unit —W_{w}—), when present, or to a drug moiety (-D), depending on whether another part of the linker is present or not. Still more preferably,

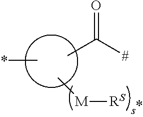

is

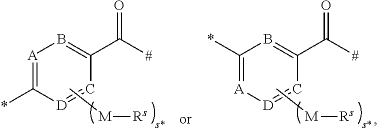

wherein each of A, B, C and D is C—H; R^{S} is, each independently, a second poly(alkylene)glycol unit as defined herein; preferably, each R^{S} is, independently, a second polyethylene glycol unit as defined herein; M is, each independently, as defined herein; preferably each M is —O—; the integer s* is 1 or 2, preferably s* is 1; as indicated by the

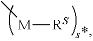

in two C—H, independently the H is replaced with -M-R^{S} when s* is 2, or in one C—H the H is replaced with -M-R^{S} when s* is 1; * denotes the attachment point to the —Y—; and # denotes the attachment point to another part of the linker, when present, or to a drug moiety (-D), depending on whether another part of the linker is present or not. Still more preferably,

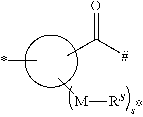

is

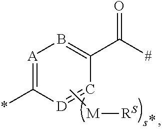

wherein each of A, B, C and D is C—H; R^{S} is, each independently, a second poly(alkylene)glycol unit as defined herein; preferably, each R^{S} is, independently, a second polyethylene glycol unit as defined herein; M is, each independently, as defined herein; preferably each M is —O—; the integer s* is 1 or 2, preferably s* is 1; as indicated by the

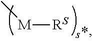

in two C—H, independently, the H is replaced with -M-R^{S} when s* is 2, or in one C—H the H is replaced with -M-R^{S} when s* is 1; * denotes the attachment point to the —Y—; and # denotes the attachment point to another part of the linker, when present, or to a drug moiety (-D), depending on whether another part of the linker is present or not. In very preferred embodiments, the group Z

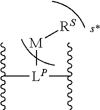

is

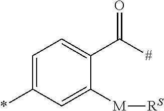

wherein R^{S} is a second polyalkylene glycol unit as described herein; preferably, R^{S} is a second polyethylene glycol unit as defined herein; M is as described herein; preferably M is —O—; * denotes the attachment point to the —Y—; and # denotes the attachment point to another part of the linker, when present, or to a drug moiety (-D), depending on whether another part of the linker is present or not.

In some embodiments, the group Z

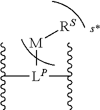

is

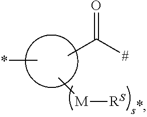

wherein

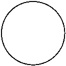

is a five- or six-membered heterocyclic ring comprising 1 or 2 heteroatoms independently selected from the group consisting of N, O or S; the heterocyclic ring may be aromatic or non-aromatic; M is, each independently, as defined herein; preferably each M is —O—; R^{S} is, each independently, a second poly(alkylene) glycol unit as defined herein; preferably, each R^{S} is, independently, a second polyethylene glycol unit as defined herein; s* is 1 or 2 (in particular, in case of a six-membered heterocyclic ring), preferably s* is 1 (in particular, in case of a five-membered or six-membered heterocyclic ring); * denotes the attachment point to the —Y—; and # denotes the attachment point to another part of the linker, when present, or to a drug moiety (-D), depending on whether another part of the linker is present or not.

In some embodiments,

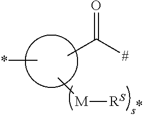

is selected from the group consisting of

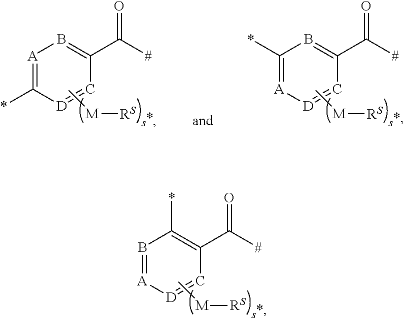

wherein three of A, B, C and D are, independently, C—H, and one of A, B, C and D is, independently, N; R^{S} is, each independently, a second poly(alkylene)glycol unit as defined herein; preferably, each R^{S} is, independently, a second polyethylene glycol unit as defined herein; M is, each independently, as defined herein; preferably each M is —O—; the integer s* is 1 or 2, preferably s* is 1; as indicated by the

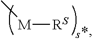

in two C—H, independently, the H is replaced with -M-R^{S} when s* is 2, or in one C—H the H is replaced with -M-R^{S} when s* is 1; * denotes the attachment point to the —Y—; and # denotes the attachment point to another part of the linker, when present, or to a drug moiety (-D), depending on whether another part of the linker is present or not. In some embodiments,

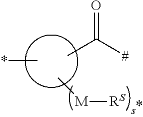

is

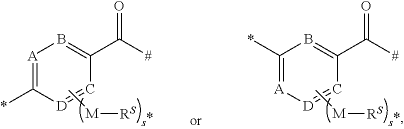

wherein three of A, B, C and D are, independently, C—H, and one of A, B, C and D is, independently, N; R^{S} is, each independently, a second poly(alkylene)glycol unit as defined herein; preferably, each R^{S} is, independently, a second polyethylene glycol unit as defined herein; M is, each independently, as defined herein; preferably each M is —O—; the integer s* is 1 or 2, preferably s* is 1; as indicated by the

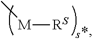

in two C—H, independently the H is replaced with -M-R^{S} when s* is 2, or in one C—H the H is replaced with -M-R^{S} when s* is 1; * denotes the attachment point to the —Y—; and # denotes the attachment point to another part of the linker, when present, or to a drug moiety (-D), depending on whether another part of the linker is present or not. In some embodiments,

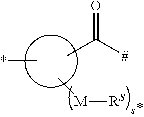

is

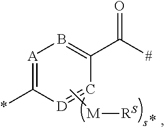

wherein three of A, B, C and D are, independently, C—H, and one of A, B, C and D is, independently, N; R^{S} is, each independently, a second poly(alkylene)glycol unit as defined herein; preferably, each R^{S} is, independently, a second polyethylene glycol unit as defined herein; M is, each independently, as defined herein; preferably each M is —O—; the integer s* is 1 or 2, preferably s* is 1; as indicated by the

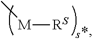

in two C—H, independently, the H is replaced with -M-R^{S} when s* is 2, or in one C—H the H is replaced with -M-R^{S} when s* is 1; * denotes the attachment point to the —Y—; and # denotes the attachment point to another part of the linker, when present, or to a drug moiety (-D), depending on whether another part of the linker is present or not.

In some embodiments,

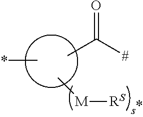

is selected from the group consisting of

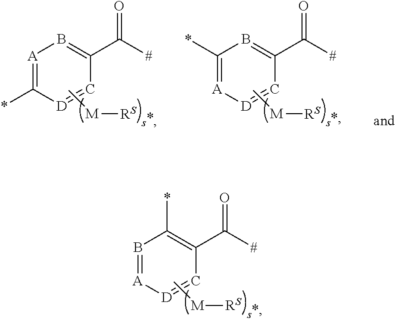

wherein two of A, B, C and D are, independently, C—H, and two of A, B, C and D are, independently, N; R^{S} is, each independently, a second poly(alkylene)glycol unit as defined herein; preferably, each R^{S} is, independently, a second polyethylene glycol unit as defined herein; M is, each independently, as defined herein: preferably each M is —O—; the integer s* is 1 or 2, preferably s* is 1; as indicated by the

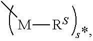

in two C—H, independently, the H is replaced with -M-R^{S} when s* is 2, or in one C—H the H is replaced with R^{S} when s* is 1; * denotes the attachment point to the —Y—; and # denotes the attachment point to another part of the linker, when present, or to a drug moiety (-D), depending on whether another part of the linker is present or not. In some embodiments,

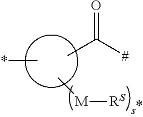

is

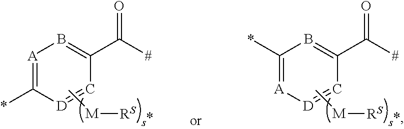

wherein two of A, B, C and D are, independently, C—H, and two of A, B, C and D are, independently, N; R^{S} is, each independently, a second polyalkylene glycol unit as defined herein; preferably, each R^{S} is, independently, a second polyethylene glycol unit as defined herein; M is, each independently, as defined herein; preferably each M is —O—; the integer s* is 1 or 2, preferably s* is 1; as indicated by the

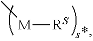

in two C—H, independently the H is replaced with -M-R^{S} when s* is 2, or in one C—H the H is replaced with -M-R^{S} when s* is 1; * denotes the attachment point to the —Y—; and # denotes the attachment point to another part of the linker, when present, or to a drug moiety (-D), depending on whether another part of the linker is present or not. In some embodiments,

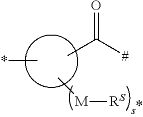

is

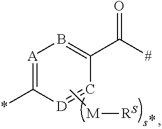

wherein two of A, B, C and D are, independently, C—H, and two of A, B, C and D are, independently, N; R^{S} is, each independently, a second poly(alkylene)glycol unit as defined herein; preferably, each R^{S} is, independently, a second polyethylene glycol unit as defined herein; M is, each independently, as defined herein; preferably each M is —O—; the integer s* is 1 or 2, preferably s* is 1; as indicated by the

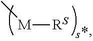

in two C—H, independently, the H is replaced with -M-R^{S} when s* is 2, or in one C—H the H is replaced with -M-R^{S} when s* is 1; * denotes the attachment point to the —Y—; and # denotes the attachment point to another part of the linker, when present, or to a drug moiety (-D), depending on whether another part of the linker is present or not.

### Second Polyalkylene Glycol Unit R^{S}

The term “second polyalkylene glycol unit”, as used herein, refers to a polyalkylene glycol unit bound to the parallel connector unit (L^{P}), which is present in the group Z, via M. The second polyalkylene glycol unit comprises at least one alkylene glycol subunit. Preferably, the second polyalkylene glycol unit R^{S} comprises one or more alkylene glycol subunits having the following structure:

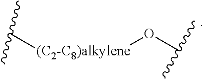

More preferably, the second polyalkylene glycol unit R^{S} comprises one or more alkylene glycol subunits having the following structure:

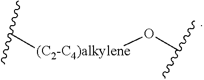

Accordingly, the second polyalkylene glycol unit R^{S} may be a poly(tetramethyleneglycol) unit, a poly(propyleneglycol) unit, or a poly(ethylenglycol) unit. Still more preferably, the second polyalkylene glycol unit comprises one or more alkylene glycol subunits having the following structure:

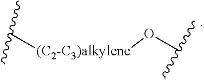

Preferably, the second polyalkylene glycol unit R^{S}, each independently, comprises of from 1 to 100 alkylene glycol subunits as described herein. More preferably, the second polyalklyene glycol unit R^{S}, each independently, comprises of from 2 to 50 alkylene glycol subunits. Still more preferably, the second polyalkylene glycol unit comprises, each independently, of from 3 to 45 alkylene glycol subunits as described herein. Still more preferably, the second polyalkylene glycol unit, each independently, comprises of from 4 to 40 alkylene glycol subunits as described herein. Still more preferably, the second polyalkylene glycol unit, each independently, comprises of from 6 to 35 alkylene glycol subunits as described herein. Even more preferably, the second polyalkylene glycol unit, each independently comprises of from 8 to 30 alkylene glycol subunits as described herein.

Preferably, the second polyalkylene glycol unit R^{S}, each independently, comprises of from 1 to 20 alkylene glycol subunits as described herein. More preferably, the second polyalkylene glycol unit R^{S} comprises, each independently, of from 2 to 12 alkylene glycol subunits. Still more preferably, the second polyalkylene glycol unit comprises, each independently, of from 3 to 11 alkylene glycol subunits as described herein.

The second polyalkylene glycol unit R^{S} may be, each independently, a polyalkylene glycol unit comprising of from 1 to 100, preferably of from 2 to 50, more preferably of from 3 to 45, still more preferably of from 4 to 40, still more preferably of from 6 to 35, even more preferably of from 8 to 30 subunits having the structure:

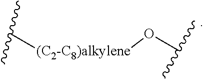

Preferably, the second polyalkylene glycol unit R^{S} may be, each independently, a polyalkylene glycol unit comprising of from 1 to 100, preferably of from 2 to 50, more preferably of from 3 to 45, more preferably of from 4 to 40, still more preferably of from 6 to 35, even more preferably of from 8 to 30 subunits having the structure:

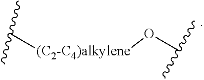

More preferably, the second polyalkylene glycol unit R^{S} may be, each independently, a polyalkylene glycol unit comprising of from 1 to 100, more preferably of from 2 to 50, still more preferably of from 3 to 45, still more preferably of from 4 to 40, still more preferably of from 6 to 35, even more preferably of from 8 to 30 subunits having the structure:

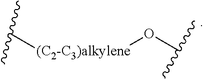

In very preferred embodiments, the second polyalkylene glycol unit R^{S} may be, each independently, a polyethylene glycol unit comprising of from 1 to 100, preferably of from 2 to 50, more preferably 3 to 45, still more preferably of from 4 to 40, still more preferably of from 6 to 35, even more preferably of from 8 to 30 subunits having the structure:

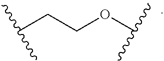 [SMILES: *CCO*]

The second polyalkylene glycol unit R^{S} may be, each independently, a polyalkylene glycol unit comprising of from 1 to 20, preferably of from 2 to 12, more preferably of from 3 to 11 subunits having the structure:

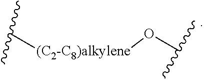

Preferably, the second polyalkylene glycol unit R^{S} may be, each independently, a polyalkylene glycol unit comprising of from 1 to 20, preferably of from 2 to 12, more preferably of from 3 to 11 subunits having the structure:

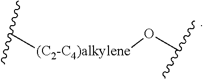

More preferably, the second polyalkylene glycol unit R^{S} may be, each independently, a polyalkylene glycol unit comprising of from 1 to 20, more preferably of from 2 to 12, still more preferably of from 3 to 11 subunits having the structure:

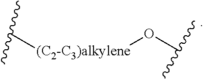

In very preferred embodiments, the second polyalkylene glycol unit R^{S} may be, each independently, a polyethylene glycol unit comprising of from 1 to 20, preferably of from 2 to 12, more preferably 3 to 11 subunits having the structure:

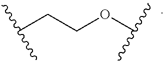 [SMILES: *CCO*]

Preferably, the second polyalkylene glycol unit R^{S} is, each independently,

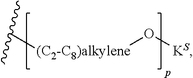

wherein:
[symbol] indicates the position of the M in group Z;
K^{S} is H or a second capping group; preferably K^{S} is selected from the group consisting of —H (hydrogen), —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₃)alkyl and —(C₂-C₁₀)alkyl-N((C₁-C₃)alkyl)₂; more preferably K^{S} is H; and
p is an integer ranging from 1 to 100.

The “second capping group”, when referred to herein, may be any moiety which is capable to function as a terminal group of the second polyalkylene glycol unit. Examples for second capping groups, which can be used in the present disclosure, include —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, -(C₂-C₁₀)alkyl-NH(C₁-C₃)alkyl and —(C₂-C₁₀)alkyl-N((C₁-C₃)alkyl)₂. In some embodiments, the first capping group may be —(C₁-C₁₀)alkyl, in particular methyl.

Preferably, K^{S} is H (hydrogen).

The integer p denotes the number of repeating units

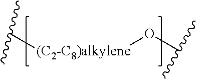

in the second polyalkylene glycol unit. The integer p may range from 1 to 100. Preferably, p ranges from 2 to 50. More preferably, p ranges from 3 to 45. More preferably, p ranges from 4 to 40. Still more preferably, p ranges from 6 to 35. Even more preferably, p ranges from 8 to 30. Even more preferably, p ranges from 4 to 16. Even more preferably, p ranges from 8 to 16. Even more preferably, p is 10, 11, 12, 13 or 14. Even more preferably, p is 11, 12 or 13. In preferred embodiments, p is 12 or about 12. Even more preferably, p ranges from 16 to 30. Even more preferably, p ranges from 20 to 28. Even more preferably, p is 22, 23, 24, 25 or 26. Even more preferably, p is 23, 24 or 25. In preferred embodiments, p is 24 or about 24. Preferably, the repeating unit is

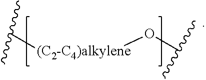

More preferably, the repeating unit is

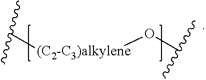

In the second polyalkylene glycol unit, the integer p may range from 1 to 20. Preferably, p ranges from 2 to 12. More preferably, p ranges from 3 to 11. Preferably, the repeating unit is

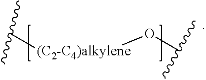

More preferably, the repeating unit is

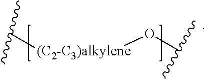

Preferably, the second polyalkylene glycol unit R^{S} comprises ethylene glycol subunits each having the following structure:

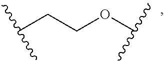 [SMILES: *CCO*]

i.e. this subunit is denoted an “ethylene glycol subunit”. Accordingly, preferably the second polyalkylene glycol unit is a second polyethylene glycol unit. The second polyethylene glycol unit comprises at least one ethylene glycol subunit.

Preferably, the second polyalkylene glycol unit R^{S} may be, each independently, a second polyethylene glycol unit comprising of from 1 to 100, preferably of from 2 to 50, more preferably of from 3 to 45, more preferably of from 4 to 40, still more preferably of from 6 to 35, even more preferably of from 8 to 30 subunits having the structure:

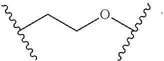 [SMILES: *CCO*]

Preferably, the second polyalkylene glycol unit R^{S} may be, each independently, a second polyethylene glycol unit comprising of from 1 to 20, preferably of from 2 to 12, more preferably of from 3 to 11 subunits having the structure:

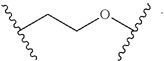 [SMILES: *CCO*]

Preferably, the second polyalkylene glycol unit R^{S} is, each independently, a second polyethylene glycol unit having the structure:

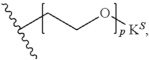

[symbol] indicates the position of the M in group Z;
K^{S} is H (hydrogen) or a second capping group as described herein; preferably K^{S} is selected from the group consisting of —H (hydrogen), —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₃)alkyl and —(C₂-C₁₀)alkyl-N((C₁-C₃)alkyl)₂; more preferably K^{S} is H; and
p is an integer ranging from 1 to 100.

The integer p denotes the number of repeating units

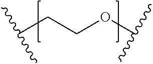

in the second polyethylene glycol unit. The integer p may range from 1 to 100. Preferably, p ranges from 2 to 50. More preferably, p ranges from 3 to 45. Still more preferably, p ranges from 4 to 40. Still more preferably, p ranges from 6 to 35. Even more preferably, p ranges from 8 to 30. Even more preferably, p ranges from 4 to 16. Even more preferably, p ranges from 8 to 16. Even more preferably, p is 10, 11, 12, 13 or 14. Even more preferably, p is 11, 12 or 13. In preferred embodiments, p is 12 or about 12. Even more preferably, p ranges from 16 to 30. Even more preferably, p ranges from 20 to 28. Even more preferably, p is 22, 23, 24, 25 or 26. Even more preferably, p is 23, 24 or 25. In preferred embodiments, p is 24 or about 24.

In the second polyethylene glycol unit, the integer p may range from 1 to 20. Preferably, p ranges from 2 to 12. More preferably, p ranges from 3 to 11.

In general, in the second polyalkylene glycol unit R^{S}, (preferably, second polyethylene glycol unit), polydisperse polyalkylene glycols (preferably, polydisperse polyethylene glycols), monodisperse polyalkylene glycols (preferably, monodisperse polyethylene glycol), and discrete polyalkylene glycols (preferably, discrete polyethylene glycols) can be used. Polydisperse polyalkylene glycols (preferably, polydisperse polyethylene glycols) are a heterogenous mixture of sizes and molecular weights, whereas monodisperse polyalkylene glycols (preferably, monodisperse polyethylene glycols) are typically purified from heterogenous mixtures and therefore provide a single chain length and molecular weight. Preferred second polyalkylene glycols units are discrete polyalkylene glycols (preferably, discrete polyethylene glycols), i.e. compounds that are synthesized in step-wise fashion and not via a polymerization process. Discrete polyalkylene glycols (preferably, discrete polyethylene glycols) provide a single molecule with defined and specified chain length.

The second polyalkylene glycol unit (preferably, second polyethylene glycol unit) provided herein comprises one or multiple polyalkylene glycol chains (preferably, polyethylene glycol chains). The polyalkylene glycol chains (preferably, polyethylene glycol chains) can be linked together, for example, in a linear, branched or star shaped configuration. Optionally, at least one of the polyalkylene glycol chains (preferably, polyethyleneglycol chains) may be derivatized at one end for covalent attachment to the M in group Z.

The second polyalkylene glycol unit (preferably, second polyethylene glycol unit) will be attached to the conjugate (or intermediate thereof) at the M in group Z. The other terminus (or termini) of the second polyalkylene glycol unit (preferably, second polyethylene glycol unit) will be free and untethered and may take the form of a hydrogen, methoxy, carboxylic acid, alcohol or other suitable functional group, such as e.g. any second capping group as described herein. The methoxy, carboxylic acid, alcohol or other suitable functional group acts as a cap for the terminal polyalkylene glycol subunit (preferably, polyethylene glycol subunit) of the second polyalkylene glycol unit (preferably, second polyethylene glycol unit). By untethered, it is meant that the second polyalkylene glycol unit (preferably, second polyethylene glycol unit) will not be attached at that untethered site to a drug moiety (D), to a receptor binding molecule, or to a component of the linker (L) linking a drug moiety and/or a receptor binding molecule. For those embodiments wherein the second polyalkylene glycol unit (preferably, second polyethylene glycol unit) comprises more than one polyalkylene glycol chain (preferably, polyethylene glycol chain), the multiple polyalkylene glycol chains (preferably, polyethylene glycol chains) may be the same or different chemical moieties (e.g., polyalkylene glycols, in particular polyethylene glycols, of different molecular weight or number of subunits). The multiple second polyalkylene glycol chains (preferably, second polyethylene glycol chains) are attached to the M in group Z at a single attachment site. The skilled artisan will understand that the second polyalkylene glycol unit (preferably, second polyethylene glycol unit) in addition to comprising repeating polyalkylene glycol subunits (preferably, polyethylene glycol subunits) may also contain non-polyalkylene glycol material (preferably, non-polyethylene glycol material) (e.g., to facilitate coupling of multiple polyalkylene glycol chains (preferably, polyethylene glycol chains) to each other or to facilitate coupling to the M in group Z. Non-polyalkylene glycol material (preferably, non-polyethelyne glycol material) refers to the atoms in the second polyalkylene glycol unit (preferably, second polyethylene glycol unit) that are not part of the repeating alkylene glycol subunits (preferably, —CH₂CH₂O— subunits). In embodiments provided herein, the second polyalkyleneglycol unit (preferably, second polyethyleneglycol unit) can comprise two monomeric polyalkylene glycol chains (preferably, polyethylene glycol chains) linked to each other via non-polyalkylene glycol (preferably, non-polyethylene glycol) elements. In other embodiments provided herein, the second polyalkylene glycol unit (preferably, second polyethylene glycol unit) can comprise two linear polyalkylene glycol chains (preferably, polyethylene glycol chains) attached to a central core that is attached to the M in group Z (i.e., the polyalkylene glycol unit (preferably, polyethyleneglycol unit) is branched).

There are a number of polyalkylene glycol (preferably, polyethylene glycol) attachment methods available to those skilled in the art, [see, e.g., EP 0 401 384 (coupling PEG to G-CSF); U.S. Pat. No. 5,757,078 (PEGylation of EPO peptides); U.S. Pat. No. 5,672,662 (Polyethylene glycol) and related polymers mono substituted with propionic or butanoic acids and functional derivatives thereof for biotechnical applications); U.S. Pat. No. 6,077,939 (PEGylation of an N-terminal .alpha.-carbon of a peptide); and Veronese (2001) Biomaterials 22:405-417 (Review article on peptide and protein PEGylation)].

For example, polyalkylene glycol (preferably, polyethylene glycol) may be covalently bound to amino acid residues via a reactive group. Reactive groups are those to which an activated polyalkylene glycol molecule (preferably, polyethylene glycol molecule) may be bound (e.g., a free amino or carboxyl group). For example, N-terminal amino acid residues and lysine (K) residues have a free amino group; and C-terminal amino acid residues have a free carboxyl group. Sulfhydryl groups (e.g., as found on cysteine residues) may also be used as a reactive group for attaching polyalkylene glycol (preferably, polyethylene glycol). In addition, enzyme-assisted methods for introducing activated groups (e.g., hydrazide, aldehyde, and aromatic-amino groups) specifically at the C-terminus of a polypeptide have been described (see Schwarz, et al. (1990) Methods Enzymol. 184:160; Rose, et al. (1991) Bioconjugate Chem. 2: 154; and Gaertner, et al. (1994) J. Biol. Chem. 269:7224].

In some embodiments, at least one of the polyalkylene glycol chains (preferably, polyethylene glycol chains) that make up the second polyalkylene glycol unit (preferably, second polyethylene glycol unit) may be functionalized so that it can attach to the M in group Z, or to the parallel connector unit L^{P} in group Z when M is a bond. Functionalization can be, for example, via an amine, thiol, NHS ester, alkyne, azide, carbonyl, or other functional group. The polyalkylene glycol unit (preferably, polyethylene glycol unit) can further comprise non-polyalkylene glycol material (preferably, non-polyethylene glycol material, i.e., material not comprised of —CH₂CH₂O—) to facilitate coupling to the M in group Z or to the parallel connector unit, when M is a bond, or to facilitate coupling of two or more polyalkylene glycol chains (preferably, polyethylene glycol chains).

In preferred embodiments, the second polyalkylene glycol unit, more preferably the second polyethylene glycol unit, is directly attached to the M in group Z. In these embodiments, the second polyalkylene glycol unit, preferably second polyethylene glycol unit, does not comprise a functional group for attachment to the M in group Z, i.e. the M is directly bound to a carbon atom of the second polyalkylene glycol unit, more preferably to a CH₂ of the second polyethylene glycol unit. Preferably, in any one of these embodiments M is not a bond.

In one group of embodiments, the second polyalkylene glycol unit comprises at least 1 alkylene glycol subunit, preferably at least 2 alkylene glycol subunits, more preferably at least 3 alkylene glycol subunits, still more preferably at least 4 alkylene glycol subunits, still more preferably at least 6 alkylene glycol subunits, even more preferably at least 8 alkylene glycol subunits. In some such embodiments, the second polyalkylene glycol unit comprises no more than about 100 alkylene glycol subunits, preferably no more than about 50 alkylene glycol units, more preferably no more than about 45 alkylene glycol subunits, more preferably no more than about 40 alkylene glycol subunits, more preferably no more than about 35 subunits, even more preferably no more than about 30 alkylene glycol subunits. In any one of these embodiments, the alkylene glycol subunit may be any alkylene glycol subunit as described herein. Preferably, in any one of these embodiments, each alkylene glycol subunit is an ethylene glycol subunit having the following structure:

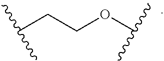 [SMILES: *CCO*]

Preferably, when each alkylene glycol subunit is an ethylene glycol subunit, in any one of these embodiments the second polyalkylene glycol unit is a second polyethylene glycol unit.

In one group of embodiments, the second polyalkylene glycol unit comprises one or more linear polyalkylene glycol chains each having at least 1 alkyleneglycol subunits, preferably at least 2 alkyleneglycol subunits, more preferably at least 3 alkyleneglycol subunits, still more preferably at least 4 alkyleneglycol subunits, still more preferably at least 6 alkylene glycol subunits, even more preferably at least 8 alkylene glycol subunits. In preferred embodiments, the second polyalkylene glycol unit comprises a combined total of at least 1 alkylene glycol subunits, preferably at least 2 alkylene glycol subunits, more preferably at least 3, still more preferably at least 4, still more preferably at least 6, or even more preferably at least 8 alkylene glycol subunits. In some such embodiments, the second polyalkylene glycol unit comprises no more than a combined total of about 100 alkylene glycol subunits, preferably no more than a combined total of about 50 alkylene glycol subunits, more preferably no more than a combined total of about 45 subunits, still more preferably no more than a combined total of about 40 subunits, still more preferably no more than a combined total of about 35 subunits, even more preferably no more than a combined total of about 30 subunits. In any one of these embodiments, the alkylene glycol subunit may be any alkylene glycol subunit as described herein. Preferably, in any one of these embodiments, each alkylene glycol subunit is an ethylene glycol subunit having the following structure:

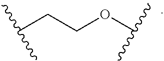 [SMILES: *CCO*]

Preferably, when each alkylene glycol subunit is an ethylene glycol subunit, in any one of these embodiments the second polyalkylene glycol unit is a second polyethylene glycol unit comprising one or more linear polyethylene glycol chains.

In another group of embodiments, the second polyalkylene glycol unit comprises a combined total of from 1 to 100, preferably of from 2 to 50, more preferably of from 3 to 45, still more preferably of from 4 to 40, still more preferably of from 6 to 35, even more preferably of from 8 to 30 alkylene glycol subunits. In any one of these embodiments, the alkylene glycol subunit may be any alkylene glycol subunit as described herein. Preferably, in any one of these embodiments, each alkylene glycol subunit is an ethylene glycol subunit having the following structure:

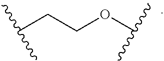 [SMILES: *CCO*]

Preferably, when each alkylene glycol subunit is an ethylene glycol subunit, in any one of these embodiments the second polyalkylene glycol unit is a second polyethylene glycol unit.

In another group of embodiments, the second polyalkylene glycol unit comprises one or more linear polyalkylene glycol chains having a combined total of from 1 to 100, preferably 2 to 50, more preferably 3 to 45, still more preferably 4 to 40, still more preferably 6 to 35, even more preferably 8 to 30 alkylene glycol subunits. In any one of these embodiments, the alkylene glycol subunit may be any alkylene glycol subunit as described herein. Preferably, in any one of these embodiments, each alkylene glycol subunit is an ethylene glycol subunit having the following structure:

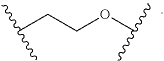 [SMILES: *CCO*]

Preferably, when each alkylene glycol subunit is an ethylene glycol subunit, in any one of these embodiments the second polyalkylene glycol unit is a second polyethylene glycol unit comprising one or more linear polyethylene glycol chains.

In another group of embodiments, the second polyalkylene glycol unit is a linear single polyalkylene glycol chain having at least 1 subunit, preferably at least 2 subunits, more preferably at least 3 subunits, still more preferably at least 6 subunits, even more preferably at least 8 subunits. In any one of these embodiments, the alkylene glycol subunit may be any alkylene glycol subunit as described herein. Preferably, in any one of these embodiments, each alkylene glycol subunit is an ethylene glycol subunit having the following structure:

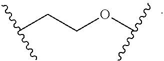 [SMILES: *CCO*]

Preferably, when each alkylene glycol subunit is an ethylene glycol subunit, in any one of these embodiments the second polyalkylene glycol unit is a second polyethylene glycol unit which is a linear single polyethylene glycol chain. Optionally, in any one of these embodiments the linear single polyalkylene glycol chain may be derivatized.

In another group of embodiments, the second polyalkylene glycol unit is a linear single polyalkylene glycol chain having from 1 to 100, preferably 2 to 50, more preferably 3 to 45, more preferably 4 to 40, more preferably 6 to 35, more preferably 8 to 30 alkylene glycol subunits. In any one of these embodiments, the alkylene glycol subunit may be any alkylene glycol subunit as described herein. Preferably, in any one of these embodiments, each alkylene glycol subunit is an ethylene glycol subunit having the following structure:

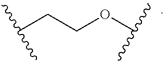 [SMILES: *CCO*]

Preferably, when each alkylene glycol subunit is an ethylene glycol subunit, in any one of these embodiments the second polyalkylene glycol unit is a second polyethylene glycol unit which is a linear single polyethylene glycol chain. Optionally, in any one of these embodiments the linear single polyalkylene glycol chain may be derivatized.

Exemplary linear polyethylene glycol units that can be used as second polyalkylene glycol unit, in particular as a second polyethylene glycol unit, in any one of the embodiments provided herein are as follows:

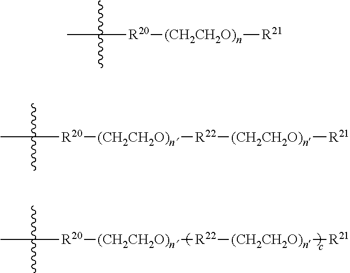

wherein the wavy line indicates the site of attachment to the M in group Z;
R²⁰ is a PEG attachment unit; preferably, R²⁰ is absent; more preferably, M is not a bond;
R²¹ is a PEG capping unit (herein, R²¹ is also denoted as “K^{S}”);
R²² is a PEG coupling unit (i.e. for coupling multiple PEG subunit chains together;
n is independently selected from 1 to 100, preferably from 2 to 50, more preferably from 3 to 45, more preferably from 4 to 40, still more preferably from 6 to 35, even more preferably from 8 to 30;
e is 2 to 5;
each n′ is independently selected from 1 to 100, preferably from 2 to 50, more preferably from 3 to 45, more preferably from 4 to 40, still more preferably from 6 to 35, even more preferably from 8 to 30. In preferred embodiments, there are at least 1, preferably at least 2, more preferably at least 3, more preferably at least 4, more preferably at least 6, even more preferably at least 8 ethylene glycol subunits in the polyethylene glycol unit. In some embodiments, there are no more than 100, preferably no more than 50, more preferably no more than 45, more preferably no more than 40, more preferably no more then 35, even more preferably no more than 30 ethylene glycol subunits in the polyethylene glycol unit. When R²⁰ is absent, a (CH₂CH₂O) subunit is directly bound to the M in group Z; more preferably, in such embodiments M is not a bond.

Preferably, the linear polyethylene glycol unit is

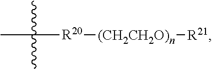

wherein the wavy line indicates the site of attachment to the M in group Z; R²⁰, R²¹ (also denoted herein as “K^{S}”) and n are as defined herein; more preferably R²⁰ is absent; still more preferably, M is not a bond. In preferred embodiments, n is 12 or about 12. In preferred embodiments, n is 24 or about 24. Preferably, R²¹ is H.

The polyethylene glycol attachment unit R²⁰, when present, is part of the second polyethylene glycol unit and acts to link the second polyethylene glycol unit to the M. In these embodiments, preferably M is not a bond and forms a bond with the second polyethylene glycol unit. In exemplary embodiments, the PEG attachment unit R²⁰, when present, is selected from the group consisting of *—C(O)—^{#}, *—S(O)—^{#}, *—C(O)O—^{#}, *—C(O)—(C₁-C₁₀)alkyl-^{#}, *—C(O)—(C₁-C₁₀)alkyl-O—^{#}, *—C(O)—(C₁-C₁₀)alkyl-CO₂—^{#}, *—C(O)—(C₁-C₁₀)alkyl-NH—^{#}, *—C(O)—(C₁-C₁₀)alkyl-S—^{#}; *—C(O)—(C₁-C₁₀)alkyl-C(O)—NH—^{#}; *—C(O)—(C₁-C₁₀)alkyl-NH—C(O)—^{#}; —(C₁-C₁₀)alkyl-^{#}, *—(C₁-C₁₀)alkyl-O—^{#}, *—(C₁-C₁₀)alkyl-C(O)—^{#}, *—(C₁-C₁₀)alkyl-C(O)O #, *—(C₁-C₁₀)alkyl-NH—^{#}, *—(C₁-C₁₀)alkyl-S—^{#}, *—(C₁-C₁₀)alkyl-C(O)—NH—^{#}, *—(C₁-C₁₀)alkyl-NH—C(O) #, and *—CH₂—CH₂SO₂—(C₁-C₁₀)alkyl-^{#}, *—CH₂—C(O)—(C₁-C₁₀)alkyl #; wherein * denotes the attachment point to the M in group Z, and # denotes the attachment point to an ethylene glycol unit.

The PEG coupling unit R²², when present, is part of the second polyethylene glycol unit and is non-PEG material that acts to connect two or more chains of repeating —CH₂CH₂O— subunits. In exemplary embodiments, the PEG coupling unit R²², when present, is independently selected from the group consisting of *—(C₁-C₁₀)alkyl-C(O)—NH—^{#}, *—(C₁-C₁₀)alkyl-NH—C(O)—^{#}, *—(C₂-C₁₀)alkyl-NH—^{#}, *—(C₂-C₁₀)alkyl-O—^{#}, *—(C₁-C₁₀)alkyl-S—^{#}, or *—(C₂-C₁₀)alkyl-NH—^{#}; wherein * denotes the attachment point to an oxygen atom of an ethylene glycol subunit, and # denotes the attachment point to a carbon atom of another ethylene glycol subunit.

The group R²¹, also denoted herein as “K^{S}”, in exemplary embodiments is H (hydrogen), or may be a second capping group, as described herein; preferably, R²¹ is independently selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₃)alkyl and —(C₂-C₁₀)alkyl-N((C₁-C₃)alkyl)₂. In some embodiments R21 may be —(C₁-C₁₀)alkyl, in particular methyl. More preferably R²¹ is H.

Illustrative linear second polyethylene glycol units, which can be used as second polyalkylene glycol units in any one of the embodiments provided herein, are as follows.

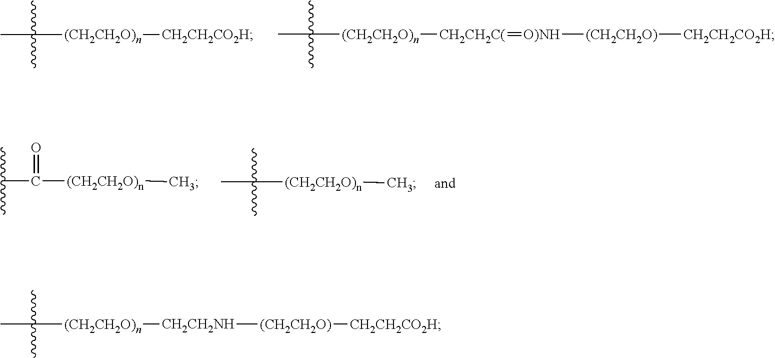

wherein the wavy line indicates the site of attachment to the M in group Z; preferably, M is not a bond; and each n is from 1 to 100, preferably from 2 to 50, more preferably from 3 to 45, still more preferably from 4 to 40, still more preferably from 6 to 35, even more preferably from 8 to 30. In some embodiments, n is about 12. In some embodiments, n is about 24.

In some embodiments, the second polyalkylene glycol unit is from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the second polyalkylene glycol unit has at least 6 alkylene glycol subunits or at least 8 alkylene glycol subunits. In some such aspects, the second polyalkylene glycol unit may have at least 6 alkylene glycol subunits or at least 8 alkylene glycol subunits but no more than 100 alkylene glycol subunits, preferably no more than 50 alkylene glycol subunits. In some embodiments, the second polyalkylene glycol unit is a second polyethylene glycol unit being from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the second polyethylene glycol unit may have at least 6 ethylene glycol subunits or at least 8 ethylene glycol subunits. In some such aspects, the second polyethylene glycol unit may have at least 6 ethylene glycol subunits or at least 8 ethylene glycol subunits but no more than 100 ethylene glycol subunits, preferably no more than 50 ethylene glycol subunits.

In some embodiments, when a second polyalkylene glycol unit R^{S} is present, there are no other alkylene glycol subunits present in the conjugate of formula (I) (i.e., no alkylene glycol subunits are present in any of the other components of the conjugate, such as e.g. in another part of the linker L as provided herein). In other aspects, when a second polyalkylene glycol unit R^{S} is present, there are no more than 8, no more than 7, no more than 6, no more than 5, no more than 4, no more than 3, no more than 2 or no more than 1 other alkylene glycol subunits present in the conjugate of formula (I) (i.e., no more than 8, 7, 6, 5, 4, 3, 2, or 1 other alkylene glycol subunits are present in other components of the conjugate, such as e.g. in another part of the linker L as provided herein).

Preferably, in other embodiments, when a second polyalkylene glycol unit R^{S} is present, the conjugate further comprises a first polyalkylene glycol unit R^{F} as R¹, as described herein. Preferably, when R^{S} is a second polyethylene glycol unit and the conjugate further comprises a first polyalkylene glycol unit R^{F}, the first polyalkylene glycol unit is a first polyethyleneglycol unit, as described herein.

It will be appreciated that when referring to alkylene glycol subunits, in particular ethylene glycol subunits, and depending on context, the number of subunits can represent an average number, e.g., when referring to a population of conjugates or intermediate compounds, and using polydisperse polyalkylene glycols, in particular polydisperse polyethylene glycols.

Linker *-Aₐ-W_{w}—B_{b}—^{##}

In some embodiments, the Linker L has the formula: *-Aₐ-W_{w}—B_{b}—^{##}, wherein: -A- is a second spacer unit, as described herein; a is 0 or 1; each —W— is independently an amino acid; w is independently an integer ranging from 0 to 12; —B— is a first spacer unit; and
b is 0 or 1; * denotes the attachment point to the —Y—; and ## denotes the attachment point to the drug moiety. Herein, the notation “W_{w}”, or “—W_{w}—”, or the like, i.e. the combination of W and the associated integer w, is also denoted as “amino acid unit”. Examples for suitable second spacer units, amino acid units and first spacer units are described, e.g., in WO 2004/010957 A2.

In the linker having the structure *-Aₐ-W_{w}—B_{b}—^{##}, the second spacer unit serves to connect a —Y— to the amino acid unit —W_{w}—. The second spacer unit (-A-) may be any second spacer unit as described herein. When present, the second spacer unit (-A-) may be any chemical group or moiety which is capable to link a —Y— to the amino acid unit. Alternatively, the second spacer unit may link the —Y— to the first spacer unit, in case no amino acid unit is present. Alternatively, the second spacer unit may link the —Y— to the drug moiety (-D), in case no first spacer unit and no amino acid unit are present. In this regard, the —Y—, as described herein, is bonded to the second spacer unit (-A-). The second spacer unit (-A-) may comprise or may be a functional group that is capable to form a bond to an amino acid unit (—_{w}—), or to a first spacer unit (—B_{b}—), or to a drug moiety (-D), depending on whether an amino acid unit (—W_{w}—) and/or a first spacer unit (—B—) is present or not. Preferably, the functional group, which is capable to form a bond to an amino acid unit (—W_{w}—), in particular to the N terminus of the amino acid unit, or to a first spacer unit (—B—), or to a d moiety (-D), is a carbonyl group which is depicted as, e.g.

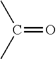

or —C(O)—. The integer a associated with the second spacer unit may be 0 or 1. Preferably, the integer a is 1. Alternatively, in other embodiments the second spacer unit is absent (a=0).

In the linker *-A-W_{w}—B_{b}—^{##}, the second spacer unit -A-, when present, may be any second spacer unit as described herein. In preferred embodiments of the linker *-Aₐ-W_{w}—B_{b}—^{##}, the second spacer unit -A-, when present (a=1), may be a group Z having the structure

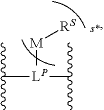

wherein

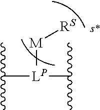

is as defined herein. Accordingly, in preferred embodiments, the linker (L) may have the structure

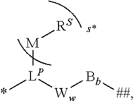

wherein L^{P}, R^{S}, s*, M, W, w, B and b are as defined herein; * denotes the attachment point to the —Y—; and ## denotes the attachment point to the drug moiety (-D).

The amino acid unit (—W_{w}—), when present, may link the second spacer unit A to the first spacer unit B in case the first spacer unit is present. Alternatively, the amino acid unit may link the second spacer unit to the drug moiety (D) in case the first spacer unit is absent. Alternatively, the amino acid unit may link the Y to the first spacer unit in case the second spacer unit is absent. Alternatively, the amino acid unit may link the Y to the drug moiety in case the first spacer unit and the second spacer unit are absent.

The amino acid unit —W_{w}— may be a dipeptide (w=2), a tripeptide (w=3), a tetrapeptide (w=4), a pentapeptide (w=5), a hexapeptide (w=6), a heptapeptide (w=7), an octapeptide (w=8), a nonapeptide (w=9), a decapeptide (w=10), an undecapeptide (w=11) or a dodecapeptide (w=12).

In some embodiments, the amino acid unit can comprise natural amino acids. In some embodiments, the amino acid unit can comprise non-natural amino acids.

In any one of the embodiments described herein, each amino acid of the amino acid unit, except for amino acids which are not chiral such as e.g. glycine, may be independently in the L configuration or in the D configuration. Preferably, in any one of the embodiments described herein each amino acid of the amino acid unit, except for amino acids which are not chiral such as e.g. glycine, is in the L configuration (i.e., in the naturally occurring configuration).

Preferably, when a second spacer unit (-A-) is present, in any one of the embodiments described herein the N terminus of the amino acid unit —W_{w}— is bound to the second spacer unit (A), more preferably via a carbonyl group of the second spacer unit.

Preferably, in any one of the embodiments described herein, the C terminus of the amino acid unit —W_{w}— is bound to a first spacer unit (B) in case a first spacer unit is present. Alternatively, in any one of the embodiments described herein, the C terminus of the amino acid unit —W_{w}— may be bound to the drug moiety (-D) in case a first spacer unit is absent. In other embodiments, the N-terminus of the amino acid unit —W_{w}— may be bound to the first spacer unit (B), when present, and the C-terminus may be bound to the second spacer unit A, when present.

Each —W— unit independently may have the formula denoted below in the square brackets, and w is an integer ranging from 0 to 12; preferably w is an integer ranging from 1 to 5; more preferably w is an integer ranging from 2 to 4; still more preferably w is 2 or 3; in very preferred embodiments w is 2:

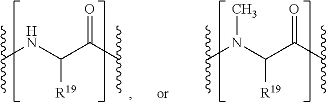

wherein R¹⁹ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

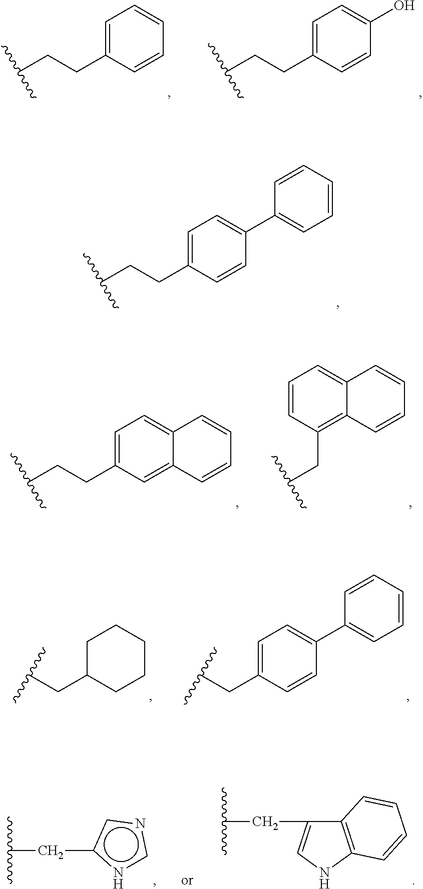

The amino acid unit can be enzymatically cleaved by one or more enzymes, including but not limited to a tumor-associated protease, preferably a cathepsin, more preferably cathepsin B, to liberate the drug moiety (-D), which in one embodiment is protonated in vivo upon release to provide a free drug moiety (D). Illustrative —W_{w}— units are represented by formulae (VII) to (IX).

Accordingly, the —W_{w}— unit may be a dipeptide of formula (VII):

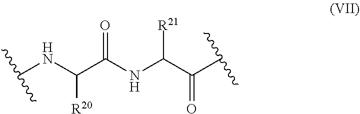

wherein R²⁰ and R²¹ are as follows:

| R²⁰ | R²¹ |
|---|---|
| benzyl | (CH₂)₄NH₂; |
| methyl | (CH₂)₄NH₂; |
| isopropyl | (CH₂)₄NH₂; |
| isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH₂; |
| 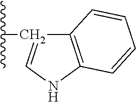 [SMILES: *c1c[nH]c2ccccc12] | (CH₂)₃NHCONH₂; |
| benzyl | methyl; and |
| benzyl | (CH₂)₃NHC(═NH)NH₂;. |

The —W_{w}— unit may be a tripeptide of formula (VIII):

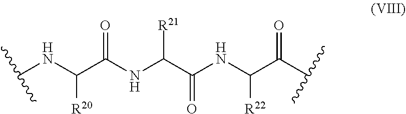

wherein R²⁰, R²¹ and R²² are as follows:

| R²⁰ | R²¹ | R²² |
|---|---|---|
| benzyl | benzyl | (CH₂)₄NH₂; |
| isopropyl | benzyl | (CH₂)₄NH₂; and |
| H | benzyl | (CH₂)₄NH₂;. |

The W_{w} unit may be a tetrapeptide of formula (IX):

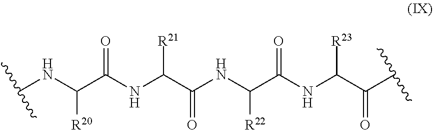

wherein R²⁰, R²¹, R²² and R²³ are as follows:

| R²⁰ | R²¹ | R²² | R²³ |
|---|---|---|---|
| H | benzyl | isobutyl | H; and |
| methyl | isobutyl | methyl | isobutyl.. |

Exemplary amino acid units include, but are not limited to, units of formula (VII) where: R²⁰ is benzyl and R²¹ is -(CH₂)₄NH₂ (Phe-Lys); R²⁰ is isopropyl and R²¹ is -(CH₂)₄NH₂ (Val-Lys); R²⁰ is isopropyl and R²¹ is -(CH₂)₃NHCONH₂ (Val-Cit). Another exemplary Amino Acid unit is a unit of formula (VIII) wherein R²⁰ is benzyl, R²¹ is benzyl, and R²² is -(CH₂)₄NH₂ (Phe-Phe-Lys).

Useful —W_{w}— units can be designed and optimized in their selectivity for enzymatic cleavage by a particular enzyme, for example, a tumor-associated protease. In one embodiment, a —W_{w}— unit is that whose cleavage is catalyzed by cathepsin B, C and/or D, or a plasmin protease (“tumor-associated proteases”). Preferably, the —W_{w}— unit is cleaved by cathepsin B. Suitable linkers, which can be cleaved by a protease, are described, e.g., in G. M. Dubowchik et al., “Cathepsin B-Labile Dipeptide Linkers forLysosomal Release ofDoxorubicin from Internalizing Immunoconjugates; Model Studies of Enzymatic Drug Release and Antigen-Specific In Vitro Anticancer Activity”, Bioconjugate Chem., Vol. 13, No. 4, 2002, 855-869; S. C. Jeffrey et al., “Dipeptide-based highly potent doxorubicin antibody conjugate”, Bioorg. Med. Chem. Lett. 16 (2006), 358-362; and M. S. Kung Sutherland et al., “SGN-CD33A: a novel CD33-targeting antibody-drug conjugate using a pyrrolobenzodiazepine dimer is active in models of drug-resistant AML”, Blood, 22 Aug. 2013, volume 122, number 8, 1455-1463.

In one embodiment, —W_{w}— is a dipeptide, a tripeptide, a tetrapeptide or a pentapeptide. Preferably, —W_{w}— is a dipeptide, a tripeptide or a tetrapeptide. More preferably, —W_{w}— is a dipeptide or a tripeptide. In very preferred embodiments, —W_{w}— is a dipeptide (i.e. w=2).

When R¹⁹, R²⁰, R²¹, R²² or R²³ is other than hydrogen, the carbon atom to which R¹⁹, R²⁰, R²¹, R²² or R²³ is attached is chiral. Each carbon atom to which R¹⁹, R²⁰, R²¹, R²² or R²³ is attached may be independently in the (S) or (R) configuration. Preferably, each carbon atom to which R¹⁹, R²⁰, R²¹, R²² or R²³ is attached is in the (S) configuration.

In one preferred embodiment, the amino acid unit is valine-citrulline (i.e. Val-Cit or VC). In another preferred embodiment, the amino acid unit is valine-alanine (i.e. Val-Ala or VA). In another preferred embodiment, the amino acid unit is alanine-alanine (i.e. Ala-Ala or AA). In another preferred embodiment, the amino acid unit is phenylalanine-lysine (i.e. Phe-Lys or FK). Such linkers are illustrative examples for a linker which can be cleaved by a protease, such as e.g. cathepsin B.

The notation of peptides used herein throughout this specification follows the conventional nomenclature. Accordingly, the N-terminus of a peptide is written on the left, and the C-terminus of the peptide is written on the right. As an illustrative but non-limiting example, in the dipeptide valine-citrulline (i.e. Val-Cit or VC), the valine has the N-terminus, and the citrulline has the C-terminus. Preferably, in any one of the embodiments described herein, when a second spacer unit (-A-) is present, the N-terminus of a peptide, such as e.g. of a dipeptide (as illustrative non-limiting example: Val-Cit), is bound to the second spacer unit (-A-), more preferably via a carbonyl group of the second spacer unit, and the C-terminus of the peptide is bound to a first spacer unit (—B—), in case a first spacer unit (—B—) is present, or to the drug moiety (-D) in case a first spacer unit (—B—) is absent.

In yet another embodiment, the amino acid unit is N-methylvaline-citrulline. In yet another embodiment, the amino acid unit is selected from the group consisting of 5-aminovaleric acid, homophenylalanine-lysine, tetraisoquinolinecarboxylate-lysine, cyclohexylalanine-lysine, isonepecotic acid-lysine, betaalanine-lysine, glycine-serine-valine-glutamine, and isonepecotic acid.

Preferably, the amino acid unit is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, the amino acid unit is valine-citrulline (i.e. Val-Cit or VC) or valine-alanine (i.e. Val-Ala or VA). Even more preferably, the amino acid unit is valine-citrulline (i.e. Val-Cit or VC).

In some embodiments, the amino acid unit is selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). Preferably, the amino acid unit is selected from the group consisting of valine-glutamine (i.e. Val-Gln or VQ), leucine-glutamine (i.e. Leu-Gln or LQ), and phenylalanine-glutamin (i.e. Phe-Gln or FQ). More preferably, the amino acid unit is valine-glutamine (i.e. Val-Gln or VQ) or leucine-glutamine (i.e. Leu-Gln or LQ). Linkers which comprise amino acid units according to these embodiments can be illustrative examples for a linker which is cleavable, in particular by a protease, such as e.g. a cathepsin (e.g., cathepsin B). The amino acid units of these embodiments and further suitable amino acid units are disclosed, e.g., in Salomon et al., “Optimizing Lysosomal Activation of Antibody-Drug Conjugates (ADCs) by Incorporation of Novel Cleavable Dipeptide Linkers”, Mol. Pharmaceutics 2019, 16, 12, 4817-4825.

The first spacer unit (—B—), when present, may link an amino acid unit (W_{w}) to the drug moiety when an amino acid unit is present. Alternatively, the first spacer unit (B) may link the second spacer unit (A) to the drug moiety (C) when the amino acid unit is absent. The first spacer unit may link the drug moiety to the Y when both the amino acid unit and second spacer unit are absent.

The integer b may be 0 or 1. In preferred embodiments, the integer b is 1. Alternatively, in other embodiments, the integer b is 0, and the first spacer unit is absent.

The first spacer unit (—B—) may be of two general types: self-immolative and non-self-immolative. A non-self-immolative first spacer unit is one in which part or all of the first spacer unit remains bound to the drug moiety (D) after cleavage, particularly enzymatic, of an amino acid unit (—W_{w}-) of the linker (L). Examples of a non-self-immolative first spacer unit include, but are not limited to a (glycine-glycine) first spacer unit and a glycine first spacer unit (both depicted in Scheme 1) (infra). When an exemplary compound containing a glycine-glycine first spacer unit or a glycine first spacer unit undergoes enzymatic cleavage via a tumor-cell associated-protease, a cancer-cell-associated protease or a lymphocyte-associated protease, a glycine-glycine-drug moiety (“D” represent a drug moiety) or a glycine-Drug moiety (D) is cleaved from the -Aₐ-W_{w}—. In one embodiment, an independent hydrolysis reaction takes place within the target cell, cleaving the glycine-Drug moiety bond and liberating the Drug (D).

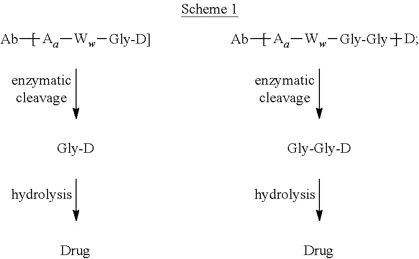

In one embodiment, a non-self-immolative first spacer unit is -Gly-Gly-. In another embodiment, a non-self-immolative first spacer unit is -Gly-.

Alternatively, an exemplary compound containing a self-immolative first spacer unit can release a drug moiety -D without the need for a separate hydrolysis step. In an exemplary embodiment, a self-immolative first spacer unit is a PAB group that is linked to —W_{w}— via the amino nitrogen atom of the PAB group, and connected directly to -D via a carbonate, carbamate or ether group. Without being bound by any particular theory or mechanism, Scheme 2 depicts a possible mechanism of drug release of a PAB group which is attached directly to -D via a carbamate or carbonate group espoused by Toki et al. (2002) J Org. Chem. 67:1866-1872.

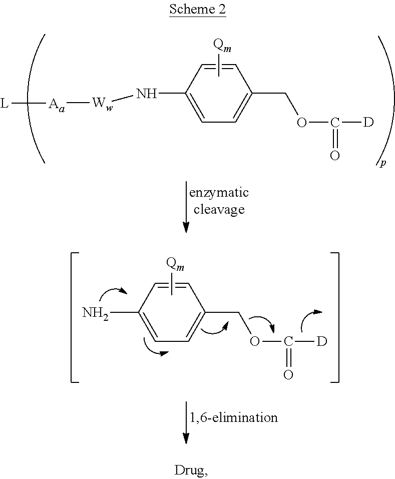

wherein Q is —(C₁-C₈)alkyl, —O—(C₁-C₈)alkyl, -halogen, -nitro or -cyano; m is an integer ranging from 0 to 4, preferably m is 0, 1 or 2, more preferably m is 0 or 1, still more preferably m is 0; and p ranges from 1 to 20.

Without being bound by any particular theory or mechanism, Scheme 3 depicts a possible mechanism of drug release of a PAB group which is attached directly to a drug moiety -D via an ether or amine linkage.

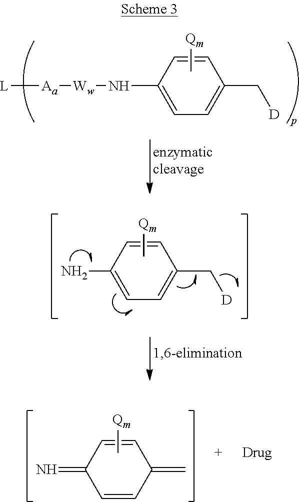

wherein Q is —(C₁-C₈)alkyl, —O—(C₁-C₈)alkyl, -halogen, -nitro or -cyano; m is an integer ranging from 0 to 4, preferably m is 0, 1 or 2, more preferably m is 0 or 1, still more preferably m is 0; and p ranges from 1 to 20.

Other examples of self-immolative spacers include, but are not limited to, aromatic compounds that are electronically similar to the PAB group such as 2-aminoimidazol-5-methanol derivatives (Hay et al. (1999) Bioorg. Med. Chem. Lett. 9:2237) and ortho or para-aminobenzylacetals. Spacers can be used that undergo cyclization upon amide bond hydrolysis, such as substituted and unsubstituted 4-aminobutyric acid amides (Rodrigues et al., Chemistry Biology, 1995, 2, 223), appropriately substituted bicyclo[2.2.1] and bicyclo[2.2.2] ring systems (Storm, et al., J. Amer. Chem. Soc., 1972, 94, 5815) and 2-aminophenylpropionic acid amides (Amsberry, et al., J. Org. Chem., 1990, 55, 5867). Elimination of amine-containing drugs that are substituted at the alpha-position of glycine (Kingsbury, et al., J. Med. Chem., 1984, 27, 1447) are also examples of self-immolative spacer useful in exemplary compounds.

In one embodiment, the first spacer unit is a branched bis(hydroxymethyl)styrene (BHMS) unit as depicted in Scheme 4, which can be used to incorporate and release multiple drugs (D).

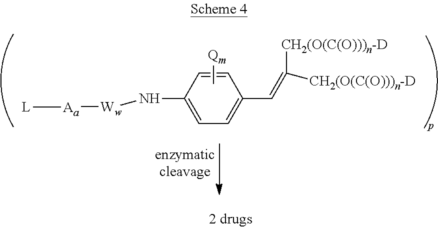

wherein Q is —(C₁-C₈)alkyl, —O—(C₁-C₈)alkyl, -halogen, -nitro or -cyano; m is an integer ranging from 0 to 4; preferably m is 0, 1 or 2; more preferably m is 0 or 1; still more preferably m is 0; and p ranges from 1 to 10; n is 0 or 1; and p ranges from 1 to 20.

In preferred embodiments, the first spacer unit is represented by formula (X):

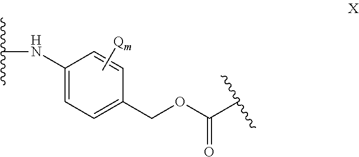

wherein Q is —(C₁-C₈)alkyl, —O—(C₁-C₈)alkyl, -halogen, -nitro or -cyano; and m is an integer ranging from 0 to 4; preferably m is 0, 1 or 2; more preferably m is 0 or 1; in very preferred embodiments m is 0.

In some embodiments, the first spacer unit is represented by formula (XI):

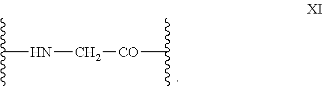 [SMILES: *NCC(*)=O]

In some embodiments, the first spacer unit is represented by formula (XII):

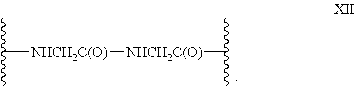

Preferably, when an amino acid unit is present, in any one of formulae (X), (XI) and (XII), in particular in formula (X), the NH group is bound to a C-terminus of the amino acid unit. Preferably, in any one of formulae (X), (XI) and (XII), in particular in formula (X), the C(O) group is bound to the drug moiety (D).

In very preferred embodiments, the first spacer unit is a PAB group having the following structure:

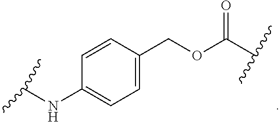 [SMILES: *Nc1ccc(COC(*)=O)cc1]

Preferably, when an amino acid unit is present, the NH group is bound to an amino acid unit (—W_{w}—), more preferably to a C-terminus of the amino acid unit. Preferably, the C(O) group is bound to the drug moiety (D).

In some embodiments, the first spacer group (—B—) is a heterocyclic “self-immolating moiety” of Formulas I, II or III bound to the drug moiety and incorporates an amide group that upon hydrolysis by an intracellular protease initiates a reaction that ultimately cleaves the first spacer unit (—B—) from the drug moiety such that the drug is released from the conjugate in an active form. The linker moiety further comprises an amino acid unit (—W_{w}—) adjacent to the first spacer group (—B—) that is a substrate for an intracellular enzyme, for example an intracellular protease such as a cathepsin (e.g., cathepsin B), that cleaves the peptide at the amide bond shared with the first spacer group (—B—). Heterocyclic self-immolating moieties are described, e.g., in WO 2019/236954.

In some embodiments, the first spacer unit (—B—) is a heterocyclic self-immolating group selected from Formulas I, ∥ and Ill:

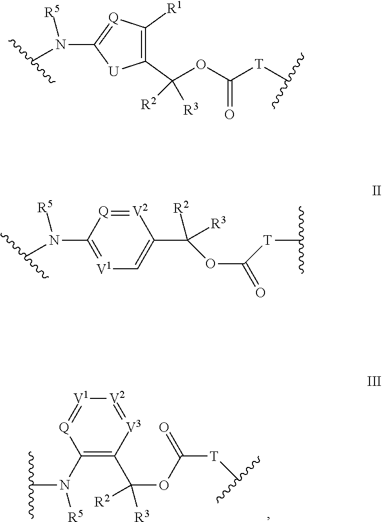

wherein the wavy lines indicate the covalent attachment sites to the amino acid unit —W_{w}— and the drug moiety, and wherein U is O, S or NR⁶; Q is CR⁴ or N; V¹, V² and V³ are independently CR⁴ or N provided that for formula II and III at least one of Q, V¹ and V² is N; T is O pending from a drug moiety (-D); R¹, R², R³ and R⁴ are independently selected from the group consisting of H, F, Cl, Br, I, OH, —N(R⁵)₂, —N(R⁵)₃⁺, —(C₁-C₈)alkylhalide, carboxylate, sulfate, sulfamate, sulfonate, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, —(C₁-C₈)alkoxy, —(C₁-C₈)halosubstituted alkyl, polyethyleneoxy, phosphonate, phosphate, —(C₁-C₈)alkyl, —(C₁-C₈)substituted alkyl, —(C₂-C₃)alkenyl, —(C₂-C₃)substituted alkenyl, —(C₂-C₃)alkynyl, —(C₂-C₃)substituted alkynyl, —(C₆-C₂₀)aryl, —(C₆-C₂₀)substituted aryl, —(C₃-C₂₀)heterocycle, and —(C₃-C₂₀)substituted heterocycle; or when taken together, R² and R³ form a carbonyl (═O), or spiro carbocyclic ring of 3 to 7 carbon atoms; and R⁵ and R⁶ are independently selected from H, —(C₁-C₈)alkyl, —(C₁-C₈)substituted alkyl, —(C₂-C₃)alkenyl, —(C₂-C₃)substituted alkenyl, —(C₂-C₃)alkynyl, —(C₂-C₃)substituted alkynyl, —(C₆-C₂₀)aryl, —(C₆-C₂₀)substituted aryl, —(C₃-C₂₀)heterocycle, and —(C₃-C₂₀)substituted heterocycle; wherein —(C₁-C₈)substituted alkyl, —(C₂-C₈) substituted alkenyl, —(C₂-C₃)substituted alkynyl, —(C₆-C₂₀)substituted aryl, and —(C₃-C₂₀)substituted heterocycle are independently substituted with one or more substituents selected from the group consisting of F, Cl, Br, I, OH, —N(R⁵)₂, —N(R⁵)₃+, —(C₁-C₈)alkylhalide, carboxylate, sulfate, sulfamate, sulfonate, —(C₁-C₈)alkylsulfonate, —(C₁-C₈)alkylamino, 4-dialkylaminopyridinium, —(C₁-C₈)alkylhydroxyl, —(C₁-C₈)alkylthiol, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, —(C₁-C₈)alkoxy, —(C₁-C₈)trifluoroalkyl, —(C₁-C₈)alkyl, —(C₃-C₁₂)carbocycle, —(C₆-C₂₀)aryl, —(C₃-C₂₀)heterocycle, polyethyleneoxy, phosphonate, and phosphate.

The conjugate comprising a heterocyclic self-immolative is stable extracellularly, or in the absence of an enzyme capable of cleaving the amide bond of the self-immolative moiety. However, upon entry into a cell, or exposure to a suitable enzyme, an amide bond is cleaved initiating a spontaneous self-immolative reaction resulting in the cleavage of the bond covalently linking the self-immolative moiety to the drug moiety, to thereby effect release of the drug in its underivatized or pharmacologically active form.

The self-immolative moiety in conjugates either incorporates one or more heteroatoms and thereby may provide improved solubility, may improve the rate of cleavage and/or may decrease propensity for aggregation of the conjugate. Thus, the heterocyclic self-immolative linker constructs in some instances may result in increased efficacy, decreased toxicity, and/or desirable pharmacokinetic and/or pharmacodynamic properties.

It is understood that T in formulae I-III is O, as it is derived from the tertiary hydroxyl (—OH) on the lactone ring portion of a drug moiety.

Not to be limited by theory or any particular mechanism, the presence of electron-withdrawing groups on the heterocyclic ring of formula I, II or III may moderate the rate of cleavage.

In one embodiment, the self-immolative moiety is the group of formula I in which Q is N, and U is O or S. Such a group has a non-linearity structural feature which improves solubility of the conjugates. In this context R is sometimes H, methyl, nitro, or CF₃. In one embodiment, Q is N and U is O thereby forming an oxazole ring and R is H. In another embodiment, Q is N and U is S thereby forming a thiazole ring optionally substituted at R with an Me or CF₃ group.

In another exemplary embodiment, the self-immolative moiety is the group of formula II in which Q is N and V¹ and V² are independently N or CH. In another embodiment, Q, V¹ and V² are each N. In another embodiment, Q and V¹ are N while V² is CH. In another embodiment, Q and V² are N while V¹ is CH. In another embodiment, Q and V¹ are both CH and V² is N. In another embodiment, Q is N while V¹ and V² are both CH.

In another embodiment, the self-immolative moiety is the group of formula III in which Q, V¹, V² and V³ are each independently N or CH. In another embodiment Q is N while V¹, V² and V³ are each N. In another embodiment, Q, V¹, and V² are each CH while V³ is N. In another embodiment Q, V² and V³ are each CH while V¹ is N. In another embodiment, Q, V¹ and V³ are each CH while V² is N. In another embodiment, Q and V² are both N while V¹ and V³ are both CH. In another embodiment Q and V² are both CH while V¹ and V³ are both N. In another embodiment, Q and V³ are both N while V¹ and V² are both CH.

Preferably, the linker (L) has the formula: *-Aₐ-W_{w}—B_{b}—^{##}, wherein the integer a is 1, the integer b is 1, and the integer w is 2, 3 or 4, more preferably the integer w is 2 or 3; in very preferred embodiments the integer w is 2; and -A-, each —W— and —B— are as defined herein; * denotes the attachment point to the Y; and ## denotes the attachment point to the drug moiety (D).

Preferably, the linker (L) has the following structure:

*-Aₐ-W_{w}—B_{b}—^{##}
wherein -A- is a second spacer unit as described herein; a is an integer as described herein; preferably a is 1;
—B— is a first spacer unit as described herein; b is an integer as described herein; preferably b is 1;
* denotes the attachment point to the Y; and ## denotes the attachment point to the drug moiety (-D);
—W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). Preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). Linkers according to these embodiments can be illustrative examples for a linker which is cleavable, in particular by a protease, such as e.g. a cathepsin (e.g., cathepsin B).

Preferably, the linker L has the following structure:

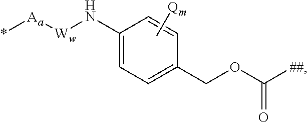

wherein -Aₐ is a second spacer unit as described herein; a is an integer as described herein; preferably a is 1;
—W_{w}— is an amino acid unit as described herein; w is an integer as described herein; preferably w is 2, 3 or 4 (i.e. preferably —W_{w}— is a dipeptide, a tripeptide or a tetrapeptide), more preferably w is 2 or 3 (i.e. more preferably —W_{w}— is a dipeptide or a tripeptide), in very preferred embodiments w is 2 (i.e. still more preferably —W_{w}— is a dipeptide);
Q is as defined herein;
m is an integer as defined herein, preferably m is 0;
* denotes the attachment point to the Y; and ## denotes the attachment point to the drug moiety (-D). Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). Linkers according to these embodiments can be illustrative examples for a linker which is cleavable, in particular by a protease, such as e.g. a cathepsin (e.g., cathepsin B).

More preferably, the linker L has the following structure:

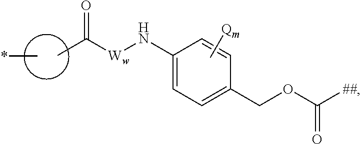

wherein:

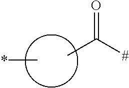

is as defined herein; * denotes the attachment point to the Y; and # denotes the attachment point to the amino acid unit —W_{w}—, when present, or to the NH group; —W_{w}— is an amino acid unit as described herein; w is an integer as described herein, preferably w is 2, 3 or 4 (i.e. preferably —W_{w}— is a dipeptide, a tripeptide or a tetrapeptide), more preferably w is 2 or 3 (i.e. more preferably —W_{w}— is a dipeptide or a tripeptide), in very preferred embodiments w is 2 (i.e. still more preferably —W_{w}— is a dipeptide); Q is as defined herein;
m is an integer as defined herein, preferably m is 0;
* denotes the attachment point to the Y; and ## denotes the attachment point to the drug moiety (-D). Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). Linkers according to these embodiments can be illustrative examples for a linker which is cleavable, in particular by a protease, such as e.g. a cathepsin (e.g., cathepsin B).

Still more preferably, the linker L has the following structure:

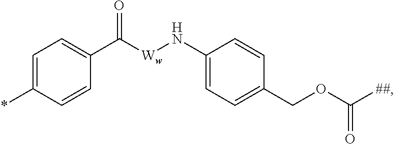

wherein:
—W_{w}— is an amino acid unit as described herein; w is an integer as described herein, preferably w is 2, 3 or 4 (i.e. preferably —W_{w}— is a dipeptide, a tripeptide or a tetrapeptide), more preferably w is 2 or 3 (i.e. more preferably —W_{w}— is a dipeptide or a tripeptide), in very preferred embodiments w is 2 (i.e. still more preferably —W_{w}— is a dipeptide);
* denotes the attachment point to the Y; and ## denotes the attachment point to the drug moiety (-D). Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). Linkers according to these embodiments can be illustrative examples for a linker which is cleavable, in particular by a protease, such as e.g. a cathepsin (e.g., cathepsin B).

In a preferred embodiment, the linker L has the following structure:

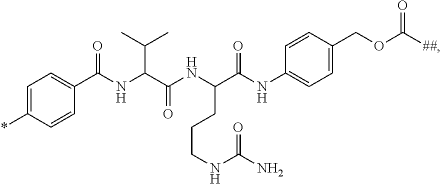

which comprises the dipeptide valine-citrullin as the amino acid unit —W_{w}—; and
wherein * denotes the attachment point to the Y; and ## denotes the attachment point to the drug moiety (-D). Such linker is an illustrative example for a linker which is cleavable, in particular by a protease, such as e.g. a cathepsin (e.g., cathepsin B).

In another preferred embodiment, the linker L has the following structure:

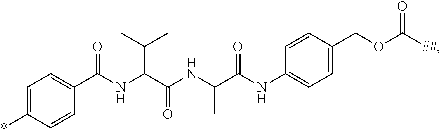

which comprises the dipeptide valine-alanine as the amino acid unit —W_{w}—; and
wherein * denotes the attachment point to the Y; and ## denotes the attachment point to the drug moiety (-D). Such linker is an illustrative example for a linker which is cleavable, in particular by a protease, such as e.g. a cathepsin (e.g., cathepsin B).

Preferably, the linker (L) has the formula:

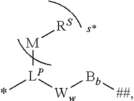

wherein the integer b is 1, and the integer w is 2, 3 or 4, more preferably the integer w is 2 or 3, in very preferred embodiments the integer w is 2; and

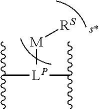

is as described herein; R^{S} is, each independently, a second polyalkylene glycol unit as described herein; preferably each R^{S} is, independently, a second polyethylene glycol unit as described herein; M is, each independently, as described herein, preferably each M is —O—; s* is an integer as described herein; preferably, s* is 1; each —W—, and —B— are as defined herein; * denotes the attachment point to the Y; and ## denotes the attachment point to the drug moiety (-D). Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). Preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). More preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). Linkers according to these embodiments can be illustrative examples for a linker which is cleavable, in particular by a protease, such as e.g. a cathepsin (e.g., cathepsin B).

Preferably, the linker L has the following structure:

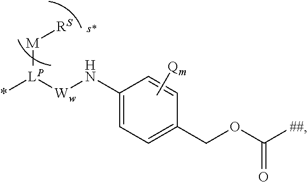

wherein

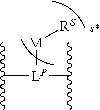

is as described herein; R^{S} is, each independently, a second polyalkylene glycol unit as described herein; preferably, each R^{S} is, independently, a second polyethylene glycol unit as described herein; M is, each independently, as described herein, preferably each M is —O—; s* is an integer as described herein; preferably, s* is 1;
—W_{w}— is an amino acid unit as described herein; w is an integer as described herein; preferably w is 2, 3 or 4 (i.e. preferably —W_{w}— is a dipeptide, a tripeptide or a tetrapeptide), more preferably w is 2 or 3 (i.e. more preferably —W_{w}— is a dipeptide or a tripeptide), in very preferred embodiments w is 2 (i.e. still more preferably —W_{w}— is a dipeptide);
Q is as described herein;
m is an integer as described herein, preferably m is 0;
* denotes the attachment point to the Y; and ## denotes the attachment point to the drug moiety (-D). Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). Linkers according to these embodiments can be illustrative examples for a linker which is cleavable, in particular by a protease, such as e.g. a cathepsin (e.g., cathepsin B).

More preferably, the linker L has the following structure:

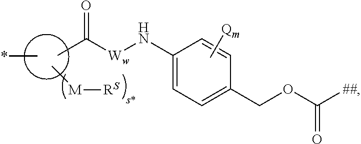

wherein:

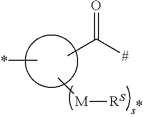

is as defined herein; R^{S} is, each independently, a second poly(alkylene) glycol unit as defined herein; preferably each R^{S} is, independently, a second polyethylene glycol unit as defined herein; M is, each independently, as defined herein; preferably each M is —O—; s* is an integer as defined herein; preferably s* is 1; * denotes the attachment point to the Y; and # denotes the attachment point to the amino acid unit —W_{w}—, when present, or to the NH group;
—W_{w}— is an amino acid unit as described herein; w is an integer as described herein, preferably w is 2, 3 or 4 (i.e. preferably —W_{w}— is a dipeptide, a tripeptide or a tetrapeptide), more preferably w is 2 or 3 (i.e. more preferably —W_{w}— is a dipeptide or a tripeptide), in very preferred embodiments w is 2 (i.e. still more preferably —W_{w}— is a dipeptide);
Q is as defined herein;
m is an integer as defined herein, preferably m is 0;
* denotes the attachment point to the Y; and ## denotes the attachment point to the drug moiety (-D). Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). Linkers according to these embodiments can be illustrative examples for a linker which is cleavable, in particular by a protease, such as e.g. a cathepsin (e.g., cathepsin B).

Still more preferably, the linker L has the following structure:

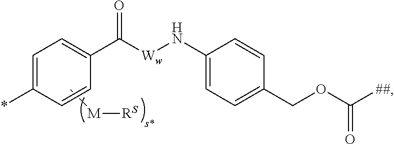

wherein:
R^{S} is, each independently a second poly(alkylene) glycol unit as defined herein; preferably, each R^{S} is, independently, a second polyethylene glycol unit as defined herein; M is, each independently, as defined herein, preferably each M is —O—; s* is an integer as defined herein; preferably s* is 1;
—W_{w}— is an amino acid unit as described herein; w is an integer as described herein, preferably w is 2, 3 or 4 (i.e. preferably —W_{w}— is a dipeptide, a tripeptide or a tetrapeptide), more preferably w is 2 or 3 (i.e. more preferably —W_{w}— is a dipeptide or a tripeptide), still more preferably w is 2 (i.e. still more preferably —W_{w}— is a dipeptide);
* denotes the attachment point to the Y; and ## denotes the attachment point to the drug moiety (-D). Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). Linkers according to these embodiments can be illustrative examples for a linker which is cleavable, in particular by a protease, such as e.g. a cathepsin (e.g., cathepsin B).

In a preferred embodiment, the linker L has the following structure:

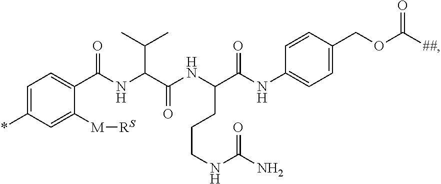

which comprises the dipeptide valine-citrullin as the amino acid unit —W_{w}—;
wherein R^{S} is a second poly(alkylene) glycol unit as defined herein; preferably R^{S} is a second polyethylene glycol unit as defined herein; M is as defined herein; preferably M is —O—; and
* denotes the attachment point to the Y; and ## denotes the attachment point to the drug moiety (-D). Linkers according to these embodiments can be illustrative examples for a linker which is cleavable, in particular by a protease, such as e.g. a cathepsin (e.g., cathepsin B).

In another preferred embodiment, the linker L has the following structure:

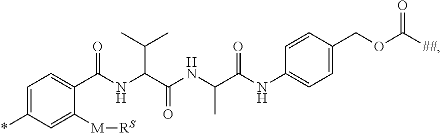

which comprises the dipeptide valine-alanine as the amino acid unit —W_{w}—; and
wherein R^{S} is a second poly(alkylene) glycol unit as defined herein; preferably R^{S} is a second polyethylene glycol unit as defined herein; M is as defined herein; preferably M is —O—; and
* denotes the attachment point to the Y; and ## denotes the attachment point to the drug moiety (-D). Linkers according to these embodiments can be illustrative examples for a linker which is cleavable, in particular by a protease, such as e.g. a cathepsin (e.g., cathepsin B).

In some embodiments, the linker L has the formula: *-Aₐ-W_{w}-^{##}, wherein -Aₐ- is a second spacer unit as defined herein; the integer a associated with the second spacer unit is as defined herein; —W_{w}— is an amino acid unit as defined herein; the integer w associated with the amino acid unit W is a defined herein; the first spacer unit (—B_{b}—) is absent; * denotes the attachment point to the Y; and # denotes the attachment point to the drug moiety (-D).

Preferably, the integer a is 1. Preferably, the integer w is 2, 3 or 4, more preferably the integer w is 2 or 3, still more preferably the integer w is 2. Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). In any one of these embodiments, the second spacer unit -A- may be a group Z having the structure

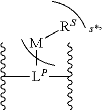

wherein

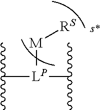

is as defined herein.

The linker L may have the following structure:

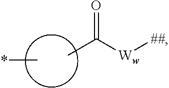

wherein:

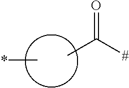

is as defined herein; * denotes the attachment point to the Y; and # denotes the attachment point to the amino acid unit —W_{w}—;
—W_{w}— is an amino acid unit as described herein; w is an integer as described herein, preferably w is 2, 3 or 4 (i.e. preferably —W_{w}— is a dipeptide, a tripeptide or a tetrapeptide), more preferably the integer w is 2 or 3 (i.e. more preferably —W_{w}— is a dipeptide or a tripeptide), still more preferably w is 2 (i.e. still more preferably —W_{w}— is a dipeptide);
* denotes the attachment point to the Y; and ## denotes the attachment point to the drug moiety (-D). Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ).

In some embodiments, the linker L may have the following structure:

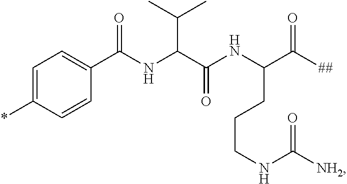

which comprises the dipeptide valine-citrulline as the amino acid unit —W_{w}—; and
wherein * denotes the attachment point to the Y; and ## denotes the attachment point to the drug moiety (-D).

In some embodiments, the linker L may have the following structure:

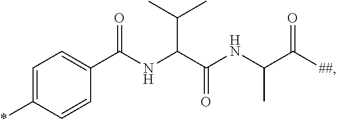

which comprises the dipeptide valine-alanine as the amino acid unit —W_{w}—; and
wherein * denotes the attachment point to the Y; and ## denotes the attachment point to the drug moiety (-D).

In some embodiments, the linker (-L-) has the formula: *-Aₐ##, wherein -A- is a second spacer unit as defined herein; the integer a associated with the second spacer unit is 1; the amino acid unit —W_{w}— is absent; the first spacer unit (—B—) is absent; * denotes the attachment point to the Y; and ## denotes the attachment point to the drug moiety (-D). In any one of these embodiments, the second spacer unit -Aₐ- may be a group Z having the structure

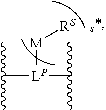

wherein

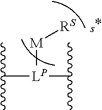

is as defined herein.

The linker (-L-) may have the following structure:

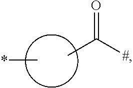

wherein:

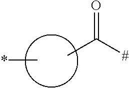

is as defined herein; * denotes the attachment point to the Y; and # denotes the attachment point to the drug moiety (-D).

In some embodiments, the linker L may have the following structure:

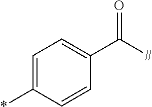

wherein * denotes the attachment point to the Y; and ## denotes the attachment point to the drug moiety (-D).
   Linker *-Aₐ-Q^{CO}_{q}-G-^{##}

In some embodiments, the linker L has the following structure: *-Aₐ-Q^{CO}_{q}-G-##, wherein: -A- is a second spacer unit, as described herein; a is 0 or 1, as described herein; each -Q^{CO}- is independently a connector unit; q is 0 or 1; and -G- is a first spacer unit comprising a sugar moiety; * denotes the attachment point to the Y; and ## denotes the attachment point to the drug moiety (-D). Linkers comprising a sugar moiety, such as e.g. a glucuronic acid moiety, are described, e.g., in Jeffrey et al., “Development and Properties of beta-Glucuronide Linkers for Monoclonal Antibody-Drug Conjugates”, Bioconjugate Chem. 2006, 17, 831-840, doi: 10.1021/bc0600214; WO 2019/236954; and WO 2015/057699.

In the linker *-AₐQ^{CO}_{q}-G-^{##}, the second spacer unit -A-, when present, may be any second spacer unit as described herein. In the linker having the structure *-Aₐ-Q^{CO}_{q}-G-##, the second spacer unit serves to connect the Y with the connector unit Q^{CO}, when present, or with the first spacer unit comprising a sugar moiety. The second spacer unit (-A-), when present, may be any chemical group or moiety which is capable to link a Y to the connector unit (Q^{CO}). Alternatively, the second spacer unit (-A-) may link the Y to the first spacer unit comprising a sugar moiety (-G-), in case no connector unit Q^{CO} is present. In this regard, the Y, as described herein, is bonded to the second spacer unit (-A-). The second spacer unit (-A-) comprises or is a functional group that is capable to form a bond to a connector unit (-Q^{CO}-), or to a first spacer unit having a sugar moiety (-G-), depending on whether a connector unit (-Q^{CO}-) is present or not. Preferably, the functional group, which is capable to form a bond to a connector unit (-Q^{CO}-), or to a first spacer unit comprising a sugar moiety (-G-), is a carbonyl group which is depicted as, e.g.,

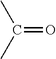

or —C(O)-. The integer a associated with the second spacer unit may be 0 or 1. Preferably, the integer a is 1. Alternatively, in other embodiments the second spacer unit is absent (a=0).

In the linker *-Aₐ-Q^{CO}_{q}G-^{##}, the second spacer unit -A-, when present, may be any second spacer unit as described herein. In some embodiments, the second spacer unit -A-, when present, may be a group Z having the structure

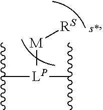

wherein

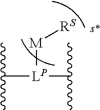

is as defined herein. Accordingly, in some embodiments, the linker (L) may have the structure

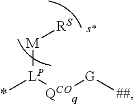

wherein L^{P}, R^{S}, s*, M, Q^{CO}, q, and G are as defined herein; * denotes the attachment point to the —Y—; and ## denotes the attachment point to the drug moiety (-D).

The connector unit (-Q^{CO}-) may be included in instances where it is desirable to add additional distance between the —Y— or, when present, between the second spacer unit (-A-) and the first spacer unit comprising a sugar moiety (-G-). In some embodiments, the extra distance may aid with activation within the first spacer unit comprising a sugar moiety (-G-). Accordingly, the connector unit (-Q^{CO}-), when present, extends the framework of the linker (-L-). In this regard, a connector unit (-Q^{CO}-) is covalently bonded with the —Y— or, when a second spacer unit -A- is present, with the second spacer unit (-A-) at one terminus, and the connector unit (-Q^{CO}-) is covalently bonded to the first spacer unit comprising a sugar moiety (-G-) at its other terminus. The integer q associated with the connector unit Q^{CO} may be 0 or 1. Preferably, the integer q is 1. Alternatively, in other embodiments, the connector unit Q^{CO} is absent (q=0).

The connector unit (-Q^{CO}-), when present, serves to link the first spacer unit comprising a sugar moiety (-G-) to the second spacer unit (-A-), when present, or to the —Y—. The connector unit Q^{CO} may be any chemical group or moiety that serves to provide for attachment of the first spacer unit comprising a sugar moiety (-G-) to the second spacer unit (-A-), when present, or to the —Y—. The connector unit may be, for example, comprised of one or more (e.g., 1-10, preferably, 1, 2, 3, or 4) natural or non-natural amino acid, amino alcohol, amino aldehyde, and diamino residues. In some embodiments, the connector unit (-Q^{CO}-) is a single natural or non-natural amino acid, amino alcohol, amino aldehyde, or diamino residue.

In some embodiments, the amino acid capable of acting as connector unit is beta-alanine. In particular, the connector unit may be a single beta-alanine.

In some embodiments, the connector unit (-Q^{CO}-) has the formula denoted below:

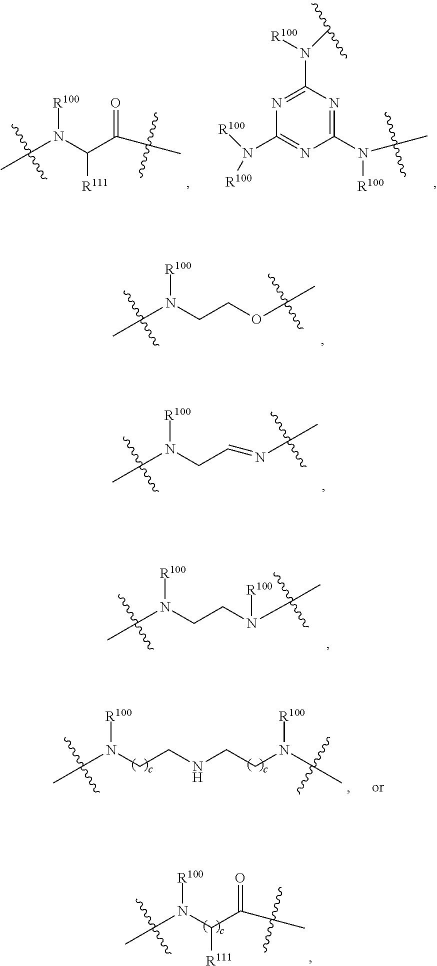

wherein the wavy lines indicate attachment of the connector unit within the linker (-L-) or to —Y—, when the second spacer unit (-A-) is absent; and wherein R¹¹¹ is independently selected from the group consisting of hydrogen, hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-,

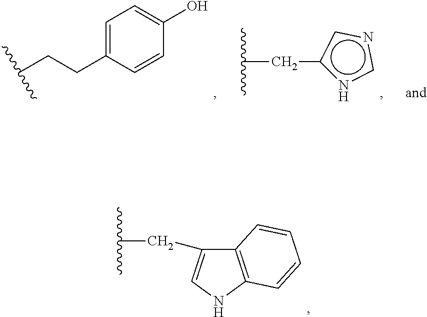

and each R¹⁰⁰ is independently selected from hydrogen or —(C₁-C₃)alkyl, preferably hydrogen or CH₃; and subscript c is an independently selected integer from 1 to 10, preferably 1 to 3.

In preferred embodiments, a connector unit has the following structure (-Q^{CO}-) having a carbonyl group for attachment to the first spacer unit comprising a sugar moiety (-G-), and an NH group for attachment to the second spacer unit (-A-), when present, as follows:

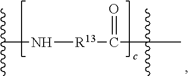

wherein in each instance R¹³ is independently selected from the group consisting of —(C₁-C₆)alkylene-, —(C₃-C₈)carbocyclo-, -arylene-, —(C₁-C₁₀)heteroalkylene-, —(C₃-C₈)heterocyclo-, -(C₁-C₁₀)alkylene-arylene-, -arylene-(C₁-C₁₀)alkylene-, —(C₁-C₁₀)alkylene-(C₃-C₈)carbocyclo)-, -(C₃-C₈)carbocyclo-(C₁-C₁₀)alkylene-, —(C₁-C₁₀)alkylene-(C₃-C₈)heterocyclo-, and —(C₃-C₃)heterocyclo-(C₁-C₁₀)alkylene-, and the subscript c is an integer ranging from 1 to 4. In some embodiments R¹³ is —(C₁-C₆)alkylene and c is an integer ranging from 1 to 4. In preferred embodiments, R¹³ is —(C₁-C₆)alkylene and c is 1.

More preferably, the connector unit (-Q^{CO}-) has the following structure of:

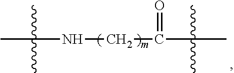

wherein the wavy line adjacent to the nitrogen indicates covalent attachment to a second spacer unit (-A-), when present, and the wavy line adjacent to the carbonyl indicates covalent attachment to the first spacer group comprising a sugar moiety (-G-); and m is an integer ranging from 1 to 6, preferably 2 to 6, more preferably 2 to 4.

Still more preferably, the connector unit (-Q^{CO}-) has the following structure of:

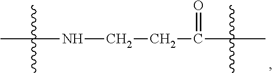 [SMILES: *NCCC(*)=O]

wherein the wavy line adjacent to the nitrogen indicates covalent attachment to a second spacer unit (-A-), when present, and the wavy line adjacent to the carbonyl indicates covalent attachment to the first spacer group comprising a sugar moiety (-G-).

Another representative connector unit (-Q^{CO}-) having a carbonyl group for attachment to the first spacer unit comprising a sugar moiety (-G-) is as follows:

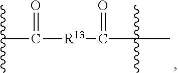

wherein R¹³ is —(C₁-C₆)alkylene-, —(C₃-C₈)carbocyclo-, -arylene-, —(C₁-C₁₀)heteroalkylene-, —(C₃-C₃)heterocyclo-, —(C₁-C₁₀)alkylene-arylene-, -arylene-(C₁-C₁₀)alkylene-, —(C₁-C₁₀)alkylene-(C₃-C₃)carbocyclo-, —(C₃-C₈)carbocyclo-(C₁-C₁₀)alkylene-, —(C₁-C₁₀)alkylene-(C₃-C₈)heterocyclo-, or —(C₃-C₈)heterocyclo-(C₁-C₁₀)alkylene-. In some embodiments R¹³ is —(C₁-C₆)alkylene.

Another representative connector unit having an NH moiety that attaches to the first spacer unit comprising a sugar moiety (-G-) is as follows:

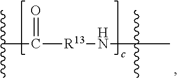

wherein in each instance, R¹³ is independently selected from the group consisting of -(C1-C6)alkylene-, —(C₃-C₈)carbocyclo-, -arylene-, —(C₁-C₁₀)heteroalkylene-, —(C₃-C₈)heterocyclo-, -(C₁-C₁₀)alkylene-arylene-, -arylene-(C₁-C₁₀)alkylene-, —(C₁-C₁₀)alkylene-(C₃-C₈)carbocyclo-, -(C₃-C₈)carbocyclo-(C₁-C₁₀)alkylene-, —(C₁-C₁₀)alkylene-(C₃-C₈)heterocyclo-, and —(C₃-C₃)heterocyclo-(C₁-C₁₀)alkylene-, and subscript c is from 1 to 14. In some embodiments R¹³ is -(C₁-C₆)alkylene and subscript c is 1.

Another representative connector unit (-Q^{CO}-) having a NH moiety that attaches to the first spacer unit comprising a sugar moiety (-G-) is as follows:

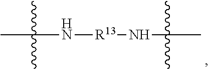

wherein R¹³ is —(C₁-C₆)alkylene-, —(C₃-C₈)carbocyclo-, -arylene-, —(C₁-C₁₀)heteroalkylene-, —(C₃-C₃)heterocyclo-, —(C₁-C₁₀)alkylene-arylene-, -arylene-(C₁-C₁₀)alkylene-, —(C₁-C₁₀)alkylene-(C₃-C₃)carbocyclo-, —(C₃-C₈)carbocyclo-(C₁-C₁₀)alkylene-, —(C₁-C₁₀)alkylene-(C₃-C₈)heterocyclo-, -(C₃-C₈)heterocyclo-(C₁-C₁₀)alkylene-, —C(═O)(C₁-C₁₀)alkylene- or —(C₁-C₆)alkylene-C(═O)-(C₁-C₆)alkylene.

The first spacer unit having a sugar moiety (-G-) is the only component of the linker having the structure *-AaQ^{C}oqG-^{##} that must be present. In some embodiments, the first spacer unit comprising a sugar moiety (-G-) forms a cleavable bond with the drug moiety (-D). In some embodiments, the first spacer unit comprising a sugar moiety (-G-) forms a cleavable bond with the connector unit (-Q^{CO}-), when present. In some embodiments, the cleavable bond is within the first spacer unit comprising a sugar moiety (-G-) but allows for release of free drug (e.g., by a 1,6-elimination reaction following cleavage). Functional groups for forming cleavable bonds can include, for example, sugars to form glycosidic bonds.

The structure and sequence of the first spacer unit comprising a sugar moiety (-G-) may be such that the unit is cleaved by the action of enzymes present at the target site. In other embodiments, the first spacer unit comprising a sugar moiety (-G-) may be cleavable by other mechanisms. The first spacer unit comprising a sugar moiety (-G-) may comprise one or multiple cleavage sites.

Preferably, the first spacer unit comprising a sugar moiety (-G-) comprises a sugar cleavage site. In some such embodiments, the first spacer unit comprising a sugar moiety (-G-) comprises a sugar moiety (Su) linked via an oxygen glycosidic bond to a self-immolative group. In such aspects, the self-immolative group is considered to be part of the first spacer unit comprising a sugar moiety (-G-). In this regard, the “self-immolative group” may be a tri-functional chemical moiety that is capable of covalently linking together three spaced chemical moieties (i.e., the sugar moiety (via a glycosidic bond), a drug moiety (-D), and a connector unit -Q^{CO}-, a second spacer unit -A-, or —Y—, depending on whether a -Q^{CO}-unit and/or an -A- unit are present or not. The glycosidic bond may be one that can be cleaved at the target site to initiate a self-immolative reaction sequence that leads to a release of the drug. Particular sugar moieties may be selected, e.g., from the group consisting of glucuronic acid, galactose, glucose, arabinose, mannose-6-phosphate, fucose, rhamnose, gulose, allose, 6-deoxy-glucose, lactose, maltose, cellobiose, gentiobiose, maltotriose, GlcNAc, GalNAc and maltohexaose.

Accordingly, the first spacer unit comprising a sugar moiety (-G-) may comprise a sugar moiety (Su) linked via a glycoside bond (—O′—) to a self-immolative group (K) of the formula:

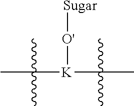

wherein the self-immolative group K forms a covalent bond with the drug moiety and a covalent bond with -Q^{CO}-, -A-, or —Y—, as the case may be.

The first spacer unit comprising a sugar moiety (-G-) may be, for example, represented by the formula:

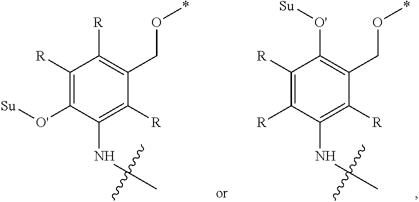

wherein Su is a Sugar moiety, —O′— represents an oxygen glycosidic bond; each R is independently hydrogen, a halogen, —CN, or —NO₂; and wherein the wavy line indicates attachment to -Q^{CO}-, -A-, or —Y—, as the case may be, and the asterisk indicates attachment to the drug moiety (either directly or indirectly via a spacer unit; the spacer unit, when present, may be, for example —(C═O)—).

In some such embodiments, the sugar cleavage site is recognized by a beta-glucuronidase and the first spacer unit comprising a sugar moiety (-G-) comprises a glucuronide unit. The glucuronide unit may comprise glucuronic acid linked via a glycoside bond (—O′—) to a self-immolative group (K) of the formula:

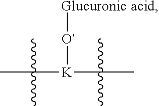

wherein the self-immolative group K forms a covalent bond with the drug moiety (either directly or indirectly via a spacer unit; the spacer unit, when present, may be, for example —(C═O)—) and a covalent bond with -Q^{CO}-, -A-, or —Y—, as the case may be.

The glucuronide unit may be, for example, represented by the formula:

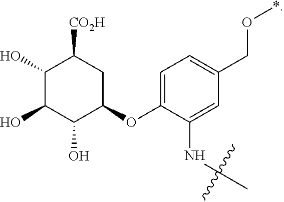

wherein the wavy line indicates covalent attachment to the -Q^{CO}-, -A- or —Y—, as the case may be, and the asterisk indicates covalent attachment to the drug moiety —C (either directly or indirectly via a spacer unit; the spacer unit, when present, may be, for example —(C═O)—).

In some embodiments the first spacer unit comprising a sugar moiety (-G-) comprises a sugar cleavage site, and —S—C, i.e. the combination of the first spacer unit comprising a sugar moiety (-G-) and the drug moiety (-D), is represented by the following formulae:

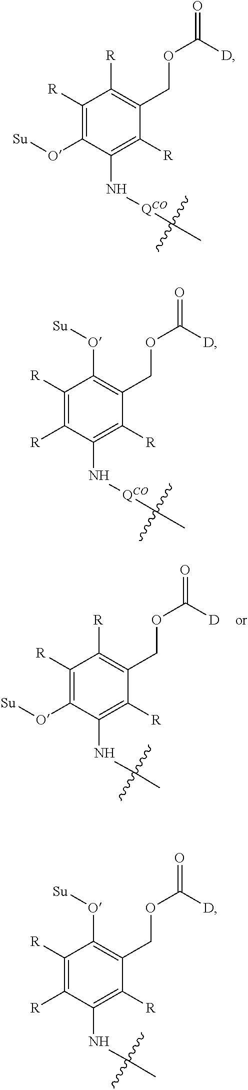

wherein Su is a sugar moiety, D is a drug moiety, —O′— represents an oxygen glycosidic bond; each R is each independently hydrogen or halogen, —CN, —NO₂ or other electron withdrawing group, -Q^{CO}- is a connector unit, as described herein; wherein the wavy bond indicates covalent attachment to the -A- or —Y—, as the case may be.

When the first spacer unit comprising a sugar moiety (-G-) comprises a glucuronide unit, then —S—C, i.e. the combination of the first spacer unit comprising a sugar moiety (-G-) and the drug moiety (-D) may be, for example, represented by the following formulae:

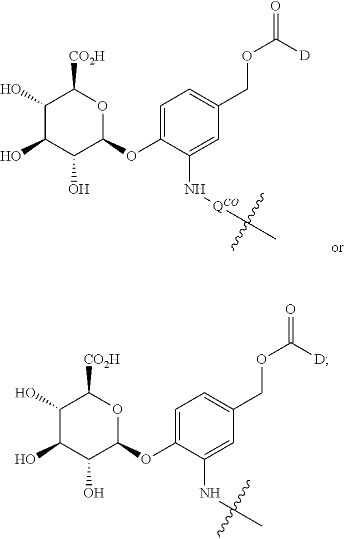

wherein the wavy bond indicates covalent attachment to the -A- or —Y—, as the case may be; D is a drug moiety; and -Q^{CO}- is a connector unit, as described herein.

In preferred embodiments, the linker (L) has the following structure:

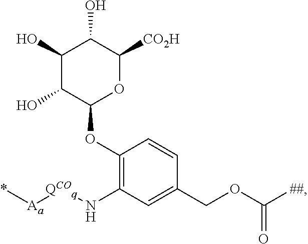

wherein:
-A- is a second spacer unit as described herein; a is an integer as described herein, preferably a is 1;
-Q^{CO}- is a connector unit as described herein; q is an integer as defined herein, preferably q is 1;
* denotes the attachment point to the Y; and ## denotes the attachment point to the drug moiety (-D). In these embodiments, the connector unit (Q^{CO}), when present, may have a carbonyl group for attachment to the first spacer unit comprising a sugar moiety (-G-) and an NH group for attachment to the second spacer unit (-A-), when present, and may be as follows:

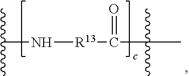

wherein in each instance R¹³ is independently selected from the group consisting of —(C₁-C₆)alkylene-, —(C₃-C₈)carbocyclo-, -arylene-, —(C₁-C₁₀)heteroalkylene-, —(C₃-C₈)heterocyclo-, —(C₁-C₁₀)alkylene-arylene-, -arylene-(C₁-C₁₀)alkylene-, —(C₁-C₁₀)alkylene-(C₃-C₈)carbocyclo)-, —(C₃-C₈)carbocyclo-(C₁-C₁₀)alkylene-, —(C₁-C₁₀)alkylene-(C₃-C₈)heterocyclo-, and —(C₃-C₈)heterocyclo-(C₁-C₁₀)alkylene-, and the subscript c is an integer ranging from 1 to 4. In some embodiments R¹³ is —(C₁-C₆)alkylene and c is an integer ranging from 1 to 4. In preferred embodiments, R¹³ is —(C₁-C₆)alkylene and c is 1; Preferably, in these embodiments, the connector unit (-Q^{CO}-), when present, may have the following structure of:

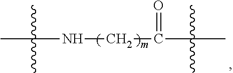

wherein the wavy line adjacent to the nitrogen indicates covalent attachment to a second spacer unit (-Aₐ-), when present, and the wavy line adjacent to the carbonyl indicates covalent attachment to the first spacer group comprising a sugar moiety (-G-); and m is an integer ranging from 1 to 6, preferably 2 to 6, more preferably 2 to 4; More preferably, in these embodiments, the connector unit (-Q^{CO}-), when present, has the following structure of:

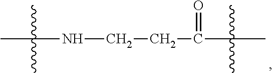 [SMILES: *NCCC(*)=O]

wherein the wavy line adjacent to the nitrogen indicates covalent attachment to a second spacer unit (-Aₐ-), when present, and the wavy line adjacent to the carbonyl indicates covalent attachment to the first spacer group comprising a sugar moiety (-G-).

More preferably, the linker (L) has the following structure:

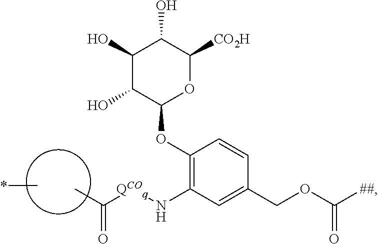

wherein:

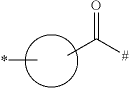

is as defined herein; * denotes the attachment point to the —Y—; and # denotes the attachment point to the connector unit (-Q^{CO}-), when present, or to the NH group; -Q^{CO}- is a connector unit as defined herein; q is an integer as defined herein, preferably q is 1; * denotes the attachment point to the Y; and ## denotes the attachment point to the drug moiety (-D). In these embodiments, the connector unit (Q^{CO}), when present, may have a carbonyl group for attachment to the first spacer unit comprising a sugar moiety (-G-) and an NH group for attachment to the second spacer unit (-A-), and may be as follows:

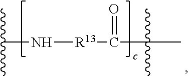

wherein in each instance R¹³ is independently selected from the group consisting of —(C₁-C₆)alkylene-, —(C₃-C₈)carbocyclo-, -arylene-, —(C₁-C₁₀)heteroalkylene-, —(C₃-C₈)heterocyclo-, —(C₁-C₁₀)alkylene-arylene-, -arylene-(C₁-C₁₀)alkylene-, —(C₁-C₁₀)alkylene-(C₃-C₈)carbocyclo)-, —(C₃-C₈)carbocyclo-(C₁-C₁₀)alkylene-, —(C₁-C₁₀)alkylene-(C₃-C₈)heterocyclo-, and —(C₃-C₈)heterocyclo-(C₁-C₁₀)alkylene-, and the subscript c is an integer ranging from 1 to 4. In some embodiments R¹³ is —(C₁-C₆)alkylene and c is an integer ranging from 1 to 4. In preferred embodiments, R¹³ is —(C₁-C₆)alkylene and c is 1. Preferably, in these embodiments, the connector unit (-Q^{CO}ₚ-), when present, may have the following structure of:

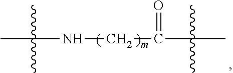

wherein the wavy line adjacent to the nitrogen indicates covalent attachment to a second spacer unit (-A-), and the wavy line adjacent to the carbonyl indicates covalent attachment to the first spacer group comprising a sugar moiety (-G-); and m is an integer ranging from 1 to 6, preferably 2 to 6, more preferably 2 to 4. More preferably, in these embodiments, the connector unit (-Q^{CO}-), when present, has the following structure of:

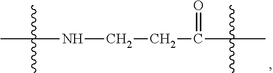 [SMILES: *NCCC(*)=O]

wherein the wavy line adjacent to the nitrogen indicates covalent attachment to a second spacer unit (-Aₐ-), when present, and the wavy line adjacent to the carbonyl indicates covalent attachment to the first spacer group comprising a sugar moiety (-G-).

Still more preferably, the linker (L) has the following structure:

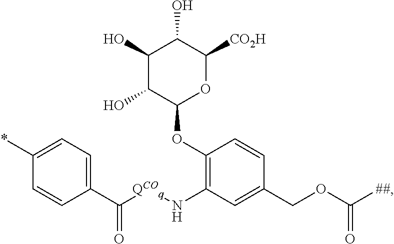

wherein:
Q^{CO} is a connector unit as defined herein; q is an integer as defined herein, preferably q is 1; * denotes the attachment point to the Y; and ## denotes the attachment point to the drug moiety (-D). In these embodiments, the connector unit (Q^{CO}), when present, may have a carbonyl group for attachment to the first spacer unit comprising a sugar moiety (-G-) and an an NH group for attachment to the second spacer unit (-A-), and may be as follows:

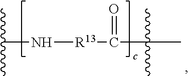

wherein in each instance R¹³ is independently selected from the group consisting of —(C₁-C₆)alkylene-, —(C₃-C₈)carbocyclo-, -arylene-, —(C₁-C₁₀)heteroalkylene-, —(C₃-C₈)heterocyclo-, —(C₁-C₁₀)alkylene-arylene-, -arylene-(C₁-C₁₀)alkylene-, —(C₁-C₁₀)alkylene-(C₃-C₈)carbocyclo)-, —(C₃-C₈)carbocyclo-(C₁-C₁₀)alkylene-, —(C₁-C₁₀)alkylene-(C₃-C₈)heterocyclo-, and —(C₃-C₈)heterocyclo-(C₁-C₁₀)alkylene-, and the subscript c is an integer ranging from 1 to 4. In some embodiments R¹³ is —(C₁-C₆)alkylene and c is an integer ranging from 1 to 4. In preferred embodiments, R¹³ is —(C₁-C₆)alkylene and c is 1. Preferably, in these embodiments, the connector unit (-Q^{CO}-), when present, may have the following structure of:

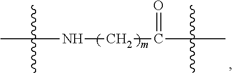

wherein the wavy line adjacent to the nitrogen indicates covalent attachment to a second spacer unit (-A-), and the wavy line adjacent to the carbonyl indicates covalent attachment to the first spacer group comprising a sugar moiety (-G-); and m is an integer ranging from 1 to 6, preferably 2 to 6, more preferably 2 to 4; More preferably, in these embodiments, the connector unit (-Q^{CO}-), when present, has the following structure of:

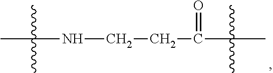 [SMILES: *NCCC(*)=O]

wherein the wavy line adjacent to the nitrogen indicates covalent attachment to a second spacer unit (-A-), and the wavy line adjacent to the carbonyl indicates covalent attachment to the first spacer group comprising a sugar moiety (-G-).

Still more preferably, the linker L may have the following structure:

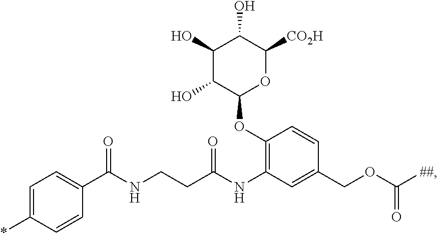

wherein * denotes the attachment point to the —Y—; and ## denotes the attachment point to the drug moiety (-D).

In preferred embodiments, the linker L has the following structure:

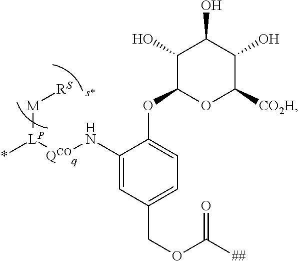

wherein:

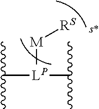

is as described herein; R^{S} is, each independently, a second polyalkylene glycol unit as described herein; preferably each R^{S} is, independently, a second polyethylene glycol unit as described herein; M is, each independently, as described herein; preferably each M is —O—; s* is an integer as described herein; preferably, s* is 1; -Q^{CO}- is a connector unit as described herein; q is an integer as defined herein, preferably q is 1;
* denotes the attachment point to the Y; and ## denotes the attachment point to the drug moiety (-D). In these embodiments, the connector unit (Q^{CO}), when present, may have a carbonyl group for attachment to the first spacer unit comprising a sugar moiety (-G-) and an NH group for attachment to the second spacer unit (-A-), and may be as follows:

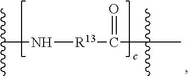

wherein in each instance R¹³ is independently selected from the group consisting of —(C₁-C₆)alkylene-, —(C₃-C₈)carbocyclo-, -arylene-, —(C₁-C₁₀)heteroalkylene-, —(C₃-C₈)heterocyclo-, —(C₁-C₁₀)alkylene-arylene-, -arylene-(C₁-C₁₀)alkylene-, —(C₁-C₁₀)alkylene-(C₃-C₈)carbocyclo)-, —(C₃-C₈)carbocyclo-(C₁-C₁₀)alkylene-, —(C₁-C₁₀)alkylene-(C₃-C₈)heterocyclo-, and —(C₃-C₈)heterocyclo-(C₁-C₁₀)alkylene-, and the subscript c is an integer ranging from 1 to 4. In some embodiments R¹³ is —(C₁-C₆)alkylene and c is an integer ranging from 1 to 4. In preferred embodiments, R¹³ is —(C₁-C₆)alkylene and c is 1. Preferably, in these embodiments, the connector unit (-Q^{CO}-), when present, may have the following structure of:

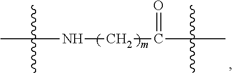

wherein the wavy line adjacent to the nitrogen indicates covalent attachment to a second spacer unit (-A-), and the wavy line adjacent to the carbonyl indicates covalent attachment to the first spacer group comprising a sugar moiety (-G-); and m is an integer ranging from 1 to 6, preferably 2 to 6, more preferably 2 to 4. More preferably, in these embodiments, the connector unit (-Q^{CO}-), when present, has the following structure of:

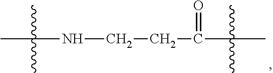 [SMILES: *NCCC(*)=O]

wherein the wavy line adjacent to the nitrogen indicates covalent attachment to a second spacer unit (-A-), and the wavy line adjacent to the carbonyl indicates covalent attachment to the first spacer group comprising a sugar moiety (-G-).

More preferably, the linker (L) has the following structure:

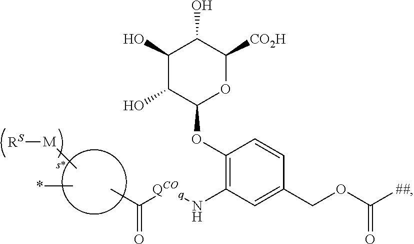

wherein:

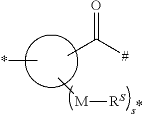

is as defined herein; R^{S} is, each independently, a second poly(alkylene) glycol unit as defined herein; preferably, each R^{S} is, independently, a second polyethylene glycol unit as defined herein; M is, each independently, as defined herein; preferably each M is —O—; s* is an integer as defined herein; preferably s* is 1; * denotes the attachment point to the Y; * denotes the attachment point to the —Y—; and # denotes the attachment point to the connector unit (-Q^{CO}-), when present, or to the NH group; -Q^{CO}- is a connector unit as defined herein; q is an integer as defined herein, preferably q is 1; * denotes the attachment point to the Y; and ## denotes the attachment point to the drug moiety (-D). In these embodiments, the connector unit (Q^{CO}), when present, may have a carbonyl group for attachment to the first spacer unit comprising a sugar moiety (-G-) and an NH group for attachment to the second spacer unit (-A-), and may be as follows:

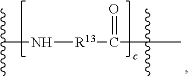

wherein in each instance R¹³ is independently selected from the group consisting of —(C₁-C₆)alkylene-, —(C₃-C₈)carbocyclo-, -arylene-, —(C₁-C₁₀)heteroalkylene-, —(C₃-C₈)heterocyclo-, —(C₁-C₁₀)alkylene-arylene-, -arylene-(C₁-C₁₀)alkylene-, —(C₁-C₁₀)alkylene-(C₃-C₈)carbocyclo)-, —(C₃-C₈)carbocyclo-(C₁-C₁₀)alkylene-, —(C₁-C₁₀)alkylene-(C₃-C₈)heterocyclo-, and —(C₃-C₈)heterocyclo-(C₁-C₁₀)alkylene-, and the subscript c is an integer ranging from 1 to 4. In some embodiments R¹³ is —(C₁-C₆)alkylene and c is an integer ranging from 1 to 4. In preferred embodiments, R¹³ is —(C₁-C₆)alkylene and c is 1. Preferably, in these embodiments, the connector unit (-Q^{CO}-) may have the following structure of:

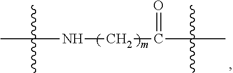

wherein the wavy line adjacent to the nitrogen indicates covalent attachment to a second spacer unit (-A-), and the wavy line adjacent to the carbonyl indicates covalent attachment to the first spacer group comprising a sugar moiety (-G-); and m is an integer ranging from 1 to 6, preferably 2 to 6, more preferably 2 to 4. More preferably, in these embodiments, the connector unit (-Q^{CO}-), when present, has the following structure of:

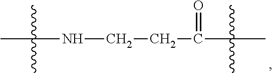 [SMILES: *NCCC(*)=O]

wherein the wavy line adjacent to the nitrogen indicates covalent attachment to a second spacer unit (-A-), and the wavy line adjacent to the carbonyl indicates covalent attachment to the first spacer group comprising a sugar moiety (-G-).

More preferably, the linker (L) has the following structure:

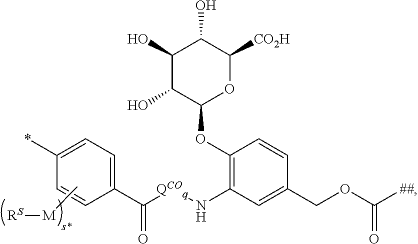

wherein:
R^{S} is, each independently, a second polyalkylene glycol unit as defined herein; preferably each R^{S} is, independently, a second polyethylene glycol unit as described herein; M is, each independently, as defined herein; preferably each M is —O—; s* is an integer as defined herein; preferably s* is 1.

Q^{CO} is a connector unit as defined herein; q is an integer as defined herein, preferably q is 1;
* denotes the attachment point to the Y; and ## denotes the attachment point to the drug moiety (-D). In these embodiments, the connector unit (Q^{CO}), when present, may have a carbonyl group for attachment to the first spacer unit comprising a sugar moiety (-G-) and an NH group for attachment to the second spacer unit (-A-), and may be as follows:

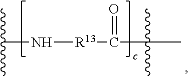

wherein in each instance R¹³ is independently selected from the group consisting of —(C₁-C₆)alkylene-, —(C₃-C₈)carbocyclo-, -arylene-, —(C₁-C₁₀)heteroalkylene-, —(C₃-C₈)heterocyclo-, —(C₁-C₁₀)alkylene-arylene-, -arylene-(C₁-C₁₀)alkylene-, —(C₁-C₁₀)alkylene-(C₃-C₈)carbocyclo)-, —(C₃-C₈)carbocyclo-(C₁-C₁₀)alkylene-, —(C₁-C₁₀)alkylene-(C₃-C₈)heterocyclo-, and —(C₃-C₈)heterocyclo-(C₁-C₁₀)alkylene-, and the subscript c is an integer ranging from 1 to 4. In some embodiments R¹³ is —(C₁-C₆)alkylene and c is an integer ranging from 1 to 4. In preferred embodiments, R¹³ is —(C₁-C₆)alkylene and c is 1. Preferably, in these embodiments, the connector unit (-Q^{CO}-), when present, may have the following structure of:

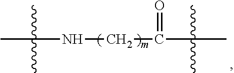

wherein the wavy line adjacent to the nitrogen indicates covalent attachment to a second spacer unit (-A-), and the wavy line adjacent to the carbonyl indicates covalent attachment to the first spacer group comprising a sugar moiety (-G-); and m is an integer ranging from 1 to 6, preferably 2 to 6, more preferably 2 to 4. More preferably, in these embodiments, the connector unit (-Q^{CO}-), when present, has the following structure of:

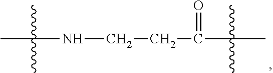 [SMILES: *NCCC(*)=O]

wherein the wavy line adjacent to the nitrogen indicates covalent attachment to a second spacer unit (-A-), and the wavy line adjacent to the carbonyl indicates covalent attachment to the first spacer group comprising a sugar moiety (-G-).

Still more preferably, the linker L has the following structure:

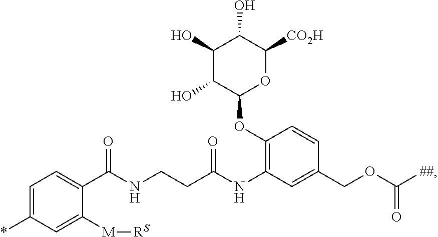

wherein:
R^{S} is, each independently, a second polyalkylene glycol unit as defined herein; preferably each R^{S} is, independently, a second polyethylene glycol unit as described herein; M is, each independently, as defined herein; preferably each M is —O—; s* is an integer as defined herein; preferably s* is 1.

* denotes the attachment point to the —Y—; and ## denotes the attachment point to the drug moiety (-D).

Also preferably, the linker L has the following structure:

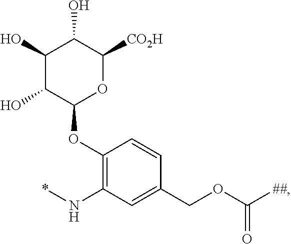

wherein * denotes the attachment point to the —Y—; and ## denotes the attachment point to the drug moiety (-D).

In one embodiment, the linker L has the following structure:

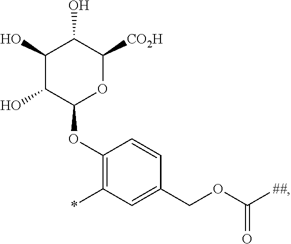

wherein * denotes the attachment point to the —Y—; and ## denotes the attachment point to the drug moiety (-D). In this embodiment, Y may be as defined herein; preferably, Y may be NH.
Linker *-Aₐ-U^{AT}ᵤ-Sulf-^{##}

In some embodiments, the linker L has the following structure: *-Aₐ-U^{AT}ᵤ-Sulf-^{##} wherein: -A- is a second spacer unit; a is 0 or 1; each -U^{AT}ᵤ- is independently an attachment unit; u is 0 or 1; and -Sulf- is a first spacer unit comprising a sulfatase-cleavable moiety; * denotes the attachment point to the Y; and ## denotes the attachment point to the drug moiety (-D). For sulfatase-cleavable linkers, see e.g. Bargh et al., “Sulfatase-cleavable linkers for antibody-drug conjugates”, Chemical Science, 2020, 11, 2375-2380, doi: 10.1039/c9sc06410a.

In the linker having the structure *-Aₐ-U^{AT}ᵤ-Sulf-^{##}, the second spacer unit (-A-), when present, serves to connect a Y to the attachment unit (U^{AT}), when present, or to the first spacer unit comprising a sulfatase-cleavable moiety. The second spacer unit (-A-) may be any chemical group or moiety which is capable to link a Y to the attachment unit (U^{AT}). Alternatively, the second spacer unit (-A-) may link the Y to the first spacer unit comprising a sulfatase-cleavable moiety (-Sulf-), in case no attachment unit (U^{AT}) is present. In this regard, the Y, as described herein, is bonded to the second spacer unit (-A-). The second spacer unit (-A-) comprises or is a functional group that is capable to form a bond to an attachment unit (—U^{AT}—), or to a first spacer unit having a sulfatase-cleavable moiety (-Sulf-), depending on whether an attachment unit (—U^{AT}-) is present or not. Preferably, the functional group, which is capable to form a bond to an attachment unit (—U^{AT}-), or to a first spacer unit comprising a sulfatase-cleavable moiety (-Sulf-), is a carbonyl group which is depicted as, e.g.,

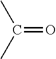

or —C(O)—. The integer a associated with the second spacer unit may be 0 or 1. Preferably, the integer a is 1. Alternatively, in other embodiments the second spacer unit is absent (a=0).

In the linker *-Aₐ-U^{AT}ᵤ-Sulf-^{##}, the second spacer unit -A- may be any second spacer unit as described herein. In some embodiments, the second spacer unit -A-, when present, may be a group Z having the structure

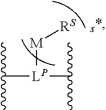

wherein

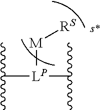

is as defined herein. Accordingly, in some embodiments, the linker (L) may have the structure,

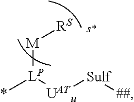

wherein L^{P}, R^{S}, s*, M, U^{AT}, u, and Sulf are as defined herein; * denotes the attachment point to the —Y—; and ## denotes the attachment point to the drug moiety (-D).

The attachment unit (—U^{AT}-) may be included in instances where it is desirable to add additional distance between the —Y— or, when present, the second spacer unit (-A-) and the first spacer unit comprising a sulfatase-cleavable moiety (-Sulf-). Accordingly, the attachment unit (—U^{AT}—), when present, extends the framework of the linker (-L-). In this regard, an attachment unit (—U^{AT}—) may be covalently bonded with the —Y— or, when a second spacer unit -A- is present, with the second spacer unit (-A-) at one terminus, and the attachment unit (—U^{AT}—) is covalently bonded to the first spacer unit comprising a sulfatase-cleavable group (-Sulf-) at its other terminus.

The attachment unit (—U^{AT}—) may be any chemical group or moiety that serves to provide for attachment of the first spacer unit comprising a sulfatase-cleavable moiety (-Sulf-) to the second spacer unit (-A-), when present, or to the —Y—.

In some embodiments, the attachment unit (U^{AT}) has the formula denoted below:

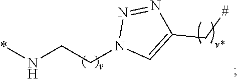

wherein v is an integer ranging from 1 to 6; preferably, v is 1 or 2; more preferably v is 2; and v* is an integer ranging from 1 to 6; preferably, v* is 1 or 2; more preferably v* is 1;
wherein * denotes the attachment point to the second spacer unit (-A-), when present, and # denotes the attachment point to the sulfatase-cleavable moiety (Sulf).

Preferably, the first spacer unit comprising a sulfatase-cleavable moiety (Sulf) has the formulae denoted below:

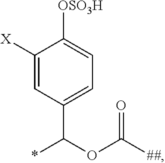

wherein X is hydrogen (H) or an electron withdrawing group, such as e.g. NO₂; * denotes the attachment point to the attachment unit (U^{AT}), when present, or (-A-), when present; and # denotes the attachment point to the drug moiety (-D).

In some embodiments, the linker L may have the following structure:

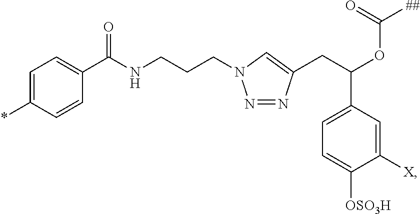

wherein X is H or NO₂;
* denotes the attachment point to the —Y—; and ## denotes the attachment point to the drug moiety (-D).

In some embodiments, the linker L may have the following structure:

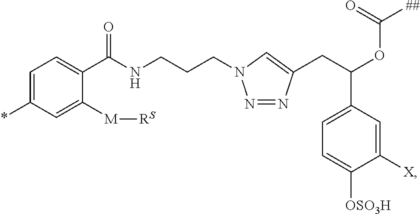

wherein:
X is H or NO₂;
R^{S} is a second polyalkylene glycol unit as defined herein; preferably R^{S} is a second polyethylene glycol unit as defined herein;
M is as defined herein; preferably M is —O—;
* denotes the attachment point to the —Y—; and ## denotes the attachment point to the drug moiety (-D).

### Third Spacer Unit

In some embodiments, when a first spacer unit (—B—), or a first spacer unit comprising a sugar moiety (-G-), or a first spacer unit comprising a sulfatase-cleavable moiety (Sulf) is present, the linker (L) may comprise an optional third spacer unit (-E-), which is arranged between the first spacer unit (—B—), or the first spacer unit comprising a sugar moiety (-G-), or the first spacer unit comprising a sulfatase-cleavable moiety (Sulf) and the drug moiety (-D). The third spacer unit can be a functional group which may facilitate attachment of the first spacer unit (—B—), or the first spacer unit comprising a sugar moiety (-G-), or the first spacer unit comprising a sulfatase-cleavable moiety (Sulf) to the drug moiety (-D), or it can provide additional structural components which may facilitate release of the drug moiety (-D) from the remainder of the conjugate. Suitable third spacer units are described, e.g., in WO 2019/236954.

In some embodiments, the third spacer unit (-E-) is bound to the first spacer unit (—B—) and to the drug moiety (-D). Accordingly, the linker (-L-) may have the structure *-Aₐ-W_{q}—B_{b}-E-^{##}; wherein -E- is a third spacer unit as described herein; wherein -A-, a, —W—, w, and —B— are as described herein, in particular as with regard to the linker (L) having the structure *-Aₐ-W_{w}—B_{b}-^{##}; b is 1; wherein, in each instance, * denotes the attachment point to the —Y—; and ## denotes the attachment point to the drug moiety (-D).

In other embodiments, the third spacer unit (-E-) is bound to the first spacer unit comprising a sugar moiety (-G-) and to the drug moiety (-D-). Accordingly, the linker (-L-) may have the structure *-Aₐ-Q^{CO}_{q}-G-E-^{##}; wherein -E- is a third spacer unit as described herein; wherein -A-, a, -Q^{CO}-, q, and G are as described herein, in particular as with regard to the linker (-L-) having the structure *-Aₐ-Q^{CO}_{q}G-^{##; wherein, in each instance, * denotes the attachment point to the —Y—; and ## denotes the attachment point to the drug moiety (-D).}

In other embodiments, the third spacer unit (-E-) is bound to the first spacer unit comprising a sulfatase-cleavable moiety (-Sulf-) and to the drug moiety (-D). Accordingly, the linker (-L-) may have the structure *-Aₐ-U^{AT}ᵤ-Sulf-E-^{##}; wherein -E- is a third spacer unit as described herein; wherein -A-, a, -U^{AT}, u, and Sujf are as described herein, in particular as with regard to the linker (-L-) having the structure *-Aₐ-U^{AT}ᵤ-Sulf-^{##}; wherein, in each instance, * denotes the attachment point to the —Y—; and ## denotes the attachment point to the drug moiety (-D).

In some embodiments, exemplary third spacer units -E- are represented by the formulae:

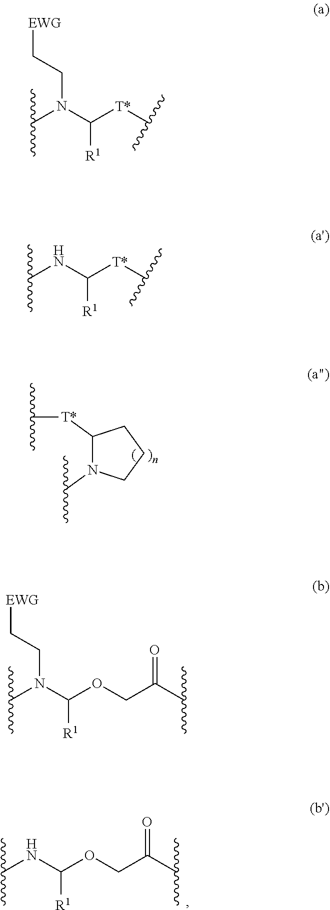

wherein EWG represents an electron-withdrawing group, R¹ is —H or (C₁-C₄)alkyl and subscript n is 1 or 2. In some embodiments, EWG is selected from the group consisting of —CN, —NO₂, —CX₃, —X, C(═O)OR′, —C(═O)N(R′)₂, —C(═O)R′, —C(═O)X, —S(═O)₂R′, —S(═O)₂OR′, —S(═O)₂NHR′, —S(═O)₂N(R′)₂, —P(═O)(OR′)₂, —P(═O)(CH₃)NHR′, —NO, —N(R′)₃+, wherein X is —F, —Br, —Cl, or —I, and R′ is independently selected from the group consisting of hydrogen and (C₁-C₆)alkyl, and wherein the wavy line adjacent to the nitrogen atom in each of formula (a), (a′), (a″), (b) and (b′) is the point of covalent attachment to the first spacer unit (—B—) and the wavy line adjacent to the carbonyl carbon atom of formula (b) and formula (b′) is the point of covalent attachment to a heteroatom of a hydroxyl or primary or secondary amine of a drug moiety (-D); and wherein formula (a), formula (a′) and formula (a″) represents exemplary units in which T* is the heteroatom from a hydroxyl or primary or secondary amine functional group of a drug moiety (-D); and wherein the wavy line adjacent to T* is the point of covalent attachment to the remainder of the drug moiety. In these embodiments, the third spacer unit -E- may facilitate release of the drug moiety as free drug.

In still other embodiments, third spacer units are represented by the formulae:

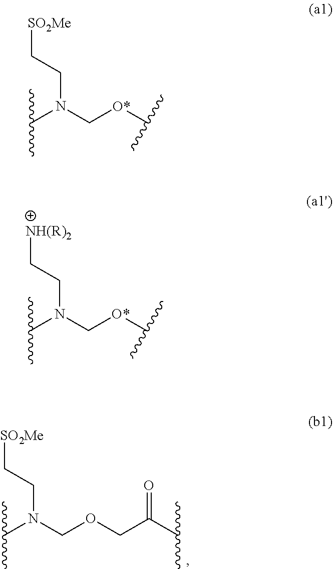

wherein formula (a1) and formula (a1′) in which each R is independently —H or (C₁-C₄)alkyl represents units in which 0* is the oxygen atom from a hydroxyl substituent of a drug moiety (-D); and the wavy lines of formula (a1), formula (a1′) and formula (bl) retain their previous meanings from formulae (a), (a′) and (b), respectively. In formula (a1′) the —CH₂CH₂N+(R)₂ moiety represents exemplary basic units in protonated form.

### Drug Moiety (-D)

The present disclosure provides conjugates, such as e.g. antibody drug conjugates, comprising a drug moiety. The term “drug moiety” or “payload”, both of which can be used interchangeably, as used herein refers to a chemical or biochemical moiety that is conjugated to a receptor binding molecule (RBM), such as e.g. an antibody or antigen binding fragment. In this regard, it is again referred to the conjugate of formula (I) described herein. The receptor binding molecule (RBM) can be conjugated to several identical or different drug moieties using any methods described herein or known in the art. In some embodiments, the drug moiety may be a molecule which has a cytotoxic effect on mammalian cells, may lead to apoptosis, and/or may have a modulating effect on malignant cells. The drug moiety may be hydrophobic.

In some preferred embodiments the drug moiety is an anti-cancer agent. Accordingly, the drug may be selected from the group consisting of maytansinoids, calicheamycins, tubulysins, amatoxins, dolastatins and auristatins such as monomethyl auristatin E (MMAE) or monomethyl auristatin F (MMAF), pyrrolobenzodiazepine dimers, indolino-benzodiazepine dimers, emetine, radioisotopes, therapeutic proteins and peptides (or fragments thereof), kinase inhibitors, CDK inhibitors, histone deacetylase (HDAC) inhibitors, MEK inhibitors, KSP inhibitors, and analogues or prodrugs thereof. In preferred embodiments, the drug moiety is MMAE or MMAF. More preferably, the drug moiety is MMAE.

In some embodiments the drug moiety is a maytansinoid drug moiety, including those having the structure:

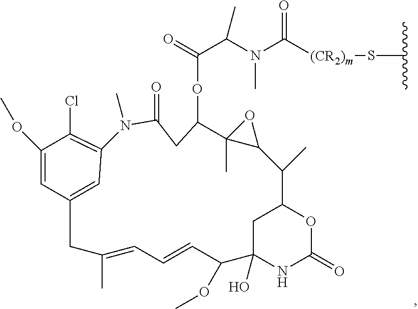

where the wavy line indicates the covalent attachment of the sulfur atom of the maytansinoid to a linker of a conjugate, such as e.g. an antibody drug conjugate. R at each occurrence is independently H or a C1-C6 alkyl. The alkylene chain attaching the amide group to the sulfur atom may be methanyl, ethanyl, or propanyl, i.e. m is 1, 2, or 3. (U.S. Pat. No. 633,410, U.S. Pat. No. 5,208,020, Chari et al. (1992) Cancer Res. 52; 127-131, Lui et al. (1996) Proc. Natl. Acad. Sci. 93:8618-8623).

All stereoisomers of the maytansinoid drug moiety are contemplated for the conjugates, disclosed herein, i.e. any combination of R and S configurations at the chiral carbons of the maytansinoid. In some embodiments the maytansinoid drug moiety has the following stereochemistry:

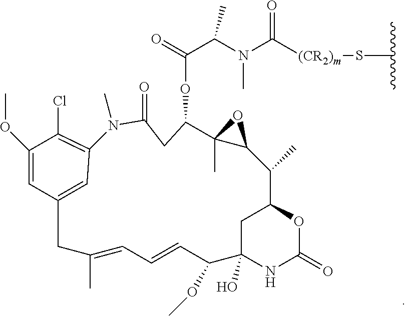

In some embodiments the maytansinoid drug moiety is N^{2′}-deacetyl-N^{2′}-(3-mercapto-1-oxopropyl)-maytansine (also known as DM1). DM1 is represented by the following structural structure:

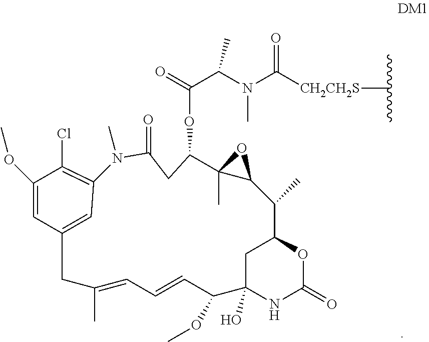

In some embodiments the maytansinoid drug moiety is N^{2′}-deacetyl-N^{2′}-(4-mercapto-1-oxopentyl)-maytansine (also known as DM3). DM3 is represented by the following structural structure:

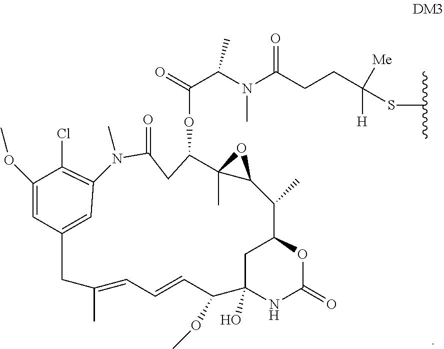 [SMILES: *SC(C)CCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In some embodiments the maytansinoid drug moiety is N′-deacetyl-N′-(4-methyl-4-mercapto-1-oxopentyl)-maytansine (also known as DM4). DM4 is represented by the following structural structure:

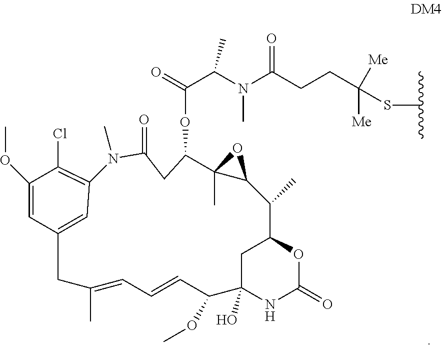 [SMILES: *SC(C)(C)CCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

Preferably, in conjugates, disclosed herein comprising a maytansinoid drug moiety the maytansinoid is N^{2′}-deacetyl-N^{2′}-(3-mercapto-1-oxopropyl)-maytansine (DM1) or N^{2′}-deacetyl-N^{2′}-(4-mercapto-4-methyl-1-oxopentyl)-maytansine (DM4).

The drug moiety may be a calicheamicin. “Calicheamicins” as used herein relate to a class of enediyne antitumor antibiotics derived from the bacterium Micromonospora echinospora, with calicheamicin γ1 being the most notable. It was isolated originally in the mid-1980s from the chalky soil, or “caliche pits”, located in Kerrville, Texas. It is extremely toxic to all cells. Accordingly, the drug moiety may be Calicheamicin γ1 exemplified by the following structure, which may be optionally substituted or derivatized for coupling to a linker and/or a receptor binding molecule:

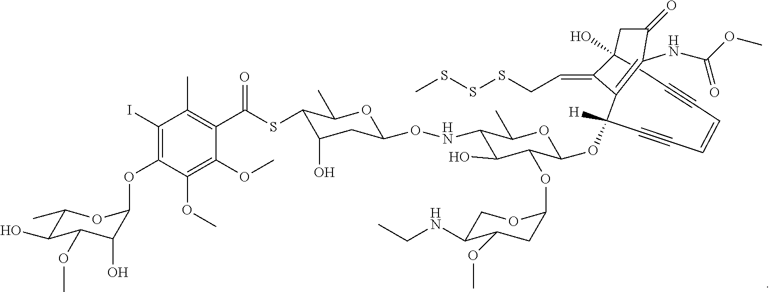 [SMILES: CCNC1COC(OC2C(O[C@H]3C#C/C=C\C#CC[C@]4(O)CC(=O)C(NC(=O)OC)=C3/C4=C\CSSSC)OC(C)C(NOC3CC(O)C(SC(=O)c4c(C)c(I)c(OC5OC(C)C(O)C(OC)C5O)c(OC)c4OC)C(C)O3)C2O)CC1OC]

The drug moiety may be a tubulysin. Tubulysins have functions as being anti-microtubule, anti-mitotic, apoptosis inducer, anticancer, anti-angiogenic, and anti proliferative. Tubulysins are cytotoxic peptides, which include 9 members (A-I). Preferably, the tubulysin is Tubulysin A. Tubulysin A has potential application as an anticancer agent. It arrests cells in the G2/M phase. Tubulysin A has the following structure:

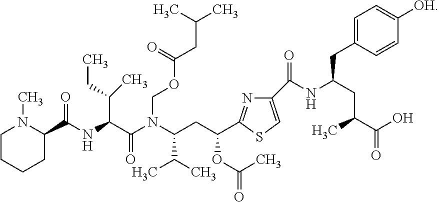 [SMILES: CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)O)cs1)C(C)C]

The drug moiety may be an amatoxin. Amatoxin is the collective name of a subgroup of at least eight related toxic compounds found in several genera of poisonous mushrooms, most notably the death cap (Amanita phalloides) and several other members of the genus Amanita, as well as some Conocybe, Galerina and Lepiota mushroom species. Amatoxins are lethal in even small doses. The compounds have a similar structure, that of eight amino-acid residues arranged in a conserved macrobicyclic motif (an overall pentacyclic structure when counting the rings inherent in the proline and tryptophan-derived residues). All amatoxins are oligopeptides that are synthesized as 35-amino-acid proproteins, from which the final eight amino acids are cleaved by a prolyl oligopeptidase. The schematic amino acid sequence of amatoxins is Ile-Trp-Gly-Ile-Gly-Cys-Asn-Pro with cross-linking between Trp and Cys via the sulfoxide (S═O) moiety and hydroxylation in variants of the molecule. There are currently ten known amatoxins, which might be the drug moiety:

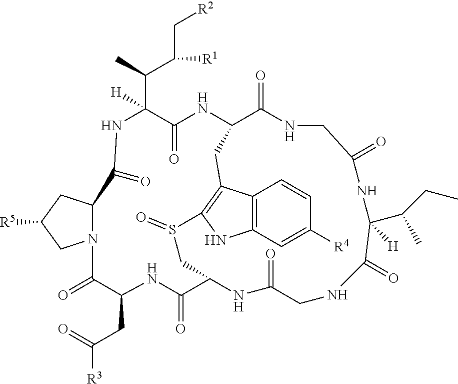

| Name | R¹ | R² | R³ | R⁴ | R⁵ |
|---|---|---|---|---|---|
| α-Amanitin | OH | OH | NH₂ | OH | OH |
| β-Amanitin | OH | OH | OH | OH | OH |
| γ-Amanitin | OH | H | NH₂ | OH | OH |
| ε-Amanitin | OH | H | OH | OH | OH |
| Amanullin | H | H | NH₂ | OH | OH |
| Amanullinic acid | H | H | OH | OH | OH |
| Amaninamide | OH | OH | NH₂ | H | OH |
| Amanin | OH | OH | OH | H | OH |
| Proamanullin | H | H | NH₂ | OH | H |

The drug moiety may be a dolastatin such as Dolastatin 10 or dolastatin 15. Both are marine natural products isolated from the Indian Ocean sea hare Dollabella auricularia. This potent antitumor agent is also isolated from the marine cyanobacterium Symploca sp. VP642 from Palau. Being a small linear peptide molecules, dolastatin 10 and 15 are considered anti-cancer drugs showing potency against breast and liver cancers, solid tumors and some leukemias. Preclinical research indicated potency in experimental antineoplastic and tubulin assembly systems. The dolastatins are mitotic inhibitors. They inhibit microtubule assembly by interfering with tubulin formation and thereby disrupt cell division by mitosis and induces apoptosis and Bcl-2 phosphorylation in several malignant cell types. Dolostatin 10 (N,N-Dimethyl-L-valyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]-1-pyrrolidinyl}-5-methyl-1-oxo-4-heptanyl]-N-methyl-L -valinamide) has the following structure:

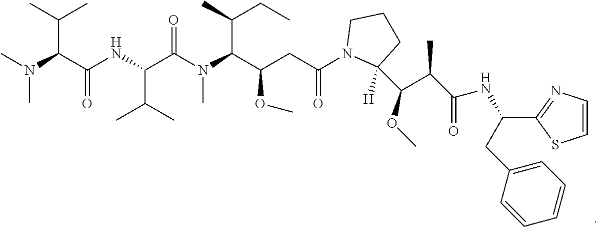 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C)C(C)C]

Dolastatin 15 ((2S)-1-[(2S)-2-Benzyl-3-methoxy-5-oxo-2,5-dihydro-1H-pyrrol-1-yl]-3-methyl-1-oxo-2-butanyl N,N-dimethyl-L-valyl-L-valyl-N-methyl-L-valyl-L-prolyl-L-prol) has the following structure:

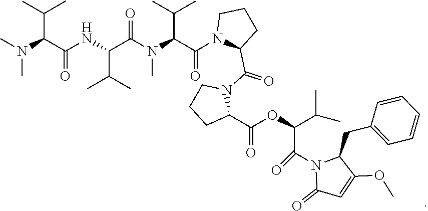 [SMILES: COC1=CC(=O)N(C(=O)[C@@H](OC(=O)[C@@H]2CCCN2C(=O)[C@@H]2CCCN2C(=O)[C@H](C(C)C)N(C)C(=O)[C@@H](NC(=O)[C@H](C(C)C)N(C)C)C(C)C)C(C)C)[C@H]1Cc1ccccc1]

In preferred embodiments of the conjugates, such as e.g. antibody drug conjugates, disclosed herein the drug moiety is an auristatin. Preferably, the auristatin is monomethyl auristatin F (MMAF) or monomethyl auristatin E (MMAE). More preferably, the auristatin is monomethyl auristatin E (MMAE).

In some embodiments of the antibody drug conjugates described herein the drug moiety is monomethyl auristatin F (also known as MMAF). MMAF is represented by the following structural formula:

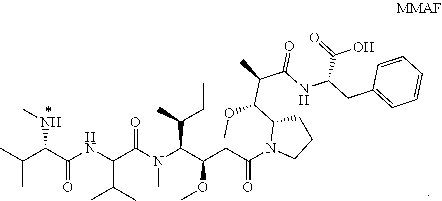 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)C(NC(=O)[C@@H](NC)C(C)C)C(C)C]

Preferably, MMAF is bound to the linker L via the N terminus indicated with an asterisk (“*”).

In some embodiments the auristatin drug moiety is monomethyl auristatin E (also known as MMAE). MMAE is represented by the following structural formula:

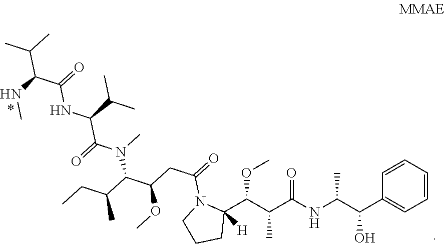 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

Preferably, MMAE is bound to the linker L via the N terminus indicated with an asterisk (“*”)

These molecules noncompetitively inhibit binding of vincristine to tubulin (at a location known as the vinca/peptide region) but have been shown to bind to the RZX/MAY region.

The drug moiety may be a Pyrrolobenzodiazepine Dimer such as a compound having the following structure, which may be optionally substituted or derivatized for coupling to a linker and/or a receptor binding molecule:

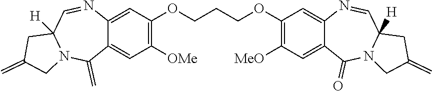

The drug moiety may be a Indolinobenzodiazepin Dimer such as a compound having the following structure:

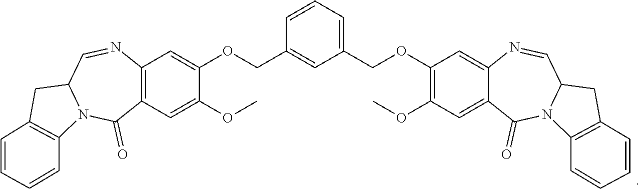 [SMILES: COc1cc2c(cc1OCc1cccc(COc3cc4c(cc3OC)C(=O)N3c5ccccc5CC3C=N4)c1)N=CC1Cc3ccccc3N1C2=O]

The drug moiety may be emetine. Emetine exhibits its anti-tumor effect by apoptosis through such mechanisms as inhibition of protein biosynthesis, DNA interaction and regulation of pro-apoptotic factors (see, e.g., Uzor, “Recent Developments on Potential New Applications of Emetine as Anti-Cancer Agent”, EXCLI Journal 2016; 15:323-238). Emetine has the following structure:

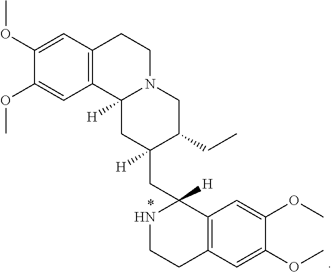 [SMILES: CC[C@H]1CN2CCc3cc(OC)c(OC)cc3[C@@H]2C[C@@H]1C[C@H]1NCCc2cc(OC)c(OC)cc21]

Emetine may be bound to the linker L via the nitrogen atom marked with an asterisk (“*”).

The drug moiety may be a radioisotope. Typical radioisotopes as described herein may relate to a small radiation source, usually a gamma or beta emitter such as iodine-125, iodine-131, iridium-192 or palladium-103.

The drug moiety may be a therapeutic protein or peptide or a fragment thereof.

Typical examples are cytokines such as interleukines, ricin, diphtheria toxin, Pseudomonas exotoxin PE38.

The drug moiety may be a kinase inhibitor, preferably an inhibitor of a kinase associated with a pro-tumorigenic function. Exemplary kinase inhibitors include imatinib, nilotinib, dasatinib, bosutinib, ponatinib, gefitinib, erlotinib, afatinib, osimertinib, lapatinib, neratinib, sorafenib, sunitinib, pazopanib, axitinib, lenvatinib, cabozatinib, vandetanib, regorafenib, vemurafenib, dabrafenib, trametinib, cobimetinib, crizotinib, certinib, alectinib, brigatinib, lorlatinib, ibrutinib, acalibrutinib, midostaurin, ruxolitinib, idelalisib, copanlisib, palbociclib, ribociclib or abemaciclib.

The drug moiety may be a CDK (cyclin-dependent kinase) inhibitor. A “CDK inhibitor” as used herein denotes any chemical or drug that inhibits the function of CDKs (cyclin-dependent kinases). A CDK inhibitor which can be used as drug moiety in the present disclosure is AT7519 (see, e.g., Santo et al., AT7519, “A novel small molecule multi-cyclin- dependent kinase inhibitor, induces apoptosis in multiple myeloma via GSK-3beta activation and RNA polymerase II inhibition”, Oncogene (2010) 29, 2325-2336, doi: 10.1038/onc.2009.510). AT7519 has the following structure:

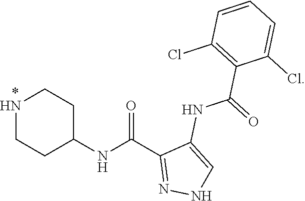 [SMILES: O=C(NC1CCNCC1)c1n[nH]cc1NC(=O)c1c(Cl)cccc1Cl]

AT7519 may be bound to the linker L via the nitrogen atom marked with an asterisk (“*”).

The drug moiety may be a histone deacetylase (HDAC) inhibitor. Histone deacetylase inhibitors are chemical compounds that inhibit histone deacetylases (HDACs). A histone decetylase inhibitor which can be used as drug moiety in the present disclosure is panobinostat (see, e.g., Rasmussen et al., “Panobinostat, a histone deacetylase inhibitor, for latent-virus reactivation in HIV-infected patients on suppressive antiretroviral therapy: a phase 1/2, single groups, clinical trial”, Lancet HIV 2014, 1:e13-21). Panobinostat has the following structure:

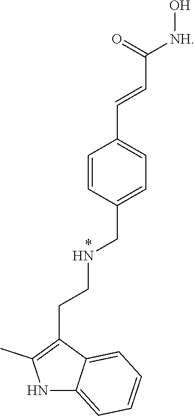 [SMILES: Cc1[nH]c2ccccc2c1CCNCc1ccc(/C=C/C(=O)NO)cc1]

AT7519 may be bound to the linker L via the nitrogen atom marked with an asterisk (“*”).

The drug moiety may be a MEK inhibitor. A MEK inhibitor as used herein describes a chemical or drug that inhibits the mitogen-activated protein kinase kinase enzymes MEK1 and/or MEK2. They can be used to affect the MAPK/ERK pathway which is often overactive in some cancers. Hence MEK inhibitors have potential for treatment of some cancers, especially BRAF-mutated melanoma, and KRAS/BRAF mutated colorectal cancer. Typical MEK inhibitors include Trametinib (GSK1120212), Cobimetinib or XL518, Binimetinib (MEK162), Selumetinib, PD-325901, CI-1040, PD035901, or TAK-733.

The drug moiety may be a KSP (kinesin spindle protein) inhibitor. Examples of KSP inhibitors include Ispinesib (SB-715992), SB743921, AZ 3146, GSK923295, BAY 1217389, MPI-0479605 and ARQ 621. In some embodiments, SB743921 can be used as drug moiety in the present disclsoure (see, e.g., Song et al., “KSP inhibitor SB743921 induces death of multiple myeloma cells via inhibition of the NF-kB signaling pathway”, BMB Reports 2015, 48(10): 571-576). SB743921 has the following structure:

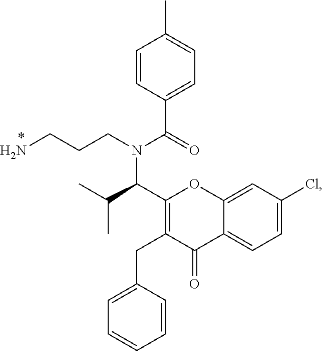 [SMILES: Cc1ccc(C(=O)N(CCCN)[C@@H](c2oc3cc(Cl)ccc3c(=O)c2Cc2ccccc2)C(C)C)cc1]

SB743921 may be bound to the linker L via the nitrogen atom marked with an asterisk (“*”).

The present invention also relates to a conjugate having the formula (I):

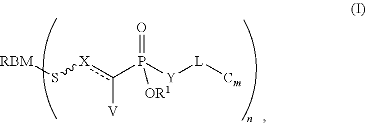

or a pharmaceutically acceptable salt or solvate thereof;
wherein:
RBM is an antibody;
[symbol] is a double bond; or
[symbol] is a bond;
V is absent when [symbol] is a double bond; or
V is H when [symbol] is a bond;
X is R₃—C when [symbol] is a double bond; or
X is

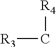

when [symbol] is a bond;
Y is NH;
R¹ is a polyethylene glycol unit having the structure:

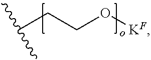

wherein:
[symbol] indicates the position of the O;
K^{F} is as defined herein; preferably K^{F} is H; and
o is an integer as defined herein;
   preferably o is an integer ranging from 8 to 30;
   more preferably, o is an integer ranging from 4 to 16; still more preferably from 8 to 16; still more preferably o is 10, 11, 12, 13 or 14; still more preferably o is 11, 12 or 13; even more preferably o is 12; or
   more preferably o is an integer ranging from 16 to 30; still more preferably from 20 to 28; still more preferably o is 22, 23, 24, 25 or 26; still more preferably, o is 23, 24 or 25; even more preferably o is 24;
R³ is H;
R⁴ is H;
L is a linker having the following structure:

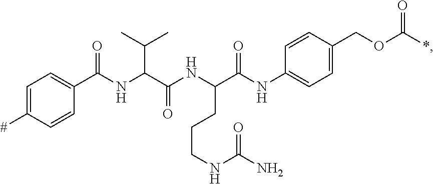

wherein # indicates the attachment point to the Y and * indicates the attachment point to the drug moiety (D);
D is a drug moiety;
m is 1; and
n is an integer as defined herein;
   preferably n is an integer ranging from 1 to 10; more preferably from 2 to 10; still more preferably from 4 to 10; still more preferably from 6 to 10, still more preferably from 7 to 10, even more preferably n is 8; or
   preferably n is an integer ranging from 1 to 10, more preferably from 2 to 8, still more preferably from 3 to 6, still more preferably n is 4 or 5, even more preferably n is 4.

Preferably, the drug moiety D is monomethyl auristatin E (MMAE) or monomethyl auristatin F (MMAF).

More preferably, the drug moiety D is monomethyl auristatin E (MMAE).

The present invention also relates to a conjugate having the following formula (Ia):

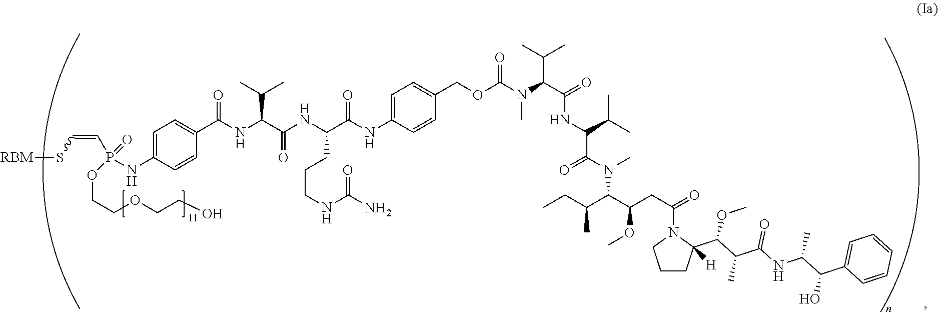

wherein:
REM is an antibody; and
n is an integer as defined herein;
   preferably n is an integer ranging from 1 to 10; more preferably from 2 to 10; still more preferably from 4 to 10; still more preferably from 6 to 10, still more preferably from 7 to 10, even more preferably n is 8; or
   preferably n is an integer ranging from 1 to 10, more preferably from 2 to 8, still more preferably from 3 to 6, still more preferably n is 4 or 5, even more preferably n is 4.

The present invention also relates to a conjugate having the following formula (Ib):

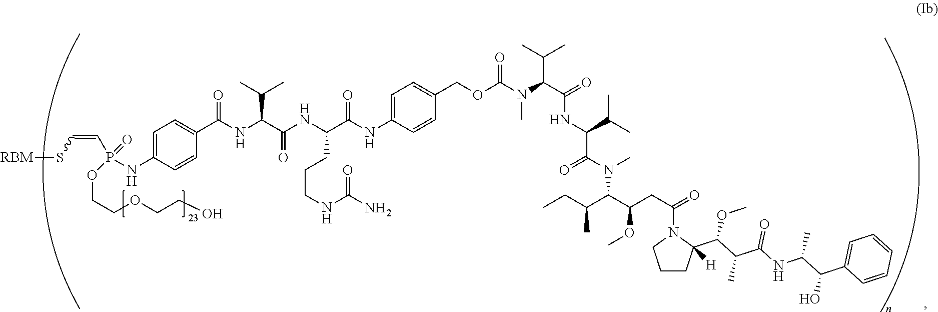

wherein:
RBM is an antibody; and
n is an integer as defined herein; preferably n is an integer ranging from 1 to 10; more preferably from 2 to 10; still more preferably from 4 to 10; still more preferably from 6 to 10, still more preferably from 7 to 10, even more preferably n is 8; or
   preferably n is an integer ranging from 1 to 10, more preferably from 2 to 8, still more preferably from 3 to 6, still more preferably n is 4 or 5, even more preferably n is 4.

### Compound of Formula (II)

The present invention also relates to a compound having the formula (II):

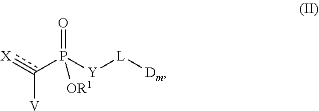

or a pharmaceutically acceptable salt or solvate thereof,
wherein:
[symbol] is a triple bond; or
[symbol] is a double bond;
V is absent when [symbol] is a triple bond; or
V is H or (C₁-C₈)alkyl when [symbol] is a double bond;
X is R₃—C when [symbol] is a triple bond; or
X is

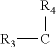

when [symbol] is a double bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic or optionally substituted aromatic residue;
R⁴ is H; or an optionally substituted aliphatic or optionally substituted aromatic residue;
R⁵ is H; or an optionally substituted aliphatic or optionally substituted aromatic residue;
R⁶ is H; or an optionally substituted aliphatic or optionally substituted aromatic residue;
R⁷ is H; or an optionally substituted aliphatic or optionally substituted aromatic residue;
L is a linker;
D is a drug moiety; and
m is an integer ranging from 1 to 10.

Preferably R³ is H or (C₁-C₈)alkyl; more preferably R³ is H. Preferably R⁴, when present, is H or (C₁-C₈)alkyl; more preferably R⁴, when present, is H. Preferably R⁵, when present is H or (C₁-C₈)alkyl; more preferably R⁵, when present, is H. Preferably R⁶, when present is H or (C₁-C₈)alkyl; more preferably R⁶, when present, is H. Preferably R⁷, when present is H or (C₁-C₈)alkyl; more preferably R⁷, when present, is H.

Preferably, [symbol] is a triple bond; V is absent; X is R₃—C; and R³ is H or an optionally substituted aliphatic residue or an optionally substituted aromatic residue; preferably R³ is H or (C₁-C₈)alkyl; more preferably R³ is H.

More preferably, [symbol] represents a triple bond; V is absent; X represents R₃—C, and R₃ represents H or (C₁-C₈)alkyl. Preferably, R₃ represents H or (C₁-C₆)alkyl, more preferably H or (C₁-C₄)alkyl, still more preferably H or (C₁-C₂)alkyl. Even more preferably, R₃ is H.

In some embodiments, [symbol] may be a double bond; V is H or (C₁-C₈)alkyl, preferably V is H; X is

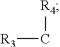

R₃ is H or an optionally substituted aliphatic residue or an optionally substituted aromatic residue; more preferably R³ is H or (C₁-C₈)alkyl, more preferably R³ is H; R⁴ is H or an optionally substituted aliphatic residue or an optionally substituted aromatic residue; preferably, R⁴ is H or (C₁-C₈)alkyl, preferably R⁴ is H.

In some embodiments, [symbol] may represent a double bond; V may be H or (C₁-C₈)alkyl; X may represent

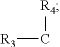

and R₃ and R₄ may independently represent H or (C₁-C₈)alkyl. Preferably, R₃ and R₄ independently represent H or (C₁-C₆)alkyl, more preferably H or (C₁-C₄)alkyl, still more preferably H or (C₁-C₂)alkyl. Preferably, R₃ and R₄ are the same; even more preferably, R₃, R₄ and V are the same. More preferably, R₃ and R₄ are both H. Preferably, V is H or (C₁-C₆)alkyl, more preferably H or (C₁-C₄)alkyl, still more preferably H or (C₁-C₂)alkyl. Even more preferably, V is H. In preferred embodiments, R₃, R₄ and V are each H.

In any one of the compounds of formula (II), any variable may be defined as described herein, in particular as with regard to the conjugates of formula (I) and/or the thiol-containing molecule of formula (III). Accordingly, RBM, [symbol] , V, X, Y, R¹, R³, R⁴, R⁵, R⁶, R⁷, L, D, m and n may be as defined herein. Preferably, Y is NH.

### Method of Preparing a Conjugate of Formula (I)

The present invention also relates to a method of preparing a conjugate of formula (I), said method comprising:
reacting a compound of formula (II)

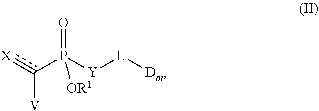

or a pharmaceutically acceptable salt or solvate thereof,
wherein:
[symbol] is a triple bond; or
[symbol] is a double bond;
V is absent when [symbol] is a triple bond; or
V is H or (C₁-C₈)alkyl when [symbol] is a double bond;
X is R₃—C when [symbol] is a triple bond; or
X is

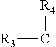

when [symbol] is a double bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic or optionally substituted aromatic residue;
R⁴ is H; or an optionally substituted aliphatic or optionally substituted aromatic residue;
R⁵ is H; or an optionally substituted aliphatic or optionally substituted aromatic residue;
R⁶ is H; or an optionally substituted aliphatic or optionally substituted aromatic residue;
R⁷ is H; or an optionally substituted aliphatic or optionally substituted aromatic residue;
L is a linker;
D is a drug moiety; and
m is an integer ranging from 1 to 10;
with a thiol-containing molecule of formula (III)

RBM [symbol] SH)ₙ (III),
wherein RBM is a receptor binding molecule; and
n is an integer ranging from 1 to 20;
resulting in a compound of formula (I)

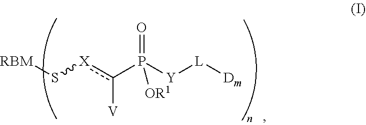

or a pharmaceutically acceptable salt or solvate thereof;
wherein:
[symbol] is a double bond when [symbol] in a compound of formula (II) is a triple bond; or
[symbol] is a bond when [symbol] in a compound of formula (II) is a double bond;
V is absent when [symbol] is a double bond; or
V is H or (C₁-C₈)alkyl when [symbol] is a bond;
X is R₃—C when [symbol] is a double bond; or
X is

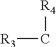

when [symbol] is a bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic or optionally substituted aromatic residue;
R⁴ is H; or an optionally substituted aliphatic or optionally substituted aromatic residue;
R⁵ is H; or an optionally substituted aliphatic or optionally substituted aromatic residue;
R⁶ is H; or an optionally substituted aliphatic or optionally substituted aromatic residue;
R⁷ is H; or an optionally substituted aliphatic or optionally substituted aromatic residue; L is a linker;
D is a drug moiety;
m is an integer ranging from 1 to 10; and
n is an integer ranging from 1 to 20.

Preferably R³ is H or (C₁-C₈)alkyl; more preferably R³ is H. Preferably R⁴, when present, is H or (C₁-C₈)alkyl; more preferably R⁴, when present, is H. Preferably R⁵, when present is H or (C₁-C₈)alkyl; more preferably R⁵, when present, is H. Preferably R⁶, when present is H or (C₁-C₈)alkyl; more preferably R⁶, when present, is H. Preferably R⁷, when present is H or (C₁-C₈)alkyl; more preferably R⁷, when present, is H.

Preferably, [symbol] is a triple bond; V is absent; X is R₃—C; and R³ is H or an optionally substituted aliphatic residue or an optionally substituted aromatic residue; preferably R³ is H or (C₁-C₈)alkyl; more preferably R³ is H; and [symbol] represents a double bond.

More preferably, [symbol] represents a triple bond; V is absent; X represents R₃—C, R₃ represents H or (C₁-C₈)alkyl; and [symbol] represents a double bond. Preferably, R₃ represents H or (C₁-C₆)alkyl, more preferably H or (C₁-C₄)alkyl, still more preferably H or (C₁-C₂)alkyl. Even more preferably, R₃ is H.

In some embodiments, [symbol] may be a double bond; V is H or (C₁-C₈)alkyl, preferably V is H; X is

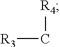

R₃—C; R₃ is H or an optionally substituted aliphatic residue or an optionally substituted aromatic residue; and [symbol] may represent a bond; more preferably R³ is H or (C₁-C₈)alkyl, more preferably R³ is H; R⁴ is H or an optionally substituted aliphatic residue or an optionally substituted aromatic residue; preferably, R⁴ is H or (C₁-C₈)alkyl, preferably R⁴ is H.

In some embodiments, [symbol] may represent a double bond; V may be H or (C₁-C₈)alkyl; X may represent

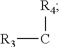

R₃ and R₄ may independently represent H or (C₁-C₈)alkyl; and [symbol] may represents a bond. Preferably, R₃ and R₄ independently represent H or C₁-C₆-alkyl, more preferably H or C₁-C₄-alkyl, still more preferably H or C₁-C₂-alkyl. Preferably, R₃ and R₄ are the same; even more preferably, R₃, R₄ and V are the same. More preferably, R₃ and R₄ are both H. Preferably, V is H or C₁-C₆-alkyl, more preferably H or C₁-C₄-alkyl, still more preferably H or C₁-C₂-alkyl. Even more preferably, V is H. In preferred embodiments, R₃, R₄ and V are each H.

With regard to the representations [symbol] and [symbol] used herein, it is noted that, as commonly known to a person skilled in the art, each carbon atom is tetravalent. Accordingly, a structure

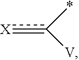

wherein X and V are as defined herein and the asterisk (*) indicates attachment to the phosphorus, includes the structures R₃≡* and

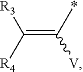

wherein R₃, R₄ and V are as defined herein. A structure

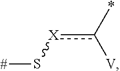

wherein X and V are as defined herein, the asterisk (*) indicates attachment to the phosphorus and # indicates attachment to the receptor binding molecule (RBM), includes the structures

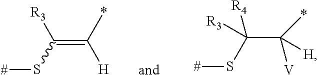

wherein R₃, R₄ and V are as defined herein, and H is hydrogen. A wavy bond indicates that the configuration of the double bond may be E or Z. It is also possible that the compound is present as a mixture of the E and Z isomers.

When the receptor binding molecule comprises one or more disulfide bridges, such as e.g. an antibody, the method may further comprise reducing at least one disulfide bridges of the receptor binding molecule in the presence of a reducing agent to form a thiol group (SH). The resulting compound of formula (III) may then be reacted with a compound of formula (II) to yield a conjugate of formula (I). The reducing agent may be selected from the group consisting of tris(2-carboxyethyl)phosphine (TCEP), dithiothreitol (DTT), sodium dithionite, sodium thiosulfate, and sodium sulfite. Accordingly, the reducing agent may be dithiothreitol (DTT). The reducing agent may be sodium dithionite. The reducing agent may be sodium sulfite. Preferably, the reducing agent is tris(2-carboxyethyl)phosphine (TCEP).

Preferably, the reducing of at least one disulfide bridge comprises using about 1 to about 3 equivalents, preferably about 1 to about 2 equivalents, more preferably about 1 equivalent of the reducing agent per 1 disulfide bridge to be reduced. In this context, it is noted that in theory 1 eq. of the reducing agent, in particular of a reducing agent as described herein, is necessary to reduce 1 disulfide bridge to give 2 thiol groups (SH).

Preferably, the thiol-containing molecule of formula (III) is reacted with about 1 to about 4 equivalents, preferably about 1 to about 3 equivalents, more preferably about 1 to about 2 equivalents, still more preferably about 1.5 equivalents of the compound of formula (II) per thiol group (SH).

Preferably, the reaction of a compound of formula (II) with a thiol-containing molecule of formula (III) is carried out in an aqueous medium.

Preferably, the reaction of the compound of formula (II) with the thiol-containing molecule of formula (III) is performed under neutral pH or slightly basic conditions. Still more preferably the reaction is performed at a pH of from 6 to 10. Even more preferably, the reaction is performed at a pH of from 7 to 9.

In any one of the methods, any variable may be defined as described herein, in particular as with regard to the conjugates of formula (I) and/or the compound of formula (II). Accordingly, RBM, [symbol] , [symbol] , V, X, Y, R¹, R³, R⁴, R⁵, R⁶, R′, L, D, m and n may be as defined herein. Preferably, Y is NH.

Methods of preparing compounds of formula (II) are known in the art. As illustrative examples, compounds of formula (II), wherein the group Y is NH, may be prepared by using techniques and conditions, e.g. a Staudinger phosphonite reaction, as e.g. described in WO 2018/041985 A1, which is hereby incorporated by reference. Compounds of formula (II), wherein Y is S or O, may be prepared by using techniques and conditions as e.g. described in WO 2019/170710, which is hereby incorporated by reference. In an analogous manner to the compounds of formula (I), wherein Y is S or O, as described in WO 2019/170710, compounds of formula (II), wherein Y is CR⁶R⁷, may be prepared, as illustrative examples, by substitution at the phosphorus atom using, e.g., a suitable organometallic compound, such as e.g. a Grignard compound or an organolithium compound. A person skilled in the art readily selects suitable methods and conditions to prepare compounds of formula (II). The Examples section of the present disclosure also comprises guidance on how to prepare or obtain compounds of formula (II) and/or conjugates of formula (I).

The present invention also relates to a conjugate of formula (I) obtainable or being obtained by any method of preparing a conjugate of formula (I) as described herein.

### Pharmaceutical Composition

The present invention further relates to a pharmaceutical composition comprising a conjugate of formula (I).

The pharmaceutical composition may comprise a population of a conjugate of formula (I), wherein the average number of drug moieties D per receptor binding molecule in the composition is from more than 0 to about 14, preferably from about 1 to about 14, more preferably from about 2 to about 14, still more preferably from about 4 to about 14, still more preferably from about 5 to about 12, still more preferably from about 6 to about 12, still more preferably from about 7 to about 10, even more preferably about 8. Accordingly, the pharmaceutical composition may comprise a population of a conjugate of formula (I), wherein the average number of drug moieties D per receptor binding molecule in the composition is from more than 0 to about 14. Preferably, the pharmaceutical composition comprises a population of a conjugate of formula (I), wherein the average number of drug moieties D per receptor binding molecule in the composition is from about 1 to about 14. More preferably, the pharmaceutical composition comprises a population of a conjugate of formula (I), wherein the average number of drug moieties D per receptor binding molecule in the composition is from about 2 to about 14. Still more preferably, the pharmaceutical composition comprises a population of a conjugate of formula (I), wherein the average number of drug moieties D per receptor binding molecule in the composition is from about 4 to about 14. Still more preferably, the pharmaceutical composition comprises a population of a conjugate of formula (I), wherein the average number of drug moieties D per receptor binding molecule in the composition is from about 5 to about 12. Still more preferably, the pharmaceutical composition comprises a population of a conjugate of formula (I), wherein the average number of drug moieties D per receptor binding molecule in the composition is from about 6 to about 12. Still more preferably, the pharmaceutical composition comprises a population of a conjugate of formula (I), wherein the average number of drug moieties D per receptor binding molecule in the composition is from about 6 to about 10. Even more preferably, the pharmaceutical composition comprises a population of a conjugate of formula (I), wherein the average number of drug moieties D per receptor binding molecule in the composition is about 8. When the receptor binding molecule is, in some preferred embodiments, an antibody or an antibody fragment, such average number is also denoted as “average drug to antibody ratio (DARav)”. In this context, a person skilled in the art understands that a composition may comprise a population of conjugates, which may differ in the number of drug moieties per receptor binding molecule, and which may optionally also comprise unconjugated receptor binding molecule, so that the result is an average number of drugampto moieties per receptor binding molecule.

The pharmaceutical composition may comprise a population of a conjugate of formula (I), wherein the average number of drug moieties D per receptor binding molecule is from more than 0 to about 14, preferably from about 1 to about 14, more preferably from about 1 to about 12, still more preferably from about 2 to about 10, still more preferably from about 2 to about 8, still more preferably from about 2 to about 6, still more preferably from about 3 to about 5, even more preferably about 4. Accordingly, the pharmaceutical composition may comprise a population of a conjugate of formula (I), wherein the average number of drug moieties D per receptor binding molecule is from more than 0 to about 14. Preferably, the pharmaceutical composition comprises a population of a conjugate of formula (I), wherein the average number of drug moieties D per receptor binding molecule is from about 1 to about 14.

More preferably, the pharmaceutical composition comprises a population of a conjugate of formula (I), wherein the average number of drug moieties D per receptor binding molecule is from about 1 to about 12. Still more preferably, the pharmaceutical composition comprises a population of a conjugate of formula (I), wherein the average number of drug moieties D per receptor binding molecule is from about 2 to about 10. Still more preferably, the pharmaceutical composition comprises a population of a conjugate of formula (I), wherein the average number of drug moieties D per receptor binding molecule is from about 2 to about 8. Still more preferably, the pharmaceutical composition comprises a population of a conjugate of formula (I), wherein the average number of drug moieties D per receptor binding molecule is from about 2 to about 6. Still more preferably, the pharmaceutical composition comprises a population of a conjugate of formula (I), wherein the average number of drug moieties D per receptor binding molecule is from about 3 to about 5. Even more preferably, the pharmaceutical composition comprises a population of a conjugate of formula (I), wherein the average number of drug moieties D per receptor binding molecule is about 4. When the receptor binding molecule is an antibody or an antibody fragment, such average number is also denoted as “average drug to antibody ratio (DARav)”.

The pharmaceutical composition may further comprise one or more pharmaceutically acceptable carriers. In a specific embodiment, the term “pharmaceutically acceptable” means approved by a regulatory agency or other generally recognized pharmacopoeia for use in animals, and more particularly in humans. Pharmaceutically acceptable carriers are well known in the art and include, for example, aqueous solutions such as water, 5% dextrose, or physiologically buffered saline or other solvents or vehicles such as glycols, glycerol, oils such as olive oil, or injectable organic esters that are suitable for administration to a human or non-human subject. Particular exemplary pharmaceutically acceptable carriers include (biodegradable) liposomes; microspheres made of the biodegradable polymer poly(D,L-lactic-coglycolic acid (PLGA), albumin microspheres; synthetic polymers (soluble); nanofibers, protein-DNA complexes; protein conjugates; erythrocytes; or virosomes. Various carrier based dosage forms comprise solid lipid nanoparticles (SLNs), polymeric nanoparticles, ceramic nanoparticles, hydrogel nanoparticles, copolymerized peptide nanoparticles, nanocrystals and nanosuspensions, nanocrystals, nanotubes and nanowires, functionalized nanocarriers, nanospheres, nanocapsules, liposomes, lipid emulsions, lipid microtubules/microcylinders, lipid microbubbles, lipospheres, lipopolyplexes, inverse lipid micelles, dendrimers, ethosomes, multicomposite ultrathin capsules, aquasomes, pharmacosomes, colloidosomes, niosomes, discomes, proniosomes, microspheres, microemulsions and polymeric micelles. Other suitable pharmaceutically acceptable carriers and excipients are inter alia described in Remington's Pharmaceutical Sciences, 15^{th} Ed., Mack Publishing Co., New Jersey (1991) and Bauer et al., Pharmazeutische Technologie, 5^{th} Ed., Govi-Verlag Frankfurt (1997). See, e.g., Remington: The Science and Practice of Pharmacy, 21^{st} edition; Lippincott Williams & Wilkins, 2005.

In some embodiments, a pharmaceutically acceptable carrier or composition is sterile. A pharmaceutical composition can comprise, in addition to the active agent, physiologically acceptable compounds that act, for example, as bulking agents, fillers, solubilizers, stabilizers, osmotic agents, uptake enhancers, etc. Physiologically acceptable compounds include, for example, carbohydrates, such as glucose, sucrose, lactose; dextrans; polyols such as mannitol; antioxidants, such as ascorbic acid or glutathione; preservatives; chelating agents; buffers; or other stabilizers or excipients.

The choice of a pharmaceutically acceptable carrier(s) and/or physiologically acceptable compound(s) can depend for example, on the nature of the active agent, e.g., solubility, compatibility (meaning that the substances can be present together in the composition without interacting in a manner that would substantially reduce the pharmaceutical efficacy of the pharmaceutical composition under ordinary use situations) and/or route of administration of the composition.

Pharmaceutical compositions of the invention may comprise a therapeutically effective amount of the conjugate of formula (I) described herein and can be structured in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for topical or oral administration. A variety of routes are applicable for administration of the conjugate of formula (I), including, but not limited to, orally, topically, transdermally, subcutaneously, intravenously, intraperitoneally, intramuscularly or intraocularly. However, any other route may readily be chosen by the person skilled in the art if desired.

### Use in Methods of Treatment

As shown in the Examples, conjugates of formula (I) of the present invention can be used for the treatment, in particular the treatment of cancer. Accordingly, the present invention further relates to a conjugate of formula (I) of the invention for use in a method of treating a disease, optionally comprising the administration of an effective amount of the conjugate of the invention or the pharmaceutical composition of the invention to a subject or patient in need thereof. Also, the present invention relates to a pharmaceutical composition of the invention for use in a method of treating a disease, optionally comprising the administration an effective amount of the conjugate of the invention or the pharmaceutical composition of the invention to a subject or patient in need thereof. The disease may be associated with overexpression of CD30. The disease may by associated with overexpression of Her2. The disease may be cancer. The cancer may be a solid tumor. The disease may be a cancer associated with overexpression of CD30. The disease may be a cancer associated with overexpression of Her2.

The present invention also relates to the use of a conjugate of formula (I) of the invention for the manufacture of a medicament for treating a disease. The present invention also relates to the use of a pharmaceutical composition of the invention for the manufacture of a medicament for treating a disease. The disease may be associated with overexpression of CD30. The disease may by associated with overexpression of Her2. The disease may be cancer. The cancer may be a solid tumor. The disease may be a cancer associated with overexpression of CD30. The disease may be a cancer associated with overexpression of Her2.

The present invention also relates to a method of treating a disease, comprising the administration of an effective amount of a conjugate of formula (I) of the invention to a subject or patient in need thereof. The present invention also relates to a method of treating a disease, comprising the administration of an effective amount of a pharmaceutical composition of the invention to a subject or patient in need thereof. The disease may be associated with overexpression of CD30. The disease may by associated with overexpression of Her2. The disease may be cancer. The cancer may be a solid tumor. The disease may be a cancer associated with overexpression of CD30. The disease may be a cancer associated with overexpression of Her2.

The phrase “effective amount” in general refers to an amount of a therapeutic agent (e.g., the conjugate of the invention) that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays. The exact amount will depend on the purpose of the treatment, and will be ascertainable by one skilled in the art using known techniques (see, for example, Lloyd (1999) The Art, Science and Technology of Pharmaceutical Compounding).

Further, the present invention relates to a conjugate of formula (I) as described herein for use in a method of treating cancer in a patient. The present invention also relates to a pharmaceutical composition as described herein for use in a method of treating cancer in a patient. The term “patient” means according to the invention a human being, a non-human primate or another animal, in particular a mammal such as a cow, horse, pig, sheep, goat, dog, cat or a rodent such as a mouse and rat. In a particularly preferred embodiment, the patient is a human being. Except when noted, the terms “patient” or “subject” are used herein interchangeably. The term “treatment” in all its grammatical forms includes therapeutic or prophylactic treatment. A “therapeutic or prophylactic treatment” comprises prophylactic treatments aimed at the complete prevention of clinical and/or pathological manifestations or therapeutic treatment aimed at amelioration or remission of clinical and/or pathological manifestations. The term “treatment” thus also includes the amelioration or prevention of diseases.

The term “cancer”, as used herein, can denote any cancer, e.g., said cancer may be selected from the group consisting of Cancer in Adolescents, Adrenocortical Carcinoma, Anal Cancer, Astrocytomas, Atypical Teratoid/Rhabdoid Tumor, Basal Cell Carcinoma, Bile Duct Cancer, Bladder Cancer, Bone Cancer, Brain Tumors, Breast Cancer, Bronchial Tumors, Cervical Cancer, Chordoma, Chronic Myeloproliferative Neoplasms, Colorectal Cancer, Craniopharyngioma, Embryonal Tumors, Medulloblastoma and Other Central Nervous System, Childhood (Brain Cancer), Endometrial Cancer (Uterine Cancer), Ependymoma, Childhood (Brain Cancer), Esophageal Cancer, Esthesioneuroblastoma (Head and Neck Cancer), Ewing Sarcoma (Bone Cancer), Extracranial Germ Cell Tumor, Childhood, Extragonadal Germ Cell Tumor, Fallopian Tube Cancer, Gallbladder Cancer, Gastric (Stomach) Cancer, Gastrointestinal Carcinoid Tumor, Gastrointestinal Stromal Tumors (GIST), Gestational Trophoblastic Disease, Head and Neck Cancer, Heart Tumors, Hepatocellular (Liver) Cancer, Histiocytosis, Langerhans Cell, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Lip and Oral Cavity Cancer (Head and Neck Cancer), Liver Cancer, Lung Cancer, Male Breast Cancer, Melanoma, Melanoma, Intraocular (Eye), Merkel Cell Carcinoma (Skin Cancer), Mesothelioma, Malignant, Mouth Cancer (Head and Neck Cancer), Multiple Endocrine Neoplasia Syndromes, Nasal Cavity and Paranasal Sinus Cancer (Head and Neck Cancer), Nasopharyngeal Cancer (Head and Neck Cancer), Neuroblastoma, Non-Small Cell Lung Cancer, Oral Cancer, Lip and Oral Cavity Cancer and Oropharyngeal Cancer (Head and Neck Cancer), Osteosarcoma, Ovarian Cancer, Pancreatic Cancer, Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Pleuropulmonary Blastoma (Lung Cancer), Primary Central Nervous System (CNS) Lymphoma, Primary Peritoneal Cancer, Prostate Cancer, Pulmonary Inflammatory Myofibroblastic Tumor (Lung Cancer), Rectal Cancer, Renal Cell (Kidney) Cancer, Retinoblastoma, Rhabdomyosarcoma, Childhood (Soft Tissue Sarcoma), Salivary Gland Cancer (Head and Neck Cancer), Skin Cancer, Small Cell Lung Cancer, Small Intestine Cancer, Soft Tissue Sarcoma, Squamous Cell Carcinoma of the Skin - see Skin Cancer, Squamous Neck Cancer with Occult Primary, Metastatic (Head and Neck Cancer), Stomach (Gastric) Cancer, Testicular Cancer, Thymoma and Thymic Carcinoma, Thyroid Cancer, Tracheobronchial Tumors (Lung Cancer), Transitional Cell Cancer of the Renal Pelvis and Ureter (Kidney (Renal Cell) Cancer), Ureter and Renal Pelvis, Transitional Cell Cancer (Kidney (Renal Cell) Cancer, Urethral Cancer, Uterine Cancer, Endometrial, Uterine Sarcoma, Vaginal Cancer, Vascular Tumors (Soft Tissue Sarcoma), Vulvar Cancer, and Wilms Tumor and Other Childhood Kidney Tumors. Said cancer may be selected, e.g., also from the group consisting of lymphoma, such as Hodgkin's lymphoma (HL), non-Hodgkin lymphoma (NHL), anaplastic large-cell lymphoma (ALCL), large B-cell lymphoma, paediatric lymphoma, T-cell lymphoma and enteropathy-associated T-cell lymphoma (EATL), leukaemia, such as acute myeloid leukaemia (AML), acute lymphoblastic leukaemia (ALL) and mast cell leukaemia, germ cell cancer, graft-versus-host disease (GvHD) and lupus, in particular systemic lupus erythematosus (SLE), preferably Hodgkin Lymphoma (HL) or anaplastic large cell lymphoma (ALCL). The cancer may be selected from the group consisting of peripheral T cell lymphoma—not otherwise specified (PTCL-NOS), angioimmunoblastic T-cell lymphoma (AITL), enteropathy associated T cell lymphoma (EATL), adult T-cell leukemia/lymphoma (ATLL), extranodal natural killer/T-cell lymphoma (ENKTCL), hepatosplenic and intestinal γ/δ-T cell lymphoma, nodal peripheral T-cell lymphoma with TFH phenotype, and follicular T cell lymphoma. The cancer may be peripheral T cell lymphoma (PTCL), including anaplastic large cell lymphoma (ALCL). The cancer may be cutaneous T cell lymphoma (CTCL), including primary cutaneous anaplastic large cell lymphoma (pcALCL). The cancer may be Hodgkin lymphoma (HL).

By “tumor” is meant a group of cells or tissue that is formed by misregulated cellular proliferation, in particular cancer. Tumors may show partial or complete lack of structural organization and functional coordination with the normal tissue, and usually form a distinct mass of tissue, which may be either benign or malignant. In particular, the term “tumor” refers to a malignant tumor. The term “tumor” may refer to a solid tumor. According to one embodiment, the term “tumor” or “tumor cell” also refers to non-solid cancers and cells of non-solid cancers such as leukemia cells. According to another embodiment, respective non-solid cancers or cells thereof are not encompassed by the terms “tumor” and “tumor cell”.

By “metastasis” is meant the spread of cancer cells from its original site to another part of the body. The formation of metastasis is a very complex process and normally involves detachment of cancer cells from a primary tumor, entering the body circulation and settling down to grow within normal tissues elsewhere in the body. When tumor cells metastasize, the new tumor is called a secondary or metastatic tumor, and its cells normally resemble those in the original tumor. This means, for example, that, if breast cancer metastasizes to the lungs, the secondary tumor is made up of abnormal breast cells, not of abnormal lung cells. The tumor in the lung is then called metastatic breast cancer, not lung cancer.

### Items of the Invention

The invention further relates to the following items:

1. A conjugate having the formula (I):

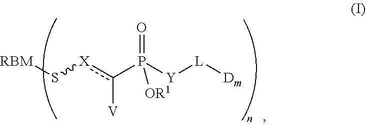

or a pharmaceutically acceptable salt or solvate thereof;
wherein:
RBM is a receptor binding molecule;
[symbol] is a double bond; or
[symbol] is a bond;
V is absent when [symbol] is a double bond; or
V is H or (C₁-C₈)alkyl when [symbol] is a bond;
X is R₃—C when [symbol] is a double bond; or
X is

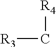

when [symbol] is a bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
L is a linker;
D is a drug moiety;
m is an integer ranging from 1 to 10; and
n is an integer ranging from 1 to 20.

2. The conjugate of item 1, wherein:
R³ is H or (C₁-C₈)alkyl; preferably R³ is H;
R⁴ when present is H or (C₁-C₈)alkyl; preferably R⁴, when present, is H;
R⁵ when present is H or (C₁-C₈)alkyl; preferably R⁵, when present, is H;
R⁶ when present is H or (C₁-C₈)alkyl; preferably R⁶, when present, is H; and
R⁷ when present is H or (C₁-C₈)alkyl; preferably R⁷, when present, is H.

3. The conjugate of item 1 or 2, wherein [symbol] is a double bond; V is absent; X is R₃—C; and R³ is H or an optionally substituted aliphatic residue or an optionally substituted aromatic residue; preferably R³ is H or (C₁-C₈)alkyl; more preferably R³ is H.

4. The conjugate of item 1 or 2, wherein [symbol] is a bond; V is H or (C₁-C₈)alkyl, preferably V is H; X is

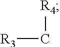

R₃ is H or an optionally substituted aliphatic residue or an optionally substituted aromatic residue; preferably R³ is H or (C₁-C₈)alkyl; more preferably R³ is H; R⁴ is H or an optionally substituted aliphatic residue or an optionally substituted aromatic residue; preferably R⁴ is H or (C₁-C₈)alkyl; more preferably R⁴ is H.

5. The conjugate of any one of the preceding items, wherein the receptor binding molecule is selected from the group consisting of an antibody, an antibody fragment, and a proteinaceous binding molecule with antibody-like binding properties.

6. The conjugate of item 5, wherein the receptor binding molecule is an antibody, preferably wherein the antibody is selected from the group consisting of a monoclonal antibody, a chimeric antibody, a humanized antibody, a human antibody, and a single domain antibody, such as a camelid or shark single domain antibody.

7. The conjugate of item 5, wherein the receptor binding molecule is an antibody fragment,
preferably wherein the antibody fragment is a divalent antibody fragment,
more preferably wherein the divalent antibody fragment is selected from the group consisting of a (Fab)₂′-fragment, a divalent single-chain Fv fragment, a dual affinity re-targeting (DART) antibody, and a diabody; or
preferably wherein the antibody fragment is a monovalent antibody fragment, more preferably wherein the monovalent antibody fragment is selected from the group consisting of a Fab fragment, a Fv fragment, and a single-chain Fv fragment (scFv).

8. The conjugate of item 5, wherein the receptor binding molecule is a proteinaceous binding molecule with antibody-like binding properties,
preferably wherein the proteinaceous binding molecule with antibody-like binding properties is selected from the group consisting of a mutein based on a polypeptide of the lipocalin family, a glubody, a protein based on the ankyrin scaffold, a protein based on the crystalline scaffold, an adnectin, an avimer, a DARPin, and an affibody.

9. The conjugate of any one of the preceding items, wherein Y is NH.

9a. The conjugate of item 9, wherein the receptor binding molecule is an antibody.

10. The conjugate of any one of the preceding items, wherein the first polyalkylene glycol unit R^{F} comprises 3 to 100 subunits having the structure:

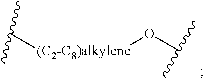

preferably wherein R^{F} is

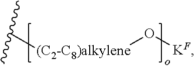

wherein:
[symbol] indicates the position of the O;
K^{F} is selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, -(C₂—C₁₀)alkyl-NH(C₁-C₃)alkyl and —(C₂-C₁₀)alkyl-N((C₁-C₃)alkyl)₂; preferably K^{F} is H; and
o is an integer ranging from 3 to 100.

11. The conjugate of item 10, wherein R^{F} is a first polyethylene glycol unit comprising at least 3 ethylene glycol subunits.

12. The conjugate of item 11, wherein the first polyethylene glycol unit R^{F} comprises 3 to 100 subunits having the structure:

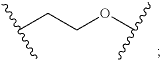 [SMILES: *CCO*]

preferably wherein R^{F} is:

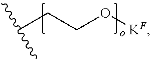

wherein
[symbol] indicates the position of the O;
K^{F} is selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₃)alkyl and —(C₂-C₁₀)alkyl-N((C₁-C₃)alkyl)₂; preferably K^{F} is H; and
o is an integer ranging from 3 to 100.

12a. The conjugate of item 12, wherein K^{F} is H.

12b. The conjugate of item 12 or 12a, wherein o ranges from 8 to 30.

12c. The conjugate of item 12b, wherein o ranges from 8 to 16.

12d. The conjugate of item 12c, wherein o is 10, 11, 12, 13 or 14.

12e. The conjugate of item 12d, wherein o ranges from 20 to 28.

12f. The conjugate of item 12e, wherein o is 22, 23, 24, 25 or 26.

13. The conjugate of any one of items 1 to 12f, wherein the linker L comprises a second spacer unit A, said second spacer unit being a group Z, said group Z having the structure:

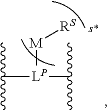

wherein:
L^{P} is a parallel connector unit;
R^{S} is, each independently, a second polyalkylene glycol unit;
M is, each independently, a bond or a moiety that binds R^{S} with L^{P};
s* is an integer ranging from 1 to 4; preferably, s* is 1; and
the wavy lines indicate the attachment point to the —Y— and to another part of the linker,
when present, or to a drug moiety (-D).

14. The conjugate of item 13, wherein M is each independently selected from the group consisting of —NH—, —O—, —S—, —C(O)—O—, —C(O)—NH— and —(C₁-C₁₀)alkylene; preferably each M is —O—.

15. The conjugate of item 13 or 14, wherein R^{S}, each independently, comprises 1 to 100 subunits having the structure:

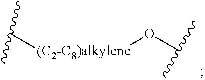

preferably wherein R^{S} is, each independently,

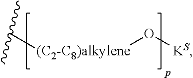

wherein:
[symbol] indicates the position of the M in group Z;
K^{S} is selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₃)alkyl and —(C₂-C₁₀)alkyl-N((C₁-C₃)alkyl)₂; preferably K^{S} is H; and
p is an integer ranging from 1 to 100.

16. The conjugate of any one of items 13 to 15, wherein R^{S} is, each independently, a second polyethylene glycol unit comprising at least one ethyleneglycol subunit.

17. The conjugate of item 16, wherein the second polyethylene glycol unit R^{S}, each independently, comprises 1 to 100 subunits having the structure:

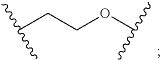 [SMILES: *CCO*]

preferably wherein R^{S} is, each independently:

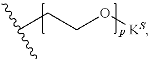

wherein
[symbol] indicates the position of the M in group Z;
K^{S} is selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₃)alkyl and —(C₂-C₁₀)alkyl-N((C₁-C₃)alkyl)₂; preferably K^{S} is H; and
p is an integer ranging from 1 to 100.

18. The conjugate of any one of the preceding items, wherein the linker L is cleavable.

19. The conjugate of item 18, wherein the linker L is cleavable by a protease, a glucuronidase, a sulfatase, a phosphatase, an esterase, or by disulfide reduction.

20. The conjugate of item 19, wherein the linker L is cleavable by a protease, preferably by a cathepsin such as cathepsin B.

21. The conjugate of any one of the preceding items, wherein the linker L comprises a valine-citrulline or a valine-alanine moiety.

22. The conjugate of item 21, wherein the linker L is:

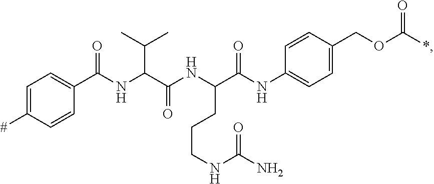

wherein # indicates the attachment point to the Y and * indicates the attachment point to the drug moiety.

23. The conjugate of item 21, wherein the linker L is

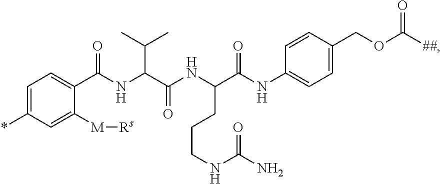

wherein:
R^{S} is a second polyalkylene glycol unit as defined in any one of items 13 to 17; preferably R^{S} is a second polyethylene glycol unit as defined in any one of items 16 or 17;
M is as defined in any one of items 13 or 14; preferably M is —O—; and
* indicates the attachment point to the Y; and
## indicates the attachment point to the drug moiety.

24. The conjugate of any one of the preceding items, wherein the drug moiety D is hydrophobic.

25. The conjugate of any one of the preceding items, wherein the drug moiety is selected from the group consisting of maytansinoids, calicheamycins, tubulysins, amatoxins, dolastatins and auristatins such as monomethyl auristatin E (MMAE) or monomethyl auristatin F (MMAF), pyrrolobenzodiazepine dimers, indolino-benzodiazepine dimers, emetine, radioisotopes, therapeutic proteins and peptides (or fragments thereof), kinase inhibitors, CDK inhibitors, histone deacetylase (HDAC) inhibitors, MEK inhibitors, KSP inhibitors, and analogues or prodrugs thereof.

26. The conjugate of item 25, wherein the drug moiety D is an auristatin, preferably monomethyl auristatin E (MMAE) or monomethyl auristatin F (MMAF), more preferably monomethyl auristatin E (MMAE).

27. The conjugate of any one of the preceding items, wherein the number of drug moieties D per receptor binding molecule is from 1 to 14, preferably from 2 to 14, more preferably from 4 to 14, still more preferably from 5 to 12, still more preferably from 6 to 12, still more preferably from 7 to 10, even more preferably 8.

28. The conjugate of any one of items 1 to 26, wherein the number of drug moieties D per receptor binding molecule is from 1 to 14, preferably from 1 to 12, more preferably from 2 to 10, still more preferably from 2 to 8, still more preferably from 2 to 6, still more preferably from 3 to 5, even more preferably 4.

29. The conjugate of any one of items 1 to 26, wherein m is an integer ranging from 1 to 4, preferably 1 or 2, more preferably 1; and
n is an integer ranging from 1 to 20, preferably from 1 to 10, more preferably from 2 to 10, still more preferably from 4 to 10, still more preferably from 6 to 10, still more preferably from 7 to 10, even more preferably 8.

30. The conjugate of any one of items 1 to 26, wherein m is an integer ranging from 1 to 4, preferably 1 or 2, more preferably 1; and
n is an integer ranging from 1 to 20, preferably from 1 to 10, more preferably from 2 to 8, still more preferably from 3 to 6, still more preferably 4 or 5, even more preferably 4.

30a. A conjugate having the formula (I):

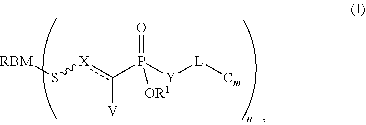

or a pharmaceutically acceptable salt or solvate thereof;
wherein:
RBM is an antibody;
[symbol] is a double bond; or
[symbol] is a bond;
V is absent when [symbol] is a double bond; or
V is H when [symbol] is a bond;
X is R₃—C when [symbol] is a double bond; or
X is

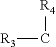

when [symbol] is a bond;
Y is NH;
R¹ is a polyethylene glycol unit having the structure:

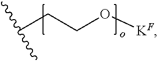

wherein:
[symbol] indicates the position of the O;
K^{F} is H; and
o is an integer ranging from 8 to 30;
R³ is H;
R⁴ is H;
L is a linker having the following structure:

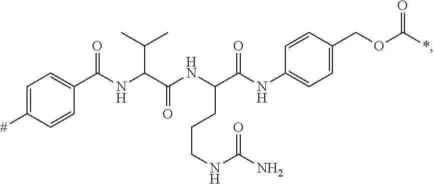

wherein # indicates the attachment point to the Y and * indicates the attachment point to the drug moiety (D);
D is a drug moiety;
m is 1; and
n is an integer ranging from 1 to 10.

30b. The conjugate of item 33a, wherein the drug moiety D is monomethyl auristatin E (MMAE) or monomethyl auristatin F (MMAF).

30c. The conjugate of item 30b, wherein the drug moiety D is monomethyl auristatin E (MMAE).

30d. The conjugate of any one of items 30a to 30c, wherein o ranges from 8 to 16.

30e. The conjugate of item 30d, wherein o is 10, 11, 12, 13 or 14.

30f. The conjugate of any one of items 30a to 30c, wherein o ranges from 20 to 28.

30g. The conjugate of item 30g, wherein o is 22, 23, 24, 25 or 26.

30h. The conjugate of any one of items 30a to 30g, wherein n ranges from 2 to 10.

30i. The conjugate of item 30h, wherein n is 4.

30j. The conjugate of item 30h, wherein n is 8.

30k. A conjugate having the following formula (Ia):

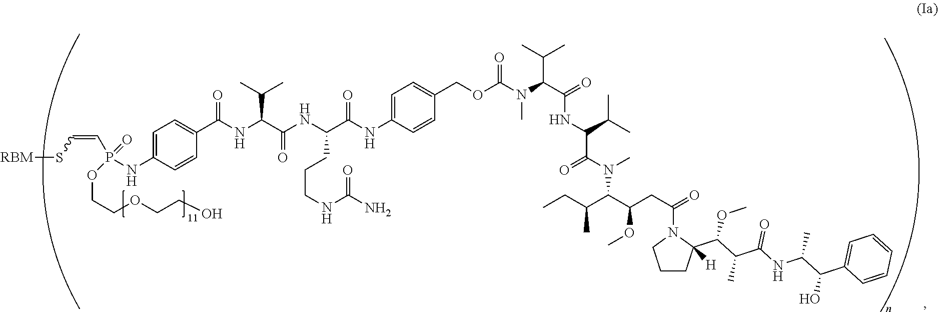

wherein RBM is an antibody.

30k. A conjugate having the following formula (Ib):

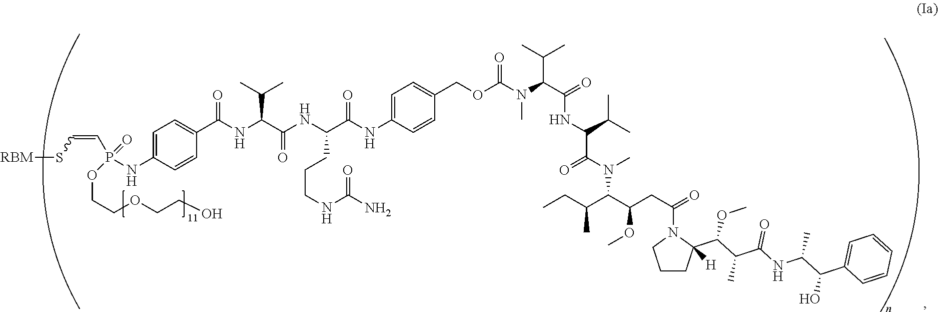

wherein RBM is an antibody.

31. A compound having the formula (II):

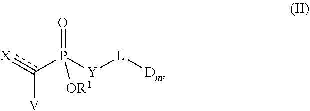

or a pharmaceutically acceptable salt or solvate thereof;
wherein:
[symbol] is a triple bond; or
[symbol] is a double bond;
V is absent when [symbol] is a triple bond; or
V is H or (C₁-C₈)alkyl when [symbol] is a double bond;

X is R₃—C when [symbol] is a triple bond; or
X is

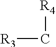

when [symbol] is a double bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
L is a linker;
D is a drug moiety; and
m is an integer ranging from 1 to 10.

32. The compound of item 31, wherein:
R³ is H or (C₁-C₈)alkyl; preferably R³ is H;
R⁴ when present is H or (C₁-C₈)alkyl; preferably R⁴, when present, is H;
R⁵ when present is H or (C₁-C₈)alkyl; preferably R⁵, when present, is H;
R⁶ when present is H or (C₁-C₈)alkyl; preferably R⁶, when present, is H; and
R⁷ when present is H or (C₁-C₈)alkyl; preferably R⁷, when present, is H.

33. The compound of item 31 or 32, wherein [symbol] is a triple bond; V is absent; X is R₃—C and R³ is H or an optionally substituted aliphatic residue or an optionally substituted aromatic residue; preferably R³ is H or (C₁-C₈)alkyl; more preferably R³ is H.

34. The compound of item 31 or 32, wherein [symbol] is a double bond; V is H or (C₁-C₈)alkyl, preferably V is H; X is

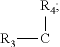

R³ is H or an optionally substituted aliphatic residue or an optionally substituted aromatic residue; preferably R³ is H or (C₁-C₈)alkyl; more preferably R³ is H; R⁴ is H or an optionally substituted aliphatic residue or an optionally substituted aromatic residue; preferably R⁴ is H or (C₁-C₈)alkyl; more preferably R⁴ is H.

35. The compound of any one of items 31 to 33, wherein RBM, V, X, Y, R¹, R³, R⁴, R⁵, R⁶, R⁷, L, D, m and n are as defined in any one of items 1 to 30i; preferably, Y is NH.

36. A method of preparing a conjugate of formula (I), said method comprising:
reacting a compound of formula (II)

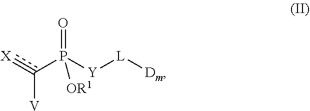

or a pharmaceutically acceptable salt of solvate thereof;
wherein:
[symbol] is a triple bond; or
[symbol] is a double bond;
V is absent when [symbol] is a triple bond; or
V is H or (C₁-C₈)alkyl when [symbol] is a double bond;
X is R₃—C when [symbol] is a triple bond; or
X is

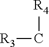

when [symbol] is a double bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
L is a linker;
D is a drug moiety; and
m is an integer ranging from 1 to 10;
with a thiol-containing molecule of formula (III)

RBM [symbol] SH)ₙ (III),
wherein RBM is a receptor binding molecule; and
n is an integer ranging from 1 to 20;
resulting in a compound of formula (I)

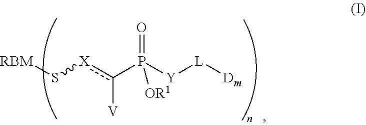

or a pharmaceutically acceptable salt or solvate thereof;
wherein:
[symbol] is a double bond when [symbol]
in a compound of formula (II) is a triple bond; or
[symbol] is a bond when [symbol] in a compound of formula (II) is a double bond;
V is absent when [symbol] is a double bond; or
V is H or (C₁-C₈)alkyl when [symbol] is a bond;
X is R₃—C when [symbol] is a double bond; or
X is

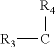

when [symbol] is a bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
L is a linker;
D is a drug moiety;
m is an integer ranging from 1 to 10; and
n is an integer ranging from 1 to 20.

37. The method of item 36, wherein:
R³ is H or (C₁-C₈)alkyl; preferably R³ is H;
R⁴ when present is H or (C₁-C₈)alkyl; preferably R⁴, when present, is H;
R⁵ when present is H or (C₁-C₈)alkyl; preferably R⁵, when present, is H;
R⁶ when present is H or (C₁-C₈)alkyl; preferably R⁶, when present, is H; and
R⁷ when present is H or (C₁-C₈)alkyl; preferably R⁷, when present, is H.

38. The method of item 36 or 37, wherein [symbol] is a triple bond; V is absent; X is R₃—C; R³ is H or an optionally substituted aliphatic residue or an optionally substituted aromatic residue, preferably R³ is H or (C₁-C₈)alkyl; more preferably R³ is H, and [symbol] is a double bond.

39. The method of item 36 or 37, wherein [symbol] is a double bond; V is H or (C₁-C₈)alkyl, preferably V is H; X is

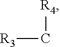

R³ is H or an optionally substituted aliphatic residue or an optionally substituted aromatic residue, preferably R³ is H or (C₁-C₈)alkyl, more preferably R³ is H; R⁴ is H or an optionally substituted aliphatic residue or an optionally substituted aromatic residue; preferably R4 is H or (C₁-C₈)alkyl, more preferably R⁴ is H; and [symbol] is a bond.

40. The method of any one of items 36 to 39, wherein the reaction is performed under neutral pH or slightly basic conditions, preferably at a pH of from 6 to 10.

41. The method of any one of items 36 to 40, further comprising reducing at least one disulfide bridge of the receptor binding molecule in the presence of a reducing agent to form a thiol group (SH).

42. The method of item 41, wherein the reducing agent is selected from the group consisting of tris(2-carboxyethyl)phosphine (TCEP), dithiothreitol (DTT), sodium dithionite, sodium thiosulfate, and sodium sulfite;
preferably wherein the reducing agent is tris(2-carboxyethyl)phosphine (TCEP).

43. The method of item 41 or 42, wherein the reducing of at least one disulfide bridge comprises using about 1 to about 3 equivalents, preferably about 1 to about 2 equivalents, more preferably about 1 equivalent of the reducing agent per disulfide bridge to be reduced.

44. The method of any one of items 36 to 43, wherein the thiol-containing molecule of formula (III) is reacted with about 1 to about 4 equivalents, preferably about 1 to about 3 equivalents, more preferably about 1 to about 2 equivalents, still more preferably about 1.5 equivalents of the compound of formula (II) per thiol group (SH).

45. The method of any one of items 36 to 44, wherein the reacting a compound of formula (II) with a thiol-containing molecule of formula (III) is carried out in an aqueous medium.

46. The method of any one of items 36 to 45, wherein RBM, V, X, Y, R¹, R³, R⁴, R⁵, R⁶, R⁷, L, D, m and n are as defined in any one of items 1 to 35; preferably Y is NH.

47. A conjugate of formula (I) obtainable or being obtained by a method of any one of items 36 to 46.

48. A pharmaceutical composition comprising a conjugate of any one of items 1 to 30k or 47.

49. The pharmaceutical composition of item 48, wherein the pharmaceutical composition comprises a population of a conjugate of any one of item 1 to 30, and wherein the average number of drug moieties D per receptor binding molecule in the composition is from more than 0 to about 14, preferably from about 1 to about 14, more preferably from about 2 to about 14, still more preferably from about 4 to about 14, still more preferably from about 5 to about 12, still more preferably from about 6 to about 12, still more preferably from about 7 to about 10, even more preferably about 8.

50. The pharmaceutical composition of item 48, wherein the pharmaceutical composition comprises a population of a conjugate of any one of item 1 to 30, and wherein the average number of drug moieties D per receptor binding molecule in the composition is from more than 0 to about 14, preferably from about 1 to about 14, more preferably from about 1 to about 12, still more preferably from about 2 to about 10, still more preferably from about 2 to about 8, still more preferably from about 2 to about 6, still more preferably from about 3 to about 5, even more preferably about 4.

51. A conjugate of any one of items 1 to 30k or 47 for use in a method of treating a disease.

52. The conjugate for use of item 51, wherein the disease is cancer.

53. A pharmaceutical composition of any one of items 48 to 50 for use in a method of treating a disease.

54. The pharmaceutical composition for use of item 53, wherein the disease is cancer.

54a. The pharmaceutical composition for use of item 54, wherein the cancer is a solid tumor.

55. Use of a conjugate of any one of items 1 to 30k or 47 for the manufacture of a medicament for treating a disease.

56. The use of item 55, wherein the disease is cancer.

56a. The use of item 56, wherein the cancer is a solid tumor.

57. Use of a pharmaceutical composition of any one of items 48 to 50 for the manufacture of a medicament for treating a disease.

58. The use of item 57, wherein the disease is cancer.

58a. The us of item 58, wherein the cancer is a solid tumor.

59. A method of treating a disease, comprising the administration of an effective amount of a conjugate of any one of items 1 to 30k or 47 to a subject or patient in need thereof.

60. The method of item 59, wherein the disease is cancer.

60a. The method of item 60, wherein the cancer is a solid tumor.

61. A method of treating a disease, comprising the administration of an effective amount of a pharmaceutical composition of any one of items 48 to 50 to a subject or patient in need thereof.

62. The method of item 61, wherein the disease is cancer.

62a. The method of item 62, wherein the cancer is a solid tumor.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number. For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

As used herein the terms “about”, “approximately” or “essentially” mean within 20%, preferably within 15%, preferably within 10%, and more preferably within 5% of a given value or range. It also includes the concrete number, i.e. “about 20” includes the number of 20.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

### EXAMPLES

An even better understanding of the present invention and of its advantages will be evident from the following examples, offered for illustrative purposes only. The examples are not intended to limit the scope of the present invention in any way.

### General Information, Materials and Methods

### Chemicals, Solvents and Antibodies

Chemicals and solvents were purchased from Merck (Merck group, Germany), TCI (Tokyo chemical industry CO., LTD., Japan), Iris Biotech (Iris Biotech GmbH, Germany), MCE (MedChemExpress, USA) and Carl Roth (Carl Roth GmbH+Co. KG, Germany) and used without further purification. Amino acids all had their naturally occurring configuration (i.e., the L configuration), unless indicated to the contrary. Dry solvents were purchased from Merck (Merck group, Germany). Trastuzumab was purchased from Roche (Hoffmann-La Roche AG, Switzerland). Brentuximab was produced by Evitria (evitria AG, Switzerland). PEG24 was purchased from BiochemPEG (Pure Chemistry Scientific Inc., United States)

### Preparative HPLC

Preparative HPLC was performed on a BUCHI Pure C-850 Flash-Prep system (BUCHI Labortechnik AG, Switzerland) using a VP 250/10 Macherey-Nagel Nucleodur C18 HTec Spum column (Macherey-Nagel GmbH & Co. Kg, Germany) for smaller scales. The following gradients were used: Method C: (A=H2O+0.1% TFA (trifluoroacetic acid), B=MeCN (acetonitrile)+0.1% TFA, flow rate 6 ml/min, 30% B 0-5 min, 30-70% B 5-35 min, 99% B 35-45 min. For bigger scales, a VP 250/21 Macherey-Nagel Nucleodur C18 HTec Spum column (Macherey-Nagel GmbH & Co. Kg, Germany) was used with the following gradients were used: Method D: (A=H2O+0.1% TFA (trifluoroacetic acid), B=MeCN (acetonitrile)+0.1% TFA, flow rate 14 ml/min, 30% B 0-5 min, 30-70% B 5-35 min, 99% B 35-45 min.

### LC/MS

Small molecules were analyzed using a Water Acquity UPLC equipped with a binary solvent manager, a sample manager and a TUV detector, using a Waters XSelect Peptide HSS T3 XP column (2.1×150 mm, 100 Å, 2.5 μm). Samples were eluted at a column temperature of 45° C. The following gradient was used: A: 0.1% formic acid in H₂O; B: 0.1% formic acid in MeCN. 5% B 0-0.5 min, 5-95% B 0.5-3.5 min, 95% B 3.5-4 min, 95-5% B 4-4.01 min, 5% B 4.01-5 min. Compounds were detected at 220 and 254 nm. Mass analysis was conducted with a waters 3100 mass detector.

### LC/MS (High Resolution)

Small molecules, linker-payloads, antibodies and ADCs were analyzed using a Waters H-class instrument equipped with a quaternary solvent manager, a Waters sample manager-FTN, a Waters PDA detector and a Waters column manager with an Acquity UPLC protein BEH C4 column (300 Å, 1.7 μm, 2.1 mm×50 mm) for antibodies and ADCs. Here, samples were eluted at a column temperature of 80° C. The following gradient was used: A: 0.1% formic acid in H₂O; B: 0.1% formic acid in MeCN. 25% B 0-1 min, 0.4 mL/min, 25-95% B 1-3.5 min 0.2 mL/min, 95% B 3.5-4.5 min 0.2 mL/min, 95-25% B 4.5-5 min 0.4 mL/min, 25-95% B 5-5.5 min 0.4 mL/min, 95-25% B 5.5-7.5 min 0.4 mL/min. Mass analysis was conducted with a Waters XEVO G2-XS QTof analyzer. Proteins were ionized in positive ion mode applying a cone voltage of 40 kV. Raw data was analyzed with MaxEnt 1. Small molecules and linker-payloads were analyzed with an Acquity UPLC-BEH C18 column (300 Å, 1.7 μm, 2.1 mm×50 mm). Here, samples were eluted at a column temperature of 45° C. with a flow rate of 0.4 mL/min. The following gradient was used: A: 0.1% formic acid in H₂O; B: 0.1% formic acid in MeCN. 2% B 0-1 min, 2-98% B 1-5 min, 98% B 5-5.5 min, 98-2% B 5.5-6 min, 2% B 6-7 min.

### NMR

NMR spectra were recorded with a Bruker Ultrashield 300 MHz spectrometer and a Bruker Avance Ill 600 MHz spectrometer (Bruker Corp., USA) at ambient temperature. Chemical shifts δ are reported in ppm relative to residual solvent peak (CDCl₃: 7.26 [ppm]; DMSO-d6: 2.50 [ppm] for ¹H-spectra and CDCl₃: 77.16 [ppm]; DMSO-d6: 39.52 [ppm] for ¹³C-spectra. Coupling constants J are stated in Hz. Signal multiplicities are abbreviated as follows: s: singlet; d: doublet; t: triplet; q: quartet; m: multiplet.

### Preparative Size-Exclusion-Chromatography

Protein purification by size-exclusion chromatography was conducted with an AKTA Pure FPLC system (GE Healthcare, United States) equipped with a F9-C-fraction collector.

### Adc Concentration Determination

The ADC concentrations were determined in a 96-well plate with a Pierce™ Rapid Gold BCA Protein Assay Kit (Thermo Fisher Scientific, USA) and a Bradford reagent B6916 (Merck, Germany) with pre-diluted protein assay standards of bovine gamma globulin (Thermo Fisher Scientific, USA). Results of both Assays were arithmetically averaged.

### Sample Preparation of ADCs and Antibodies for MS

0.5 μl PNGase-F solution (Pomega, Germany, Recombinant, cloned from Elizabethkingia miricola 10 u/μl) and 5 μL of a 100 mM solution of DTT in water were added to 50 μl of 0.2 mg/mL antibody or ADC in PBS and the solution was incubated at 37° C. for at least 2 hours. Samples were subjected to LC/MS, injecting 2 μl for each sample.

### Analytical Size-Exclusion Chromatography

Analytical size-exclusion chromatography (A-SEC) of the ADCs was conducted on a Vanquish Flex UHPLC System with a DAD detector, Split Sampler FT (4° C.), Column Compartment H (25° C.) and binary pump F (Thermo Fisher Scientific, USA) using a MAbPac SEC-1 300 Å, 4×300 mm column (Thermo Fisher Scientific, USA) with a flow rate of 0.15 mL/min. Separation of different ADC/mAb populations have been achieved during a 30 minute isocratic gradient using a phosphate buffer at pH 7 (20 mM Na2HPO4/NaH2PO4, 300 mM NaCl, 5% v/v isopropyl alcohol as a mobile phase. 8 μg ADC/mAb where loaded onto the column for A-SEC analysis. UV chromatograms were recorded at 220 and 280 nm.

### Analytical Hydrophobic Interaction Chromatography

The measurements were conducted on a Vanquish Flex UHPLC System (2.9) with a MabPac HIC Butyl 4.6×100 mm column (Thermo Fischer Scientific, USA). Separation of different ADCs/antibodies have been achieved with the following gradient: A: 1 M (NH4)2SO4, 500 mM NaCl, 100 mM NaH2PO4 pH 7.4 B: 20 mM NaH2PO4, 20% (v/v) Isopropyl alcohol, pH 7.4. 0% B: 0-1 min, 0-95% B: 1-15 min, 95% B: 15-20 min, 95-0% B: 20-23 min, 0% B: 23-25 min, with a flow of 700 uL/min. 15 μg sample where loaded onto the column for each analysis. UV chromatograms were recorded at 220 and 280 nm.

### General Method for the Conjugation of P5-Based Linker-Payload Constructs to Antibodies to Achieve DAR8.

50 μl of the antibody solution of 10.0 mg/ml in a buffer containing 100 mM NH₄HCO₃ were mixed with 3.33 μl of a 10 mM TCEP solution in P5-conjugation buffer. Directly afterwards, 1.67 μl of a 40 mM solution of the P5-linker-payload construct dissolved in DMSO were added. The mixture was shaken at 350 rpm and 25° C. for 16 hours. The reaction mixtures were purified by preparative size-exclusion chromatography with a 25 ml Superdex™ 200 Increase 10/300GL (Cytiva, Sweden) and a flow of 0.8 ml/min eluting with sterile PBS (Merck, Germany). The antibody containing fractions were pooled and concentrated by spin-filtration (Amicon® Ultra- 2 mL MWCO: 30 kDa, Merck, Germany).

### In Vitro Cytotoxicity

To investigate direct cytotoxicity of ADCs, respective cells were incubated for 4 days for brentuximab ADCs and 7 days for trastuzumab ADCs with increasing concentrations of ADCs (0-3 μg/ml) to generate a dose-response curve. Killing was analysed using resazurin cell viability dye at a final concentration of 55 μM (Sigma-Aldrich) by dividing the fluorescence from control cells in medium by the flourescence of ADC-treated cells. Fluorescence emission at 590 nM was measured on a Microplate reader Infinite M1000 Pro (Tecan).

### In Vitro Bystander Capacity

To analyze bystander activity of ADCs on target-negative cells, 20.000 target-positive cells (L-540 for brentuximab ADCs) were incubated with increasing concentrations of ADCs (0-3 μg/ml). After 5 days, half of the cell culture supernatant volumes was transferred to 5.000 target-negative cells (HL-60 for brentuximab ADCs) and incubated for another 5 days. Killing was analyzed by a resazurin-based viability measurement as described above.

### In Vivo PK Study

Female Sprague-Dawley rats were treated intravenously via the tail-vein with 5 mg ADC per Kilogram bodyweight (bolus) with the respective ADC. Approximately 1 mL of blood was collected after 0.5h, 1 h, 4h, 24h. 48h. 96h. 168h, 336h, and 504h. Blood samples were allowed to stand at room temperature to clot for 30 minutes. Blood serum was isolated from the samples after centrifugation and collection of the supernatant. Blood samples were analyzed via ELISA as follows.

### Analysis of the In Vivo Samples by ELISA for Total Antibody

To evaluate the PK of the ADCs in vivo, the total antibody concentration was measured at different time points in serum of ADC-treated SD rats. Total humanized anti-CD30 antibody was analyzed in rat serum over the range 2000-15.6 ng/ml. Nunc 96-well plate with (100 μl/well) were coated with Recombinant Human CD30/TNFRSF8 diluted in PBS (required concentration: 0.25 μg/ml) and sealed with PCR Foil. Plates were incubated in a fridge to maintain a temperature between 2-8° C. overnight. The coated plates were washed 3× with 300 μl PBST. 200 μl/well of blocking solution (2% Albumin in PBST) was added, the plate was sealed and an incubated at room temperature for 1 hour. The coated plates were washed 3× with 300 μl PBST. 100 μl/well of prepared standards (2000-15.6 ng/ml of the respective ADCs, QCs and test samples were added, the plates were sealed and incubated at room temperature for 1 hour. The plates were washed 3× with 300 μl PBST. 100 μl/well Anti-Human IgG (y-chain specific)-Peroxidase antibody (dilution 1:60000 in PBS) was added and incubated for 1 h at rt. The plates were washed 3× with 300 μl PBST. 50 μl/well TMB was added, the plates were sealed and incubated at room temperature for 15 min. 50 μl/well of 1 M Sulfuric Acid was added. Using a Tecan Plate Reader, the absorbance at a wavelength of 450 nm was measured.

### Analysis of the In Vivo Samples by ELISA for Intact ADC

To evaluate the stability of the ADCs in vivo, the intact ADC concentration was measured at different time points in serum of ADC-treated SD rats. Intact ADC was analyzed in rat serum over the range 2000-15.6 ng/ml. Nunc 96-well plate with (100 μl/well) were coated with rabbit anti-vc-PAB-MMAE pAb diluted in PBS (required concentration: 1 μg/ml) and sealed with PCR Foil. Plates were incubated in a fridge to maintain a temperature between 2-8° C. overnight. The coated plates were washed 3× with 300 μl PBST. 200 μl/well of blocking solution (2% Albumin in PBST) was added, the plate was sealed and an incubated at room temperature for 1 hour. The coated plates were washed 3× with 300 μl PBST. 100 μl/well of prepared standards (2000-15.6 ng/ml of the respective ADCs, QCs and test samples were added, the plates were sealed and incubated at room temperature for 1 hour. The plates were washed 3× with 300 μl PBST. 100 μl/well Goat Anti-Human IgG (H+L) Preabsorbed (dilution 1:25000 in PBS was added and incubated for 1 h at rt. The plates were washed 3× with 300 μl PBST. 100 μl/well TMB was added, the plates were sealed and incubated at room temperature for 10 min. 100 μl/well of 1 M Sulfuric Acid was added. Using a Tecan Plate Reader, the absorbance at a wavelength of 450 nm was measured.

### Example 1: Chemical Synthesis

### General Method 1 for the Synthesis of PEGylated Phenyl Azides

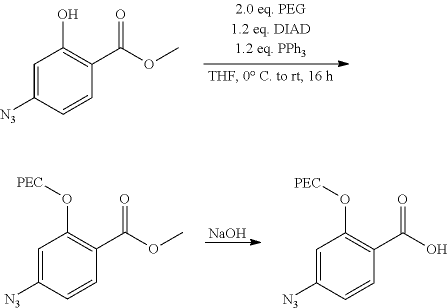

In a 25-mL round-bottom flask, 50 mg Methyl-4-azido-2-hydroxybenzoate (0.259 mmol, 1.0 eq.), 0.518 mmol of the desired PEG-alcohol (2.0 eq.) and 82 mg triphenylphosphine (0.311 mmol, 1.2 eq.) were dissolved in 5 mL of dry THF and the reaction mixture was cooled to 0° C. 54 mg diisopropyl azodicarboxylate (0.311 mmol, 1.2 eq.) were added drop-wise and the solution was allowed warm to room temperature over night while stirring. All volatiles were removed in an N2-Stream and the solids were dissolved in 1 mL of 2N NaOH. The mixture was stirred for 30 min at room temperature, neutralized with 2N HCL and the crude product was purified by preparative HPLC.

Methyl 4-azido-2-(dodecaethyleneplycol)benzoate

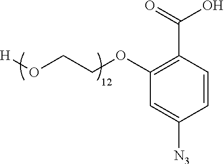

The title compound was synthesized in accordance to general Method 1 from 18 mg Methyl-4-azido-2-hydroxybenzoate (91 μmol, 1.00 eq.), 100 mg of dodecaethylene glycol (183 μmol, 2.0 eq), 29 mg triphenylphosphine (110 μmol, 1.2 eq.), 19 mg diisopropyl azodicarboxylate (110 μmol, 1.2 eq.). The product was obtained as colourless oil after preparative HPLC (Method D) and lyophilization. (6.2 mg, 8.8 μmol, 10%). HR for C₃₁H₅₄N₃O₁₅⁺ [M+H]⁺ calcd.: 708.3550, found 708.74.

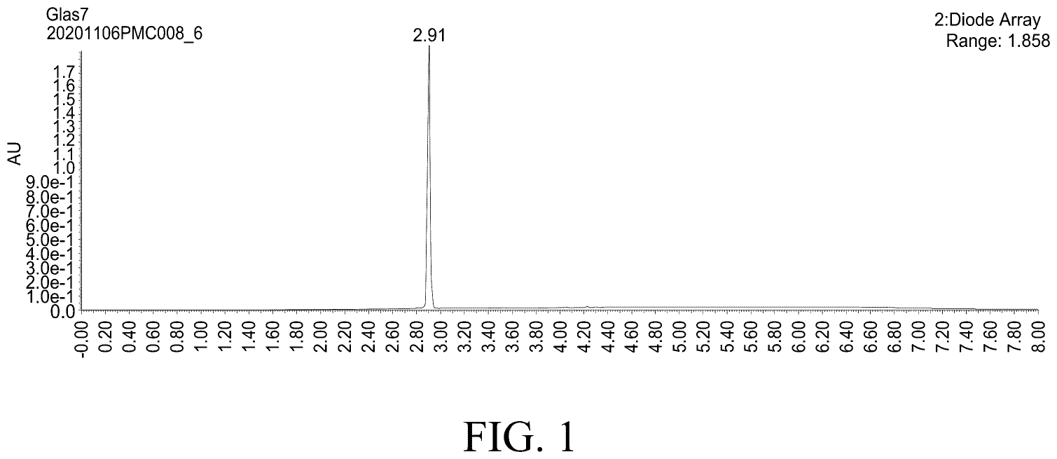

FIG. 1 shows an analytical HPLC chromatogram of the compound methyl 4-azido-2-(dodecaethyleneglycol)benzoate. The horizontal axis depicts the retention time in minutes.

### Methyl 4-azido-2-(tetracosaethyleneplycol)benzoate

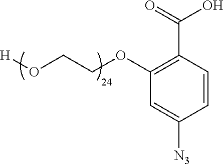

The title compound was synthesized in accordance to general Method 1 from 36 mg Methyl-4-azido-2-hydroxybenzoate (186 μmol, 1.00 eq.), 400 mg of PEG24 (372 μmol, 2.0 eq), 59 mg triphenylphosphine (223 μmol, 1.2 eq.), 39 mg diisopropyl azodicarboxylate (223 μmol, 1.2 eq.). The product was obtained as colourless oil after preparative HPLC (Method D) and lyophilization. (58 mg, 46.9 μmol, 10%). MS for C₅₅H₁₀₂N₃O₂₇⁺ [M+H]⁺ calcd.: 1236.6696, found 1237.05.

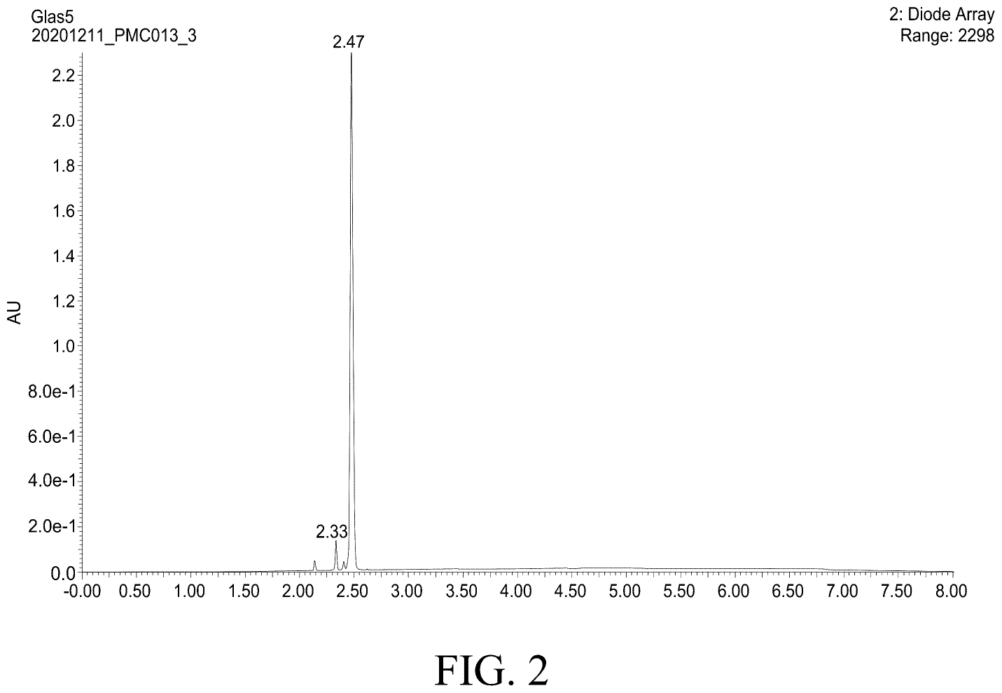

FIG. 2 shows an analytical HPLC chromatogram of the compound methyl methyl 4-azido-2-(tetracosaethyleneglycol)benzoate. The horizontal axis depicts the retention time in minutes.

### General Method 2 for the Synthesis of PEGylated P5 Building Blocks Via the Staudinger Phosphonite Reaction

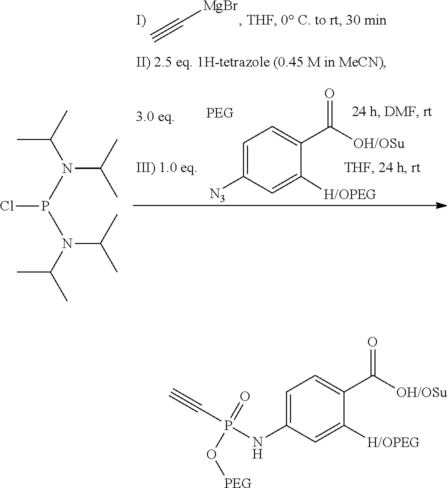

A 25-ml Schlenk flask was charged with 267 mg bis(diisopropylamino)chlorophosphine (1.00 mmol, 1.00 eq.) under an argon atmosphere, cooled to 0° C. and 2.20 mL ethynylmagnesium bromide solution (0.5 M in THF, 1.10 mmol, 1.10 eq.) was added drop wise. The yellowish solution was allowed to warm to room temperature and stirred for further 30 minutes. 3.00 mmol (3.0 eq.) of the desired PEG-alcohol, dissolved in 5.56 mL 1H tetrazole solution (0.45 M in MeCN, 2.50 mmol, 2.50 eq.) were added and the white suspension was stirred overnight at room temperature. The formation of the desired phosphonite was monitored by ³¹P-NMR. 1.0 mmol (1.0 eq.) of the desired azide dissolved in 2 mL of DMF, THF or MeCN was added and the suspension further stirred for 24 h at room temperature. The crude reaction mixture was purified using preparative HPLC.

### P5(PEG12)-OSu

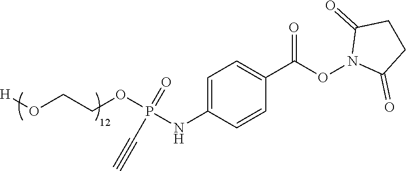

The title compound was synthesized in accordance to general Method 2 from 19.5 mg bis(diisopropylamino)chlorophosphine (73 μmol, 1.00 eq.), 146 μL ethynylmagnesium bromide solution (0.5 M in THF, 73 μmol, 1.00 eq.), 100 mg of dodecaethylene glycol (183 μmol, 2.50 eq), 400 μL 1H-tetrazole solution (0.45 M in MeCN, 183 μmol) and 19 mg 4-azidobenzoic-acid-N-hydroxysuccinimide ester (73 μmol, 1.00 eq.). The product was obtained as colourless oil after preparative HPLC (Method D) and lyophilization. (42.5 mg, 50 μmol, 68%). ¹H NMR (300 MHz, Acetonitrile-d3) δ 8.06 (d, J=8.7 Hz, 2H), 7.32 (d, J=8.8 Hz, 2H), 4.40-4.14 (m, 2H), 3.79-3.69 (m, 2H), 3.66-3.47 (m, 40H), 3.21 (d, J=13.1 Hz, 1H), 2.86 (s, 4H), 1.30 (m, 2H), 1.13-0.79 (m, 2H). ¹³C NMR (151 MHz, CDCl₃) δ 169.77, 169.46, 161.66, 161.47, 152.75, 146.09, 132.90, 132.24, 117.82, 113.97, 113.29, 89.25, 88.92, 77.27, 77.06, 76.85, 74.69, 72.57, 71.19, 70.62, 70.54, 70.51, 70.47, 70.44, 70.36, 70.27, 70.20, 69.74, 69.70, 68.14, 65.77, 65.73, 61.63, 61.60, 40.72, 30.34, 25.68. ³¹P NMR (122 MHz, Acetonitrile-d₃) δ −10.87. HRMS C₃₇H₆₀N₂O₁₉P⁺ calc.: 851.3573 [M+H]⁺, 851.3571.

### P5(PEG12)-COOH

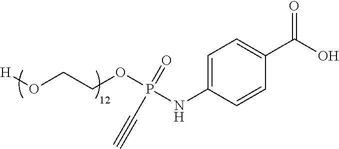

The title compound was synthesized in accordance to general Method 2 from 40 mg bis(diisopropylamino)chlorophosphine (150 μmol, 1.00 eq.), 360 μL ethynylmagnesium bromide solution (0.5 M in THF, 180 μmol, 1.2 eq.), 245 mg of PEG12 (450 μmol, 3.0 eq), 0.83 mL 1H-tetrazole solution (0.45 M in MeCN, 450 μmol, 2.5 eq.) and 39 mg 4-azidobenzoic-acid (150 μmol, 1.00 eq.). The product was obtained as colourless oil after preparative HPLC (Method D) and lyophilization. (25 mg, 34 μmol, 23%). HR-MS for C₃₃H₅₇NO₁₆P⁺ [M+H]⁺ calcd.: 754.3410, found 754.3398

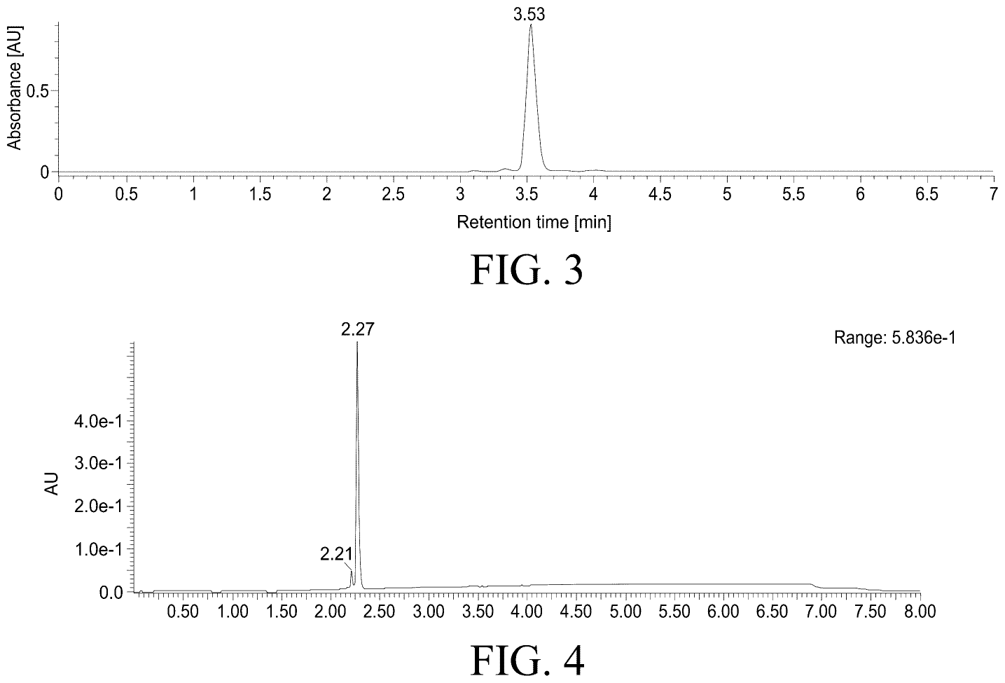

FIG. 3 shows an analytical HPLC chromatogram of the compound P5(PEG12)-COOH.

### P5(PEG24)-OSu

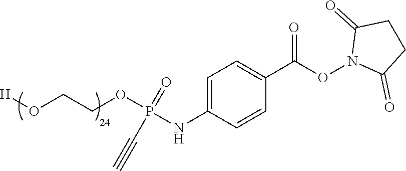

The title compound was synthesized in accordance to general Method 2 from 41 mg bis(diisopropylamino)chlorophosphine (159 μmol, 1.00 eq.), 370 μL ethynylmagnesium bromide solution (0.5 M in THF, 185 μmol, 1.2 eq.), 450 mg of PEG24 (388 μmol, 2.50 eq), 1.02 mL 1H-tetrazole solution (0.45 M in MeCN, 466 μmol, 3.0 eq.) and 40 mg 4-azidobenzoic-acid-N-hydroxysuccinimide ester (155 μmol, 1.00 eq.). The product was obtained as colourless oil after preparative HPLC (Method D) and lyophilization. (79 mg, 57 μmol, 37%). MS for C₆₁H₁₀₉N₂O₃₀P²⁺ [M+2H]²⁺ calcd.: 690.3396, found 690.81.

FIG. 4 shows an analytical HPLC chromatogram of the compound P5(PEG24)-OSu. The horizontal axis depicts the retention time in minutes.

### P5(PEG12, PEG12)-COOH

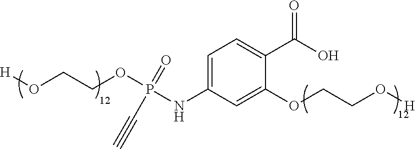

The title compound was synthesized in accordance to general Method 2 from 6.8 mg bis(diisopropylamino)chlorophosphine (25 μmol, 1.00 eq.), 61 μL ethynylmagnesium bromide solution (0.5 M in THF, 31 μmol, 1.2 eq.), 42 mg of Dodecaethyleneglycol (76 μmol, 3.0 eq), 164 μL 1H-tetrazole solution (0.45 M in MeCN, 64 μmol, 2.5 eq.) and 18.4 mg Methyl-4-azido-2-(dodecaethyleneglycol) benzoate (25 μmol, 1.00 eq.). The product was obtained as colourless oil after preparative HPLC (Method D) and lyophilization. (4.8 mg, 3.4 μmol, 13%). MS for C₅₇H₁₀₆NO₂₉P²⁺ [M+2H]²⁺ calcd.: 649.8289, found 650.22.

### P5(PEG12,PEG24)-COOH

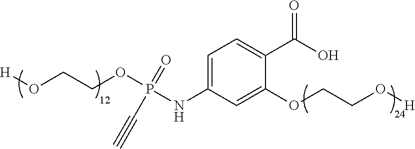

The title compound was synthesized in accordance to general Method 2 from 5.4 mg bis(diisopropylamino)chlorophosphine (20 μmol, 1.00 eq.), 50 μL ethynylmagnesium bromide solution (0.5 M in THF, 24 μmol, 1.2 eq.), 33 mg of Dodecaethyleneglycol (61 μmol, 3.0 eq), 115 μL 1H-tetrazole solution (0.45 M in MeCN, 51 μmol, 3.0 eq.) and 25 mg Methyl-4-azido-2-(tetracosaethyleneglycol) benzoate (20 μmol, 1.00 eq.). The product was obtained as colourless oil after preparative HPLC (Method D) and lyophilization. (6.6 mg, 3.6 μmol, 18%). MS for C₃₁H₁₅₄NO₄₁P²⁺ [M+2H]²⁺ calcd.: 913.9862, found 914.45.

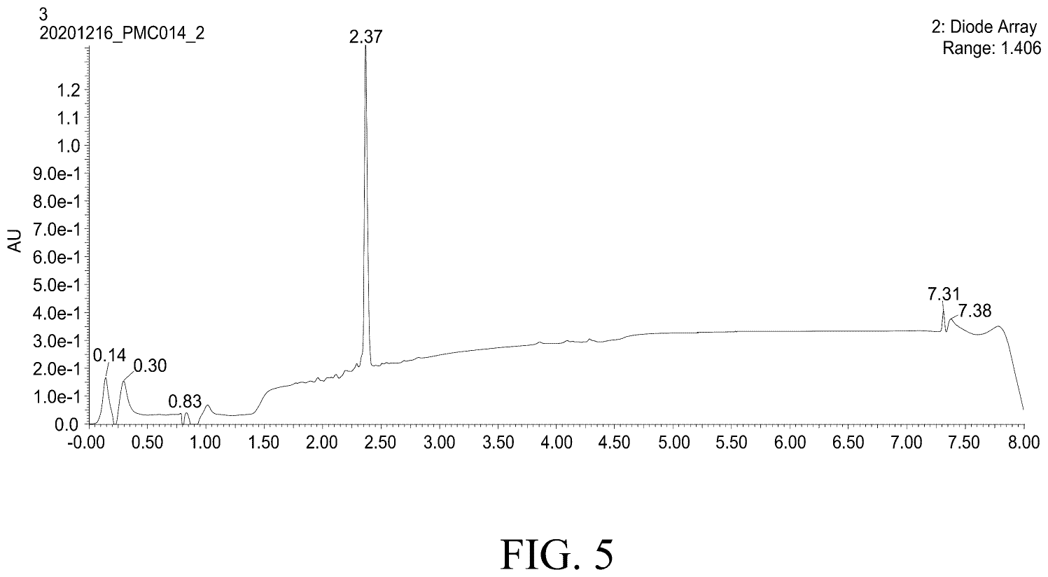

FIG. 5 shows an analytical HPLC chromatogram of the compound P5(PEG12,PEG24)-COOH. The horizontal axis depicts the retention time in minutes.

### P5(PEG24,PEG24)-COOH

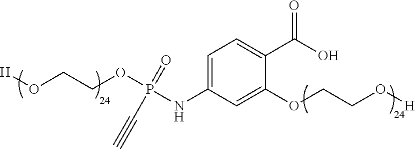

The title compound was synthesized in accordance to general Method 2 from 5.4 mg bis(diisopropylamino)chlorophosphine (20 μmol, 1.00 eq.), 50 μL ethynylmagnesium bromide solution (0.5 M in THF, 24 μmol, 1.2 eq.), 65 mg of PEG24 (61 μmol, 3.0 eq), 115 μL 1H-tetrazole solution (0.45 M in MeCN, 51 μmol, 3.0 eq.) and 25 mg Methyl-4-azido-2-(tetracosaethyleneglycol) benzoate (20 μmol, 1.00 eq.). The product was obtained as colourless oil after preparative HPLC (Method D) and lyophilization. (18.4 mg, 7.5 μmol, 37%). MS for C₁₀₅H₂₀₂NO₅₃P²⁺ [M+2H]²⁺ calcd.: 1178.6451, found 1178.69.

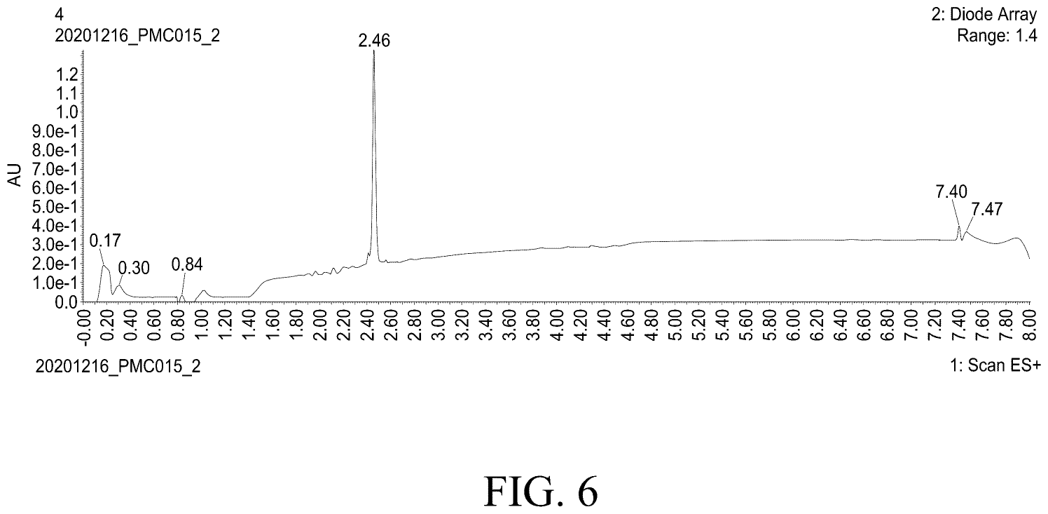

FIG. 6 shows an analytical HPLC chromatogram of the compound P5(PEG24,PEG24)-COOH. The horizontal axis depicts the retention time in minutes.

### NH₂—VC-PAB-MMAE TFA Salt

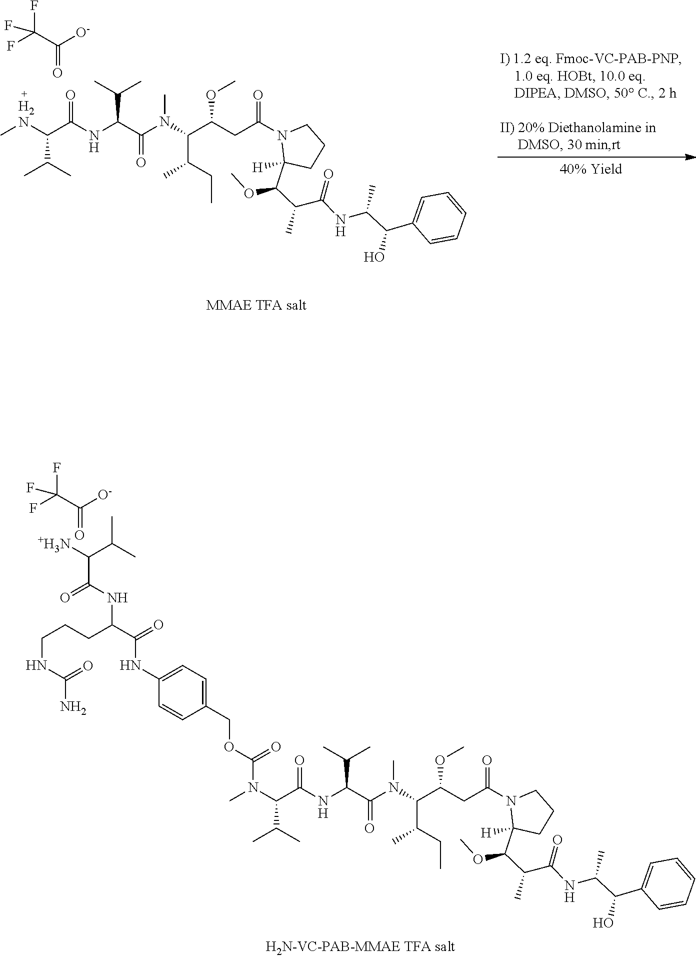

A 10-ml screw-cap vial was charged with 60 μl of a 1 mol/l solution of MMAE TFA salt in DMSO (50 mg, 0.06 mmol, 1.0 eq.), 180 μl of a 0.4 mol/L solution of Fmoc-VC-PAB-PNP solution in DMSO (0.072 mmol, 1.2 eq.) and 60 μl of a 1 mol/l solution of Hydroxybenzotriazole hydrate in DMSO (0.06 mmol, 1.0 eq.). 105 μl DIPEA (0.6 mmol, 10.0 eq.) were added and the yellow solution was stirred for three hours at room temperature. After full consumption of the MMAE starting material, monitored via UPLC/MS, 120 μl of a 50% (w/w) solution of diethanolamine in DMSO was added and the yellow solution further stirred for one hour. 1.5 ml of acetonitrile and 3 ml of water were added and the solution was purified via preparative HPLC using method D. The desired product was obtained as TFA salt as a white powder after lyophilization. (29.5 mg, 39.7%). HR-MS for C₅₈H₉₅N₁₀O₁₂⁺ [M+H]⁺ calcd.: 1123.7125, found 1123.7130.

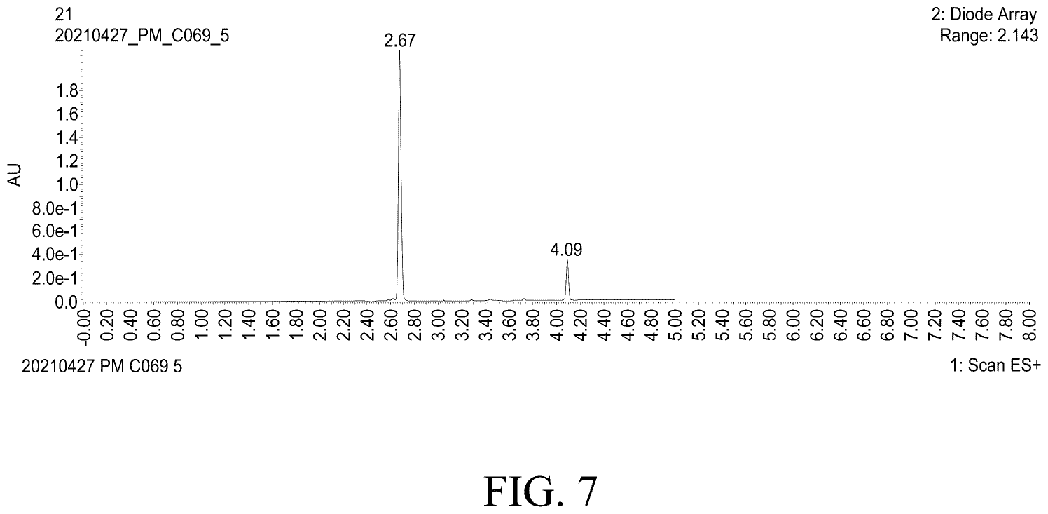

FIG. 7 shows an analytical HPLC chromatogram of the compound NH₂—VC-PAB-MMAE TFA salt. The horizontal axis depicts the retention time in minutes.

### NH₂—VC-PAB-MMAF TFA Salt

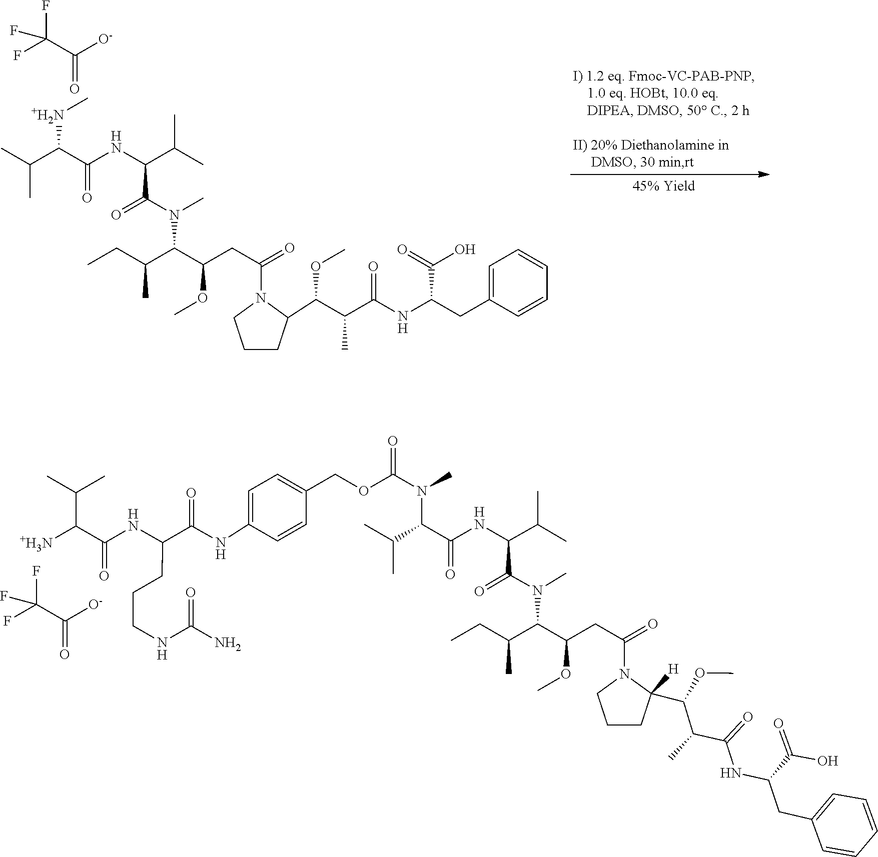

A 10-ml screw-cap vial was charged with 60 μl of a 1 mol/l solution of MMAF TFA salt in DMSO (0.06 mmol, 1.0 eq.), 180 μl of a 0.4 mol/L solution of Fmoc-VC-PAB-PNP solution in DMSO (0.072 mmol, 1.2 eq.) and 60 μl of a 1 mol/l solution of Hydroxybenzotriazole hydrate in DMSO (0.06 mmol, 1.0 eq.). 105 μl DIPEA (0.6 mmol, 10.0 eq.) were added and the yellow solution was stirred for three hours at room temperature. After full consumption of the MMAF starting material, monitored via UPLC/MS, 120 μl of a 50% (w/w) solution of diethanolamine in DMSO was added and the yellow solution further stirred for one hour. 1.5 ml of acetonitrile and 3 ml of water were added and the solution was purified via preparative HPLC using method D. The desired product was obtained as TFA salt as a white powder after lyophilization. (33.7 mg, 44.9%). MS for C₅₈H₉₃N₁₀O₁₃⁺ [M+H]⁺ calcd.: 1137.6919, found 1138.27

General Method 4 for the Synthesis of Linker-Payload Constructs from P5-OSu

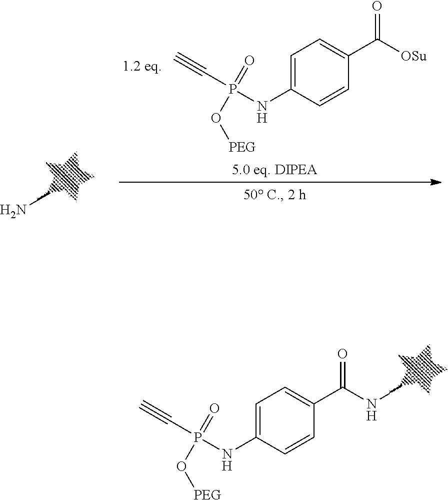

A screw-cap vial was charged with 48 μL of a 200 mM solution of the desired P5(OR,OR)—COOH in dry DMSO (9.6 μmol, 1.20 eq.) and 40 μL of a 200 mM solution of the amino containing linker payload in dry DMSO (8 μmol, 1.00 eq.) and 7 μL of DIPEA (40 μmol, 5.00 eq.) were added. The solution was agitated for 2 h at 50° C., the solution was allowed to warm to room temperature, dissolved in 1.5 mL of MeCN and 3 mL of H₂O and directly injected into preparative HPLC for purification.

### P5(PEG12)-VC-PAB-MMAF

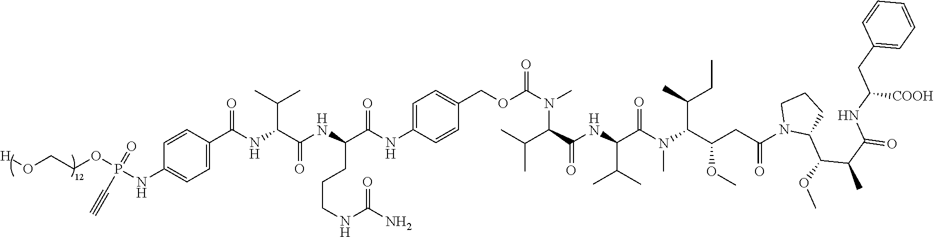

The compound was synthesized according to general method 4 from 60 μL of a 200 mM solution of P5(PEG12)-OSu in dry DMSO (12 μmol, 1.00 eq.), 60 μL of a 200 mM solution of H2N-VC-PAB-MMAF in dry DMSO (12 μmol, 1.00 eq.) and 10.5 μL of DIPEA (120 μmol, 10.00 eq.). The product was obtained as colourless oil after preparative HPLC (Method C) and lyophilization. (8.45 mg, 4.56 μmol, 38%) HR-MS for C₉₁H₁₄₈N₁₁O₂₈P²⁺ [M+2H]²⁺ calcd.: 937.0111, found 937.5

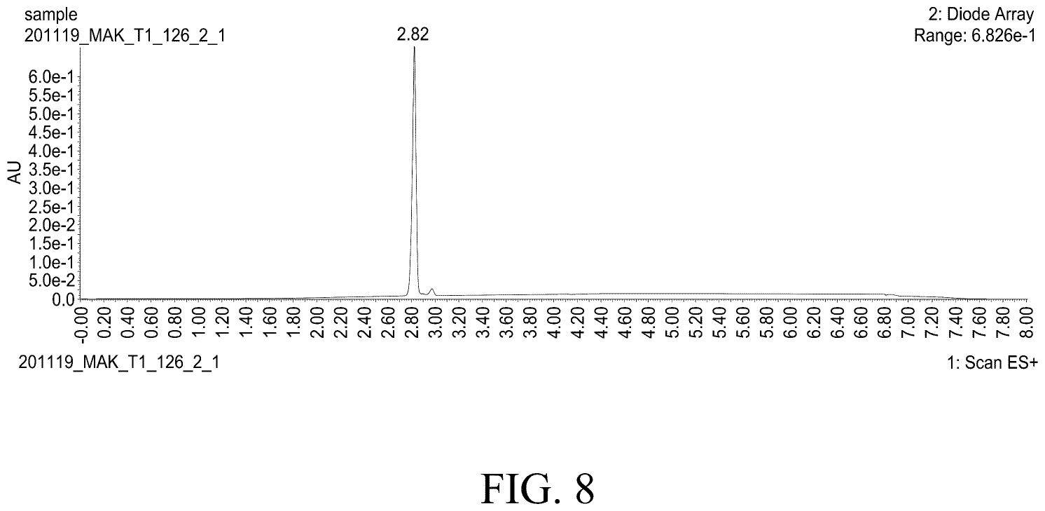

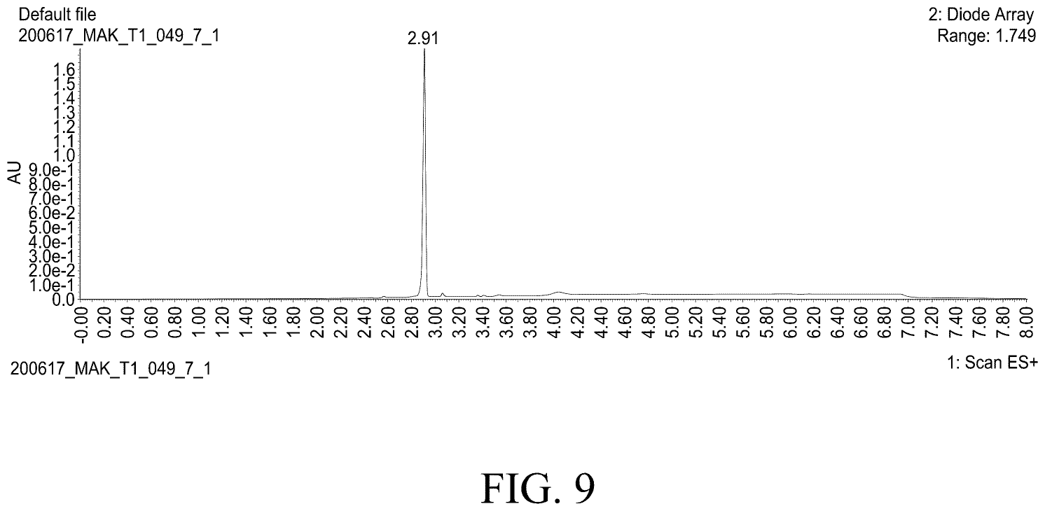

FIG. 8 shows an analytical HPLC chromatogram of the compound P5(PEG12)-VC-PAB-MMAF. The horizontal axis depicts the retention time in minutes.

### P5(PEG12)-VC-PAB-MMAE

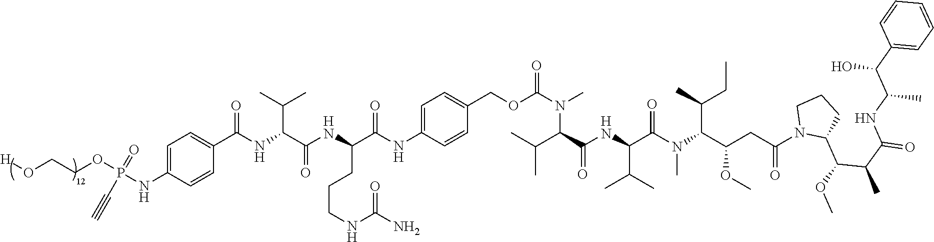

In a screw-cap-vial equipped with a stirring bar, 20.7 mg of H₂N-Val-Cit-PAB-MMAE (18.4 μmol, 1.00 eq.) and 15.7 mg P5(PEG12)-OSu (18.4 μmol, 1 eq.) were dissolved in 200 μl DMSO. 12 μL DIPEA (73.6 μmol, 4.00 eq.) were added and the solution was stirred overnight at room temperature. The solution was diluted with 3 ml 30% MeCN in H₂O and subjected to preparative HPLC purification (Method D). The desired compound was obtained as a white solid after lyophilization. (11.9 mg, 6.44 μmol, 35.2%). HRMS C₉₁H₁₅₀N₁₁O₂₇P²⁺ calcd.: 930.0215 [M+2H]²⁺, exp.: 930.0211.

FIG. 9 shows an HPLC chromatogram of the compound P5(PEG12)-VC-PAB-MMAE. The horizontal axis depicts the retention time in minutes.

### P5(PEG24)-VC-PAB-MMAE

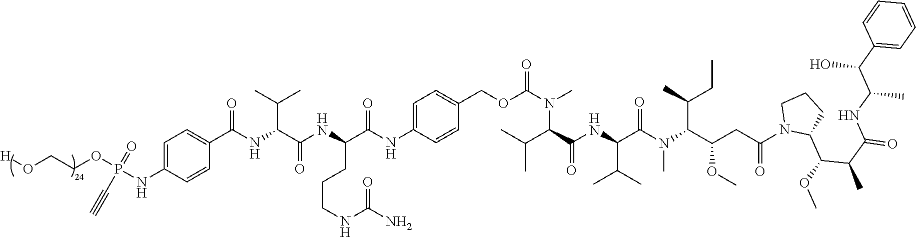

The compound was synthesized according to general method 4 from 15 μL of a 200 mM solution of P5(PEG24)-OSu in dry DMSO (3 μmol, 1.00 eq.), 15 μL of a 250 mM solution of H2N-VC-PAB-MMAE in dry DMSO (3.8 μmol, 1.25 eq.) and 5 μL of DIPEA. The product was obtained as colourless oil after preparative HPLC (Method C) and lyophilization. (1.31 mg, 0.55 μmol, 18%) HR-MS for C₁₁₅H₁₉₉N₁₁O₃₉P³⁺ [M+3H]³⁺ calcd.: 796.7894, found 796.7839

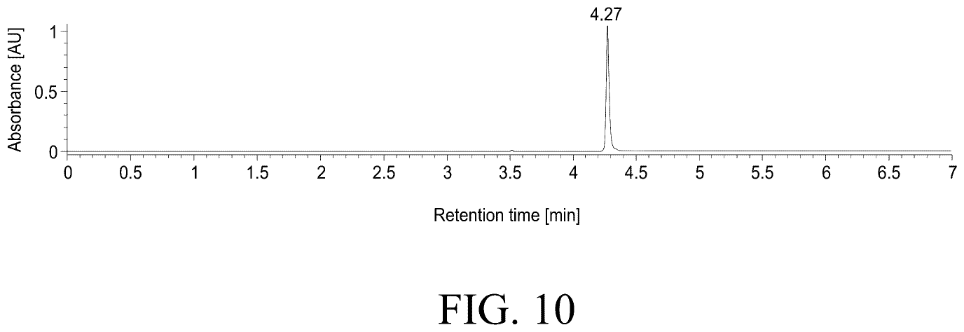

FIG. 10 shows an analytical HPLC chromatogram of the compound P5(PEG24)-VC-PAB-MMAE.

General Method 5 for the Synthesis of Linker-Payload Constructs from P5-COOH

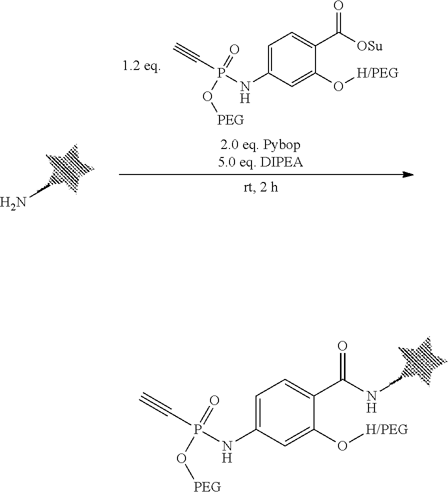

A screw-cap vial was charged with 60 μL of a 200 mM solution of the desired P5(OR,OR)—COOH in dry DMSO (12 μmol, 1.20 eq.), 50 μL of a 400 mM solution of Pybob in dry DMSO (20 μmol, 2.00 eq.) and 50 μL of a 200 mM solution of the amino containing linker payload in dry DMSO (10 μmol, 1.00 eq.) and 8.7 μL of DIPEA (50 μmol, 5.00 eq.) were added. The solution was agitated for 2 h at room temperature, dissolved in 1.5 mL of MeCN and 3 mL of H₂O and directly injected into preparative HPLC for purification.

### P5(PEG12, PEG12)-VC-PAB-MMAE

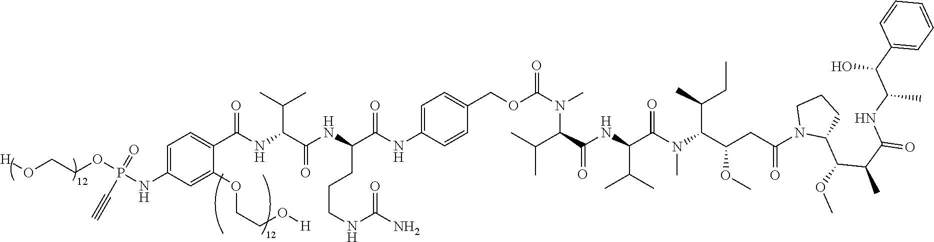

The compound was synthesized according to general method 5 from 4.8 mg P5(PEG12,PEG12)-COOH (3.7 μmol, 1.00 eq.), 27.7 μL of a 200 mM solution of Pybob in dry DMSO (5.54 μmol, 1.50 eq.), 8.9 μL of a 500 mM solution of H2N-VC-PAB-MMAE in dry DMSO (4.45 μmol, 1.20 eq.) and 2.4 μL of DIPEA (18.5 μmol, 5.00 eq.). The product was obtained as colourless oil after preparative HPLC (Method C) and lyophilization. (6.41 mg, 2.66 μmol, 72%) MS for C₁₁₅H₁₉₈N₁₁O₂₄₀P²⁺ [M+2H]²⁺ calcd.: 1202.6779, found 1202.74

### P5(PEG12 PEG24)-VC-PAB-MMAE

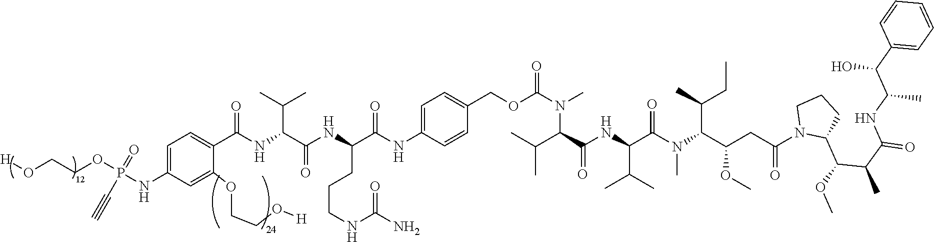

The compound was synthesized according to general method 5 from 9.8 μL of a 200 mM solution of the desired P5(PEG12)-COOH in dry DMSO (1.9 μmol, 1.20 eq.), 8.2 μL of a 400 mM solution of Pybob in dry DMSO (3.2 μmol, 2.00 eq.), 8.2 μL of a 200 mM solution of the amino containing linker payload in dry DMSO (1.6 μmol, 1.00 eq.) and 1.4 μL of DIPEA (8 μmol, 5.00 eq.). The product was obtained as colourless oil after preparative HPLC (Method C) and lyophilization. (4.16 mg, 1.42 μmol, 86%) MS for C₁₃₉H₂₄₇N₁₁O₅₂P³⁺ [M+3H]³⁺ calcd.: 978.2259, found 978.74

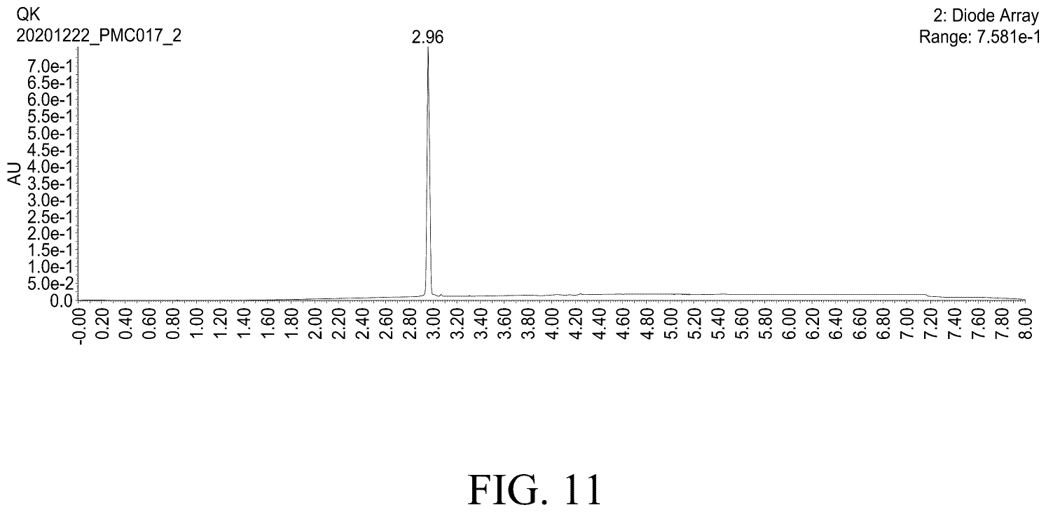

FIG. 11 shows an analytical HPLC chromatogram of the compound P5(PEG12,PEG24)-VC-PAB-MMAE. The horizontal axis depicts the retention time in minutes.

### P5(PEG24,PEG24)-VC-PAB-MMAE

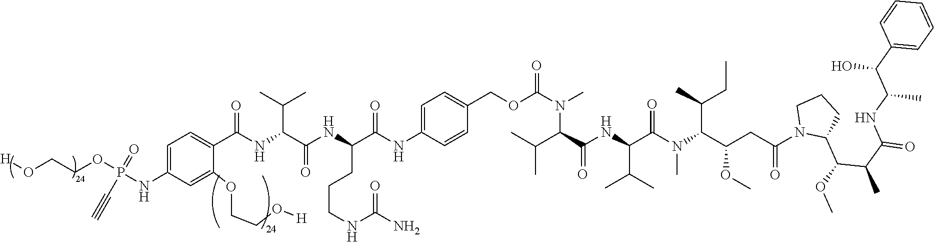

The compound was synthesized according to general method 5 from 26 μL of a 200 mM solution of the desired P5(OR,OR)—COOH in dry DMSO (5.3 μmol, 1.20 eq.), 22 μL of a 400 mM solution of Pybob in dry DMSO (8.8 μmol, 2.00 eq.), 22 μL of a 200 mM solution of the amino containing linker payload in dry DMSO (4.4 μmol, 1.00 eq.) and 3.9 μL of DIPEA (22 μmol, 5.00 eq.). The product was obtained as colourless oil after preparative HPLC (Method C) and lyophilization. (3.6 mg, 1.04 μmol, 23%) MS for C₁₆₃H₂₉₅N₁₁O₆₄P³⁺ [M+3H]³⁺ calcd.: 1154.3308, found 1154.77

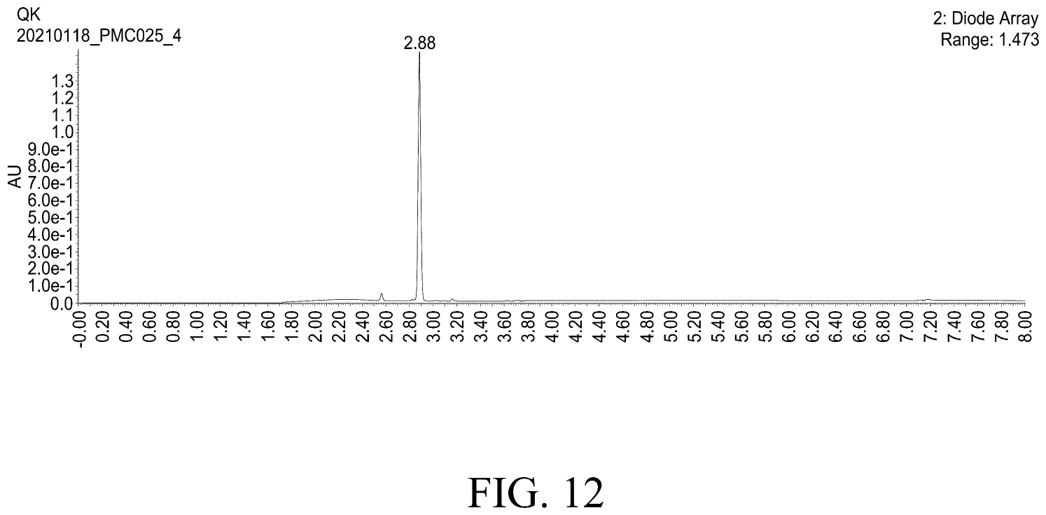

FIG. 12 shows an analytical HPLC chromatogram of the compound P5(PEG24,PEG24)-VC-PAB-MMAE. The horizontal axis depicts the retention time in minutes.

### General Procedure for the Synthesis of Valine-Citrullin-PAB- or Valine-Alanine-PAB-Drug Linker Compounds

In a screw-cap-vial equipped with a stirring bar, Fmoc-Val-Cit-PAB-PNP (1.00 eq.) or Fmoc-Val-Ala-PAB-PNP (1.00 eq.), HOBt*H₂O (1.00 eq.) and the desired amine (1.00 eq.) were dissolved in DMF or DMSO. Then DIPEA (2.00 eq.) was added and the solution was stirred for 1h or until completion at room temperature. Then 100 μL piperidin were added to reach a final concentration of 20% v/v and the solution was stirred at room temperature for 30 minutes. The solution was diluted with 3 ml 30% MeCN in H₂O and subjected to semi-preparative HPLC purification (30-90% MeCN+0.1% TFA in H₂O+0.1% TFA over 40 min).

### NH₂—VC-PAB-AT7519 TFA Salt

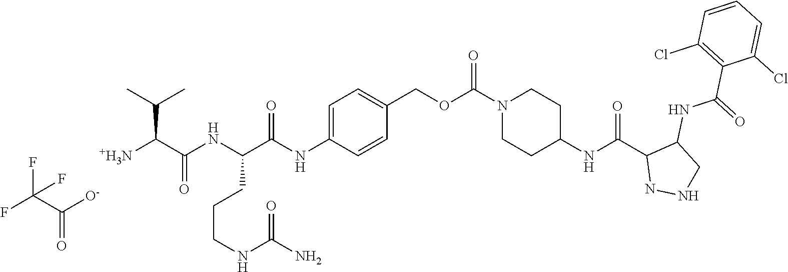 [SMILES: O=C([O-])C(F)(F)F ; CC(C)[C@H]([NH3+])C(=O)N[C@@H](CCCNC(N)=O)C(=O)Nc1ccc(COC(=O)N2CCC(NC(=O)C3NNCC3NC(=O)c3c(Cl)cccc3Cl)CC2)cc1]

The above stated compound was synthesized according to the general procedure for the synthesis of VC-PAB-drug linker from 10 mg of Fmoc-Val-Cit-PAB-PNP (13 μmol, 1.00 eq.), 2 mg HOBt*H₂O (13 μmol, 1.00 eq.) and 5 mg AT7519 (13 μmol, 1 eq.) in 500 μL DMF and 4.5 μL DIPEA (26 μmol, 2.00 eq.). Then 100 μL piperidin were added to reach a final concentration of 20% v/v and the solution was stirred at room temperature for 30 minutes. The solution was diluted with 3 ml 30% MeCN in H₂O and subjected to semi-preparative HPLC purification (30-90% MeCN+0.1% TFA in H₂O+0.1% TFA over 40 min). The desired compound was obtained as a white solid after lyophilization. (4.5 mg, 5.0 μmol, 38.4%). MS for C₃₅H₄₅Cl₂N₁₀O₇⁺, calcd.: 787.28 [M+H]⁺, exp.: 787.19.

### NH₂—VC-PAB-SB743921 TFA Salt

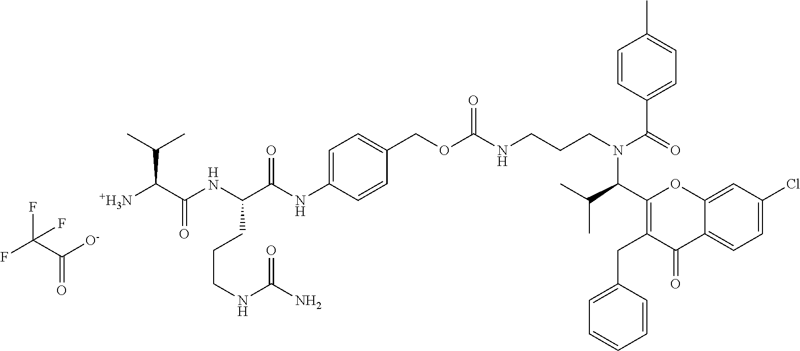 [SMILES: O=C([O-])C(F)(F)F ; Cc1ccc(C(=O)N(CCCNC(=O)OCc2ccc(NC(=O)[C@H](CCCNC(N)=O)NC(=O)[C@@H]([NH3+])C(C)C)cc2)[C@@H](c2oc3cc(Cl)ccc3c(=O)c2Cc2ccccc2)C(C)C)cc1]

The above stated compound was synthesized according to the general procedure for the synthesis of VC-PAB-drug linker from 11.3 mg of Fmoc-Val-Cit-PAB-PNP (14.8 μmol, 1.00 eq.), 1.9 mg HOBt*H₂O (12.3 μmol, 1.00 eq.) and 6.8 mg SB743921 (12.3 μmol, 1 eq.) in 500 μL DMF and 4 μL DIPEA (24.6 μmol, 2.00 eq.). Then 100 μL piperidin were added to reach a final concentration of 20% v/v and the solution was stirred at room temperature for 30 minutes. The solution was diluted with 3 ml 30% MeCN in H₂O and subjected to semi-preparative HPLC purification (30-90% MeCN+0.1% TFA in H₂O+0.1% TFA over 40 min). The desired compound was obtained as a white solid after lyophilization. (6.4 mg, 6.17 μmol, 41.7%). MS for C₅₀H₆₁ClN₇O₈⁺, calcd.: 922.43 [M+H]⁺, exp.: 922.76.

### NH₂—VA-PAB-SB743921 TFA Salt

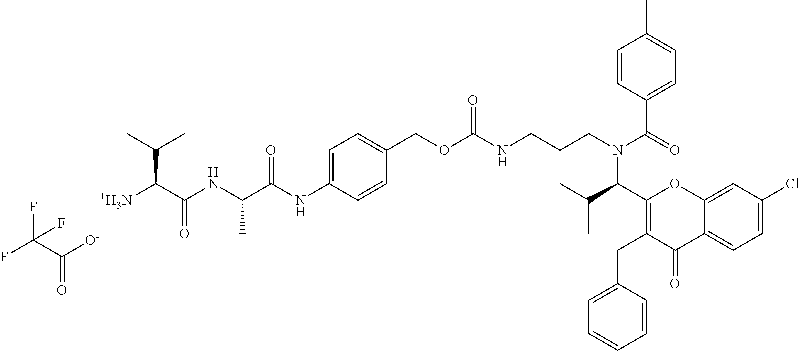 [SMILES: O=C([O-])C(F)(F)F ; Cc1ccc(C(=O)N(CCCNC(=O)OCc2ccc(NC(=O)[C@H](C)NC(=O)[C@@H]([NH3+])C(C)C)cc2)[C@@H](c2oc3cc(Cl)ccc3c(=O)c2Cc2ccccc2)C(C)C)cc1]

The above stated compound was synthesized according to the general procedure for the synthesis of VA-PAB-drug linker from 10.3 mg of Fmoc-Val-Cit-PAB-PNP (13.56 μmol, 1.00 eq.), 1.9 mg HOBt*H₂O (12.3 μmol, 1.36 eq.) and 5 mg SB743921 (9.04 μmol, 1 eq.) in 300 μL DMSO and 4 μL DIPEA (24.6 μmol, 2.72 eq.). Then 100 μL piperidin were added to reach a final concentration of 20% v/v and the solution was stirred at room temperature for 30 minutes. The solution was diluted with 3 ml 30% MeCN in H₂O and subjected to semi-preparative HPLC purification (30-90% MeCN+0.1% TFA in H₂O+0.1% TFA over 40 min). The desired compound was obtained as a white solid after lyophilization. (5.5 mg, 5.79 μmol, 64.0%). MS for Ca₄₇H₅₅ClN₅O₇⁺, calcd.: 836.3785 [M+H]⁺, exp.: 836.39416.

### NH₂—VC-PAB-Emetin TFA Salt

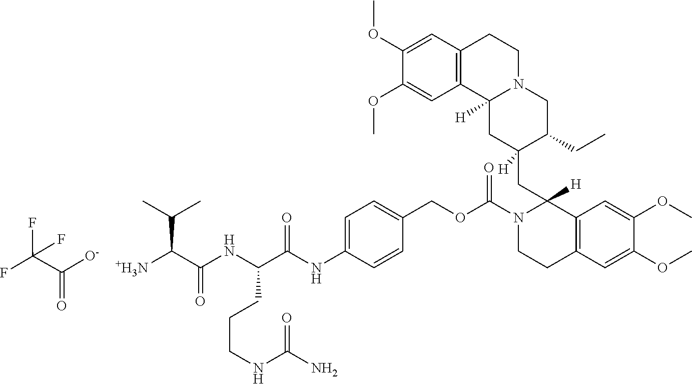 [SMILES: O=C([O-])C(F)(F)F ; CC[C@H]1CN2CCc3cc(OC)c(OC)cc3[C@@H]2C[C@@H]1C[C@@H]1c2cc(OC)c(OC)cc2CCN1C(=O)OCc1ccc(NC(=O)[C@H](CCCNC(N)=O)NC(=O)[C@@H]([NH3+])C(C)C)cc1]

The above stated compound was synthesized according to the general procedure for the synthesis of VC-PAB-drug linker from 57 mg of Fmoc-Val-Cit-PAB-PNP (74 μmol, 1.20 eq.), 9.4 mg HOBt*H₂O (61.6 μmol, 1.00 eq.) and 34 mg Emetin*HCl (61.6 μmol, 1 eq.) in 500 μL DMF and 21 μL DIPEA (123 μmol, 2.00 eq.). Then 100 μL piperidin were added to reach a final concentration of 20% v/v and the solution was stirred at room temperature for 30 minutes. The solution was diluted with 3 ml 30% MeCN in H₂O and subjected to semi-preparative HPLC purification (30-90% MeCN+0.1% TFA in H₂O+0.1% TFA over 40 min). The desired compound was obtained as a white solid after lyophilization. (30.5 mg, 30.5 μmol, 41.2%). MS for C₄₈H₆₈N₇O₉⁺, calcd.: 886.51 [M+H]⁺, exp.: 886.88.

### NH₂—VA-PAB-Panobinostat TFA Salt

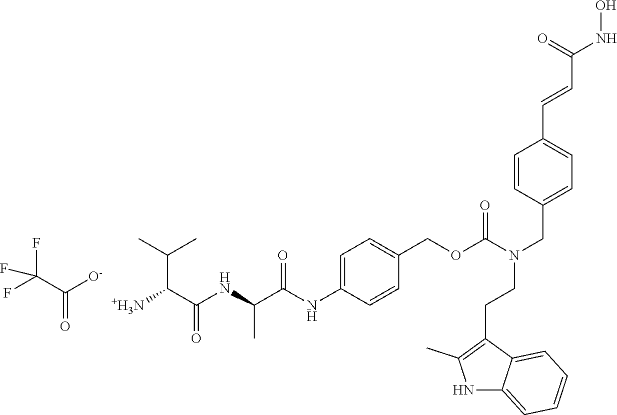 [SMILES: O=C([O-])C(F)(F)F ; Cc1[nH]c2ccccc2c1CCN(Cc1ccc(/C=C/C(=O)NO)cc1)C(=O)OCc1ccc(NC(=O)[C@@H](C)NC(=O)[C@H]([NH3+])C(C)C)cc1]

The above stated compound was synthesized according to the general procedure for the synthesis of VA-PAB-drug linker from 60 mg of Fmoc-Val-Ala-PAB-PNP (88.14 μmol, 1.54 eq.), 13.5 mg HOBt*H₂O (100 μmol, 1.74 eq.) and 20 mg Panobinostat (57.2 μmol, 1 eq.) in 300 μL DMSO and 100 μL DIPEA (588 μmol, 10.28 eq.). Then 50 μL piperidin were added and the solution was stirred at room temperature for 30 minutes. The solution was diluted with 3 ml 30% MeCN in H₂O and subjected to semi-preparative HPLC purification (30-90% MeCN+0.1% TFA in H₂O+0.1% TFA over 40 min). The desired compound was obtained as a white solid after lyophilization. (19.5 mg, 24.9 μmol, 43.5%). MS for C₃₇H₄₅N₆O₆⁺, calcd.: 669.3396 [M+H]⁺, exp.: 669.36175.

P5 Compounds with Di-Peptide Based Cleavage Sides

### P5(PEG12)-VC-PAB-AT7519

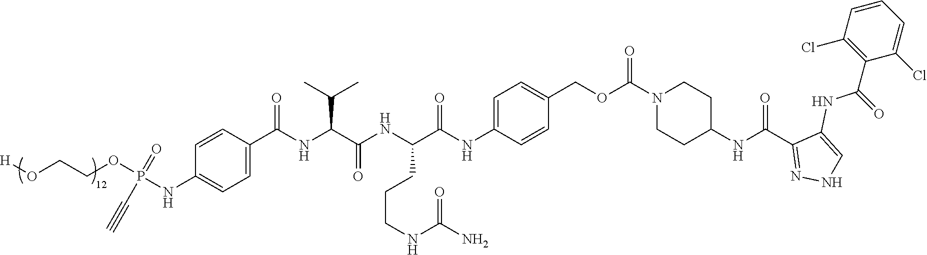

In a screw-cap-vial equipped with a stirring bar, 4.5 mg of H₂N-Val-Cit-PAB-AT7519 TFA salt (5.0 μmol, 1.00 eq.) and 4.8 mg P5(PEG12)-OSu (5.7 μmol, 1.14 eq.) were dissolved in 500 μl DMSO. 4 μL DIPEA (22.8 μmol, 4.56 eq.) were added and the solution was stirred overnight at room temperature. The solution was diluted with 3 ml 30% MeCN in H₂O and subjected to semi-preparative HPLC purification (30-90% MeCN+0.1% TFA in H2O+0 .1% TFA over 40 min). The desired compound was obtained as a white solid after lyophilization. (1.5 mg, 0.98 μmol, 19.6%). HRMS C₉₁H₁₅₀N₁₁O₂₇P²⁺ calcd.: 930.0215 [M+2H]²⁺, exp.: 930.0211.

### P5(PEG12)-VC-PAB-SB743921

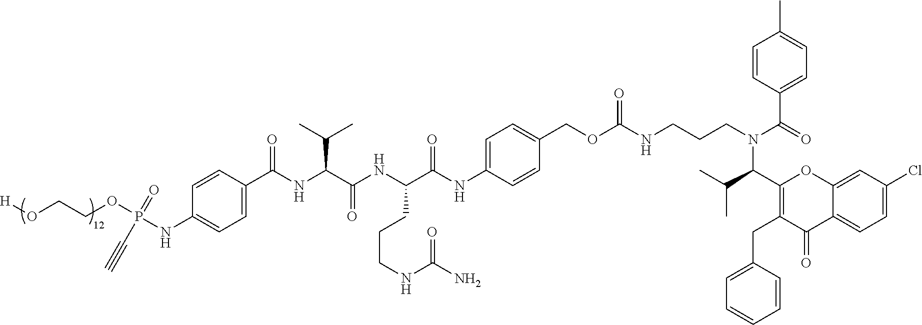

In a screw-cap-vial equipped with a stirring bar, 6.4 mg of H₂N-Val-Cit-PAB-SB743921 TFA salt (6.2 μmol, 1.00 eq.) and 7 mg P5(PEG12)-OSu (8.3 μmol, 1.34 eq.) were dissolved in 500 μl DMSO. 3 μL DIPEA (17.2 μmol, 2.77 eq.) were added and the solution was stirred overnight at 50° C. The solution was diluted with 3 ml 40% MeCN in H₂O and subjected to semi-preparative HPLC purification (40-90% MeCN+0.1% TFA in H2O+0.1% TFA over 40 min). The desired compound was obtained as a white solid after lyophilization. (5.9 mg, 3.58 μmol, 57.7%). HRMS C₈₃H₁₁₅ClN₈O₂₃P⁺ calcd.: 1657.7496 [M+H]⁺, exp.: 1657.7438.

### P5(PEG24)-VA-PAB-SB743921

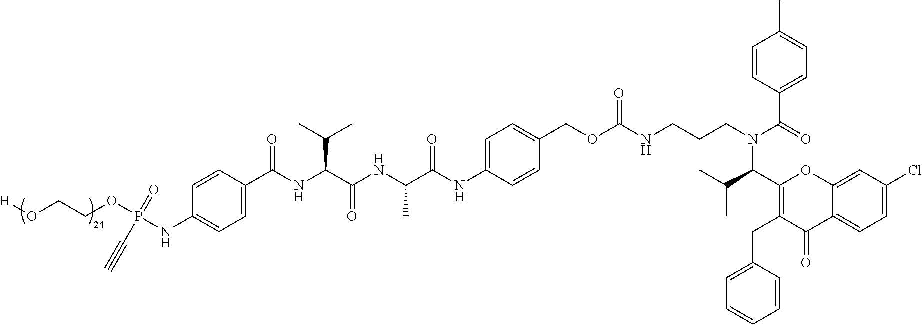

In a screw-cap-vial equipped with a stirring bar, 5.5 mg of H₂N-Val-Ala-PAB-SB743921 TFA salt (5.7 μmol, 1.00 eq.) and 9.4 mg P5(PEG24)-OSu (6.8 μmol, 1.2 eq.) were dissolved in 500 μl DMSO. 3 μL DIPEA (17.2 μmol, 3.02 eq.) were added and the solution was stirred overnight at 50° C. The solution was diluted with 3 ml 40% MeCN in H₂O and subjected to semi-preparative HPLC purification (40-90% MeCN+0.1% TFA in H2O+0.1% TFA over 40 min). The desired compound was obtained as a white solid after lyophilization. (8.79 mg, 4.27 μmol, 74.9%). HRMS C₁₀₄H₁₅₈ClN₆O₃₄P²⁺ calcd.: 1051.0134 [M+H]⁺, exp.: 1051.01605.

### P5(PEG12)-VC-PAB-Emetin

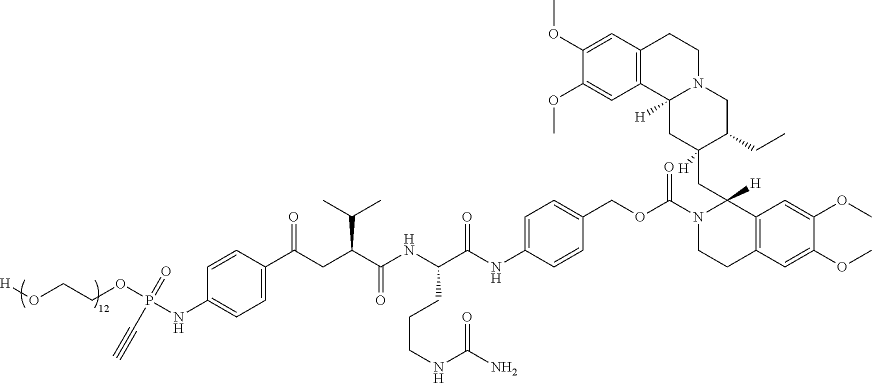

In a screw-cap-vial equipped with a stirring bar, 10.3 mg of H₂N-Val-Cit-PAB-Emetin TFA salt (10.3 μmol, 1.00 eq.) and 12 mg P5(PEG12)-OSu (14 μmol, 1.36 eq.) were dissolved in 500 μl DMSO. 5 μL DIPEA (28 μmol, 2.72 eq.) were added and the solution was stirred overnight at 50° C. The solution was diluted with 3 ml 40% MeCN in H₂O and subjected to semi-preparative HPLC purification (40-90% MeCN+0.1% TFA in H2O+0.1% TFA over 40 min). The desired compound was obtained as a white solid after lyophilization. (9.37 mg, 5.78 μmol, 456.1%). HRMS C₃₁H₁₂₂N₃O₂₄P⁺ calcd.: 1621.8304 [M+H]⁺, exp.: 1621.8298.

### P5(PEG24)-VA-PAB-Panobinostat

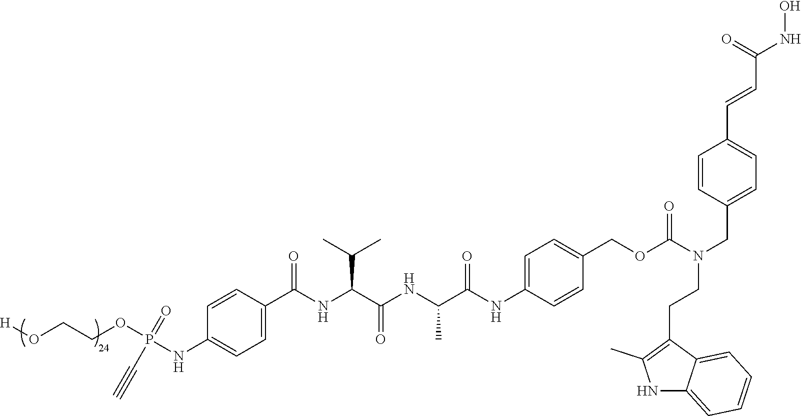

In a screw-cap-vial equipped with a stirring bar, 3.9 mg of NH2-VA-PAB-Panobinostat TFA salt (5 μmol, 1.00 eq.) and 8.2 mg P5(PEG24)-OSu (6 μmol, 1.2 eq.) were dissolved in 500 μl DMSO. 3 μL DIPEA (17.2 μmol, 3.44 eq.) were added and the solution was stirred overnight at 50° C. The solution was diluted with 3 ml 40% MeCN in H₂O and subjected to semi-preparative HPLC purification (40-90% MeCN+0.1% TFA in H2O+0.1% TFA over 40 min). The desired compound was obtained as a white solid after lyophilization. (2.51 mg, 1.30 μmol, 26.0%). HRMS C₉₄H₁₄₈N₇O₃₃P²⁺ calcd.: 967.4939 [M+2H]+, exp.: 967.5337.

### Glucoronic Acid Based Cleavage Sides

### (3-nitro-4-(2,3,4-Tri-O-acetyl-ß-D-glucopyranuronic acid methylester)phenyl)methanol

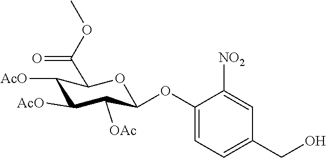

4-(2,3,4-Tri-O-acetyl-ß-D-glucopyranuronic acid methylester)-3 nitrobenzaldehyde was synthesized according to a previously published procedure (see, e.g., Walther et al., “Combatting implant-associated biofilms through localized drug synthesis”, Journal of Controlled Release 287 (2018) 94-10s, doi: 10.1016/j.jconrel.2018.08.025) and used afterwards as described. In a flame dried flask aldehyde derivative (207 μmol, 1.0 eq) was dissolved in CHCl₃/i-PrOH (2.0 mL/0.5 mL) and cooled to 0° C. under an atmosphere of N₂. Then silica gel (100 mg) was added and left to stir for 10 min. NaBH₄ (0.414 mmol, 2.0 eq) is added in one scoop. The reaction was monitored by TLC. The reaction was completed usually within 30-45 min, in case it was not, additional NaBH₄ (0.23 mmol, 1.0 eq) was added. Upon completion, the reaction was diluted with CH₂Cl₂ and filtered over a pluck of Celite®, and washed with CH₂Cl₂ several times. The filtrate was washed with brine (3×20 mL), dried over MgSO₄, filtered and the solvent removed under reduced pressure. The product was isolated as off white solid (96 mg, 198 μmol, 95.7%). ¹H NMR (600 MHz, Chloroform-d) δ 7.89 (d, J=2.2 Hz, 1H), 7.61 (dd, J=8.6, 2.2 Hz, 1H), 7.44 (d, J=8.5 Hz, 1H), 7.34 (s, 1H), 5.51-5.31 (m, 3H), 5.27 (d, J=6.9 Hz, 1H), 4.80 (s, 2H), 4.28 (d, J=8.8 Hz, 1H), 3.82 (s, 3H), 2.20 (s, 3H), 2.14 (s, 3H), 2.13 (s, 3H). ¹³C NMR (151 MHz, CDC₃) δ 172.75, 172.04, 172.02, 169.45, 150.91, 144.08, 140.07, 134.64, 125.94, 123.04, 102.65, 79.95, 79.74, 79.52, 75.29, 73.88, 72.95, 71.49, 66.20, 55.78, 55.44, 23.27, 16.92.

### (3-amino-4-(2,3,4-Tri-O-acetyl-ß-D-glucopyranuronic acid methylester)phenyl)methanol

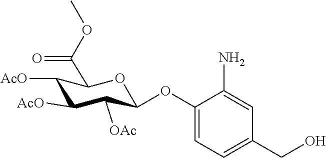

890 mg of compound (3-nitro-4-(2,3,4-Tri-O-acetyl-ß-D-glucopyranuronic acid methylester)phenyl)methanol (1.8 mmol) is dissolved in MeOH with a little EtOAc. In an argon flushed flask, 200 mg Pd—C is placed and suspended in MeOH/EtOAc containing (3-nitro-4-(2,3,4-Tri-O-acetyl-ß-D-glucopyranuronic acid methylester)phenyl)methanol. The suspension is stirred vigorously. Using the vacuum pump the flask is evacuated and flushed with argon three times. Than the argon ballon is exchanged with H₂ ballon and the suspension is again evacuated and then flushed with H₂ three times. The suspension is left to stir over night. Solids were filter off through celite and the filtrate was concentrated under reduced pressure to yield the title compound (733 mg, 1.61 mmol, 90%). ¹H NMR (600 MHz, DMSO-d₆) δ 6.87 (d, J=8.2 Hz, 1H), 6.73 (d, J=1.9 Hz, 1H), 6.52 (dd, J=8.2, 2.0 Hz, 1H), 5.54 (t, J=9.6 Hz, 1H), 5.46 (d, J=7.9 Hz, 1H), 5.25-5.10 (m, 2H), 5.03 (t, J=5.8 Hz, 1H), 4.38 (d, J=4.8 Hz, 2H), 3.71 (s, 3H), 2.11 (s, 3H), 2.07 (s, 6H). ¹³C NMR (151 MHz, DMSO) δ 172.68, 172.66, 172.48, 170.36, 145.36, 141.36, 118.93, 117.64, 116.54, 102.08, 74.09, 74.06, 74.02, 72.27, 65.97, 55.76, 51.78, 23.75, 23.68, 23.50, 23.40.

### (3-azido-4-(2,3,4-Tri-O-acetyl-ß-D-glucopyranuronic acid methylester)phenyl)methanol

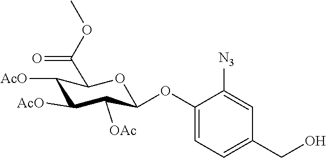

A 500-ml round-bottom flask was charged with 733 mg (3-amino-4-(2,3,4-Tri-O-acetyl-ß-D-glucopyranuronic acid methylester)phenyl)methanol (1.61 mmol), suspended in 2.5 mL water and cooled to 0° C. 800 μL concentrated aqueous HCl were added, followed by dropwise addition of 167 mg sodium nitrite (2.4 mmol, 1.50 eq.) solution in 1.5 mL water. The mixture was stirred for 20 min at 0° C., 15 mL EtOAc were added and a solution of 157 mg sodium azide (2.4 mmol, 1.5 eq.) in 750 μl water was added drop-wise. The solution was allowed to warm to room temperature and stirred for one more hour. Phases were separated, the aqueous phase was extracted two times with EtOAc, combined organic fractions were washed two times with water, dried (MgSO₄) and all volatiles were removed under reduced pressure, yielding 718 mg of the title compound (1.5 mmol, 92.7%). ¹H NMR (600 MHz, Chloroform-d) δ 7.18 (d, J=8.3 Hz, 1H), 7.15-7.11 (m, 2H), 5.44-5.37 (m, 2H), 5.36-5.32 (m, 1H), 5.14 (d, J=7.4 Hz, 1H), 4.70 (s, 2H), 4.23-4.16 (m, 2H), 3.81 (s, 3H), 2.16 (s, 3H), 2.12 (s, 3H), 2.11 (s, 3H). ¹³C NMR (151 MHz, CDC₃) δ 173.91, 172.84, 171.98, 169.52, 150.32, 140.63, 133.39, 126.73, 121.96, 102.91, 79.97, 79.76, 79.55, 75.35, 74.51, 73.59, 71.79, 71.64, 66.94, 63.13, 55.71, 25.40, 23.32, 16.90, 16.82.

### (3-azido-4-(2,3,4-Tri-O-acetyl-ß-D-glucopyranuronic acid methylester)benzyl(4-nitrophenyl) carbonate

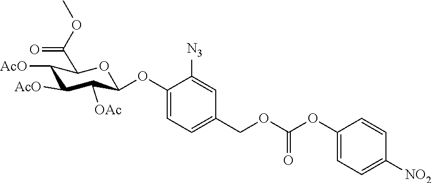

718 mg of (3-azido-4-(2,3,4-Tri-O-acetyl-ß-D-glucopyranuronic acid methylester)phenyl)methanol (1.5 mmol) were dissolved in 8.5 mL DMF and 912 mg bisnitrophenyl carbonate (3 mmol) were added together with 523 μL DIPEA (3 mmol). The reaction was stirred at room temperature for 2 hours and diluted with DCM. The organic solution was washed with brine, dried over MgSO₄ and evaporated to dryness. Coevaporation with toluene removed residual DMF. The crude was purified on silica column chromatography (hexane/EtOAc 2:1 to 1:1) and yielded 553 mg of the title compound (0.856 mmol, 57%). ¹H NMR (600 MHz, Chloroform-d) δ 8.30 (dd, J=8.8, 1.2 Hz, 2H), 7.40 (dd, J=8.9, 1.2 Hz, 2H), 7.28 (d, J=1.0 Hz, 1H), 7.22-7.12 (m, 3H), 5.41-5.29 (m, 3H), 5.24 (s, 2H), 5.17 (dd, J=7.2, 1.0 Hz, 1H), 4.27-4.10 (m, 2H), 3.77 (d, J=1.0 Hz, 3H), 2.12 (d, J=1.0 Hz, 3H), 2.08 (d, J=1.0 Hz, 3H), 2.07 (d, J=1.0 Hz, 3H). ¹³C NMR (151 MHz, CDCl₃) δ 170.09, 169.29, 169.15, 166.71, 155.40, 152.34, 148.94, 145.49, 130.91, 130.86, 118.82, 99.76, 77.22, 77.01, 76.80, 72.72, 71.70, 70.86, 69.84, 68.95, 60.38, 53.03, 21.04, 20.63, 20.60, 20.49, 14.20. HRMS C₂₇H₂₆KN₄O₁₅⁺ calcd.: 685.1026 [M+H]⁺, exp.: 685.1028.

General Procedure for the Synthesis of (3-Azido-4-(2,3,4-Tri-O-Acetyl-ß-D-Glucopyranuronic acid methylester)benzyl carbamates

(3-azido-4-(2,3,4-Tri-O-acetyl-ß-D-glucopyranuronic acid methylester) benzyl (4-nitrophenyl) carbonate (1.2 equivalents) is placed in a pear shaped flask and set under an argon atmosphere. The desired amine (1 equivalent) is dissolved in dry DMF (60 μmol/mL) and added to (3-azido-4-(2,3,4-Tri-O-acetyl-ß-D-glucopyranuronic acid methylester) benzyl (4-nitrophenyl) carbonate to dissolve everything. HOBt (0.1 equivalents) and DIPEA (2.3 equivalents) is added and the reaction is stirred for 1 hour at room temperature or until completion. DMF is removed on the rotavap and the residue is dissolved in 40% MeCN in H₂O and subjected to semi-preparative HPLC purification.

(3-azido-4-(2,3,4-Tri-O-acetyl-ß-D-glucopyranuronic acid methylester) benzyl SB743921 carbamate

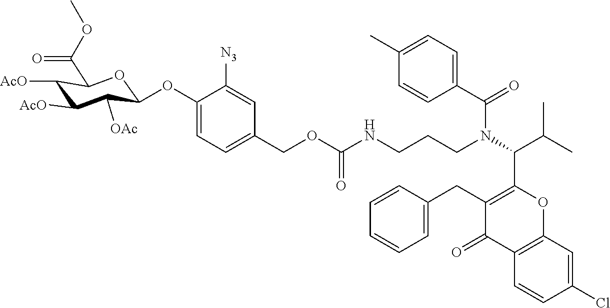

The above stated compound was synthesized following the general procedure for the synthesis of (3-azido-4-(2,3,4-Tri-O-acetyl-ß-D-glucopyranuronic acid methylester) benzyl carbamates from 10 mg SB743921 (18 μmol), 14 mg (3-azido-4-(2,3,4-Tri-O-acetyl-B-D-glucopyranuronic acid methylester) benzyl (4-nitrophenyl) carbonate (21.6 μmol), 1.8 μL HOBt solution (1 M in DMSO) and 7.3 μL DIPEA. 12.3 mg of the title compound were isolated (12 μmol, 67%).

### (3-azido-4-(2,3,4-Tri-O-acetyl-ß-D-glucopyranuronic acid methylester) benzyl AT7519 carbamate

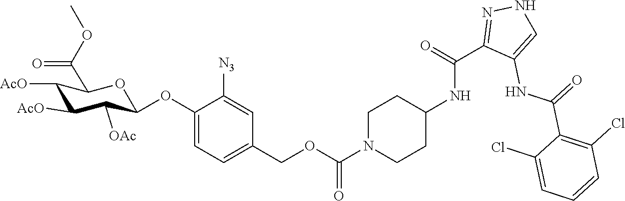

The above stated compound was synthesized following the general procedure for the synthesis of benzyl carbamates from 8.8 mg AT7519 (23.2 μmol), 15 mg (3-azido-4-(2,3,4-Tri-O-acetyl-ß-D-glucopyranuronic acid methylester) benzyl (4-nitrophenyl) carbonate (23.2 μmol), 2.3 μL HOBt solution (1 M in DMSO) and 8 μL DIPEA. 11.7 mg of the title compound were isolated (13.2 μmol, 56.7%). HRMS C37H39Cl2N8O14+ calcd.: 889.1957 [M+H]+, exp.: 889.1942.

### P5(PEG12)-GlcA-SB743921

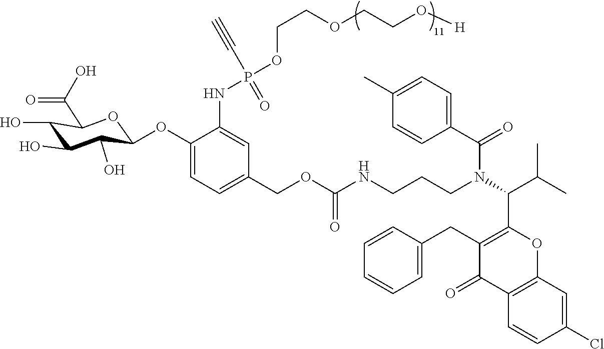

12.3 mg (3-azido-4-(2,3,4-Tri-O-acetyl-ß-D-glucopyranuronic acid methylester) benzyl SB743921 carbamate (12 μmol) were dissolved in 1 mL MeOH and 4.4 mg LiOH (104 μmol) dissolved in 1 mL H2O were added. The reaction was stirred for 15 min at 0° C. and then neutralized using acetic acid (8 μL). The solvents were evaporated and vacuum dried. The residue was placed under an argon atmosphere and dissolved in 500 μL DMF. Then 214 μL of freshly prepared Di-(dodecaethylene glycol) ethynylphosphonite (24 μmol, 111 μM in THF/MeCN) was added and stirred for 24 h. The solvents were evaporated and the crude was dissolved in 40% MeCN in H2O and subjected to semi-preparative HPLC purification. 2.2 mg of the above stated compound were isolated (1.5 μmol, 12.5%).

### P5(PEG12)-GlcA-AT7519

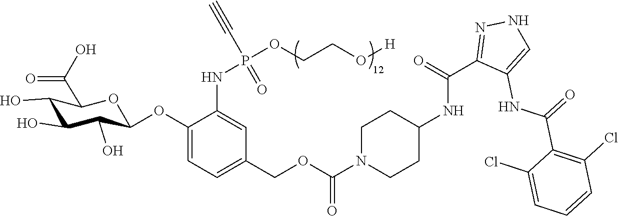

11.7 mg (3-azido-4-(2,3,4-Tri-O-acetyl-ß-D-glucopyranuronic acid methylester) benzyl AT7519 carbamate (13.2 μmol) were dissolved in 1 mL MeOH and 4.8 mg LiOH (114.4 μmol) dissolved in 1 mL H2O were added. The reaction was stirred for 15 min at 0° C. and then neutralized using acetic acid (8 μL). The solvents were evaporated and vacuum dried. The residue was placed under an argon atmosphere and dissolved in 500 μL DMF. Then 180 μL of freshly prepared Di-(dodecaethylene glycol) ethynylphosphonite (19.8 μmol, 111 μM in THF/MeCN) was added and stirred for 24 h. The solvents were evaporated and the crude was dissolved in 40% MeCN in H2O and subjected to semi-preparative HPLC purification. 2.73 mg of the above stated compound were isolated (2.04 μmol, 15%).

(3-O-dodecaethylene glycol-P-ethynyl-phosphonamidate-4-(2,3,4-Tri-O-acetyl ß-D-glucopyranuronic acid methylester) benzyl (4-nitrophenyl) carbonate

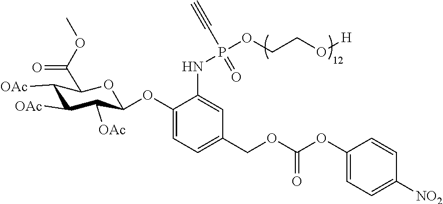

31.3 mg (3-azido-4-(2,3,4-Tri-O-acetyl-ß-D-glucopyranuronic acid methylester) benzyl (4-nitrophenyl) carbonate (48.5 μmol) were placed in a pear shaped round bottom flask and dissolved in 1 mL DMF 520 μL of freshly prepared Di-(dodecaethylene glycol) ethynylphosphonite (58.2 μmol, 111 μM in THF/MeCN) was added and stirred for 24 h. The solvents were evaporated and the crude was dissolved in 40% MeCN in H2O and subjected to semi-preparative HPLC purification. 12 mg of the above stated compound were isolated (9.7 μmol, 20%).

(3-O-dodecaethylene glycol-P-ethynyl-phosphonamidate-4-(2,3,4-Tri-O-acetyl 13-D-glucopyranuronic acid methylester) benzyl MMAE carbamate

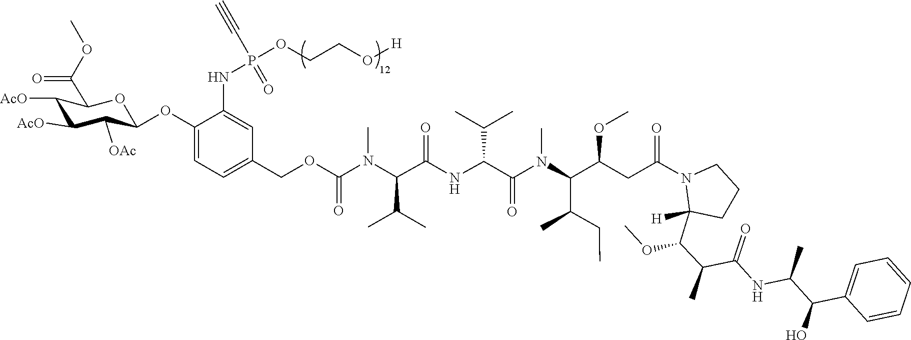

(3-O-dodecaethylene glycol-P-ethynyl-phosphonamidate-4-(2,3,4-Tri-O-acetyl ß-D-glucopyranuronic acid methylester) benzyl (4-nitrophenyl) carbonate (1.00 equivalents) is placed in a pear shaped flask and set under an argon atmosphere and dissolved in 500 μL DMF. 10 μL of a MMAE solution (10 μmol, 1M in DMSO, 1.1 equivalents) is added followed by 1 μL HOBt (1 μmol 0.1 equivalents) and 3.5 μL DIPEA (2.3 equivalents). The reaction is stirred for 1 hour at room temperature. DMF is removed on the rotavap and the residue is dissolved in 40% MeCN in H2O and subjected to semi-preparative HPLC purification to yield 4.7 mg of the title compound (2.6 μmol, 26.7%)

### P5(PEG12)-GlcA-MMAE

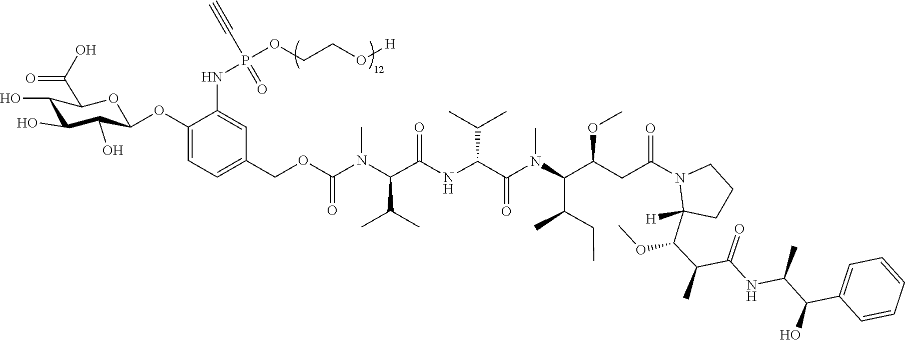

4.7 mg of (3-O-dodecaethylene glycol-P-ethynyl-phosphonamidate-4-(2,3,4-Tri-O-acetyl ß-D-glucopyranuronic acid methylester) benzyl MMAE carbamate were placed in a pear shaped flask and dissolved in 100 μL MeOH 100 μL of a LUOH solution (104.8 mM in H2O, 10.4 μmol) were added and the reaction was stirred for 30 minutes at room temperature. 3 mL of 40% MeCN in H2O with 0.1% TFA are added the mixture was subjected to semi-preparative HPLC purification to yield 2.47 mg of the title compound (1.47 μmol, 57%).

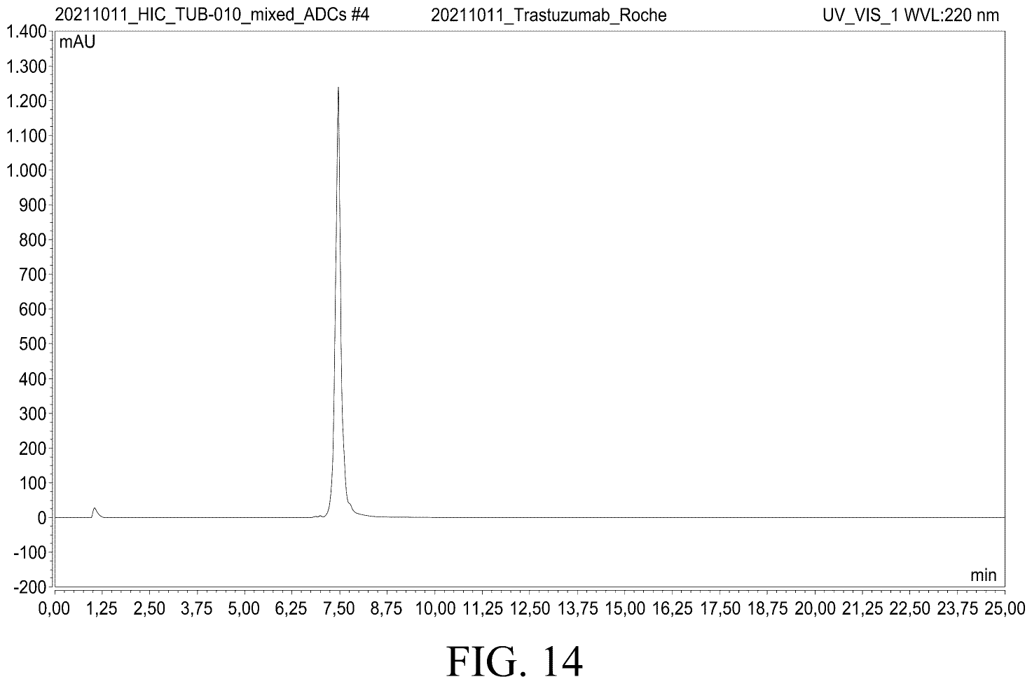

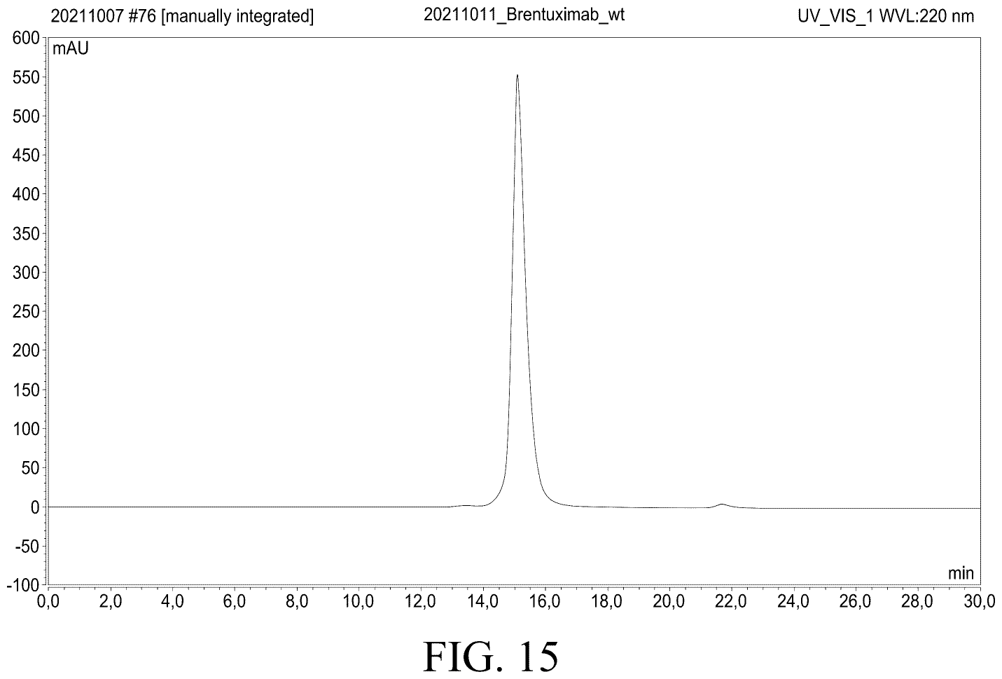

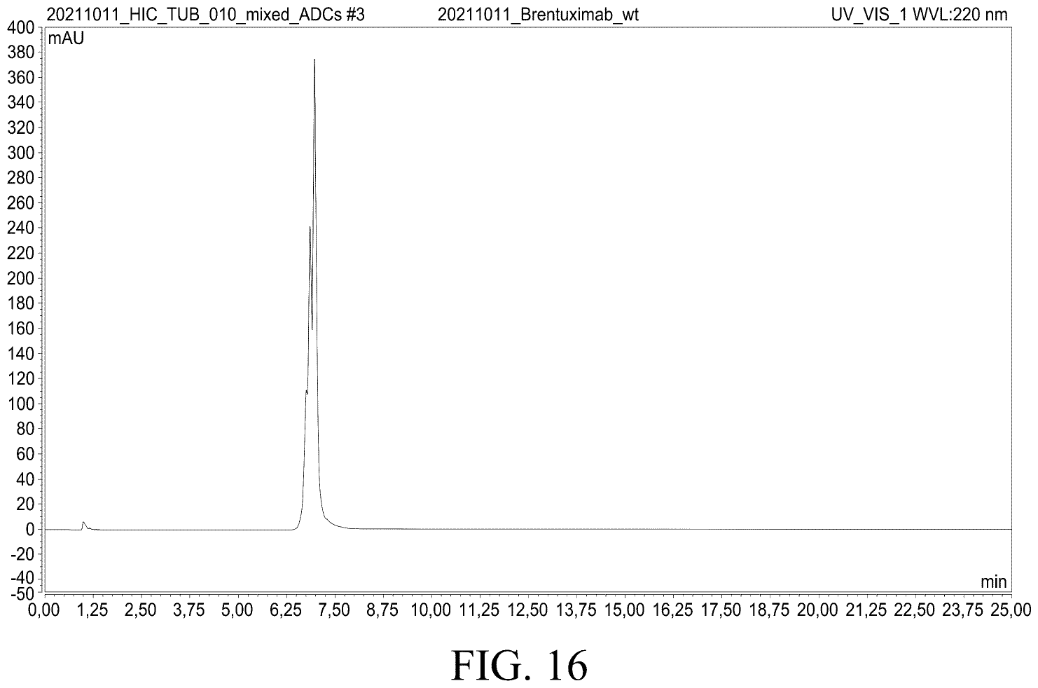

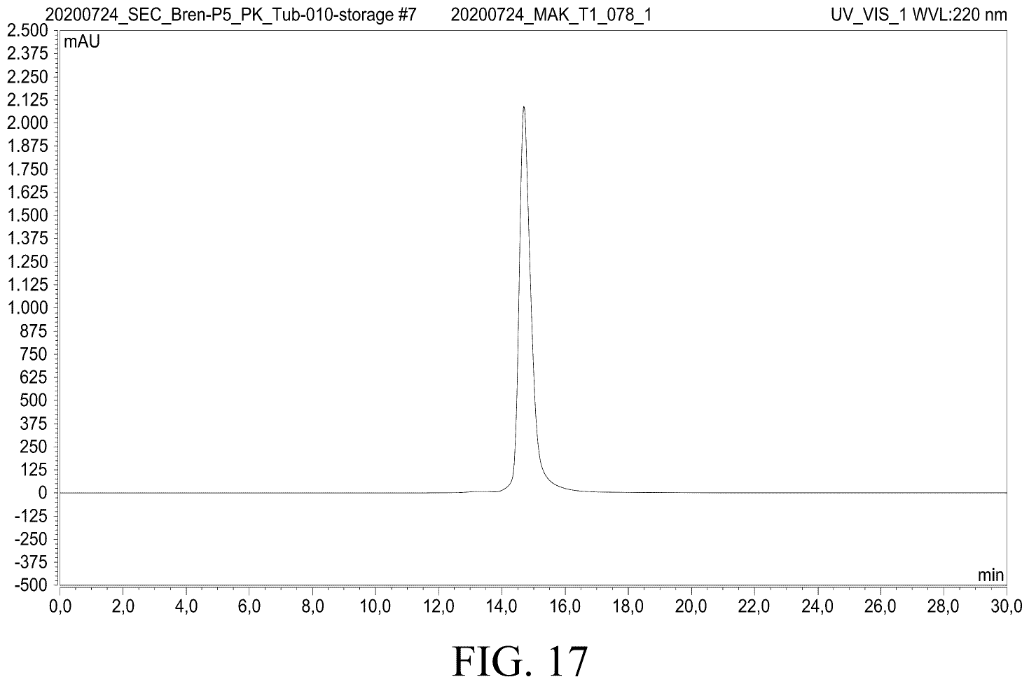

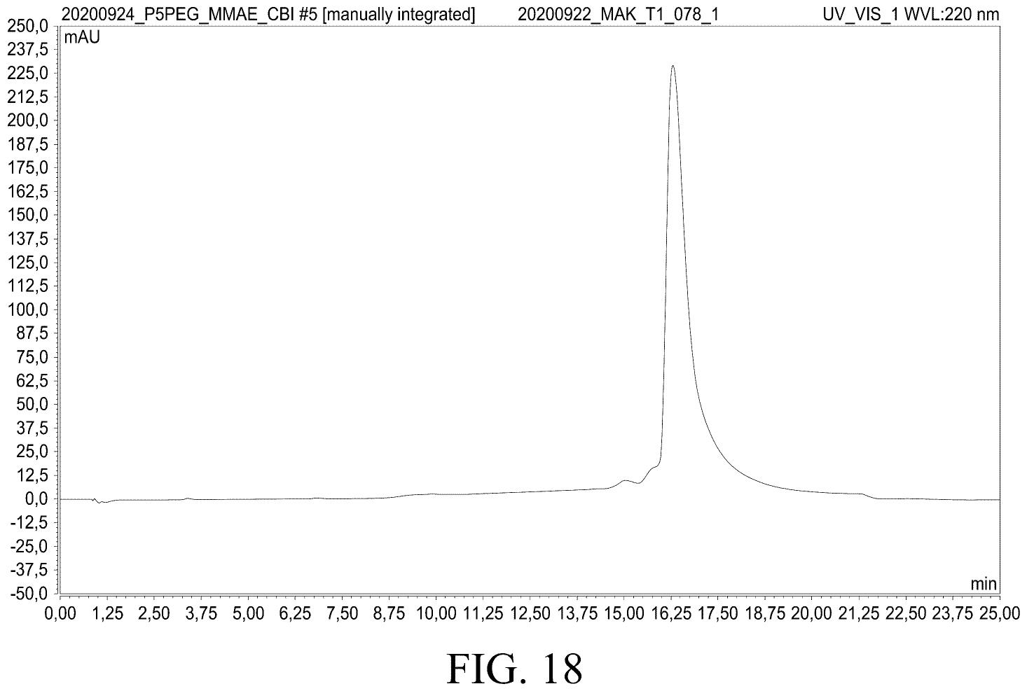

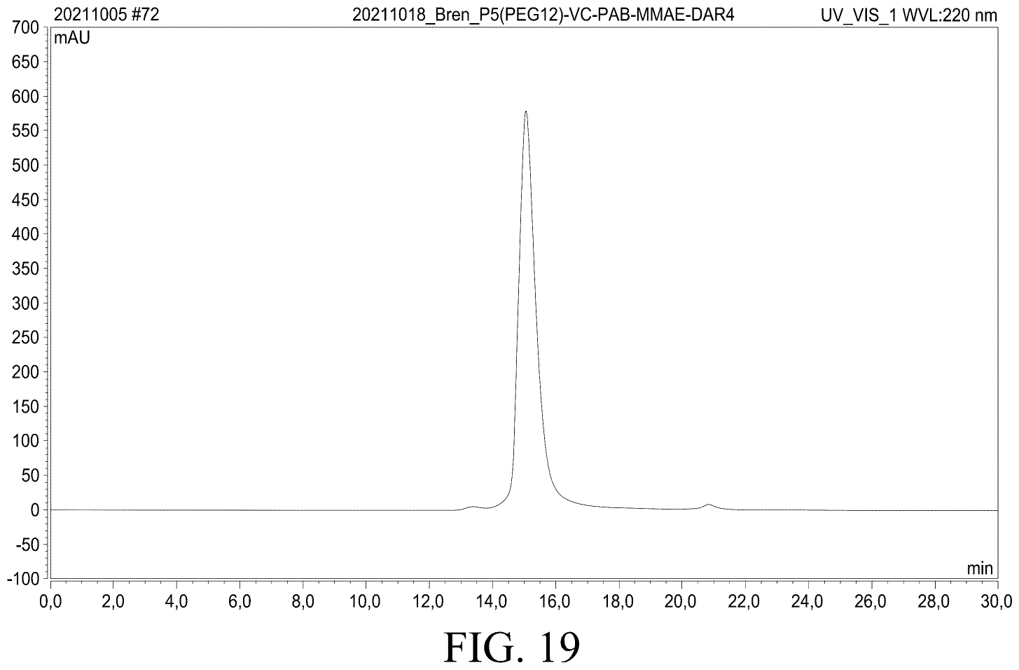

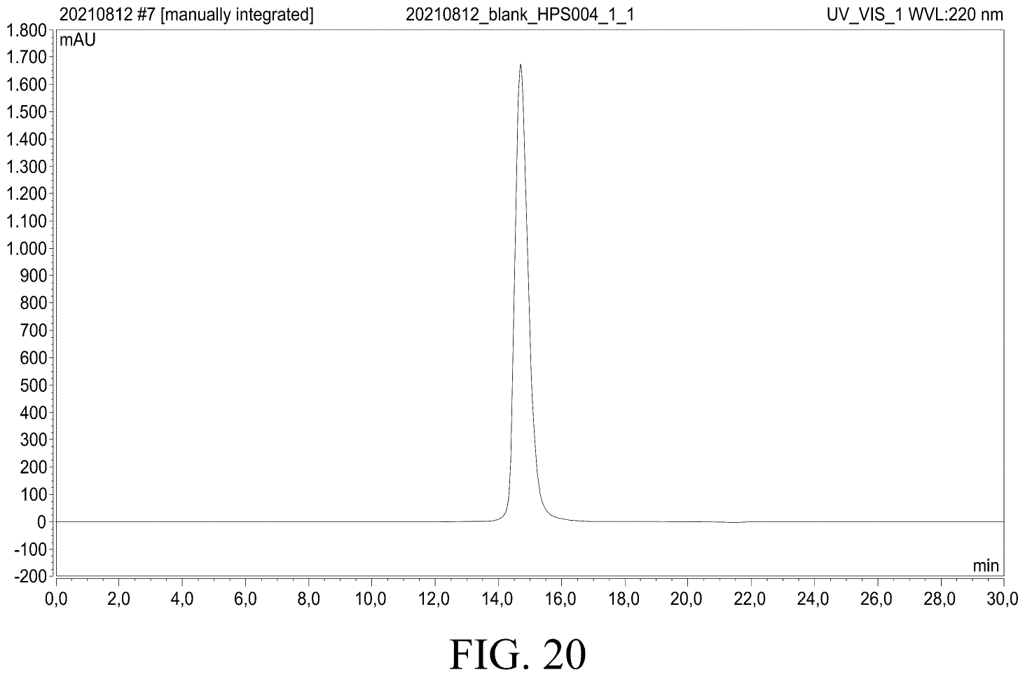

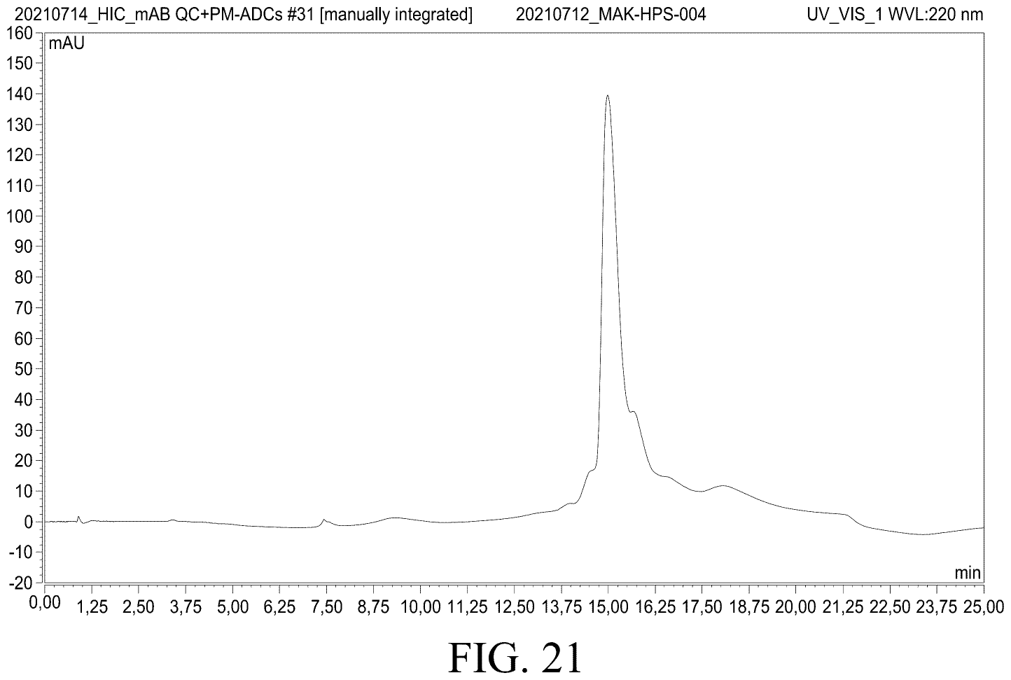

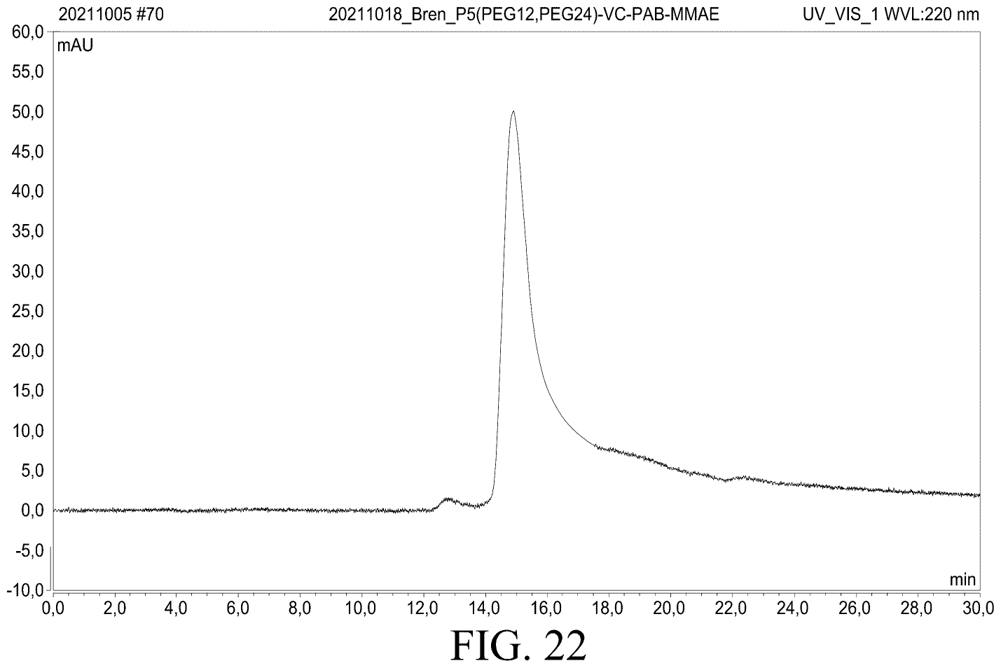

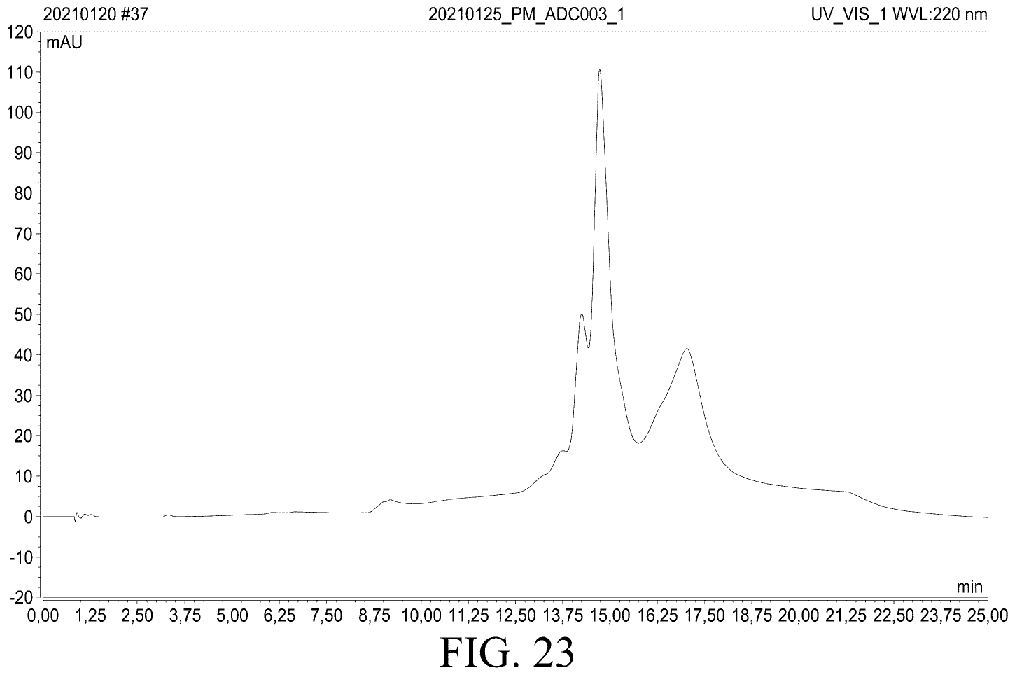

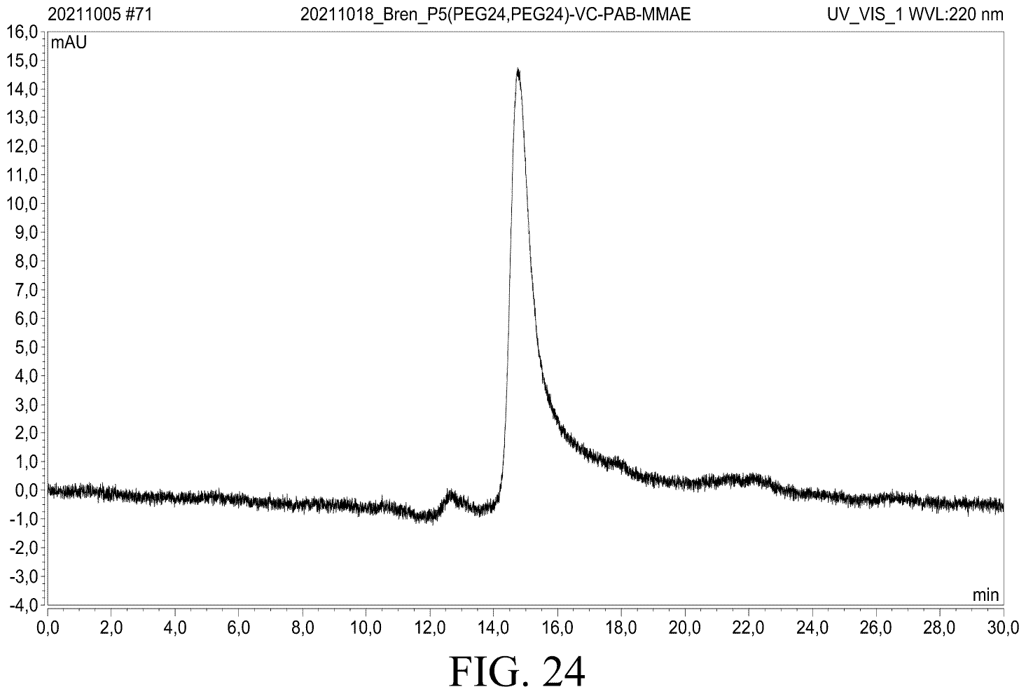

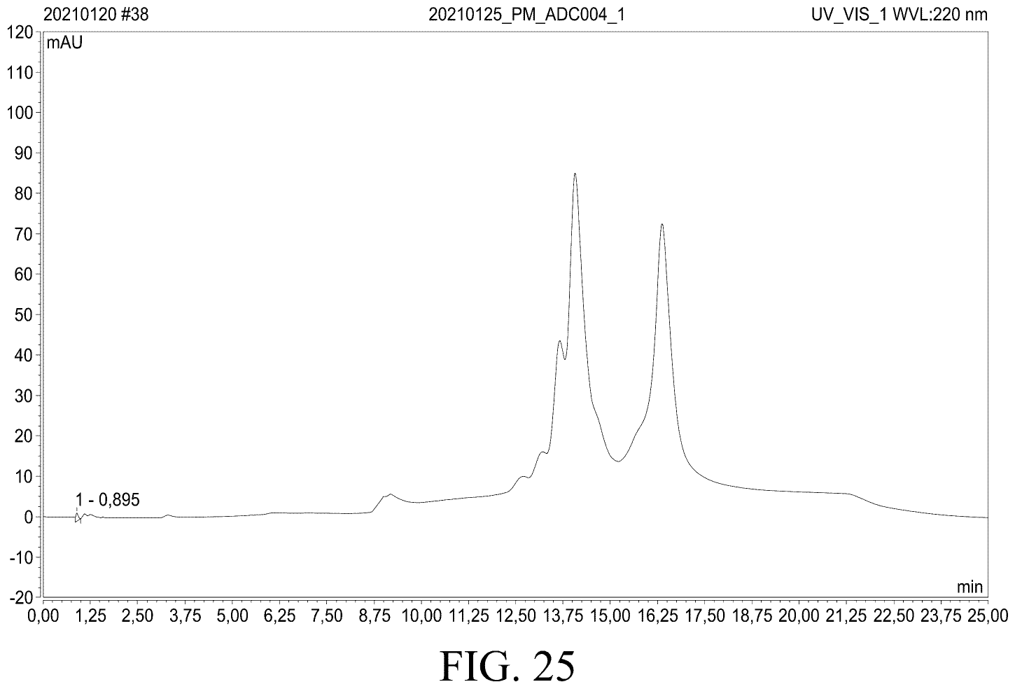

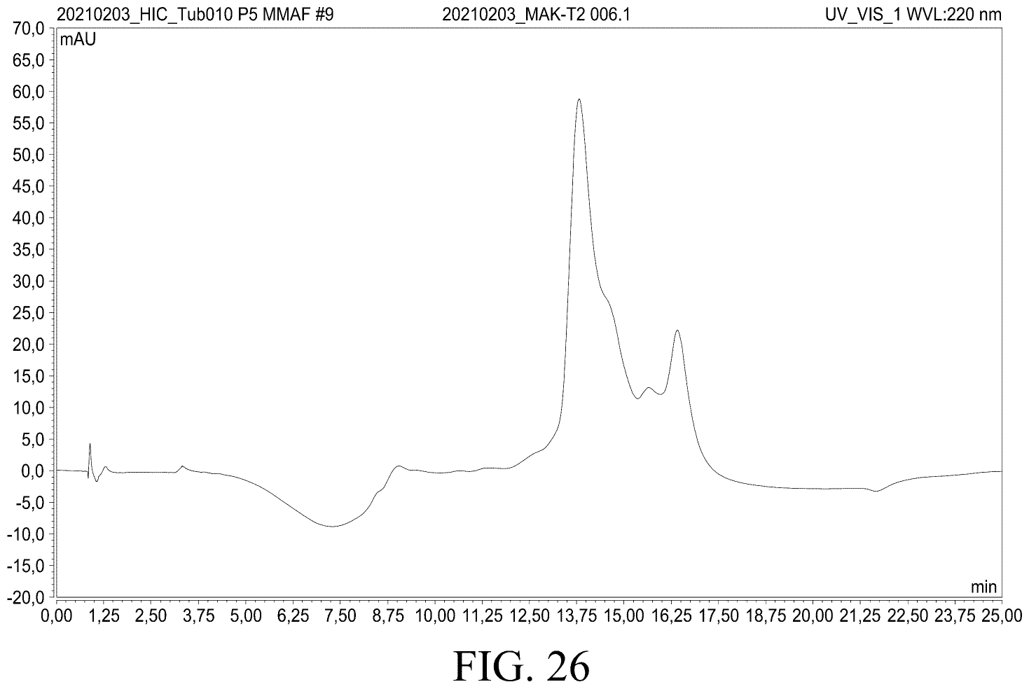

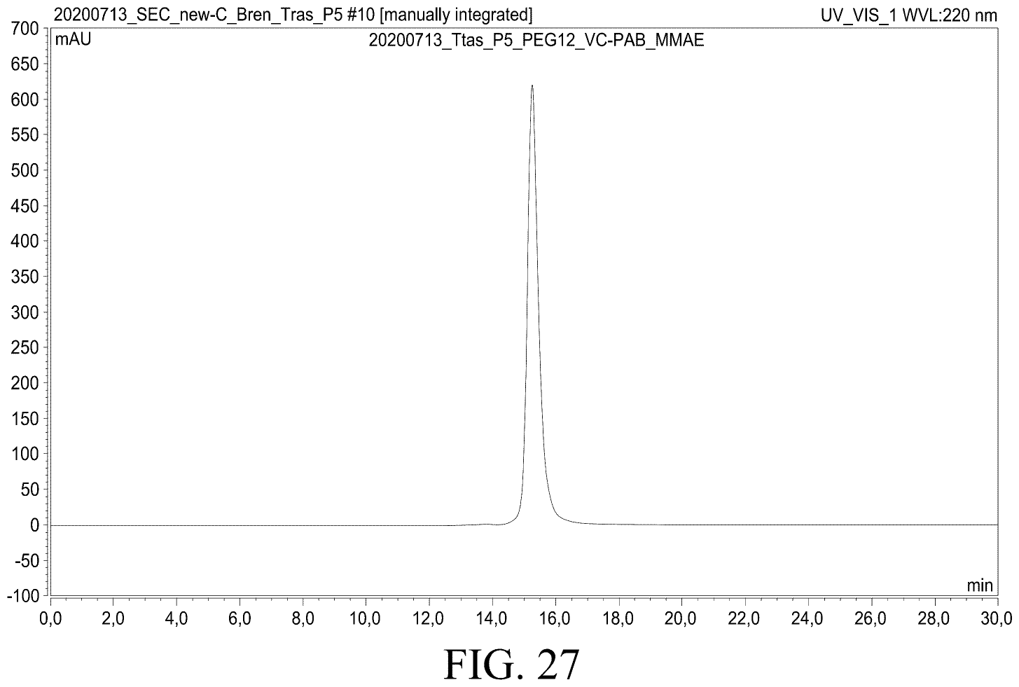

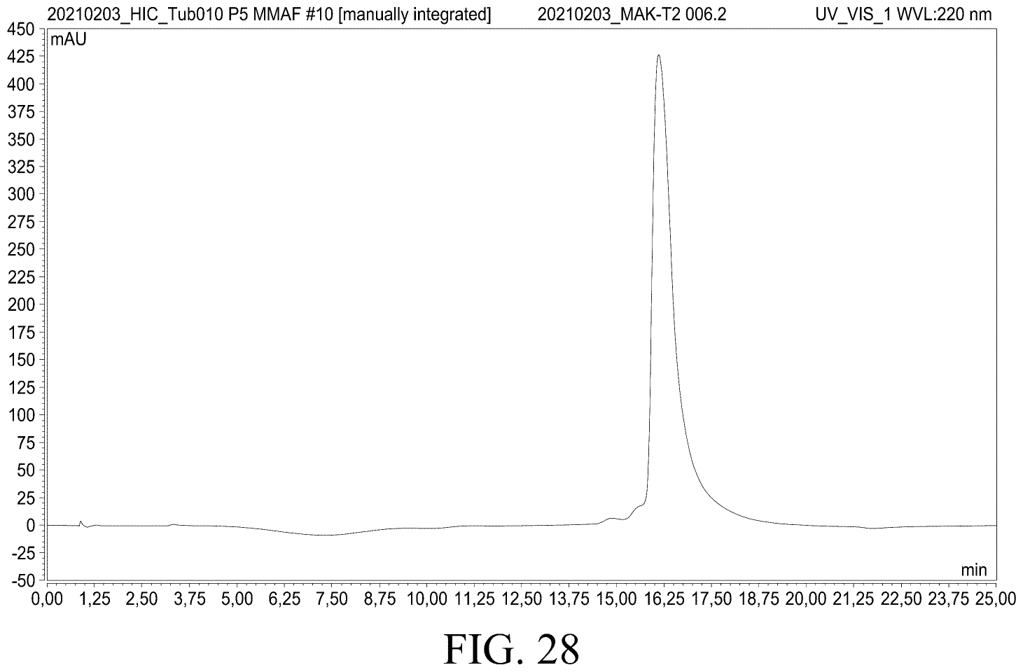

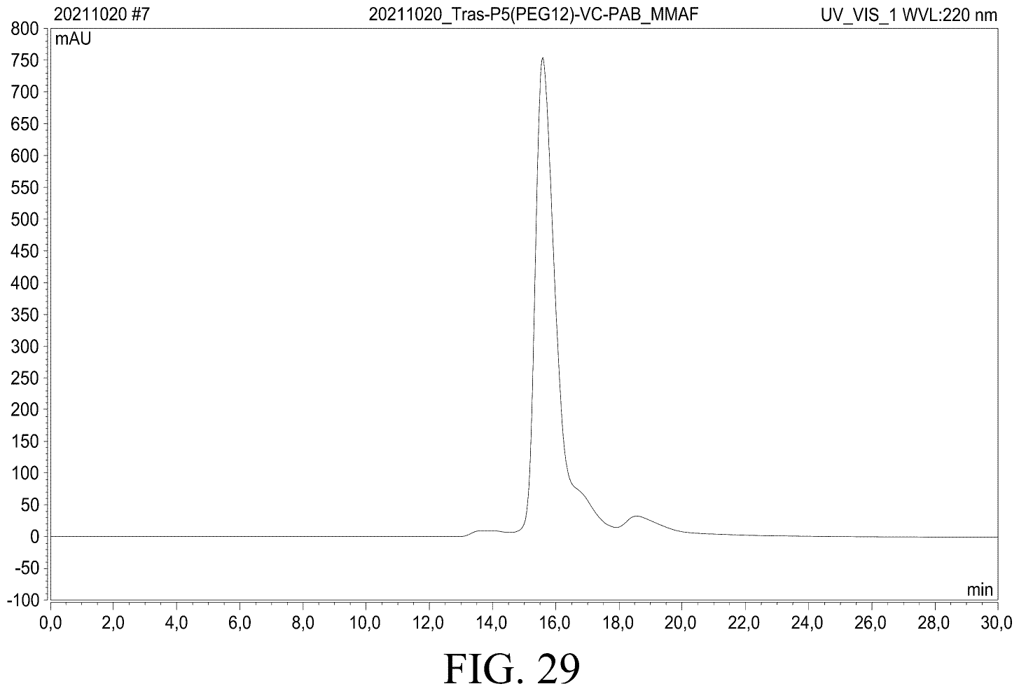

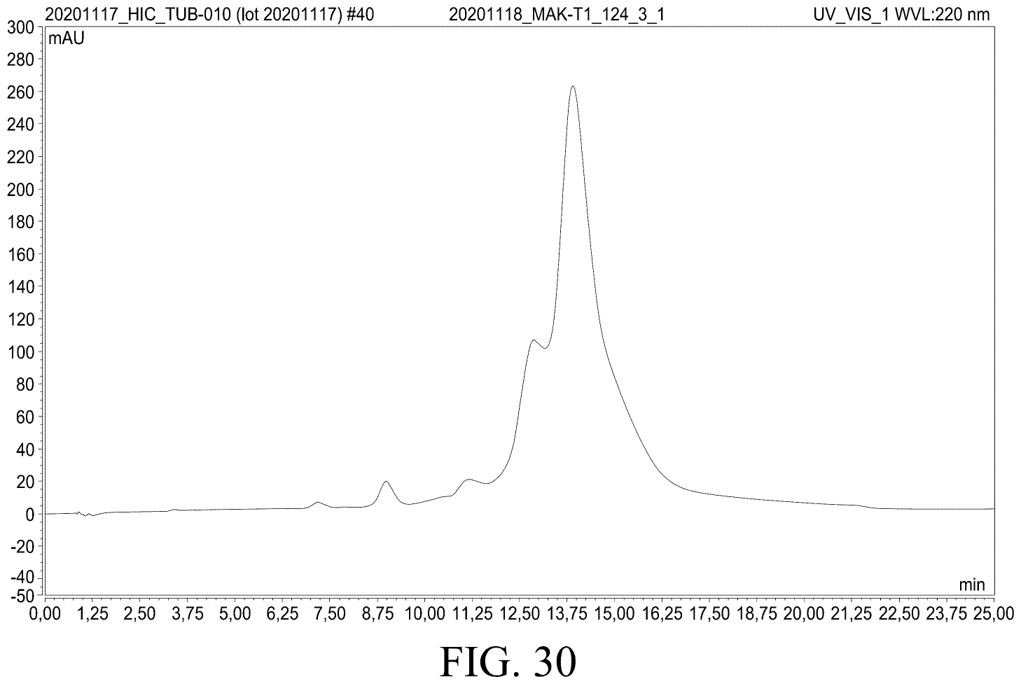

### Example 2: Analytics of the Synthesized ADCs and Antibody Starting Materials

| | | | MS analysis |
|---|---|---|---|
| | | | of the fully |
| | | | conjugated |
| Antibody/ADC | Analytical SEC | Analytical HIC | DAR8, DAR |
| 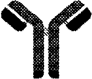 | See FIG. 13 | See FIG. 14 | LC: calcd.: 23439 found: 23438 HC: calcd.: 49146 found: 49149 |
| Trastuzumab | | | |
| 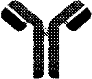 | See FIG. 15 | See FIG. 16 | LC: calcd.: 23724 found: 23724 HC: calcd.: 48878 found: 48877 |
| Brentuximab | | | |
| 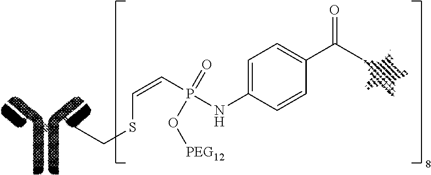 | See FIG. 17 | See FIG. 18 | DARav:8.0 LC: calcd.: 25582 found: 25583 HC: calcd.: 54452 found: 54454 |
| Brentuximab-P5(PEG12)- | | | |
| VC-PAB-MMAE | | | |
| 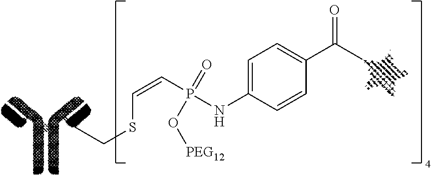 | See FIG. 19 | | DARav: 4.0 LC: calcd.: 25528 found: 25583 HC: calcd.: 54452 found: 54454 |
| Brentuximab-P5(PEG12)- | | | |
| VC-PAB-MMAE | | | |
| 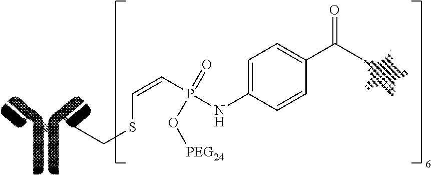 | See FIG. 20 | See FIG. 21 | DARav: 8.0 LC: calcd.: 26111 found: 26110 HC: calcd.: 56039 found: 56040 |
| Brentuximab-P5(PEG24)- | | | |
| VC-PAB-MMAE | | | |
| 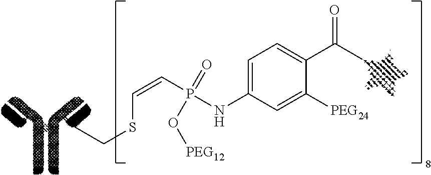 | See FIG. 22 | See FIG. 23 | DARav: 8.0 LC: calcd.: 26655 found: 26655 HC: calcd .: 57672 found: 57673 |
| Brentuximab-P5(PEG12, | | | |
| PEG24)-VC-PAB-MMAE | | | |
| 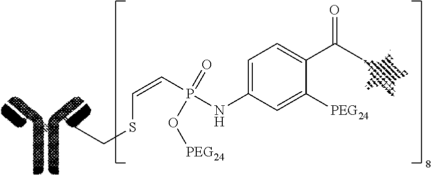 | See FIG. 24 | See FIG. 25 | DARav: 8.0 LC: calcd.: 27183 found: 27184 HC: calcd.: 59261 found: 59260 |
| Brentuximab-P5(PEG24, | | | |
| PEG24)-VC-PAB-MMAE | | | |
| 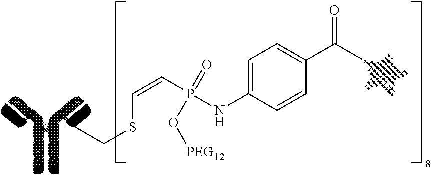 | | See FIG. 26 | DARav: 8.0 |
| Brentuximab-P5(PEG12)- | | | |
| VC-PAB-MMAF | | | |
| 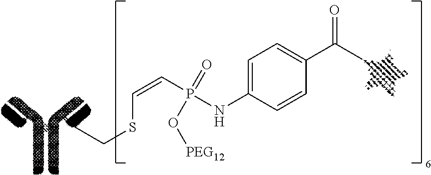 | | | DARav: 5.9 LC: calcd.: 24941 found: 24942 HC: calcd.: 52528 found: 52529 |
| Brentuximab-P5(PEG12)- | | | |
| Cy5 | | | |
| 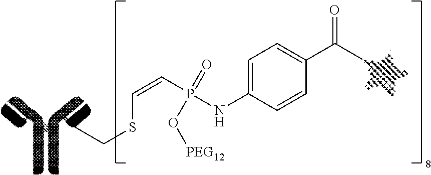 | See FIG. 27 | See FIG. 28 | DARav: 8.0 LC: calcd.: 25297 found: 25298 HC: calcd.: 54720 found: 54728 |
| Trastuzumab-P5(PEG12)- | | | |
| VC-PAB-MMAE | | | |
| 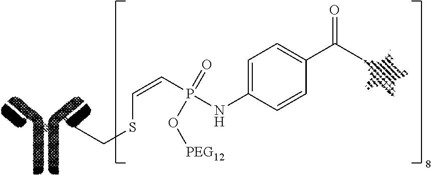 | See FIG. 29 | See FIG. 30 | DARav: 8.0 |
| Trastuzumab-P5(PEG12)- | | | |
| VC-PAB-MMAF | | | |

DARav means the average drug-to-antibody ratio.
LC: Mass of the light chain; HC: Mass of the heavy chain

FIG. 13 shows an analytical SEC chromatogram of Trastuzumab. SEC means size-exclusion chromatography.

FIG. 14 shows an analytical HIC chromatogram of Trastuzumab. HIC means hydrophobic interaction chromatography.

FIG. 15 shows an analytical SEC chromatogram of Brentuximab.

FIG. 16 shows an analytical HIC chromatogram of Brentuximab.

FIG. 17 shows an analytical SEC chromatogram of Brentuximab-P5(PEG12)-VC-PAB-MMAE.

FIG. 18 shows an analytical HIC chromatogram of Brentuximab-P5(PEG12)-VC-PAB-MMAE.

FIG. 19 shows an analytical SEC chromatogram of Brentuximab-P5(PEG12)-VC-PAB-MMAE.

FIG. 20 shows an analytical SEC chromatogram of Brentuximab-P5(PEG24)-VC-PAB-MMAE.

FIG. 21 shows an analytical HIC chromatogram of Brentuximab-P5(PEG24)-VC-PAB-MMAE.

FIG. 22 shows an analytical SEC chromatogram of Brentuximab-P5(PEG12, PEG24)-VC-PAB-MMAE.

FIG. 23 shows an analytical HIC chromatogram of Brentuximab-P5(PEG12, PEG24)-VC-PAB-MMAE.

FIG. 24 shows an analytical SEC chromatogram of Brentuximab-P5(PEG24, PEG24)-VC-PAB-MMAE.

FIG. 25 shows an analytical HIC chromatogram of Brentuximab-P5(PEG24, PEG24)-VC-PAB-MMAE.

FIG. 26 shows an analytical HIC chromatogram of Brentuximab-P5(PEG12)-VC-PAB-MMAF.

FIG. 27 shows an analytical SEC chromatogram of Trastuzumab-P5(PEG12)-VC-PAB-MMAE.

FIG. 28 shows an analytical HIC chromatogram of Trastuzumab-P5(PEG12)-VC-PAB-MMAE.

FIG. 29 shows an analytical SEC chromatogram of Trastuzumab —P5(PEG12)-VC-PAB-MMAF.

FIG. 30 shows an analytical HIC chromatogram of Trastuzumab —P5(PEG12)-VC-PAB-MMAF.

The SEC chromatograms (SEC=size exclusion chromatography) show that the conjugates exhibit low to substantially no aggregation. Our SEC Data is showing highly monomeric ADCs, with less than 5% Aggregates after purification in all tested variants. In combination with high conjugation yields, typically in the range of 80-90% based on antibody concentration determination before and after the conjugation process, this clearly shows that only minor percentages of aggregates are being formed during the conjugation process, when using the P5-conjugation technology described herein.

The HIC chromatograms (HIC=hydrophobic interaction chromatography) show that the conjugates exhibit a good hydrophilicity, combined with excellent homogeneity of only one major DAR8 ADC species being formed during the conjugation process.

### Example 3: Antibody Conjugation with PEGylated Ethynylphosphonamidates

Antibody modification with Ethynylphosphonamidates can be generally performed in a simple one-pot reaction with TCEP (as previously described, e.g., in Kasper et al., Angew. Chem. Int. Ed. 2019, 58, 11631-11636).
The inventors generally observed that the longer PEG chains enable us to use less equivalents of the cytotoxic and expensive linker payload derivatives to achieve a fully labelled DAR 8 ADC (“DAR” means drug to antibody ratio). This is more difficult for the PEG2 derivative (reference example).
A possible reason is that, the conjugation reaction to the antibody could be carried out at higher concentrations in the aqueous reaction medium.
The results of a screening experiment to find the optimal conditions for the generation of a DAR 8 ADC can be found in FIG. 31. Only a very slight linker-payload excess is needed to generate a fully modified DAR 8 ADC.

FIG. 31 shows a screening experiment to identify optimal conditions for antibody modifications with PEGylated phosphonamidates. Drug to Antibody Ratio (DAR) has been measured by MS. Left graph: 10 eq. of P5(PEG12)-VC-PAB-MMAE have been used under the above stated conditions and TCEP equivalents were varied. A maximum degree of modification has been reached with 8 equivalents of TCEP. Right graph: Those 8 equivalents have been carried over to a second experiment in which the P5(PEG12)-VC-PAB-MMAE equivalents were further increased in order to achieve a maximum DAR of 8. Optimal conditions to achieve DAR8 were Identified as 8 eq. of TCEP and 12 eq. of P5(PEG12)-VC-PAB-MMAE with respect to the antibody (=only 1.5 eq. per Cys).
Brentuximab-P5-VC-PAB-MMAE conjugates with different PEG-chain length have been synthesized and further analyzed for hydrophilicity by Hydrophobic-Interaction-Chromatography (HIC) (FIG. 32).

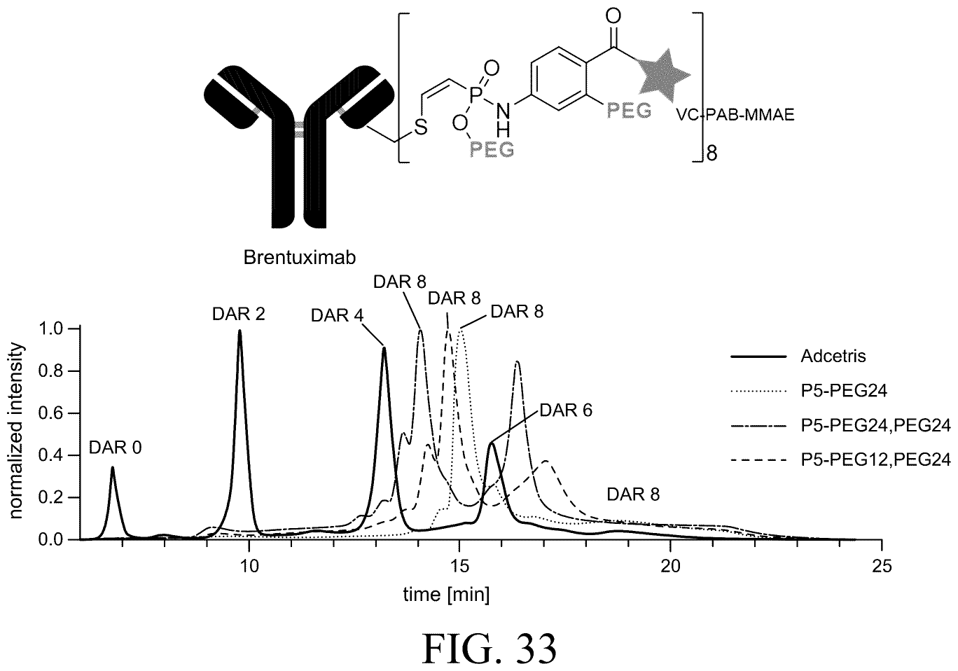

FIG. 32 shows a Hydrophobic Interaction Chromatography of Brentuximab conjugated to P5(PEG2)-, P5(PEG12)- and P5(PEG24)-VC-PAB-MMAE in direct comparison to commercially available Adcetris (Brentuximab-Maleimidocapryl-VC-PAB-MMAE, DAR4av, black).
Commercially available Adcetris is conjugated to an average of 4 MC-VC-PAB-MMAE molecules per Brentuximab and consists of a Mixture of DAR0, DAR2, DAR4, DAR6 and DAR8. Adcetris (Brentuximab-vedotin) has been chosen as reference since it is approved and marketed for treatment of relapsed or refractory Hodgkin lymphoma (HL) and systemic anaplastic large cell lymphoma (ALCL), a type of T cell non-Hodgkin lymphoma. Adcetris has an average drug to antibody ratio of 4.
P5(PEG12)- and P5(PEG24)-VC-PAB-MMAE can be used to synthesize highly homogenous DAR8 ADCs (only one sharp Peak in the chromatogram).
The DAR 8 species are shifted to shorter retention times (=more polar) the longer the PEG chain. Polarity of DAR8 Brentuximab-P5-VC-PAB-MMAE is equal to the polarity of the DAR5 species of Adcetris.
The PEG12 and PEG 24 traces show the desired pattern of one sharp major species for DAR8. In contrast, the PEG2 derivative (reference example) is very broad with two extra peaks for DAR7 and DAR6, maybe due to insufficient solubility in the conjugation process.
Dual-PEG-ADCs have been analyzed accordingly (FIG. 33). Additional PEG chains further shift the ADC towards lower retention times.

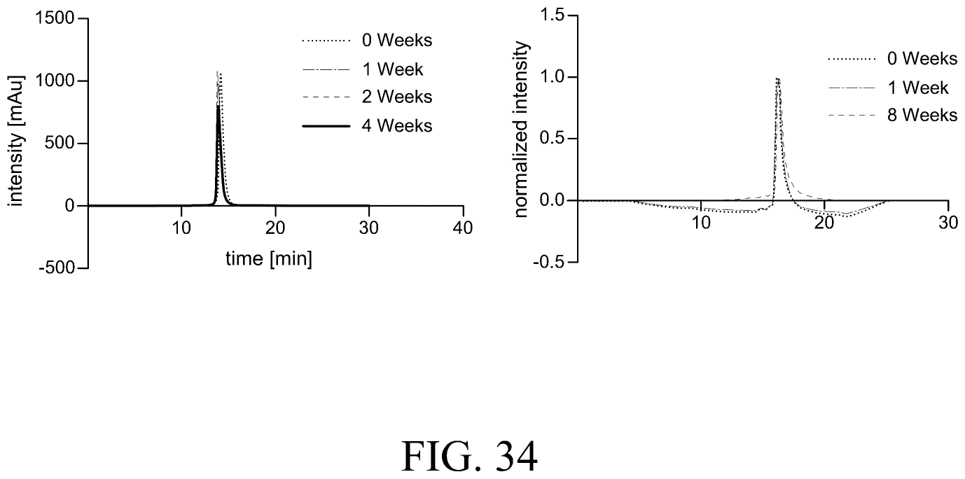

FIG. 33 shows a Hydrophobic Interaction Chromatography of Brentuximab conjugated to P5(PEG24,PEG12)-, P5(PEG24,PEG24)- and P5(PEG24)-VC-PAB-MMAE in direct comparison to commercially available Adcetris (Brentuximab-Maleimidocapryl-VC-PAB-MMAE, DAR4av, black). # Additional peaks, not identified.
Storage tests suggests a very good stability. No aggregates are formed and no payload loss is observed over long-time storage (FIG. 34)

FIG. 34 shows an Analytical Size-Exclusion-Chromatography (SEC) (left) and HIC (right) after storage of a DAR 8 Brentuximab-P5(PEG12)-VC-PAB-MMAE over several weeks. No Aggregates are visible in the SEC and no drug loss in the HIC.

### Example 4: In Vitro Cytotoxicity Evaluation of the Constructs

The ADCs, described in the last paragraph, have been evaluated for in vitro potency on antigen positive (targeted) and antigen negative (non-targeted) cell lines.
Selectivity for the antigen positive cell line has been observed for all the tested constructs.

FIG. 35 shows the in vitro cytoxicity of Brentuximab (anti-CD30) ADCs on an antigen positive cell line (Karpas 299, left) and an antigen negative cell line (HL-60, right).

Shown is a comparison of 3 different DAR8 ADCs that only differ in the length of the PEG substituent (PEG2 vs. PEG12 vs. PEG24) against unmodified Brentuximab.
The length of the PEG-chain has no influence on the cytotoxicity of the constructs.

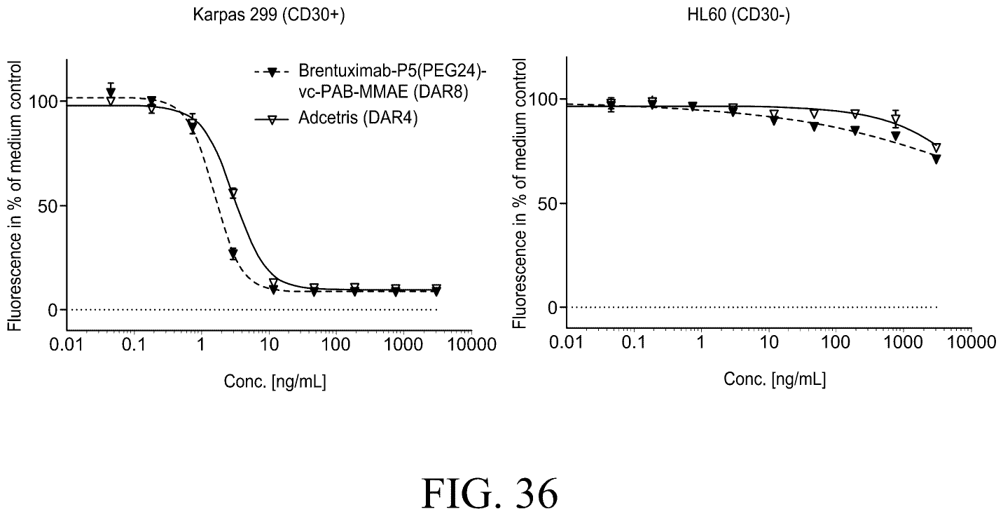

FIG. 36 shows the in vitro cytoxicity of Brentuximab (anti-CD30) ADCs on an antigen positive cell line (Karpas 299, left) and an antigen negative cell line (HL-60, right). Shown is a comparison of Brentuximab-P5(PEG24)-vc-PAB-MMAE (DAR8) against commercial Adcetris (DAR4).
Brentuximab-P5(PEG24)-vc-PAB-MMAE (DAR8) is more potent than commercial Adcetris

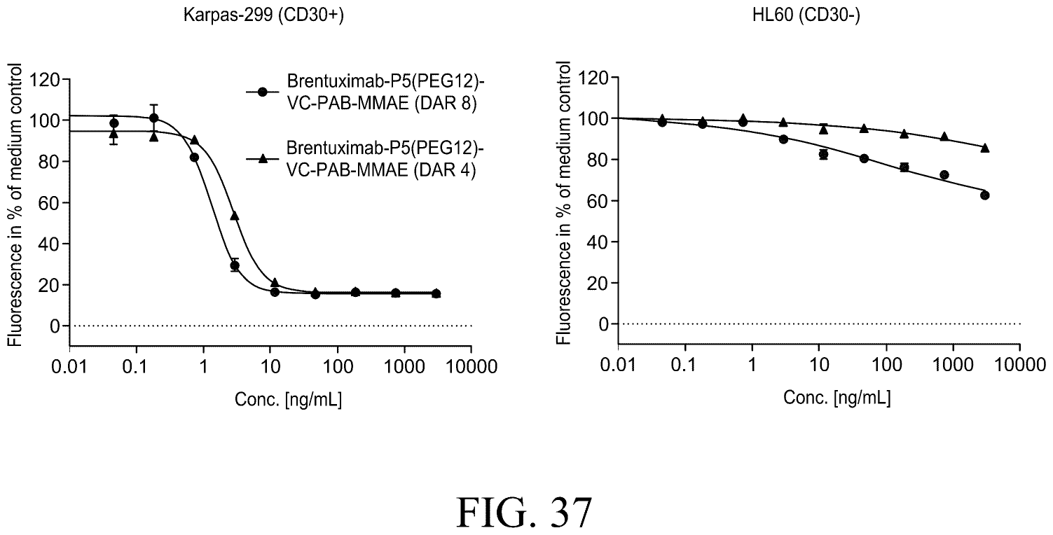

FIG. 37 shows In vitro cytoxicity of Brentuximab (anti-CD30) ADCs on an antigen positive cell line (Karpas 299, left) and an antigen negative cell line (HL-60, right). Shown is a comparison of Brentuximab-P5(PEG24)-vc-PAB-MMAE, modified with either 4 (DAR4) or 8 (DAR8) linker payload molecules per antibody.
Brentuximab-P5(PEG24)-vc-PAB-MMAE DAR8 constructs are more potent than DAR4, probably due the higher drug loading.

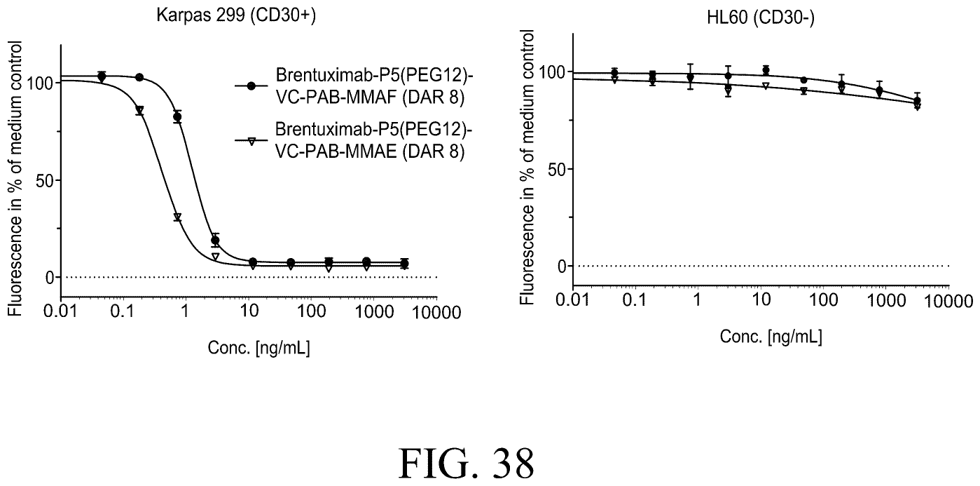

FIG. 38 shows the in vitro cytotoxicity of Brentuximab (anti-CD30) ADCs on an antigen positive cell line (Karpas 299, left) and an antigen negative cell line (HL-60, right). Shown is a comparison of Brentuximab-P5(PEG12)-vc-PAB-MMAE (DAR8) versus the same construct, carrying a MMAF payload.
The MMAF construct is more potent than the corresponding MMAE construct.

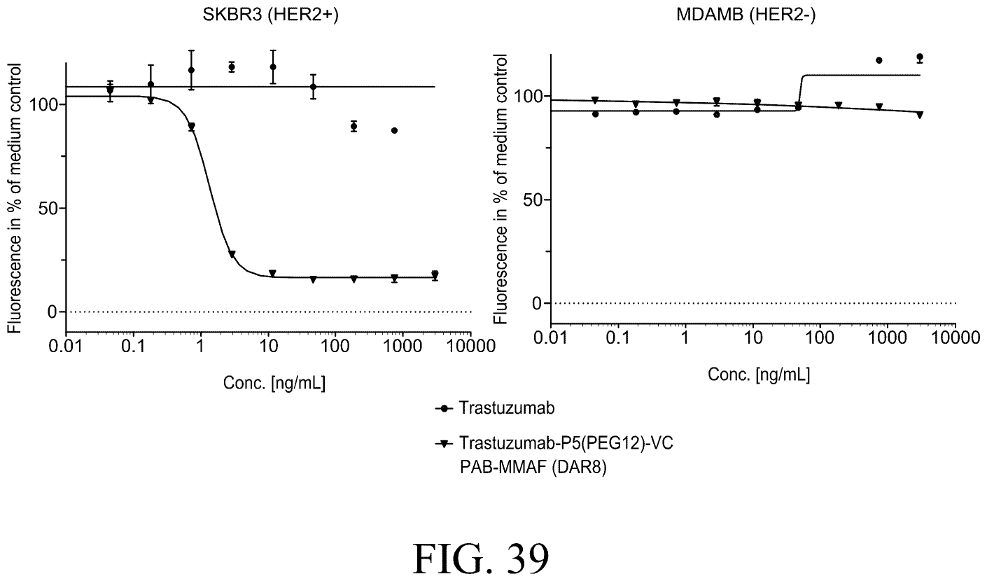

FIG. 39 shows the in vitro cytoxicity of Trastuzumab (anti-Her2) ADCs on an antigen positive cell line (SKBR3, left) and an antigen negative cell line (MDAMB, right). Shown is a comparison of Trastuzumab-P5(PEG12)-vc-PAB-MMAF (DAR8) versus unmodified Tratuzumab.
P5(PEG12)-VC-PAB-MMAF based ADCs also function in a Her2-based setting using Her2-positive solid tumor cancer cells.

### Example 5: In Vitro Bystander Effect

It was assumed that an issue of the long PEG-Substituents may arise from sterical blocking of the VC-PAB cleavage side, leading to insufficient MMAE release within the cell.
Since uncleaved MMAE is still potent, this effect cannot be assessed in a simple cell killing assay, as described above.
But cleavage is necessary to induce a bystander effect, which is a killing of non-targeted neighbouring cells.
Therefore, the inventors designed an experiment where targeted cells (L-540) were incubated with differently PEGylated VC-PAB-MMAE constructs, and non-targeted cells (HL60) were treated with the supernatant of the L-540 culture. HL60 cells can only be killed by released MMAE in this setting.
Surprisingly, no difference in Bystander effect was visible for all of the tested constructs, which suggests that the cathepsin B mediated cleavage of VC-PAB is not impaired by the long PEG substituents.

FIG. 40 shows the evaluation of the bystander effect in dependence of differently PEGylated Brentuximab-P5-VC-PAB-MMAE constructs. Top left: In vitro cytoxicity of Brentuximab (anti-CD30) ADCs on two antigen positive cell line (Karpas 299, left and L-540, right) and an antigen negative cell line (HL-60, bottom left). Transfer of the supernatant after incubation of L-540 with the ADCs to HL-60 has been performed in order to evaluate bystander killing (HL60, bottom right).

### Example 6: In Vivo Evaluation of the Constructs

Brentuximab(PEG12)-VC-PAB-MMAE has been evaluated in an in vivo efficacy study as a DAR4 and a DAR8 version.
The DAR4 already shows a superior effect over commercial Adcetris at the same dosing and equal payload amounts.
The DAR 8 can furthermore increase this effect. Surprisingly, this leads to a complete remission of the tumor in 90% of the treated mice.

FIG. 41 shows the in vivo evaluation of Brentuximab-(PEG12)-VC-PAB-MMAE (DAR8 and DAR4), Adcetris (DAR4) and a non-treated control in a Karpas299 based tumor xenograft model in SCID mice with 10 animals per group. Mice were treated with 0.5 mg/kg of the constructs four times, every four days. Left graph shows the mean tumor volume of all 10 mice per group. Last observation point of sacrificed animals has been carried forward (LOCF). Right graph shows the Kaplan-Meier plot of survival in each group.

### Example 7: In Vivo-Pharmacokinetics Evaluation of the Constructs

In vivo PK (pharmacokinetics)-experiments have been performed Brentuximab-P5(PEG24)-VC-PAB-MMAE in direct comparison to Adcetris.
Female Sprague Dawley rats have been treated with 5 mg/kg Brentuximab-P5(PEG24)-VC-PAB-MMAE or Adcetris, an approved ADC for the treatment of treat relapsed or refractory Hodgkin lymphoma (HL) and systemic anaplastic large cell lymphoma (ALCL), as comparison.
Blood sampling has been performed after different time points and the ADC amount was quantified in a total Brentuximab antibody ELISA-assay.

FIG. 42 shows the quantification of the amount of total antibody in blood circulation after treatment of female Spraque-Dawley rats with either Brentuximab-P5(PEG24)-VC-PAB-MMAE or Adcetris via ELISA.
The experiment clearly shows a good PK behaviour of the conjugate in accordance with an embodiment of the invention despite the high drug to antibody ratio (DAR) of 8 with a reasonable clearance rate, especially when compared to the approved Adcetris having a DARav of only 4.
Furthermore: Very high stability shown for Brentuximab-P5(PEG24)-VC-PAB-MMAE, since intact ADC and total antibody curves are not significantly apart from each other.

### Example 8: ADC Synthesis and In Vitro Evaluation Using P5(PEG12)-VC-PAB-SB743921 and P5(PEG24)-VA-PAB-SB743921

ADCs were synthesized from the Her2-binding antibody Trastuzumab in the reaction with both linker-payloads, as described above under General Information, Materials and Methods. Linker-payload equivalents with respect to the antibody were increased to drive the reaction.

FIG. 43A shows conjugation of P5(PEG12)-VC-PAB-SB743921 and P5(PEG24)-VA-PAB-SB743921 to Trastuzumab; FIG. 43B shows conjugation efficiency estimated via mass spectrometry (MS). The Drug-to-Antibody-Ratio was calculated from the MS-Intensities of the modified and unmodified heavy and light chain species; it is noted that in this example only little or slow conjugation was observed when using a short PEG2 residue (i.e. a PEG residue comprising 2 PEG units), while a more efficient conjugation reaction could be achieved when using PEG12 (i.e. a PEG residue comprising 12 PEG units), and even more when using PEG24 (i.e. a PEG residue comprising 24 PEG units) as indicated by the higher drug to antibody ratio (DAR); FIG. 43C shows MS spectrum of the conjugation of P5(PEG24)-VA-PAB-SB743921 to Trastuzumab. The mass spectrum shows unmodified light chain (23438 Da), modified light chain (25439 Da), unmodified heavy chain (49149 Da), triple modified heavy chain (55452 Da).

FIG. 44 shows efficacy of the ADC Trastuzumab-P5(PEG24)-VA-PAB-SB743921 on a target negative cell line (L-540) and several solid tumor Her2+ cell-lines. Trastuzumab-P5(PEG24)-VA-PAB-SB743921 shows only an effect on the non-targeted L-540 at the highest tested concentration, while a much better efficacy could be shown on all tested target-positive cell lines.

The PEG chain at the Phosphonamidate enables conjugation to the mAb. PEG2 did not show any conjugation to the mAb (data not shown), PEG12 enables the modification and PEG24 leads to a good conjugation efficiency that yield ADCs that are efficacious in vitro and selective for the targeted cell line as shown for a great variety of solid tumor cancer cells.

### Example 9: ADC Synthesis Using P5(PEG12)-VC-PAB-Emetin

ADCs were synthesized from the Her2-binding antibody Trastuzumab in the reaction with P5(PEG12)-VC-PAB-Emetin, as described above under General Information, Materials and Methods.

FIG. 45 shows A) conjugation of P5(PEG12)-VC-PAB-Emetin to Trastuzumab; B) Conjugation efficiency estimated via mass spectrometry (MS). The Drug-to-Antibody-Ratio was calculated from the MS-Intensities of the modified and unmodified heavy and light chain species; C) Exemplary HIC spectrum of the conjugation of 16 equivalents of P5(PEG12)-VC-PAB-Emetin to Trastuzumab, yielding a DAR 8.0 ADC. No unconjugated Trastuzumab was visible at the known retention time of the unmodified antibody (ca. 8-9 minutes).

FIG. 46 shows normalized HIC chromatograms of Tras-P5(PEG12)-VC-PAB-Emetin DAR8 and Adcetris (Brentuximab vedotin). As can be seen, the DAR8 Emetin ADC is as polar as the DAR3 ADC of the approved medication Adcetris. Adcetris consists of a heterogeneous mixture of different DAR species (0-8) with an average DAR in the mixture of 4. The P5(PEG12) unit facilitates formation of a DAR8 ADC which is assumed to be stable in circulation without increased clearance rates in vivo. The measured hydrophobicity by HIC is less than the one of a DAR4 Adcetris molecule.

### Example 10: ADC Synthesis Using P5(PEG12)-VC-PAB- AT7519

ADCs were synthesized from the Her2-binding antibody Trastuzumab in the reaction with P5(PEG12)-VC-PAB-AT7519, as described above under General Information, Materials and Methods.

FIG. 47 shows A) conjugation of P5(PEG12)-VC-PAB-Emetin to Trastuzumab; B) Analysis of the purified DAR8 ADCs by Size-Exclusion Chromatography (SEC) and Hydrophobic interaction chromatography (HIC). No aggregates were visible in the SEC chromatogram. Only one DAR8 species was visible in the HIC chromatogram; C) MS analysis of the conjugation of 16 equivalents of P5(PEG12)-VC-PAB-AT7519 to Trastuzumab, yielding a DAR 8.0 ADC. No unconjugated Trastuzumab was observed.

Thus, successful synthesis of an ADC having DAR8 with the AT7519 payload in combination with P5(PEG12) has been demonstrated.

### Example 11: ADC Synthesis Using P5(PEG24)-VA-PAB-Panobinostat

ADCs were synthesized from the Her2-binding antibody Trastuzumab in the reaction with P5(PEG24)-VA-PAB-Panobinostat, as described above under General Information, Materials and Methods.

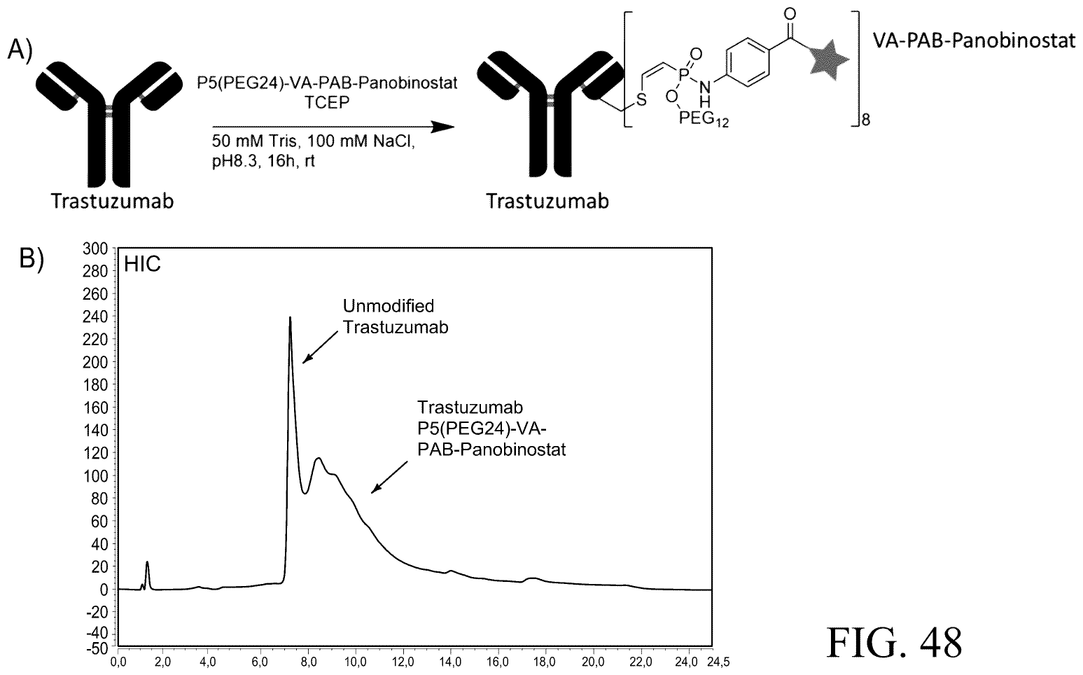

FIG. 48 shows A) conjugation of P5(PEG24)-VA-PAB-Panobinostat to Trastuzumab; B) Analysis of the purified DAR8 ADCs by Hydrophobic interaction chromatography (HIC).

Thus, successful conjugation of the Panobinostat payload in combination with a P5(PEG24) unit to Trastuzumab to form a an ADC having DAR8 has been demonstrated, as confirmed by HIC chromatography.

### Example 12: ADC Synthesis Using P5(PEG12)-GlcA-AT7519

ADCs were synthesized from the Her2-binding antibody Trastuzumab in the reaction with P5(PEG12)-GlcA-AT7519, as described above under General Information, Materials and Methods.

FIG. 49 shows A) conjugation of P5(PEG12)-GlcA-AT7519 to Trastuzumab; B) Conjugation efficiency estimated via MS in dependency on the equivalents of P5(PEG12)-GlcA-AT7519. Reaction was carried out as described herein with 8 eq. of TCEP. The Drug-to-Antibody-Ratio was calculated from the MS-Intensities of the modified and unmodified heavy and light chain species; C) Exemplary MS spectrum of the conjugation of 6 eq. P5(PEG12)-GlcA-AT7519 to Trastuzumab to yield a DAR 3.9 ADC.

FIG. 50 shows normalized HIC chromatograms of Tras-P5(PEG12)-GlcA-AT7519 and Adcetris (Brentuximab vedotin); and an SEC chromatogram of Tras-P5(PEG12)-GlcA-AT7519.

The HIC analysis shows that the DAR8 ADC with AT7519 having DAR8 (i.e., a drug to antibody ratio of 8) is as polar as the DAR3 ADC of the approved medication Adcetris. Adcetris consists of a heterogeneous mixture of different DAR species (0-8) with an average DAR in the mixture of 4. The P5(PEG12) unit side facilitates formation of a DAR8 ADC which is assumed to be stable in circulation without increased clearance rates in vivo. The measured hydrophobicity by HIC is less than the one of a DAR4 Adcetris molecule. Moreover the SEC analysis shows clearly that no aggregation is present.

### Example 13: ADC Synthesis and In Vitro Evaluation Using P5(PEG12)-GlcA-MMAE

ADCs were synthesized from the CD30-binding antibody Brentuximab in the reaction with P5(PEG12)-P5(PEG12)-GlcA-MMAE, as described above under General Information, Materials and Methods. ADCs having a drug to antibody ratio of 8 (DAR8) were isolated and tested for efficacy in vitro. Brentuximab-P5(PEG12)-VC-PAB-MMAE was also tested for comparison. It turned out that Brentuximab-P5(PEG12)-GlcA-MMAE, which has GlcA cleavage side, was as efficacious as Brentuximab-P5(PEG12)-VC-PAB-MMAE, which has as VC-PAB cleavage side that can be cleaved by cathepsin B.

FIG. 51 shows A) conjugation of P5(PEG12)-GlcA-MMAE to Brentuximab; B) Efficacy on a target negative cell line (HL-60, bottom) and a target positive cell line (Karpas299, top); Brentuximab-P5(PEG12)-GlcA-MMAE shows no effect on the target negative cell line (HL-60), while a much better efficacy could be shown on target-positive cell line Karpas299.

### Example 14: ADC Synthesis and In Vitro Evaluation Using P5(PEG12)-GlcA-SB743921

ADCs were synthesized from the Her2-binding antibody Trastuzumab in the reaction with P5(PEG12)-GlcA- SB743921, as described above under General Information, Materials and Methods.

FIG. 52 shows A) conjugation of P5(PEG12)-GlcA-SB743921 to Trastuzumab; B) Conjugation efficiency estimated via mass spectrometry (MS) in dependency on the equivalents of P5(PEG12)-GlcA-SB743921. Reaction was carried out as described in Example 3 above with 8 eq. of TCEP. The Drug-to-Antibody-Ratio was calculated from the MS-Intensities of the modified and unmodified heavy and light chain species; C) Analysis of the purified DAR8 ADCs by Size-Exclusion Chromatography (SEC) and Hydrophobic interaction chromatography (HIC). Almost no aggregates were visible in the SEC chromatogram.

FIG. 53 shows the efficacy test results of Trastuzumab-P5(PEG12)GlcA-SB743921 on a target negative cell line (MDA-MB468) and a target positive cell line (SKBR3). Trastuzumab-P5(PEG12)-GlcA-SB743921 shows no effect on the target negative cell line (MDA-MB468), while a much better efficacy could be shown on SKBR-3.

Target specific cytotoxity has been observed for all DARs of the Trastuzumab-P5(PEG12)GlcA-SB743921 ADCs tested. The results show DAR-dependent increase of the cytotoxicity and clearly show successful delivery of the payload via the P5(PEG12)-GlcA linker.

1. A method of preparing a conjugate of formula (I), said method comprising:
reacting a compound of formula (II) 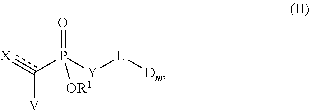
or a pharmaceutically acceptable salt of solvate thereof;
wherein: [symbol]
is a triple bond; or [symbol]
is a double bond;
V is absent when [symbol] is a triple bond; or
V is H or (C₁-C₈)alkyl when [symbol] is a double bond;
X is R₃—C when [symbol] is a triple bond; or
X is 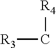
when [symbol] is a double bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
L is a linker;
D is a drug moiety; and
m is an integer ranging from 1 to 10;
with a thiol-containing molecule of formula (III)
RBM [symbol] SH)ₙ (III),
wherein RBM is a receptor binding molecule; and
n is an integer ranging from 1 to 20;
resulting in a compound of formula (I) 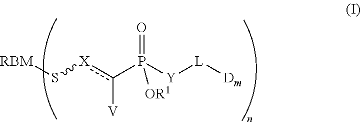
or a pharmaceutically acceptable salt or solvate thereof;
wherein: [symbol]
is a double bond when [symbol] in a compound of formula (II) is a triple bond; or [symbol]
is a bond when [symbol] in a compound of formula (II) is a double bond;
V is absent when [symbol] is a double bond; or
V is H or (C₁-C₈)alkyl when [symbol] is a bond;
X is R₃—C when [symbol] is a double bond; or
X is 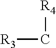
when [symbol] is a bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
L is a linker;
D is a drug moiety;
m is an integer ranging from 1 to 10; and
n is an integer ranging from 1 to 20.

2. The method of claim 1, wherein:
R³ is H or (C₁-C₈)alkyl; preferably R³ is H;
R⁴ when present is H or (C₁-C₈)alkyl; preferably R⁴, when present, is H;
R⁵ when present is H or (C₁-C₈)alkyl; preferably R⁵, when present, is H;
R⁶ when present is H or (C₁-C₈)alkyl; preferably R⁶, when present, is H; and
R⁷ when present is H or (C₁-C₈)alkyl; preferably R⁷, when present, is H.

3. The method of claim 1, wherein [symbol] is a triple bond; V is absent; X is R₃—C; R³ is H or an optionally substituted aliphatic residue or an optionally substituted aromatic residue, preferably R³ is H or (C₁-C₈)alkyl; more preferably R³ is H, and [symbol] is a double bond.

4. The method o claim 1, wherein [symbol] is a double bond; V is H or (C₁-C₈)alkyl, preferably V is H; X is 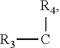
R³ is H or an optionally substituted aliphatic residue or an optionally substituted aromatic residue, preferably R³ is H or (C₁-C₈)alkyl, more preferably R³ is H; R⁴ is H or an optionally substituted aliphatic residue or an optionally substituted aromatic residue; preferably R4 is H or (C₁-C₈)alkyl, more preferably R⁴ is H; and [symbol] is a bond.

5. The method of claim 1, wherein the reaction is performed under neutral pH or slightly basic conditions, preferably at a pH of from 6 to 10.

6. The method of claim 1, further comprising reducing at least one disulfide bridge of the receptor binding molecule in the presence of a reducing agent to form a thiol group (SH).

7. The method of claim 6, wherein the reducing agent is selected from the group consisting of tris(2-carboxyethyl)phosphine (TCEP), dithiothreitol (DTT), sodium dithionite, sodium thiosulfate, and sodium sulfite; preferably wherein the reducing agent is tris(2-carboxyethyl)phosphine (TCEP).

8. The method of claim 6, wherein the reducing of at least one disulfide bridge comprises using about 1 to about 3 equivalents, preferably about 1 to about 2 equivalents, more preferably about 1 equivalent of the reducing agent per disulfide bridge to be reduced.

9. The method of claim 1, wherein the thiol-containing molecule of formula (III) is reacted with about 1 to about 4 equivalents, preferably about 1 to about 3 equivalents, more preferably about 1 to about 2 equivalents, still more preferably about 1.5 equivalents of the compound of formula (II) per thiol group (SH).

10. The method of claim 1, wherein the reacting a compound of formula (II) with a thiol-containing molecule of formula (III) is carried out in an aqueous medium.

11. The method of claim 1, wherein the receptor binding molecule is an antibody.

12. The method of claim 1, wherein Y is NH.

13. The method of claim 1, wherein R^{F} is: 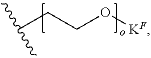
wherein [symbol]
indicates the position of the O;
K^{F} is selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, -(C2-C₁₀)alkyl-NH(C₁-C₃)alkyl and —(C₂-C₁₀)alkyl-N((C₁-C₃)alkyl)₂; and
o is an integer ranging from 3 to 100.

14. The method of claim 13, wherein K^{F} is H.

15. The method of claim 14, wherein o ranges from 8 to 30.

16. The method of claim 1, wherein the drug moiety is selected from the group consisting of a maytansinoid, a calicheamycin, a tubulysin, an amatoxin, a dolastatin, an auristatin, a pyrrolobenzodiazepine dimer, an indolino-benzodiazepine dimer, emetine, a radioisotope, a therapeutic protein, a therapeutic peptide, a kinase inhibitor, a CDK inhibitor, a histone deacetylase (HDAC) inhibitor, a MEK inhibitor, a KSP inhibitor, and prodrugs thereof.

17. The method of claim 16, wherein the drug moiety D is monomethyl auristatin E (MMAE) or monomethyl auristatin F (MMAF).

18. A method of treating cancer, comprising administering to a subject or patient in need thereof, an effective amount of a conjugate having the formula (I): 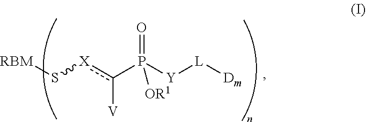
or a pharmaceutically acceptable salt or solvate thereof;
wherein:
RBM is a receptor binding molecule; [symbol]
is a double bond; or [symbol]
is a bond;
V is absent when [symbol] is a double bond; or
V is H or (C₁-C₈)alkyl when [symbol] is a bond;
X is R₃—C when [symbol] is a double bond; or
X is 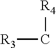
when [symbol] is a bond;
Y is NR⁵, S, O, or CR⁶R⁷;
R¹ is a first polyalkylene glycol unit R^{F} comprising at least 3 alkylene glycol subunits;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
L is a linker;
D is a drug moiety;
m is an integer ranging from 1 to 10; and
n is an integer ranging from 1 to 20.

19. The method of claim 18, wherein the cancer is a solid tumor.
